(19) United States
(12) Patent Application Publication
(10) Pub. No.: US 2021/0395239 A1
(43) Pub. Date: Dec. 23, 2021

(54) HETEROCYCLYL COMPOUNDS FOR THE TREATMENT OF AUTOIMMUNE DISEASE

(71) Applicant: Hoffmann-La Roche Inc., Little Falls, NJ (US)

(72) Inventors: Fabian DEY, Basel (CH); Jianguo CHEN, Shanghai (CN); Haixia LIU, Shanghai (CN); Yafei LIU, Shanghai (CN); Hong SHEN, Shanghai (CN); Weixing ZHANG, Shanghai (CN); Zhiwei ZHANG, Shanghai (CN); Wei ZHU, Shanghai (CN)

(73) Assignee: Hoffmann-La Roche Inc., Little Falls, NJ (US)

(21) Appl. No.: 17/278,416

(22) PCT Filed: Sep. 25, 2019
(86) PCT No.: PCT/EP2019/075791

§ 371 Date: Mar. 22, 2021

(30) Foreign Application Priority Data
Sep. 27, 2018 (CN) PCT/CN2018/108015
Aug. 21, 2019 (CN) PCT/CN2019/101852

(51) Int. Cl. C07D 413/14; C07D 498/08; C07D 498/10; C07D 471/04

(52) U.S. Cl. CPC C07D 413/14; C07D 498/08; C07B 2200/05; C07D 471/04; C07D 498/10

(57) ABSTRACT

The present invention relates to compounds of formula (I), wherein R¹ to R³, A and Q are as described herein, and their pharmaceutically acceptable salt, enantiomer or diastereomer thereof, and compositions including the compounds and methods of using the compounds.

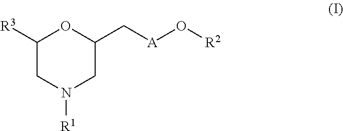

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### FIELD OF THE INVENTION

Autoimmune connective tissue disease (CTD) include prototypical autoimmune syndromes such as Systemic Lupus Erythematosus (SLE), primary Sjögren's syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SLE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long term usage of immunosuppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 377, 721.). Other biologics, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as auto-inflammation diseases.

Toll Like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7,8,9 is considered as a key node in a broad of autoimmune and auto-inflammatory diseases (Krieg, A. M. et al. Immunol. Rev. 2007, 220, 251. Jimenez-Dalmaroni, M. J. et al Autoimmun Rev. 2016, 15, 1. Chen, J. Q., et al. Clinical Reviews in Allergy & Immunology 2016, 50, 1.). Anti-RNA and anti-DNA antibodies are well established diagnostic markers of SLE, and these antibodies can deliver both self-RNA and self-DNA to endosomes. While self-RNA complexes can be recognized by TLR7 and TLR8, self-DNA complexes can trigger TLR9 activation. Indeed, defective clearance of self-RNA and self-DNA from blood and/or tissues is evident in SLE (Systemic Lupus Erythematosus) patients. TLR7 and TLR9 have been reported to be upregulated in SLE tissues, and correlate with chronicity and activity of lupus nephritis, respectively. In B cells of SLE patients, TLR7 expression correlates with anti-RNP antibody production, while TLR9 expression with IL-6 and anti-dsDNA antibody levels. Consistently, in lupus mouse models, TLR7 is required for anti-RNA antibodies, and TLR9 is required for anti-nucleosome antibody. On the other hand, overexpression of TLR7 or human TLR8 in mice promotes autoimmunity and autoinflammation. Moreover, activation of TLR8 specifically contributes to inflammatory cytokine secretion of mDC/macrophages, neutrophil NETosis, induction of Th17 cells, and suppression of Treg cells. In addition to the described role of TLR9 in promoting autoantibody production of B cells, activation of TLR9 by self-DNA in pDC also leads to induction of type I IFNs and other inflammatory cytokines. Given these roles of TLR9 in both pDC and B cells, both as key contributors to the pathogenesis of autoimmune diseases, and the extensive presence of self-DNA complexes that could readily activate TLR9 in many patients with autoimmune diseases, it may have extra benefit to further block self-DNA mediated TLR9 pathways on top of inhibition of TLR7 and TLR8 pathways. Taken together, TLR7, 8, and 9 pathways represent new therapeutic targets for the treatment of autoimmune and auto-inflammatory diseases, for which no effective steroid-free and non-cytotoxic oral drugs exist, and inhibition of all these pathways from the very upstream may deliver satisfying therapeutic effects. As such, we invented oral compounds that target and suppress TLR7, TLR8 and TLR9 for the treatment of autoimmune and auto-inflammatory diseases.

### SUMMARY OF THE INVENTION

The present invention relates to novel compounds of formula (I) or (Ia),

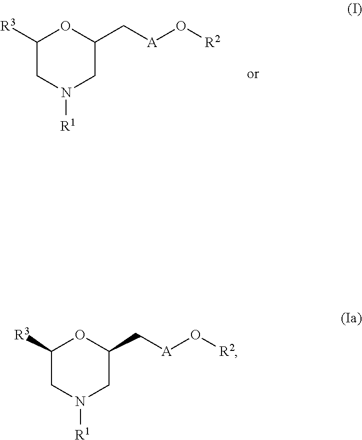

wherein
R¹ is

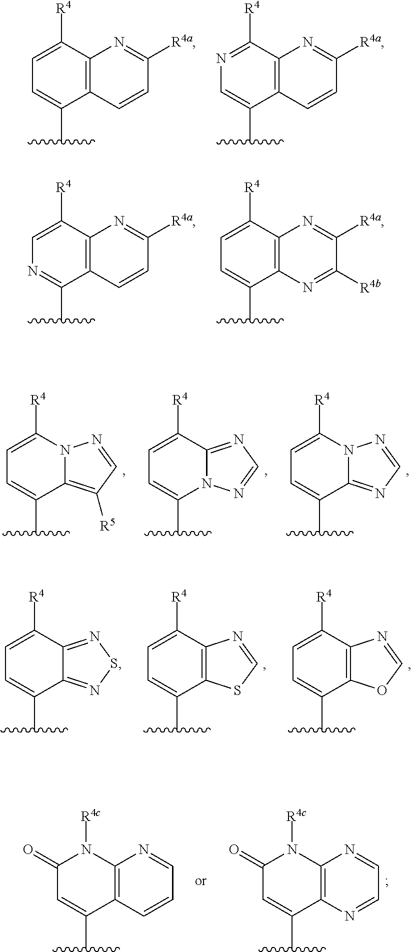

wherein R⁴ is C₁₋₆alkyl, C₁₋₆alkoxy, haloC₁₋₆alkyl, halogen, nitro or cyano; R^{4a} is H or deuterium; R^{4b} is H, deuterium or C₁₋₆alkyl; R^{4c} is C₁₋₆alkyl or C₃₋₇cycloalkyl; R⁵ is H or halogen;
R² is ((C₁₋₆alkyl)₂amino)C₁₋₆alkoxy;
   (C₁₋₆alkoxypyrrolidinyl)amino;
   (cyanopyrrolidinyl)amino;
   1,4-diazepanyl substituted by hydroxy;
   1,6-diazaspiro[3.3]heptanyl;
   2,5-diazabicyclo[2.2.1]heptanyl;
   2,6-diazaspiro[3.3]heptanyl substituted by C₁₋₆alkyl;
   3,4,4a,5,7,7a-hexahydro-2H-pyrrolo[3,4-b][1,4]oxazinyl;
   3-oxa-7,9-diazabicyclo[3.3.1]nonanyl;
   5-oxa-2,8-diazaspiro[3.5]nonanyl;
   aminoC₁₋₆alkyl;
   aminooxazepanyl;
   azetidinyl substituted once or twice by substituents independently selected from amino and C₁₋₆alkyl;
   azetidinylamino;
   halopyrrolidinylamino;
   morpholinyl unsubstituted or substituted by C₁₋₆alkyl;
   morpholinylC₁₋₆alkyl;
   piperazinyl unsubstituted or substituted by C₁₋₆alkyl, hydroxyC₁₋₆alkyl, pyrrolidinylcarbonyl, ((C₁₋₆alkyl)₂amino)C₁₋₆alkylcarbonyl or azetidinylcarbonyl;
   piperidinyl unsubstituted or substituted once or twice by substituents independently selected from amino, halogen and C₁₋₆ alkoxy; or
   pyrrolidinyl substituted once, twice or three times by substituents independently selected from amino, halogen, hydroxy, C₁₋₆alkyl and C₁₋₆ alkoxy;
R³ is C₁₋₆alkyl;
A is azetidinyl, hydroxyazetidinyl, piperazinyl, pyrrolidinyl, piperidinyl or cyanopiperidinyl;
Q is phenyl unsubstituted or substituted by halogen;
   pyrazinyl;
   pyridazinyl;
   pyridinyl unsubstituted or substituted by C₁₋₆alkyl;
   pyrimidinyl unsubstituted or substituted once or twice by substituents independently selected from halogen, C₁₋₆alkyl, C₁₋₆ alkoxy and cyano; or quinazolinyl;
      or a pharmaceutically acceptable salt thereof.

Another object of the present invention is related to novel compounds of formula (I) or (Ia), their manufacture, medicaments based on a compound in accordance with the invention and their production as well as the use of compounds of formula (I) or (Ia) as TLR7 and/or TLR8 and/or TLR9 antagonist, and for the treatment or prophylaxis of systemic lupus erythematosus or lupus nephritis. The compounds of formula (I) or (Ia) show superior TLR7 and TLR8 and TLR9 antagonism activity. In addition, the compounds of formula (I) or (Ia) also show good cytotoxicity, solubility, hPBMC, human microsome stability and SDPK profiles, as well as low CYP inhibition.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “halogen” and “halo” are used interchangeably herein and denote fluoro, chloro, bromo, or iodo.

The letter “D” shown in the molecular structure denotes “deuterium”.

The term “haloC₁₋₆alkyl” denotes an alkyl group wherein at least one of the hydrogen atoms of the alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl, trifluoromethyl and trifluoroethyl.

The term “halopyrrolidinyl” denotes a pyrrolidinyl substituted once, twice or three times by halogen. Examples of halopyrrolidinyl include, but not limited to, fluoropyrrolidinyl and difluoropyrrolidinyl.

The term “halopiperidinyl” denotes a piperidinyl substituted once, twice or three times by halogen. Examples of halopiperidinyl include, but not limited to, fluoropiperidinyl and difluoropiperidinyl.

The term “LG” denotes a leaving group, which is a molecular fragment that departs with a pair of electrons in heterolytic bond cleavage. Leaving groups can be anions or neutral molecules, but in either case it is crucial that the leaving group be able to stabilize the additional electron density that results from bond heterolysis. Common anionic leaving groups are halides, and sulfonate esters such as OTf, OTs and OMs.

The term “PG” denotes a protecting group, which is introduced into a molecule by chemical modification of a functional group to obtain chemoselectivity in a subsequent chemical reaction. Typical protecting groups are Boc, Cbz and Bn.

The term “cis-isomers” and “trans-isomers” denote the relative stereochemistry of the molecule or moiety. For example: tert-butyl N-(cis-5-fluoropiperidin-3-yl)carbamate

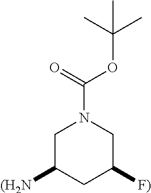

as the “cis-isomers” refers to a mixture of

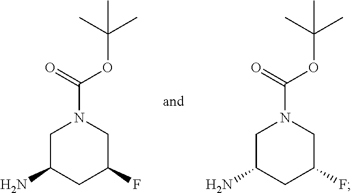

similarly, tert-butyl trans-(4-methoxypyrrolidin-3-yl)carbamate

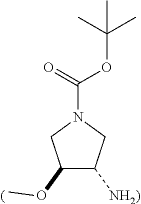

as the “trans-isomers” refers to a mixture of

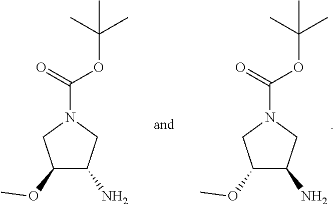

The way of showing relative stereochemistry also applies to the final compound.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Antagonist of TLR7 and/or TLR8 and/or TLR9

The present invention relates to (i) a compound of formula (I),

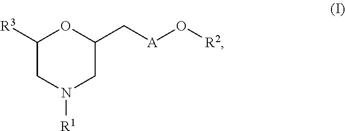

wherein

### R¹ is

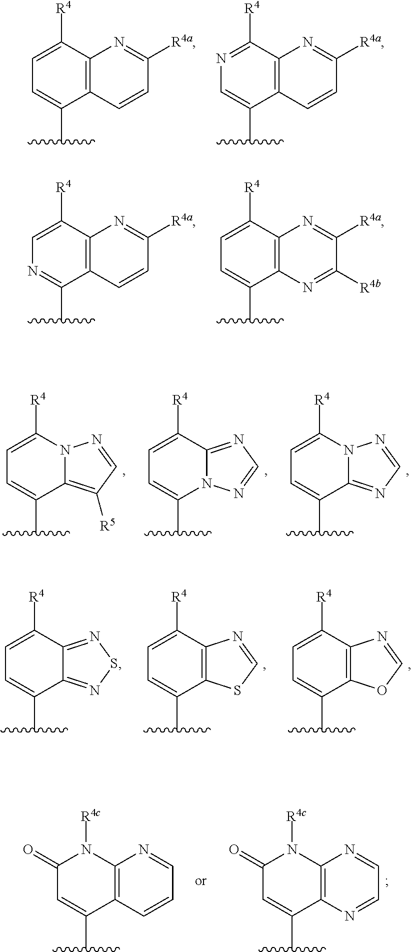

wherein R⁴ is C₁₋₆alkyl, C₁₋₆alkoxy, haloC₁₋₆alkyl, halogen, nitro or cyano; R^{4a} is H or deuterium; R^{4b} is H, deuterium or C₁₋₆alkyl; R^{4c} is C₁₋₆alkyl or C₃₋₇cycloalkyl; R⁵ is H or halogen;
R² is ((C₁₋₆alkyl)₂amino)C₁₋₆ alkoxy;
(C₁₋₆alkoxypyrrolidinyl)amino;
(cyanopyrrolidinyl)amino;
1,4-diazepanyl substituted by hydroxy;
1,6-diazaspiro[3.3]heptanyl;
2,5-diazabicyclo[2.2.1]heptanyl;
2,6-diazaspiro[3.3]heptanyl substituted by C₁₋₆alkyl;
3,4,4a,5,7,7a-hexahydro-2H-pyrrolo[3,4-b][1,4]oxazinyl;
3-oxa-7,9-diazabicyclo[3.3.1]nonanyl;
5-oxa-2,8-diazaspiro[3.5]nonanyl;
aminoC₁₋₆alkyl;
aminooxazepanyl;
azetidinyl substituted once or twice by substituents independently selected from amino and C₁₋₆alkyl;
azetidinylamino;
halopyrrolidinylamino;
morpholinyl unsubstituted or substituted by C₁₋₆alkyl;
morpholinylC₁₋₆alkyl;
piperazinyl unsubstituted or substituted by C₁₋₆alkyl, hydroxyC₁₋₆alkyl, pyrrolidinylcarbonyl, ((C₁₋₆alkyl)₂amino)C₁₋₆alkylcarbonyl or azetidinylcarbonyl;
piperidinyl unsubstituted or substituted once or twice by substituents independently selected from amino, halogen and C₁₋₆ alkoxy; or
pyrrolidinyl substituted once, twice or three times by substituents independently selected from amino, halogen, hydroxy, C₁₋₆alkyl and C₁₋₆ alkoxy;
   R³ is C₁₋₆alkyl;
   A is azetidinyl, hydroxyazetidinyl, piperazinyl, pyrrolidinyl, piperidinyl or cyanopiperidinyl;
   Q is phenyl unsubstituted or substituted by halogen;
pyrazinyl;
pyridazinyl;
pyridinyl unsubstituted or substituted by C₁₋₆alkyl;
pyrimidinyl unsubstituted or substituted once or twice by substituents independently selected from halogen, C₁₋₆alkyl, C₁₋₆ alkoxy and cyano; or
quinazolinyl;
   or a pharmaceutically acceptable salt thereof.

Another embodiment of present invention is (ii) a compound of formula (Ia),

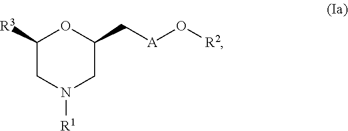

wherein

### R¹ is

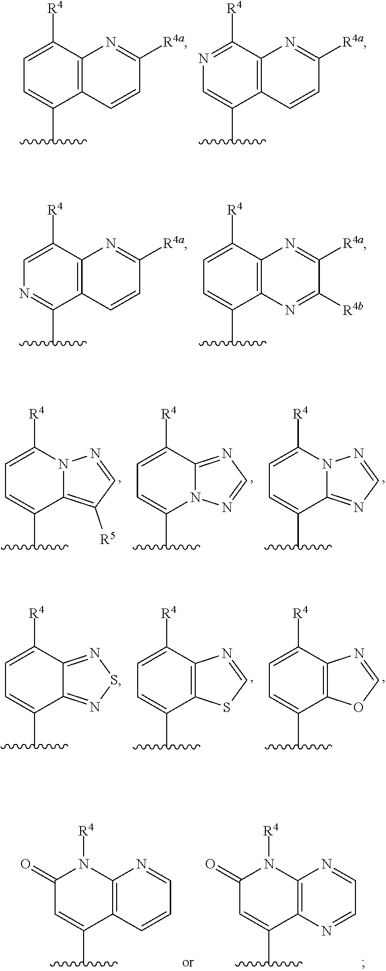

wherein R⁴ is C₁₋₆alkyl, C₁₋₆alkoxy, haloC₁₋₆alkyl, halogen, nitro or cyano; R^{4a} is H or deuterium; R^{4b} is H, deuterium or C₁₋₆alkyl; R^{4c} is C₁₋₆alkyl or C₃₋₇cycloalkyl; R⁵ is H or halogen;
R² is ((C₁₋₆alkyl)₂amino)C₁₋₆ alkoxy;
(C₁₋₆alkoxypyrrolidinyl)amino;
(cyanopyrrolidinyl)amino;
1,4-diazepanyl substituted by hydroxy;
1,6-diazaspiro[3.3]heptanyl;
2,5-diazabicyclo[2.2.1]heptanyl;
2,6-diazaspiro[3.3]heptanyl substituted by C₁₋₆alkyl;
3,4,4a,5,7,7a-hexahydro-2H-pyrrolo[3,4-b][1,4]oxazinyl;
3-oxa-7,9-diazabicyclo[3.3.1]nonanyl;
5-oxa-2,8-diazaspiro[3.5]nonanyl;
aminoC₁₋₆alkyl;
aminooxazepanyl;
azetidinyl substituted once or twice by substituents independently selected from amino and C₁₋₆alkyl;
azetidinylamino;
halopyrrolidinylamino;
morpholinyl unsubstituted or substituted by C₁₋₆alkyl;
morpholinylC₁₋₆alkyl;
piperazinyl unsubstituted or substituted by C₁₋₆alkyl, hydroxyC₁₋₆alkyl, pyrrolidinylcarbonyl, ((C₁₋₆alkyl)₂amino)C₁₋₆alkylcarbonyl or azetidinylcarbonyl;
piperidinyl unsubstituted or substituted once or twice by substituents independently selected from amino, halogen and C₁₋₆ alkoxy; or
pyrrolidinyl substituted once, twice or three times by substituents independently selected from amino, halogen, hydroxy, C₁₋₆alkyl and C₁₋₆ alkoxy;
   R³ is C₁₋₆alkyl;
   A is azetidinyl, hydroxyazetidinyl, piperazinyl, pyrrolidinyl, piperidinyl or cyanopiperidinyl;
   Q is phenyl unsubstituted or substituted by halogen;
pyrazinyl;
pyridazinyl;
pyridinyl unsubstituted or substituted by C₁₋₆alkyl;
pyrimidinyl unsubstituted or substituted once or twice by substituents independently selected from halogen, C₁₋₆alkyl, C₁₋₆ alkoxy and cyano; or
quinazolinyl;
   or a pharmaceutically acceptable salt thereof.

A further embodiment of present invention is (iii) a compound of formula (I) or (Ia) according to (i) or (ii), or a pharmaceutically acceptable salt thereof, wherein

### R¹ is

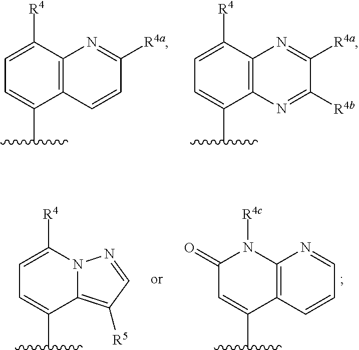

wherein R⁴ is C₁₋₆alkyl, haloC₁₋₆alkyl or cyano; R^{4a} is H or deuterium; R^{4b} is H or deuterium; R^{4c} is C₁₋₆alkyl; R⁵ is H or halogen.

A further embodiment of present invention is (iv) a compound of formula (I) or (Ia) according to any one of (i) to (iii), wherein R⁴ is methyl, trifluoromethyl or cyano; R^{4a} is H or deuterium; R^{4b} is H or deuterium; R^{4c} is methyl; R⁵ is H or fluoro.

A further embodiment of present invention is (v) a compound of formula (I) or (Ia) according to any one of (i) to (iv), or a pharmaceutically acceptable salt thereof, wherein R² is (((C₁₋₆alkyl)₂amino)C₁₋₆alkylcarbonyl)piperazinyl; ((C₁₋₆alkyl)₂amino)C₁₋₆alkoxy; (azetidinylcarbonyl)piperazinyl; (C₁₋₆ alkoxypyrrolidinyl)amino; (C₁₋₆alkyl)morpholinyl; (cyanopyrrolidinyl)amino; (hydroxyC₁₋₆alkyl)piperazinyl; (pyrrolidinylcarbonyl)piperazinyl; 1,6-diazaspiro[3.3]heptanyl; 2,5-diazabicyclo[2.2.1]heptanyl; 3,4,4a,5,7,7a-hexahydro-2H-pyrrolo[3,4-b][1,4]oxazinyl; 3-oxa-7,9-diazabicyclo[3.3.1]nonanyl; 5-oxa-2,8-diazaspiro[3.5]nonanyl; amino(C₁₋₆ alkoxy)piperidinyl; amino(C₁₋₆ alkoxy)pyrrolidinyl; amino(C₁₋₆alkyl)azetidinyl; amino(hydroxy)(C₁₋₆alkyl)pyrrolidinyl; aminoazetidinyl; aminoC₁₋₆alkyl; aminohalopiperidinyl; aminohalopyrrolidinyl; aminooxazepanyl; aminopiperidinyl; azetidinylamino; C₁₋₆alkyl-2,6-diazaspiro[3.3]heptanyl; C₁₋₆ alkylpiperazinyl; halopyrrolidinylamino; hydroxy-1,4-diazepanyl; morpholinyl; morpholinylC₁₋₆alkyl; piperazinyl or piperidinyl.

A further embodiment of present invention is (vi) a compound of formula (I) or (Ia) according to any one of (i) to (v), or a pharmaceutically acceptable salt thereof, wherein R² is (1-hydroxy-1-methyl-ethyl)piperazin-1-yl; (2-pyrrolidinylcarbonyl)piperazin-1-yl; (4-cyanopyrrolidin-3-yl)amino; (4-fluoropyrrolidin-3-yl)amino; (4-methoxypyrrolidin-3-yl)amino; (dimethylamino)ethoxy; 1,6-diazaspiro[3.3]heptan-6-yl; 2,5-diazabicyclo[2.2.1]heptan-2-yl; 2-methylmorpholin-2-yl; 2-morpholin-2-yl; 3-(hydroxymethyl)piperazin-1-yl; 3,4,4a,5,7,7a-hexahydro-2H-pyrrolo[3,4-b][1,4]oxazin-6-yl; 3-amino-3-methyl-azetidin-1-yl; 3-amino-4-fluoro-1-piperidinyl; 3-amino-4-fluoro-pyrrolidin-1-yl; 3-amino-4-methoxy-1-piperidinyl; 3-amino-4-methoxy-pyrrolidin-1-yl; 3-amino-5-fluoro-1-piperidinyl; 3-aminoazetidin-1-yl; 3-methylpiperazin-1-yl; 3-oxa-7,9-diazabicyclo[3.3.1]nonan-7-yl; 4-(azetidinyl-2-carbonyl)piperazin-1-yl; 4-[2-(dimethylamino)acetyl]piperazin-1-yl; 4-amino-1-piperidinyl; 4-amino-3,3-difluoro-1-piperidinyl; 4-amino-3,3-difluoro-pyrrolidin-1-yl; 4-amino-3-fluoro-1-piperidinyl; 4-amino-3-hydroxy-3-methyl-pyrrolidin-1-yl; 4-amino-3-methoxy-1-piperidinyl; 4-methylpiperazin-1-yl; 4-piperidinyl; 5-amino-3,3-difluoro-1-piperidinyl; 5-oxa-2,8-diazaspiro[3.5]nonan-2-yl; 5-oxa-2,8-diazaspiro[3.5]nonan-8-yl; 6-amino-1,4-oxazepan-4-yl; 6-hydroxy-1,4-diazepan-1-yl; 6-methyl-2,6-diazaspiro[3.3]heptan-2-yl; aminomethyl; azetidin-3-ylamino; morpholin-2-yl; morpholin-2-ylmethyl or piperazin-1-yl.

A further embodiment of present invention is (vii) a compound of formula (I) or (Ia) according to any one of (i) to (vi), or a pharmaceutically acceptable salt thereof, wherein A is

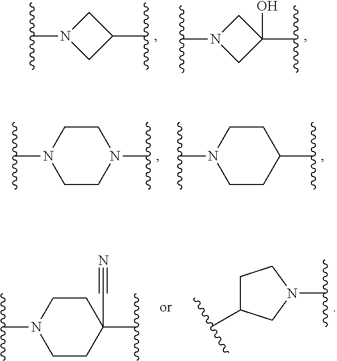

A further embodiment of present invention is (viii) a compound of formula (I) or (Ia) according to any one of (i) to (vii), or a pharmaceutically acceptable salt thereof, wherein Q is phenyl, fluorophenyl, pyrazinyl, pyridazinyl, pyridinyl, methylpyridinyl, pyrimidinyl, cyanopyrimidinyl, fluoropyrimidinyl, methoxypyrimidinyl, methylpyrimidinyl, dimethylpyrimidinyl or quinazolinyl.

A further embodiment of present invention is (ix) a compound of formula (I) or (Ia) according to any one of (i) to (viii), or a pharmaceutically acceptable salt thereof, wherein R³ is methyl.

A further embodiment of present invention is (x) a compound of formula (I) or (Ia) according to any one of (i) to (ix), or a pharmaceutically acceptable salt thereof, wherein R¹ is

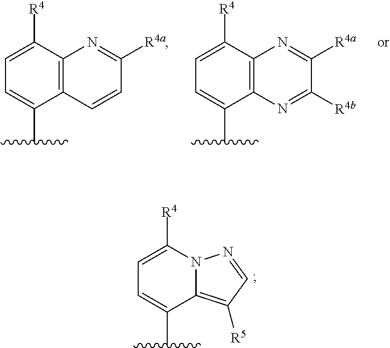

wherein R⁴ is C₁₋₆alkyl, haloC₁₋₆alkyl or cyano; R^{4a} and R^{4b} are simultaneously H or deuterium; R⁵ is H or halogen.

A further embodiment of present invention is (xi) a compound of formula (I) or (Ia) according to any one of (i) to (x), or a pharmaceutically acceptable salt thereof, wherein R⁴ is methyl, trifluoromethyl or cyano; R^{4a} and R^{4b} are simultaneously H or deuterium; R⁵ is H or fluoro.

A further embodiment of present invention is (xii) a compound of formula (I) or (Ia) according to any one of (i) to (xi), or a pharmaceutically acceptable salt thereof, wherein R² is 3,4,4a,5,7,7a-hexahydro-2H-pyrrolo[3,4-b][1,4]oxazinyl; 5-oxa-2,8-diazaspiro[3.5]nonanyl; amino(C₁₋₆ alkoxy)pyrrolidinyl; amino(C₁₋₆alkyl)azetidinyl; aminoazetidinyl; aminopiperidinyl; C₁₋₆alkyl-2,6-diazaspiro[3.3]heptanyl; halopyrrolidinylamino; morpholinyl; morpholinylC₁₋₆alkyl or piperazinyl.

A further embodiment of present invention is (xiii) a compound of formula (I) or (Ia) according to any one of (i) to (xii), or a pharmaceutically acceptable salt thereof, wherein R² is (4-fluoropyrrolidin-3-yl)amino; 3,4,4a,5,7,7a-hexahydro-2H-pyrrolo[3,4-b][1,4]oxazin-6-yl; 3-amino-3-methyl-azetidin-1-yl; 3-amino-4-methoxy-pyrrolidin-1-yl; 3-aminoazetidin-1-yl; 4-amino-1-piperidinyl; 5-oxa-2,8-diazaspiro[3.5]nonan-2-yl; 6-methyl-2,6-diazaspiro[3.3]heptan-2-yl; morpholin-2-yl; morpholin-2-ylmethyl or piperazin-1-yl.

A further embodiment of present invention is (xiv) a compound of formula (I) or (Ia) according to any one of (i) to (xiii), or a pharmaceutically acceptable salt thereof, wherein A is

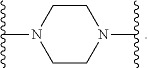 [SMILES: *N1CCN(*)CC1]

A further embodiment of present invention is (xv) a compound of formula (I) or (Ia) according to any one of (i) to (xiv), or a pharmaceutically acceptable salt thereof, wherein Q is phenyl, pyridinyl, pyrimidinyl or C₁₋₆alkylpyrimidinyl.

A further embodiment of present invention is (xvi) a compound of formula (I) or (Ia) according to any one of (i) to (xv), or a pharmaceutically acceptable salt thereof, wherein Q is phenyl, pyridinyl, pyrimidinyl or methylpyrimidinyl.

A further embodiment of present invention is (xvii) a compound of formula (I) or (Ia) according to any one of (i) to (xvi), wherein
R¹ is

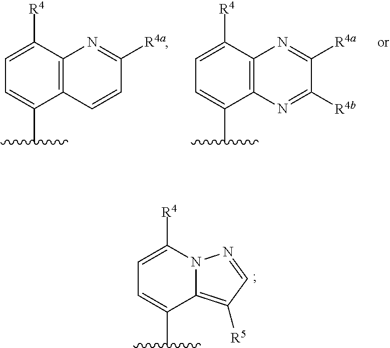

wherein R⁴ is C₁₋₆alkyl, haloC₁₋₆alkyl or cyano; R^{4a} and R^{4b} are simultaneously H or deuterium; R⁵ is H or halogen;
R² is 3,4,4a,5,7,7a-hexahydro-2H-pyrrolo[3,4-b][1,4]oxazinyl; 5-oxa-2,8-diazaspiro[3.5]nonanyl; amino(C₁₋₆ alkoxy)pyrrolidinyl; amino(C₁₋₆alkyl)azetidinyl; aminoazetidinyl; aminopiperidinyl; C₁₋₆alkyl-2,6-diazaspiro[3.3]heptanyl; halopyrrolidinylamino; morpholinyl; morpholinylC₁₋₆alkyl or piperazinyl;
R³ is C₁₋₆alkyl;
A is

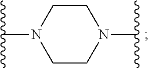 [SMILES: *N1CCN(*)CC1]

Q is phenyl, pyridinyl, pyrimidinyl or C₁₋₆alkylpyrimidinyl;
   or a pharmaceutically acceptable salt thereof.

A further embodiment of present invention is (xviii) a compound of formula (I) or (Ia) according to any one of (i) to (xvii), wherein
R¹ is

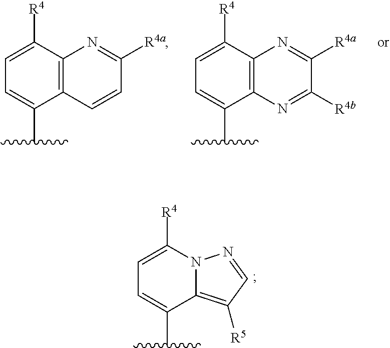

wherein R⁴ is methyl, trifluoromethyl or cyano; R^{4a} and R^{4b} are simultaneously H or deuterium; R⁵ is H or fluoro;
R² is (4-fluoropyrrolidin-3-yl)amino; 3,4,4a,5,7,7a-hexahydro-2H-pyrrolo[3,4-b][1,4]oxazin-6-yl; 3-amino-3-methyl-azetidin-1-yl; 3-amino-4-methoxy-pyrrolidin-1-yl; 3-aminoazetidin-1-yl; 4-amino-1-piperidinyl; 5-oxa-2,8-diazaspiro[3.5]nonan-2-yl; 6-methyl-2,6-diazaspiro[3.3]heptan-2-yl; morpholin-2-yl; morpholin-2-ylmethyl or piperazin-1-yl;
R³ is methyl;
A is

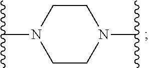 [SMILES: *N1CCN(*)CC1]

Q is phenyl, pyridinyl, pyrimidinyl or methylpyrimidinyl;
   or a pharmaceutically acceptable salt thereof.

The present invention relates to (i′) a compound of formula (I),

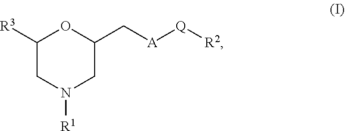

wherein
R¹ is selected from

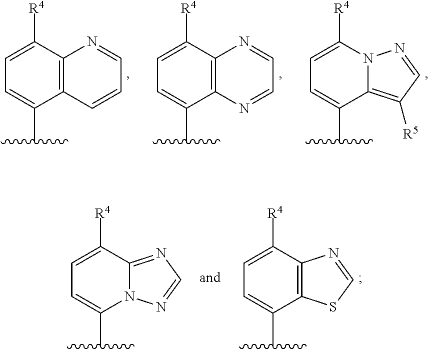

wherein R⁴ is selected from halogen, cyano, C₁₋₆alkyl, haloC₁₋₆alkyl and C₂₋₆ alkynyl; R⁵ is halogen;
R² is selected from (((C₁₋₆alkyl)₂amino)C₁₋₆alkylcarbonyl)piperazinyl; ((C₁₋₆alkyl)₂amino)C₁₋₆alkoxy; (azetidinylcarbonyl)piperazinyl; (C₁₋₆ alkoxypyrrolidinyl)amino; (cyanopyrrolidinyl)amino; (hydroxyC₁₋₆alkyl)piperazinyl; (pyrrolidinylcarbonyl)piperazinyl; 3-oxa-7,9-diazabicyclo[3.3.1]nonanyl; 5-oxa-2,8-diazaspiro[3.5]nonanyl; amino(C₁₋₆ alkoxy)pyrrolidinyl; aminoazetidinyl; aminoC₁₋₆alkyl; aminohalopiperidinyl; aminohalopyrrolidinyl; aminooxazepanyl; aminopiperidinyl; azetidinylamino; C₁₋₆alkylpiperazinyl; halopyrrolidinylamino; hydroxydiazepanyl; morpholinyl; piperazinyl and piperidinyl;
R³ is C₁₋₆alkyl;
A is selected from azetidinyl, piperazinyl, pyrrolidinyl, piperidinyl and caynopiperidinyl;
Q is selected from phenyl;
   pyrazinyl;
   pyridazinyl;
   pyridinyl;
   pyrimidinyl, said pyrimidinyl being unsubstituted or substituted once or twice by substituents independently selected from halogen, C₁₋₆alkyl, C₁₋₆alkoxy and cyano; and
   quinazolinyl;

or a pharmaceutically acceptable salt thereof.

Another embodiment of present invention is (ii′) a compound of formula (Ia),

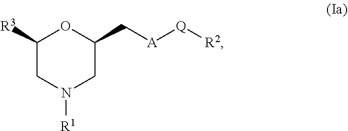

wherein
R¹ is selected from

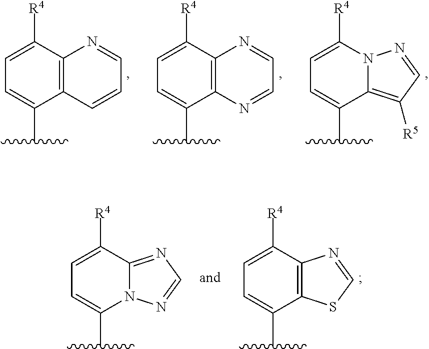

wherein R⁴ is selected from halogen, cyano, C₁₋₆alkyl, haloC₁₋₆alkyl and C₂₋₆ alkynyl; R⁵ is halogen;
R² is selected from (((C₁₋₆alkyl)₂amino)C₁₋₆alkylcarbonyl)piperazinyl; ((C₁₋₆alkyl)₂amino)C₁₋₆alkoxy; (azetidinylcarbonyl)piperazinyl; (C₁₋₆ alkoxypyrrolidinyl)amino; (cyanopyrrolidinyl)amino; (hydroxyC₁₋₆alkyl)piperazinyl; (pyrrolidinylcarbonyl)piperazinyl; 3-oxa-7,9-diazabicyclo[3.3.1]nonanyl; 5-oxa-2,8-diazaspiro[3.5]nonanyl; amino(C₁₋₆ alkoxy)pyrrolidinyl; aminoazetidinyl; aminoC₁₋₆alkyl; aminohalopiperidinyl; aminohalopyrrolidinyl; aminooxazepanyl; aminopiperidinyl; azetidinylamino; C₁₋₆alkylpiperazinyl; halopyrrolidinylamino; hydroxydiazepanyl; morpholinyl; piperazinyl and piperidinyl;
R³ is C₁₋₆alkyl;
A is selected from azetidinyl, piperazinyl, pyrrolidinyl, piperidinyl and caynopiperidinyl;
Q is selected from phenyl;
   pyrazinyl;
   pyridazinyl;
   pyridinyl;
   pyrimidinyl, said pyrimidinyl being unsubstituted or substituted once or twice by substituents independently selected from halogen, C₁₋₆alkyl, C₁₋₆alkoxy and cyano; and
   quinazolinyl;
   or a pharmaceutically acceptable salt thereof.

A further embodiment of present invention is (iii′) a compound of formula (I) or (Ia) according to (i) or (ii), or a pharmaceutically acceptable salt thereof, wherein R¹

is

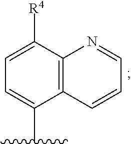

wherein R⁴ is cyano.

A further embodiment of present invention is (iv′) a compound of formula (I) or (Ia) according to any one of (i′) to (iii′), or a pharmaceutically acceptable salt thereof, wherein A is piperazinyl.

A further embodiment of present invention is (v′) a compound of formula (I) or (Ia) according to any one of (i′) to (iv′), or a pharmaceutically acceptable salt thereof, wherein Q is pyrimidinyl, said pyrimidinyl being unsubstituted or substituted by C₁₋₆alkyl.

A further embodiment of present invention is (vi′) a compound of formula (I) or (Ia) according to any one of (i′) to (v′), or a pharmaceutically acceptable salt thereof, wherein R² is selected from amino(C₁₋₆ alkoxy)pyrrolidinyl, aminopiperidinyl and piperazinyl.

A further embodiment of present invention is (vii′) a compound of formula (I) or (Ia) according to any one of (i′) to (vi′), or a pharmaceutically acceptable salt thereof, wherein R² is selected from amino(methoxy)pyrrolidinyl, aminopiperidinyl and piperazinyl.

A further embodiment of present invention is (viii′) a compound of formula (I) or (Ia) according to any one of (i′) to (vii′), wherein

R¹ is selected from

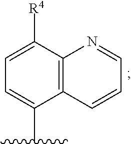

wherein R⁴ is cyano;
R² is selected from amino(C₁₋₆ alkoxy)pyrrolidinyl, aminopiperidinyl and piperazinyl;
R³ is C₁₋₆alkyl;
A is piperazinyl;
Q is pyrimidinyl, said pyrimidinyl being unsubstituted or substituted by C₁₋₆alkyl;
or a pharmaceutically acceptable salt thereof.

A further embodiment of present invention is (ix′) a compound of formula (I) or (Ia) according to any one of (i′) to (viii′), wherein

R¹ is selected from

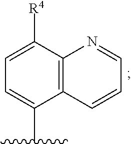

wherein R⁴ is cyano;
R² is selected from amino(methoxy)pyrrolidinyl, aminopiperidinyl and piperazinyl;
R³ is methyl;
A is piperazinyl;
Q is pyrimidinyl, said pyrimidinyl being unsubstituted or substituted by methyl;
or a pharmaceutically acceptable salt thereof.

Any of the above embodiments may be combined.

Another embodiment of present invention is that (x) a compound of formula (I) or (Ia) selected from the following:
5-[(2R,6S)-2-methyl-6-[[3-(3-piperazin-1-ylphenyl)pyrrolidin-1-yl]methyl]morpholin-4-yl]quinoline-8-carbonitrile;
5-[(2R,6S)-2-methyl-6-[[4-[3-(4-piperidyl)phenyl]-1-piperidyl]methyl]morpholin-4-yl]quinoline-8-carbonitrile;
5-[(2R,6S)-2-methyl-6-[[3-(4-piperazin-1-ylphenyl)azetidin-1-yl]methyl]morpholin-4-yl]quinoline-8-carbonitrile;
5-[(2R,6S)-2-methyl-6-[[3-(3-piperazin-1-ylphenyl)azetidin-1-yl]methyl]morpholin-4-yl]quinoline-8-carbonitrile;
5-[(2S,6R)-2-[[4-[4-(aminomethyl)phenyl]piperazin-1-yl]methyl]-6-methyl-morpholin-4-yl]quinoline-8-carbonitrile;
5-[(2R,6S)-2-methyl-6-[[4-(5-piperazin-1-ylpyrazin-2-yl)piperazin-1-yl]methyl]morpholin-4-yl]quinoline-8-carbonitrile;
5-[(2R,6S)-2-methyl-6-[[4-(5-piperazin-1-yl-3-pyridyl)piperazin-1-yl]methyl]morpholin-4-yl]quinoline-8-carbonitrile;
5-[(2R,6S)-2-methyl-6-[[4-(5-piperazin-1-yl-2-pyridyl)piperazin-1-yl]methyl]morpholin-4-yl]quinoline-8-carbonitrile;
5-[(2R,6S)-2-methyl-6-[[4-(4-piperazin-1-yl-2-pyridyl)piperazin-1-yl]methyl]morpholin-4-yl]quinoline-8-carbonitrile;
5-[(2R,6S)-2-methyl-6-[[4-(6-piperazin-1-yl-2-pyridyl)piperazin-1-yl]methyl]morpholin-4-yl]quinoline-8-carbonitrile;
5-[(2S,6R)-2-[[4-cyano-4-(4-piperazin-1-ylphenyl)-1-piperidyl]methyl]-6-methyl-morpholin-4-yl]quinoline-8-carbonitrile;
5-[(2R,6S)-2-methyl-6-[[4-(2-piperazin-1-ylpyrimidin-5-yl)piperazin-1-yl]methyl]morpholin-4-yl]quinoline-8-carbonitrile;
5-[(2R,6S)-2-methyl-6-[[4-(6-piperazin-1-yl-3-pyridyl)piperazin-1-yl]methyl]morpholin-4-yl]quinoline-8-carbonitrile;
5-[(2R,6S)-2-methyl-6-[[4-(4-piperazin-1-ylpyrimidin-2-yl)piperazin-1-yl]methyl]morpholin-4-yl]quinoline-8-carbonitrile;
5-[(2R,6S)-2-methyl-6-[[4-(2-piperazin-1-ylpyrimidin-4-yl)piperazin-1-yl]methyl]morpholin-4-yl]quinoline-8-carbonitrile;
5-[(2R,6S)-2-methyl-6-[[4-(6-piperazin-1-ylpyridazin-3-yl)piperazin-1-yl]methyl]morpholin-4-yl]quinoline-8-carbonitrile;
5-[(2R,6S)-2-methyl-6-[[4-(5-piperazin-1-ylpyrimidin-2-yl)piperazin-1-yl]methyl]morpholin-4-yl]quinoline-8-carbonitrile;
5-[(2R,6S)-2-methyl-6-[[4-(6-piperazin-1-ylpyrimidin-4-yl)piperazin-1-yl]methyl]morpholin-4-yl]quinoline-8-carbonitrile;
5-[(2R,6S)-2-methyl-6-[[4-[4-[(2R)-morpholin-2-yl]phenyl]piperazin-1-yl]methyl]morpholin-4-yl]quinoline-8-carbonitrile;
5-[(2R,6S)-2-methyl-6-[[4-[4-[(2S)-morpholin-2-yl]phenyl]piperazin-1-yl]methyl]morpholin-4-yl]quinoline-8-carbonitrile;
5-[(2R,6S)-2-methyl-6-[[4-[6-(4-methylpiperazin-1-yl)pyrimidin-4-yl]piperazin-1-yl]methyl]morpholin-4-yl]quinoline-8-carbonitrile;
5-[(2S,6R)-2-[[4-[2-(3-aminoazetidin-1-yl)pyrimidin-4-yl]piperazin-1-yl]methyl]-6-methyl-morpholin-4-yl]quinoline-8-carbonitrile;
5-[(2S,6R)-2-[[4-[2-(3-aminoazetidin-1-yl)pyrimidin-4-yl]piperazin-1-yl]methyl]-6-methyl-morpholin-4-yl]quinoline-8-carbonitrile;
5-[(2S,6R)-2-[[4-[2-[[(3S,4R)-4-fluoropyrrolidin-3-yl]amino]pyrimidin-4-yl]piperazin-1-yl]methyl]-6-methyl-morpholin-4-yl]quinoline-8-carbonitrile;
5-[(2S,6R)-2-[[4-[2-(6-hydroxy-1,4-diazepan-1-yl)pyrimidin-4-yl]piperazin-1-yl]methyl]-6-methyl-morpholin-4-yl]quinoline-8-carbonitrile;
5-[(2S,6R)-2-[[4-[2-trans-(3-amino-4-methoxy-pyrrolidin-1-yl)pyrimidin-4-yl]piperazin-1-yl]methyl]-6-methyl-morpholin-4-yl]quinoline-8-carbonitrile;
5-[(2S,6R)-2-[[4-[2-(4-amino-1-piperidyl)pyrimidin-4-yl]piperazin-1-yl]methyl]-6-methyl-morpholin-4-yl]quinoline-8-carbonitrile;
5-[(2S,6R)-2-[[4-[2-(azetidin-3-ylamino)pyrimidin-4-yl]piperazin-1-yl]methyl]-6-methyl-morpholin-4-yl]quinoline-8-carbonitrile;
5-[(2S,6R)-2-[[4-[2-[(3R)-3-(hydroxymethyl)piperazin-1-yl]pyrimidin-4-yl]piperazin-1-yl]methyl]-6-methyl-morpholin-4-yl]quinoline-8-carbonitrile;
5-[(2S,6R)-2-[[4-[2-[2-(dimethylamino)ethoxy]pyrimidin-4-yl]piperazin-1-yl]methyl]-6-methyl-morpholin-4-yl]quinoline-8-carbonitrile;
5-[(2R,6S)-2-methyl-6-[[4-[2-[4-[(2S)-pyrrolidine-2-carbonyl]piperazin-1-yl]pyrimidin-4-yl]piperazin-1-yl]methyl]morpholin-4-yl]quinoline-8-carbonitrile;
5-[(2S,6R)-2-[[4-[2-[4-[2-(dimethylamino)acetyl]piperazin-1-yl]pyrimidin-4-yl]piperazin-1-yl]methyl]-6-methyl-morpholin-4-yl]quinoline-8-carbonitrile;
5-[(2R,6S)-2-methyl-6-[[4-[6-(4-methylpiperazin-1-yl)-2-pyridyl]piperazin-1-yl]methyl]morpholin-4-yl]quinoline-8-carbonitrile;
5-[(2S,6R)-2-[[4-[2-[4-(azetidine-2-carbonyl)piperazin-1-yl]pyrimidin-4-yl]piperazin-1-yl]methyl]-6-methyl-morpholin-4-yl]quinoline-8-carbonitrile;
5-[(2S,6R)-2-[[4-[6-[[(3S,4S)-4-fluoropyrrolidin-3-yl]amino]-2-pyridyl]piperazin-1-yl]methyl]-6-methyl-morpholin-4-yl]quinoline-8-carbonitrile;
5-[(2S,6R)-2-[[4-[6-(4-amino-1-piperidyl)-2-pyridyl]piperazin-1-yl]methyl]-6-methyl-morpholin-4-yl]quinoline-8-carbonitrile;
5-[(2S,6R)-2-[[4-[6-[[(3S,4R)-4-fluoropyrrolidin-3-yl]amino]-2-pyridyl]piperazin-1-yl]methyl]-6-methyl-morpholin-4-yl]quinoline-8-carbonitrile;
5-[(2S,6R)-2-[[4-[6-(4-amino-1-piperidyl)-2-pyridyl]piperazin-1-yl]methyl]-6-methyl-morpholin-4-yl]quinoline-8-carbonitrile;
5-[(2S,6R)-2-[[4-[6-[[(3R,4R)-4-methoxypyrrolidin-3-yl]amino]-2-pyridyl]piperazin-1-yl]methyl]-6-methyl-morpholin-4-yl]quinoline-8-carbonitrile;
5-[(2S,6R)-2-[[4-[6-(3-amino-4-fluoro-pyrrolidin-1-yl)-2-pyridyl]piperazin-1-yl]methyl]-6-methyl-morpholin-4-yl]quinoline-8-carbonitrile;
5-[(2S,6R)-2-[[4-[6-(6-hydroxy-1,4-diazepan-1-yl)-2-pyridyl]piperazin-1-yl]methyl]-6-methyl-morpholin-4-yl]quinoline-8-carbonitrile;
5-[(2S,6R)-2-[[4-[6-[(3R,4S)-3-amino-4-methoxy-pyrrolidin-1-yl]-2-pyridyl]piperazin-1-yl]methyl]-6-methyl-morpholin-4-yl]quinoline-8-carbonitrile;
5-[(2S,6R)-2-[[4-[6-(3-amino-4-methoxy-pyrrolidin-1-yl)-3-pyridyl]piperazin-1-yl]methyl]-6-methyl-morpholin-4-yl]quinoline-8-carbonitrile;
5-[(2S,6R)-2-[[4-[6-(3-amino-4-fluoro-pyrrolidin-1-yl)-3-pyridyl]piperazin-1-yl]methyl]-6-methyl-morpholin-4-yl]quinoline-8-carbonitrile;
5-[(2S,6R)-2-[[4-(5-fluoro-2-piperazin-1-yl-pyrimidin-4-yl)piperazin-1-yl]methyl]-6-methyl-morpholin-4-yl]quinoline-8-carbonitrile;
5-[(2S,6R)-2-[[4-(5,6-dimethyl-2-piperazin-1-yl-pyrimidin-4-yl)piperazin-1-yl]methyl]-6-methyl-morpholin-4-yl]quinoline-8-carbonitrile;
5-[(2R,6S)-2-methyl-6-[[4-(6-methyl-2-piperazin-1-yl-pyrimidin-4-yl)piperazin-1-yl]methyl]morpholin-4-yl]quinoline-8-carbonitrile;
5-[(2S,6R)-2-[[4-(5-methoxy-2-piperazin-1-yl-pyrimidin-4-yl)piperazin-1-yl]methyl]-6-methyl-morpholin-4-yl]quinoline-8-carbonitrile;
5-[(2R,6S)-2-methyl-6-[[4-(2-piperazin-1-ylquinazolin-4-yl)piperazin-1-yl]methyl]morpholin-4-yl]quinoline-8-carbonitrile;
5-[(2S,6R)-2-[[4-(5-cyano-2-piperazin-1-yl-pyrimidin-4-yl)piperazin-1-yl]methyl]-6-methyl-morpholin-4-yl]quinoline-8-carbonitrile;
5-[(2S,6R)-2-[[4-(5-cyano-4-piperazin-1-yl-pyrimidin-2-yl)piperazin-1-yl]methyl]-6-methyl-morpholin-4-yl]quinoline-8-carbonitrile;
5-[(2S,6R)-2-[[4-[2-[(3R,4R)-3-amino-4-methoxy-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[2-[(3S,4S)-3-amino-4-methoxy-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[2-[(3S,4R)-3-amino-4-fluoro-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4S)-3-amino-4-fluoro-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-(6-amino-1,4-oxazepan-4-yl)-6-methyl-pyrimidin-4-yl]piperazin-1-yl]methyl]-6-methyl-morpholin-4-yl]quinoline-8-carbonitrile;
5-[(2S,6R)-2-[[4-[2-[(4-cyanopyrrolidin-3-yl)amino]-6-methyl-pyrimidin-4-yl]piperazin-1-yl]methyl]-6-methyl-morpholin-4-yl]quinoline-8-carbonitrile;
5-[(2R,6S)-2-methyl-6-[[4-[6-methyl-2-[(1R,5R)-3-oxa-7,9-diazabicyclo[3.3.1]nonan-7-yl]pyrimidin-4-yl]piperazin-1-yl]methyl]morpholin-4-yl]quinoline-8-carbonitrile;
5-[(2R,6S)-2-methyl-6-[[4-[6-methyl-2-[trans-3-amino-4-methoxy-pyrrolidin-1-yl]pyrimidin-4-yl]piperazin-1-yl]methyl]morpholin-4-yl]quinoline-8-carbonitrile;
5-[(2R,6S)-2-methyl-6-[[4-[6-methyl-2-[cis-3-amino-4-methoxy-pyrrolidin-1-yl]pyrimidin-4-yl]piperazin-1-yl]methyl]morpholin-4-yl]quinoline-8-carbonitrile;
5-[(2R,6S)-2-methyl-6-[[4-[6-methyl-2-(5-oxa-2,8-diazaspiro[3.5]nonan-2-yl)pyrimidin-4-yl]piperazin-1-yl]methyl]morpholin-4-yl]quinoline-8-carbonitrile;
5-[(2R,6S)-2-methyl-6-[[4-(3-morpholin-2-ylphenyl)piperazin-1-yl]methyl]-morpholin-4-yl]quinoline-8-carbonitrile;
5-[(2S,6R)-2-[[4-[2-(4-amino-3,3-difluoro-pyrrolidin-1-yl)-6-methyl-pyrimidin-4-yl]piperazin-1-yl]methyl]-6-methyl-morpholin-4-yl]quinoline-8-carbonitrile;
8-[(2R,6S)-2-methyl-6-[[4-(6-methyl-2-piperazin-1-yl-pyrimidin-4-yl)piperazin-1-yl]methyl]morpholin-4-yl]quinoxaline-5-carbonitrile;
(2R,6S)-2-methyl-6-[[4-(6-methyl-2-piperazin-1-yl-pyrimidin-4-yl)piperazin-1-yl]methyl]-4-[8-(trifluoromethyl)quinoxalin-5-yl]morpholine;
5-[(2S,6R)-2-[[4-[2-(6-hydroxy-1,4-diazepan-1-yl)-6-methyl-pyrimidin-4-yl]piperazin-1-yl]methyl]-6-methyl-morpholin-4-yl]quinoline-8-carbonitrile;
3-Fluoro-4-[(2R,6S)-2-methyl-6-[[4-(6-methyl-2-piperazin-1-yl-pyrimidin-4-yl)piperazin-1-yl]methyl]morpholin-4-yl]pyrazolo[1,5-a]pyridine-7-carbonitrile;
5-[(2R,6S)-2-methyl-6-[[4-(4-methyl-6-piperazin-1-yl-pyrimidin-2-yl)piperazin-1-yl]methyl]morpholin-4-yl]quinoline-8-carbonitrile;
5-[(2S,6R)-2-[[4-[2-trans-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3S,4S)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4R)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[[(3R,4R)-4-methoxypyrrolidin-3-yl]amino]-6-methyl-pyrimidin-4-yl]piperazin-1-yl]methyl]-6-methyl-morpholin-4-yl]quinoline-8-carbonitrile;
5-[(2S,6R)-2-[[4-[2-[[(3R,4S)-4-fluoropyrrolidin-3-yl]amino]-6-methyl-pyrimidin-4-yl]piperazin-1-yl]methyl]-6-methyl-morpholin-4-yl]quinoline-8-carbonitrile;
5-[(2S,6R)-2-[[4-[2-[(3S,4R)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[[(3S,4R)-4-fluoropyrrolidin-3-yl]amino]-6-methyl-pyrimidin-4-yl]piperazin-1-yl]methyl]-6-methyl-morpholin-4-yl]quinoline-8-carbonitrile;
5-[(2R,6S)-2-methyl-6-[[4-[6-methyl-2-[cis-3-amino-5-fluoro-1-piperidyl]pyrimidin-4-yl]piperazin-1-yl]methyl]morpholin-4-yl]quinoline-8-carbonitrile;
5-[(2R,6S)-2-methyl-6-[[4-[6-methyl-2-[trans-3-amino-5-fluoro-1-piperidyl]pyrimidin-4-yl]piperazin-1-yl]methyl]morpholin-4-yl]quinoline-8-carbonitrile;
5-[(2S,6R)-2-[[4-[2-[[(3R,4R)-4-fluoropyrrolidin-3-yl]amino]-6-methyl-pyrimidin-4-yl]piperazin-1-yl]methyl]-6-methyl-morpholin-4-yl]quinoline-8-carbonitrile;
5-[(2S,6R)-2-[[4-[2-[(3S,5R)-3-amino-5-fluoro-1-piperidyl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3S,4R)-4-amino-3-fluoro-1-piperidyl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4R)-3-amino-4-fluoro-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-(5-amino-3,3-difluoro-1-piperidyl)-6-methyl-pyrimidin-4-yl]piperazin-1-yl]methyl]-6-methyl-morpholin-4-yl]quinoline-8-carbonitrile;
5-[(2S,6R)-2-[[4-[2-[3-(1-hydroxy-1-methyl-ethyl)piperazin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4R)-4-amino-3-fluoro-1-piperidyl]-6-methyl-pyrimidin-4-yl]piperazin-1-yl]methyl]-6-methyl-morpholin-4-yl]quinoline-8-carbonitrile;
5-[(2R,6S)-2-methyl-6-[[4-[6-methyl-2-[(3S,4R)-3-amino-4-fluoro-1-piperidyl]-pyrimidin-4-yl]piperazin-1-yl]methyl]morpholin-4-yl]quinoline-8-carbonitrile;
5-[(2R,6S)-2-methyl-6-[[4-[6-methyl-2-[(3R,4R)-3-amino-4-fluoro-1-piperidyl]pyrimidin-4-yl]piperazin-1-yl]methyl]morpholin-4-yl]quinoline-8-carbonitrile;
5-[(2S,6R)-2-[[4-[2-(azetidin-3-ylamino)-6-methyl-pyrimidin-4-yl]piperazin-1-yl]methyl]-6-methyl-morpholin-4-yl]quinoline-8-carbonitrile;
5-[(2S,6R)-2-[[4-[2-(4-amino-3,3-difluoro-1-piperidyl)-6-methyl-pyrimidin-4-yl]piperazin-1-yl]methyl]-6-methyl-morpholin-4-yl]quinoline-8-carbonitrile;
5-[(2S,6R)-2-[[4-[2-(3-aminoazetidin-1-yl)-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4S)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2R,6S)-2-methyl-6-[[4-(2-morpholin-2-ylphenyl)piperazin-1-yl]methyl]morpholin-4-yl]quinoline-8-carbonitrile;
5-[(2S,6R)-2-[[4-[2-[(3S,4S)-4-amino-3-methoxy-1-piperidyl]-6-methyl-pyrimidin-4-yl]piperazin-1-yl]methyl]-6-methyl-morpholin-4-yl]quinoline-8-carbonitrile;
5-[(2S,6R)-2-[[4-(6-methoxy-2-piperazin-1-yl-pyrimidin-4-yl)piperazin-1-yl]methyl]-6-methyl-morpholin-4-yl]quinoline-8-carbonitrile;
5-[(2S,6R)-2-[[4-[2-[(3S,4R)-3-amino-4-methoxy-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[2-[(3R,4S)-3-amino-4-methoxy-pyrrolidin-1-yl]pyrimidin-4-yl]piperazin-1-yl]methyl]-6-methyl-morpholin-4-yl]quinoline-8-carbonitrile;
5-[(2S,6R)-2-[[4-(5-fluoro-4-piperazin-1-yl-pyrimidin-2-yl)piperazin-1-yl]methyl]-6-methyl-morpholin-4-yl]quinoline-8-carbonitrile;
5-[(2S,6R)-2-[[4-[2-[(3S,4R)-3-amino-4-fluoro-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[2-[(3R,4R)-3-amino-4-fluoro-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[4-[(3S,4S)-3-amino-4-methoxy-pyrrolidin-1-yl]pyrimidin-2-yl]piperazin-1-yl]methyl]-6-methyl-morpholin-4-yl]quinoline-8-carbonitrile;
8-[(2S,6R)-2-[[4-[2-[(3R,4R)-3-amino-4-methoxy-pyrrolidin-1-yl]pyrimidin-4-yl]piperazin-1-yl]methyl]-6-methyl-morpholin-4-yl]quinoxaline-5-carbonitrile;
8-[(2S,6R)-2-[[4-[2-[(3S,4S)-3-amino-4-methoxy-pyrrolidin-1-yl]pyrimidin-4-yl]piperazin-1-yl]methyl]-6-methyl-morpholin-4-yl]quinoxaline-5-carbonitrile;
5-[(2S,6R)-2-[[4-[2-[(3R,4S)-3-amino-4-fluoro-pyrrolidin-1-yl]pyrimidin-4-yl]piperazin-1-yl]methyl]-6-methyl-morpholin-4-yl]quinoline-8-carbonitrile;
5-[(2R,6S)-2-methyl-6-[[4-(5-methyl-4-piperazin-1-yl-pyrimidin-2-yl)piperazin-1-yl]methyl]morpholin-4-yl]quinoline-8-carbonitrile;
5-[(2S,6R)-2-[[4-[2-[(4aR,7aR)-3,4,4a,5,7,7a-hexahydro-2H-pyrrolo[3,4-b][1,4]oxazin-6-yl]pyrimidin-4-yl]piperazin-1-yl]methyl]-6-methyl-morpholin-4-yl]quinoline-8-carbonitrile;
8-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile;
8-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]-2,3-dideuterio-quinoxaline-5-carbonitrile;
8-[(2S,6R)-2-[[4-[2-[(3S,4S)-3-amino-4-methoxy-pyrrolidin-1-yl]-6-methyl-pyrimidin-4-yl]piperazin-1-yl]methyl]-6-methyl-morpholin-4-yl]quinoxaline-5-carbonitrile;
8-[(2S,6R)-2-[[4-[2-[(3S,4S)-3-amino-4-fluoro-pyrrolidin-1-yl]pyrimidin-4-yl]piperazin-1-yl]methyl]-6-methyl-morpholin-4-yl]quinoxaline-5-carbonitrile;
8-[(2S,6R)-2-[[4-[2-[(3R,4S)-3-amino-4-fluoro-pyrrolidin-1-yl]pyrimidin-4-yl]piperazin-1-yl]methyl]-6-methyl-morpholin-4-yl]quinoxaline-5-carbonitrile;
5-[(2S,6R)-2-[[4-[2-(3-amino-4-methoxy-1-piperidyl)pyrimidin-4-yl]piperazin-1-yl]methyl]-6-methyl-morpholin-4-yl]quinoline-8-carbonitrile;
5-[(2S,6R)-2-[[4-[2-[(3S,4S)-3-amino-4-fluoro-pyrrolidin-1-yl]pyrimidin-4-yl]piperazin-1-yl]methyl]-6-methyl-morpholin-4-yl]quinoline-8-carbonitrile;
8-[(2S,6R)-2-[[4-[2-[(3R,4S)-4-amino-3-fluoro-1-piperidyl]pyrimidin-4-yl]piperazin-1-yl]methyl]-6-methyl-morpholin-4-yl]quinoxaline-5-carbonitrile;
8-[(2S,6R)-2-[[4-[2-(3-aminoazetidin-1-yl)-6-methyl-pyrimidin-4-yl]piperazin-1-yl]methyl]-6-methyl-morpholin-4-yl]quinoxaline-5-carbonitrile;
8-[(2S,6R)-2-[[4-[2-[(3R,4R)-4-amino-3-fluoro-1-piperidyl]pyrimidin-4-yl]piperazin-1-yl]methyl]-6-methyl-morpholin-4-yl]quinoxaline-5-carbonitrile;
8-[(2R,6S)-2-methyl-6-[[4-(4-methyl-6-piperazin-1-yl-pyrimidin-2-yl)piperazin-1-yl]methyl]morpholin-4-yl]quinoxaline-5-carbonitrile;
8-[(2S,6R)-2-[[4-[4-[(3R,4S)-3-amino-4-fluoro-pyrrolidin-1-yl]-6-methyl-pyrimidin-2-yl]piperazin-1-yl]methyl]-6-methyl-morpholin-4-yl]quinoxaline-5-carbonitrile;
(2R,6S)-2-methyl-4-(8-methylquinoxalin-5-yl)-6-[[4-(4-piperazin-1-ylpyrimidin-2-yl)piperazin-1-yl]methyl]morpholine;
(2R,6S)-2-methyl-4-(8-methylquinoxalin-5-yl)-6-[[4-(2-piperazin-1-ylpyrimidin-4-yl)piperazin-1-yl]methyl]morpholine;
8-[(2S,6R)-2-[[4-[4-(3-aminoazetidin-1-yl)pyrimidin-2-yl]piperazin-1-yl]methyl]-6-methyl-morpholin-4-yl]quinoxaline-5-carbonitrile;
8-[(2S,6R)-2-[[4-[4-[(3R,4R)-3-amino-4-fluoro-pyrrolidin-1-yl]pyrimidin-2-yl]piperazin-1-yl]methyl]-6-methyl-morpholin-4-yl]quinoxaline-5-carbonitrile;
8-[(2S,6R)-2-[[4-[4-[(3S,4R)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile;
8-[(2S,6R)-2-[[4-[4-[(3R,4R)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile;
8-[(2S,6R)-2-[[4-[4-[(3R,4S)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile;
8-[(2S,6R)-2-[[4-[4-(3-aminoazetidin-1-yl)-6-methyl-pyrimidin-2-yl]piperazin-1-yl]methyl]-6-methyl-morpholin-4-yl]quinoxaline-5-carbonitrile;
8-[(2S,6R)-2-[[4-[4-(3-aminoazetidin-1-yl)-6-methyl-pyrimidin-2-yl]piperazin-1-yl]methyl]-6-methyl-morpholin-4-yl]-2,3-dideuterio-quinoxaline-5-carbonitrile;
8-[(2S,6R)-2-[[4-[4-[(3R,4R)-4-amino-3-hydroxy-3-methyl-pyrrolidin-1-yl]-6-methyl-pyrimidin-2-yl]piperazin-1-yl]methyl]-6-methyl-morpholin-4-yl]quinoxaline-5-carbonitrile;
2-[4-[4-[[(2S,6R)-6-methyl-4-(8-methylquinoxalin-5-yl)morpholin-2-yl]methyl]piperazin-1-yl]pyrimidin-2-yl]-5-oxa-2,8-diazaspiro[3.5]nonan;
(2R,6S)-2-methyl-6-[[4-[2-[(3S)-3-methylpiperazin-1-yl]pyrimidin-4-yl]piperazin-1-yl]methyl]-4-(8-methylquinoxalin-5-yl)morpholine;
(2R,6S)-2-methyl-6-[[4-[2-[(3R)-3-methylpiperazin-1-yl]pyrimidin-4-yl]piperazin-1-yl]methyl]-4-(8-methylquinoxalin-5-yl)morpholine;
(4aR,7aR)-6-[4-[4-[[(2S,6R)-6-methyl-4-(8-methylquinoxalin-5-yl)morpholin-2-yl]methyl]piperazin-1-yl]pyrimidin-2-yl]-3,4,4a,5,7,7a-hexahydro-2H-pyrrolo[3,4-b][1,4]oxazine;
1-[4-[4-[[(2S,6R)-6-methyl-4-(8-methylquinoxalin-5-yl)morpholin-2-yl]methyl]piperazin-1-yl]pyrimidin-2-yl]azetidin-3-amine;
5-[(2S,6R)-2-[[4-[4-(3-amino-3-methyl-azetidin-1-yl)-6-methoxy-pyrimidin-2-yl]piperazin-1-yl]methyl]-6-methyl-morpholin-4-yl]-2-deuterio-quinoline-8-carbonitrile;
5-[(2S,6R)-2-[[4-[6-(3-amino-3-methyl-azetidin-1-yl)-3-pyridyl]piperazin-1-yl]methyl]-6-methyl-morpholin-4-yl]-2-deuterio-quinoline-8-carbonitrile;
5-[(2S,6R)-2-[[4-[6-(3-amino-3-methyl-azetidin-1-yl)-4-methyl-3-pyridyl]piperazin-1-yl]methyl]-6-methyl-morpholin-4-yl]-2-deuterio-quinoline-8-carbonitrile;
5-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]-2-deuterio-quinoline-8-carbonitrile;
5-[(2S,6R)-2-[[4-[4-(3-amino-3-methyl-azetidin-1-yl)-6-methyl-pyrimidin-2-yl]piperazin-1-yl]methyl]-6-methyl-morpholin-4-yl]-2-deuterio-quinoline-8-carbonitrile;
5-[(2S,6R)-2-[[4-[4-(3-aminoazetidin-1-yl)-6-methyl-pyrimidin-2-yl]piperazin-1-yl]methyl]-6-methyl-morpholin-4-yl]-2-deuterio-quinoline-8-carbonitrile;
5-[(2S,6R)-2-[[4-[6-(3-amino-3-methyl-azetidin-1-yl)-2-methyl-3-pyridyl]piperazin-1-yl]methyl]-6-methyl-morpholin-4-yl]-2-deuterio-quinoline-8-carbonitrile;
5-[(2S,6R)-2-[[3-[6-(3-amino-3-methyl-azetidin-1-yl)-3-pyridyl]azetidin-1-yl]methyl]-6-methyl-morpholin-4-yl]-2-deuterio-quinoline-8-carbonitrile;
5-[(2S,6R)-2-[[4-[5-(3-amino-3-methyl-azetidin-1-yl)-3-methyl-2-pyridyl]piperazin-1-yl]methyl]-6-methyl-morpholin-4-yl]-2-deuterio-quinoline-8-carbonitrile;
5-[(2S,6R)-2-[[3-[4-[(3S,4S)-3-amino-4-methoxy-pyrrolidin-1-yl]phenyl]azetidin-1-yl]methyl]-6-methyl-morpholin-4-yl]-2-deuterio-quinoline-8-carbonitrile;
5-[(2S,6R)-2-[[3-[4-(3-amino-3-methyl-azetidin-1-yl)phenyl]azetidin-1-yl]methyl]-6-methyl-morpholin-4-yl]-2-deuterio-quinoline-8-carbonitrile;
5-[(2S,6R)-2-[[4-[5-[(3R,4R)-3-amino-4-methoxy-pyrrolidin-1-yl]-3-methyl-2-pyridyl]piperazin-1-yl]methyl]-6-methyl-morpholin-4-yl]-2-deuterio-quinoline-8-carbonitrile;
5-[(2S,6R)-2-[[4-[6-[(3R,4R)-3-amino-4-methoxy-pyrrolidin-1-yl]-3-pyridyl]piperazin-1-yl]methyl]-6-methyl-morpholin-4-yl]-2-deuterio-quinoline-8-carbonitrile;
5-[(2S,6R)-2-[[4-[4-[(3R,4R)-3-amino-4-methoxy-pyrrolidin-1-yl]-6-methyl-pyrimidin-2-yl]piperazin-1-yl]methyl]-6-methyl-morpholin-4-yl]-2-deuterio-quinoline-8-carbonitrile;
5-[(2S,6R)-2-[[3-[6-(3-amino-3-methyl-azetidin-1-yl)-4-methyl-3-pyridyl]azetidin-1-yl]methyl]-6-methyl-morpholin-4-yl]-2-deuterio-quinoline-8-carbonitrile;
5-[(2S,6R)-2-[[3-[6-[(3R,4R)-3-amino-4-methoxy-pyrrolidin-1-yl]-4-methyl-3-pyridyl]azetidin-1-yl]methyl]-6-methyl-morpholin-4-yl]-2-deuterio-quinoline-8-carbonitrile;
5-[(2S,6R)-2-[[4-[5-(3-amino-3-methyl-azetidin-1-yl)-6-methyl-2-pyridyl]piperazin-1-yl]methyl]-6-methyl-morpholin-4-yl]-2-deuterio-quinoline-8-carbonitrile;
4-[(2S,6R)-2-[[4-[5-(3-amino-3-methyl-azetidin-1-yl)-3-methyl-2-pyridyl]piperazin-1-yl]methyl]-6-methyl-morpholin-4-yl]-3-fluoro-pyrazolo[1,5-a]pyridine-7-carbonitrile;
5-[(2S,6R)-2-[[3-[6-[(3R,4R)-3-amino-4-methoxy-pyrrolidin-1-yl]-3-pyridyl]azetidin-1-yl]methyl]-6-methyl-morpholin-4-yl]-2-deuterio-quinoline-8-carbonitrile;
5-[(2S,6R)-2-[[4-[6-[(3S,4S)-3-amino-4-fluoro-pyrrolidin-1-yl]-3-pyridyl]piperazin-1-yl]methyl]-6-methyl-morpholin-4-yl]-2-deuterio-quinoline-8-carbonitrile;
2-deuterio-5-[(2S,6R)-2-[[4-[6-[(1S,4S)-2,5-diazabicyclo[2.2.1]heptan-2-yl]-3-pyridyl]piperazin-1-yl]methyl]-6-methyl-morpholin-4-yl]quinoline-8-carbonitrile;
2-deuterio-5-[(2R,6S)-2-methyl-6-[[4-[6-(5-oxa-2,8-diazaspiro[3.5]nonan-2-yl)-3-pyridyl]piperazin-1-yl]methyl]morpholin-4-yl]quinoline-8-carbonitrile;
2-deuterio-5-[(2S,6R)-2-[[4-[6-[(1R,4R)-2,5-diazabicyclo[2.2.1]heptan-2-yl]-3-pyridyl]piperazin-1-yl]methyl]-6-methyl-morpholin-4-yl]quinoline-8-carbonitrile;
4-[(2S,6R)-2-[[4-[6-(3-amino-3-methyl-azetidin-1-yl)-3-pyridyl]piperazin-1-yl]methyl]-6-methyl-morpholin-4-yl]-3-fluoro-pyrazolo[1,5-a]pyridine-7-carbonitrile;
5-[(2S,6R)-2-[[4-[6-(3-amino-3-methyl-azetidin-1-yl)-2-pyridyl]piperazin-1-yl]methyl]-6-methyl-morpholin-4-yl]-2-deuterio-quinoline-8-carbonitrile;
5-[(2S,6R)-2-[[3-[4-(3-amino-3-methyl-azetidin-1-yl)phenyl]-3-hydroxy-azetidin-1-yl]methyl]-6-methyl-morpholin-4-yl]-2-deuterio-quinoline-8-carbonitrile;
4-[(2S,6R)-2-[[3-[6-(3-amino-3-methyl-azetidin-1-yl)-3-pyridyl]azetidin-1-yl]methyl]-6-methyl-morpholin-4-yl]pyrazolo[1,5-a]pyridine-7-carbonitrile;
4-[(2S,6R)-2-[[3-[6-(3-amino-3-methyl-azetidin-1-yl)-3-pyridyl]azetidin-1-yl]methyl]-6-methyl-morpholin-4-yl]-3-fluoro-pyrazolo[1,5-a]pyridine-7-carbonitrile;
5-[(2S,6R)-2-[[3-[6-(3-amino-3-methyl-azetidin-1-yl)-2-pyridyl]azetidin-1-yl]methyl]-6-methyl-morpholin-4-yl]-2-deuterio-quinoline-8-carbonitrile;
4-[(2S,6R)-2-[[3-[4-(3-amino-3-methyl-azetidin-1-yl)phenyl]azetidin-1-yl]methyl]-6-methyl-morpholin-4-yl]-3-fluoro-pyrazolo[1,5-a]pyridine-7-carbonitrile;
2-deuterio-5-[(2R,6S)-2-methyl-6-[[4-[6-(6-methyl-2,6-diazaspiro[3.3]heptan-2-yl)-3-pyridyl]piperazin-1-yl]methyl]morpholin-4-yl]quinoline-8-carbonitrile;
2-deuterio-5-[(2S,6R)-2-[[4-[6-[[(3S,4R)-4-fluoropyrrolidin-3-yl]amino]-3-pyridyl]piperazin-1-yl]methyl]-6-methyl-morpholin-4-yl]quinoline-8-carbonitrile;
4-[(2S,6R)-2-[[3-[4-(3-amino-3-methyl-azetidin-1-yl)phenyl]azetidin-1-yl]methyl]-6-methyl-morpholin-4-yl]pyrazolo[1,5-a]pyridine-7-carbonitrile;
4-[(2S,6R)-2-[[3-[6-[(3R,4R)-3-amino-4-methoxy-pyrrolidin-1-yl]-3-pyridyl]azetidin-1-yl]methyl]-6-methyl-morpholin-4-yl]-3-fluoro-pyrazolo[1,5-a]pyridine-7-carbonitrile;
4-[(2S,6R)-2-[[3-[6-[(1S,4S)-2,5-diazabicyclo[2.2.1]heptan-2-yl]-3-pyridyl]azetidin-1-yl]methyl]-6-methyl-morpholin-4-yl]-3-fluoro-pyrazolo[1,5-a]pyridine-7-carbonitrile;
4-[(2S,6R)-2-[[4-[6-[(3R,4R)-3-amino-4-methoxy-pyrrolidin-1-yl]-3-pyridyl]piperazin-1-yl]methyl]-6-methyl-morpholin-4-yl]-3-fluoro-pyrazolo[1,5-a]pyridine-7-carbonitrile;
3-fluoro-4-[(2S,6R)-2-[[4-[6-[[(3S,4R)-4-fluoropyrrolidin-3-yl]amino]-3-pyridyl]piperazin-1-yl]methyl]-6-methyl-morpholin-4-yl]pyrazolo[1,5-a]pyridine-7-carbonitrile;
4-[(2S,6R)-2-[[4-[6-[(4aR,7aR)-3,4,4a,5,7,7a-hexahydro-2H-pyrrolo[3,4-b][1,4]oxazin-6-yl]-3-pyridyl]piperazin-1-yl]methyl]-6-methyl-morpholin-4-yl]-3-fluoro-pyrazolo[1,5-a]pyridine-7-carbonitrile;
3-fluoro-4-[(2R,6S)-2-methyl-6-[[4-[6-(5-oxa-2,8-diazaspiro[3.5]nonan-2-yl)-3-pyridyl]piperazin-1-yl]methyl]morpholin-4-yl]pyrazolo[1,5-a]pyridine-7-carbonitrile;
3-fluoro-4-[(2R,6S)-2-methyl-6-[[4-[6-(6-methyl-2,6-diazaspiro[3.3]heptan-2-yl)-3-pyridyl]piperazin-1-yl]methyl]morpholin-4-yl]pyrazolo[1,5-a]pyridine-7-carbonitrile;
1-methyl-4-[(2R,6S)-2-methyl-6-[[3-(4-piperazin-1-ylphenyl)azetidin-1-yl]methyl]morpholin-4-yl]-1,8-naphthyridin-2-one;
4-[(2S,6R)-2-[[3-[4-[(3R,4R)-3-amino-4-methoxy-pyrrolidin-1-yl]phenyl]azetidin-1-yl]methyl]-6-methyl-morpholin-4-yl]-1-methyl-1,8-naphthyridin-2-one;
1-methyl-4-[(2R,6S)-2-methyl-6-[[3-[4-(5-oxa-2,8-diazaspiro[3.5]nonan-2-yl)phenyl]azetidin-1-yl]methyl]morpholin-4-yl]-1,8-naphthyridin-2-one;
4-[(2S,6R)-2-[[3-[4-[(3R,4R)-3-amino-4-fluoro-pyrrolidin-1-yl]phenyl]azetidin-1-yl]methyl]-6-methyl-morpholin-4-yl]-1-methyl-1,8-naphthyridin-2-one;
4-[(2S,6R)-2-[[4-[6-[(3R,4R)-4-amino-3-fluoro-1-piperidyl]-3-pyridyl]piperazin-1-yl]methyl]-6-methyl-morpholin-4-yl]-3-fluoro-pyrazolo[1,5-a]pyridine-7-carbonitrile;
4-[(2S,6R)-2-[[3-[4-(3-amino-3-methyl-azetidin-1-yl)phenyl]azetidin-1-yl]methyl]-6-methyl-morpholin-4-yl]-1-methyl-1,8-naphthyridin-2-one;
3-fluoro-4-[(2R,6S)-2-methyl-6-[[4-[4-[(2R)-morpholin-2-yl]phenyl]piperazin-1-yl]methyl]morpholin-4-yl]pyrazolo[1,5-a]pyridine-7-carbonitrile;
3-fluoro-4-[(2R,6S)-2-methyl-6-[[4-[6-(5-oxa-2,8-diazaspiro[3.5]nonan-8-yl)-3-pyridyl]piperazin-1-yl]methyl]morpholin-4-yl]pyrazolo[1,5-a]pyridine-7-carbonitrile;
4-[(2S,6R)-2-[[4-[6-(1,6-diazaspiro[3.3]heptan-6-yl)-3-pyridyl]piperazin-1-yl]methyl]-6-methyl-morpholin-4-yl]-3-fluoro-pyrazolo[1,5-a]pyridine-7-carbonitrile;
1-methyl-4-[(2R,6S)-2-methyl-6-[[3-[4-(6-methyl-2,6-diazaspiro[3.3]heptan-2-yl)phenyl]azetidin-1-yl]methyl]morpholin-4-yl]-1,8-naphthyridin-2-one;
4-[(2S,6R)-2-[[3-[4-[(4aR,7aR)-3,4,4a,5,7,7a-hexahydro-2H-pyrrolo[3,4-b][1,4]oxazin-6-yl]phenyl]azetidin-1-yl]methyl]-6-methyl-morpholin-4-yl]-1-methyl-1,8-naphthyridin-2-one;
3-fluoro-4-[(2S,6R)-2-[[4-[2-fluoro-4-[(2S)-morpholin-2-yl]phenyl]piperazin-1-yl]methyl]-6-methyl-morpholin-4-yl]pyrazolo[1,5-a]pyridine-7-carbonitrile;
3-fluoro-4-[(2S,6R)-2-[[4-[3-fluoro-4-[(2R)-morpholin-2-yl]phenyl]piperazin-1-yl]methyl]-6-methyl-morpholin-4-yl]pyrazolo[1,5-a]pyridine-7-carbonitrile;
3-fluoro-4-[(2R,6S)-2-methyl-6-[[4-[4-(2-methylmorpholin-2-yl)phenyl]piperazin-1-yl]methyl]morpholin-4-yl]pyrazolo[1,5-a]pyridine-7-carbonitrile; and
3-fluoro-4-[(2R,6S)-2-methyl-6-[[4-[4-[[(2R)-morpholin-2-yl]methyl]phenyl]piperazin-1-yl]methyl]morpholin-4-yl]pyrazolo[1,5-a]pyridine-7-carbonitrile;

or a pharmaceutically acceptable salt thereof.

In another embodiment, one or more atoms of formulas or compounds given herein are replaced by its isotope. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively.

A number of compounds used as reference herein were disclosed in patent US20150105370 showing TLR7 and TLR9 potency data summarized in table 1 (TLR8 data is not available). Compounds in Table 1 are all characterized with an aromatic ring at the terminal position (phenyl or pyridinyl). However, according to the potency data disclosed, only some of the compounds in Table 1 showed good TLR7 potency, and all of which were lack of TLR9 potency. More examples disclosed in US20150105370 with same structural characteristics confirmed such trend, which suggests the terminal aryl/heteroaryl ring is not favorable for TLR9 activity.

Meanwhile, more analogues of the compounds disclosed in US20150105370, such as compound R1, compound R2 which bear some substituents on the terminal aryl ring, were synthesized to confirm the SAR (structure-activity-relationship). But the potency of compound R1 and R2 shown in Table 2 suggested that the substituents on the terminal aryl ring may not necessarily improve the potency of TLR9. Therefore, the skill of the art shall not obtain any incitation from the information disclosed in US20150105370 to further optimize such chemical structures.

Surprisingly, the compounds of this invention significantly improved TLR9 potency (>8 folds compared to ER-888286) while keeping excellent TLR7 and TLR8 potency. In another embodiment, hERG profile and safety ratio were greatly improved as compared with reference compounds from US20150105370 and reference compounds R1 and R2 synthesized herein (see table 3). The compounds of formula (I) or (Ia) also showed good hPBMC, cytotoxicity, solubility, human microsome stability and SDPK profiles, as well as low CYP inhibition.

TABLE 1
TLR7 and TLR9 potency of compounds disclosed in US20150105370

| | | HEK/hTLR7 | HEK/hTLR9 |
|---|---|---|---|
| Compound | Structure | IC50 (μM) | IC50 (μM) |
| ER-887258 | 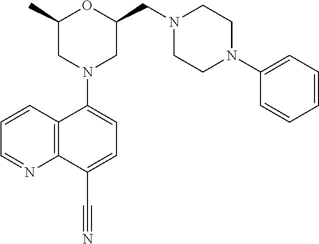 [SMILES: C[C@@H]1CN(c2ccc(C#N)c3ncccc23)C[C@H](CN2CCN(c3ccccc3)CC2)O1] | 0.0852 | >2.0 |
| ER-888285 | 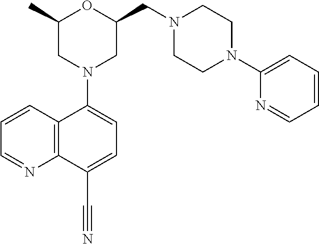 [SMILES: C[C@@H]1CN(c2ccc(C#N)c3ncccc23)C[C@H](CN2CCN(c3ccccn3)CC2)O1] | 0.120 | >2.0 |
| ER-888286 | 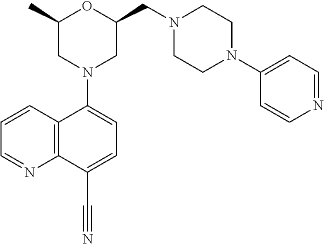 [SMILES: C[C@@H]1CN(c2ccc(C#N)c3ncccc23)C[C@H](CN2CCN(c3ccncc3)CC2)O1] | 1.370 | >2.0 |
| ER-894544 | 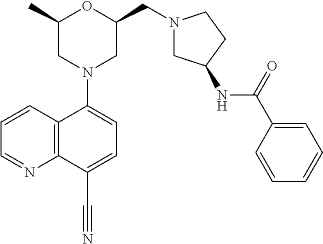 [SMILES: C[C@@H]1CN(c2ccc(C#N)c3ncccc23)C[C@H](CN2CC[C@@H](NC(=O)c3ccccc3)C2)O1] | 0.043 | >6.2 |
| ER-894160 | 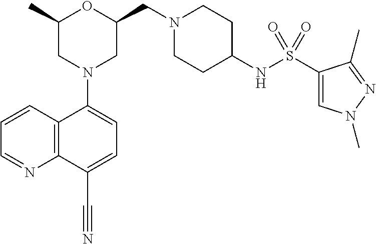 [SMILES: Cc1nn(C)cc1S(=O)(=O)NC1CCN(C[C@H]2CN(c3ccc(C#N)c4ncccc34)C[C@@H](C)O2)CC1] | 0.1990 | >10.0 |
| ER-894155 | 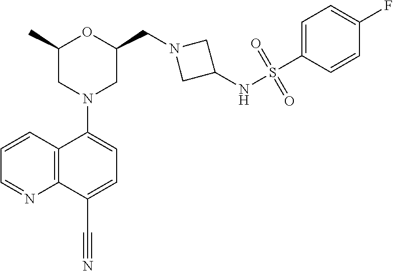 [SMILES: C[C@@H]1CN(c2ccc(C#N)c3ncccc23)C[C@H](CN2CC(NS(=O)(=O)c3ccc(F)cc3)C2)O1] | 0.2820 | >10.0 |

### SYNTHESIS

The compounds of the present invention can be prepared by any conventional means. Suitable processes for synthesizing these compounds as well as their starting materials are provided in the schemes below and in the examples. All substituents, in particular, R¹ to R³, A and Q are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) or (Ia) are shown below.

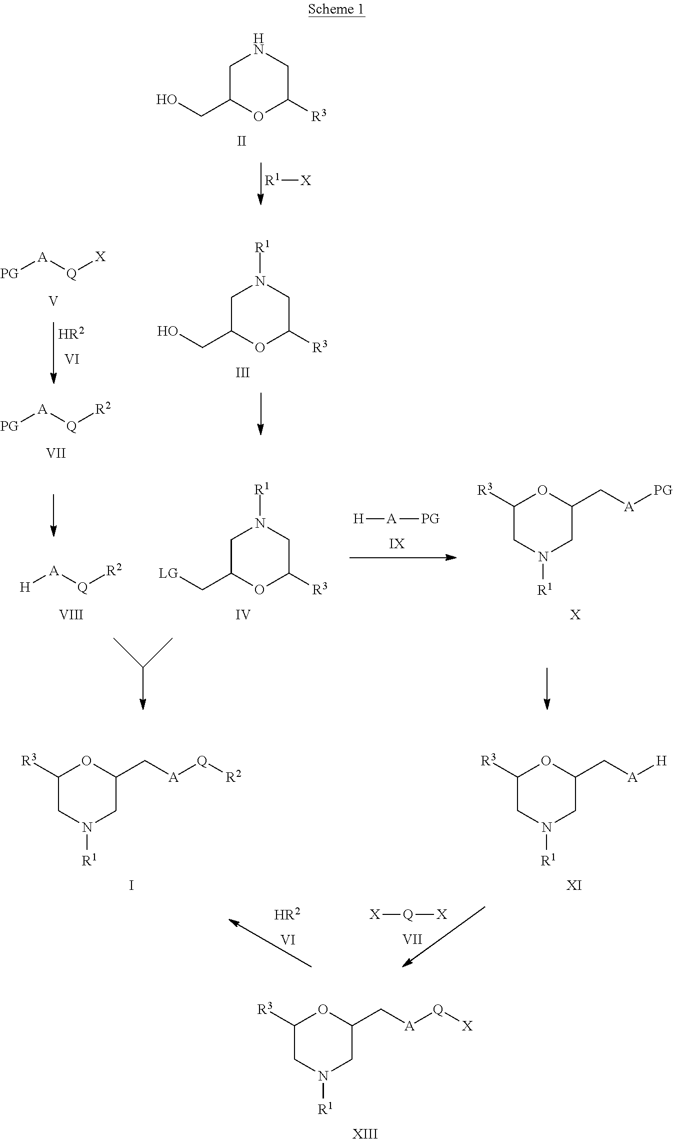

wherein X is halogen; LG is a leaving group, such as OTf, OTs and OMs; PG is a protecting group, such as Boc and Cbz.

The coupling of compound of formula (II) with R¹—X can be achieved in the presence of a base, such as DIPEA or K₂CO₃, or under Buchwald-Hartwig amination conditions (ref: Acc. Chem. Res. 1998, 31, 805-818; Chem. Rev. 2016, 116, 12564-12649; Topics in Current Chemistry, 2002, 219, 131-209; and references cited therein) with a catalyst, such as RuPhos Pd G2, and a base, such as Cs₂CO₃, to provide compound of formula (III). Subsequently the hydroxy group of compound of formula (III) is converted to a leaving group, such as OTf, OTs, and OMs, under basic condition, such as DIPEA, TEA, K₂CO₃ and 2,6-dimethylpyridine, with Tf₂O, TsCl or MsCl. The coupling of compound of formula (V) with (VI) can be achieved in the presence of a base, such as DIPEA and K₂CO₃, or under Buchwald-Hartwig amination conditions (ref: Acc. Chem. Res. 1998, 31, 805-818; Chem. Rev. 2016, 116, 12564-12649; Topics in Current Chemistry, 2002, 219, 131-209; and references cited therein) with a catalyst, such as tBuXPhos Pd G3, RuPhos Pd G2, BrettPhos Pd G3, XPhos Pd G3, Pd₂(dba)₃/BINAP and Pd₂(dba)₃/XantPhos and a base, such as Cs₂CO₃ or t-BuONa, to provide compound of formula (VII). The protecting group of compound of formula (VII) can be removed at high temperature or under acidic condition, such as TFA, or under hydrogenation condition with a catalyst, such as Pd/C and Pd(OH)₂/C. Compound of formula (VIII) is further substituted by compound of formula (IV) in the presence of a base, such as K₂CO₃, DIPEA and Cs₂CO₃, to afford compound of formula (I). On the other hand, compound of formula (IV) can reacted with (IX) in the presence of a base, such as K₂CO₃, DIPEA, and Cs₂CO₃, to give compound of formula (X). The protecting group of compound of formula (X) can be removed at high temperature or under acidic condition, such as TFA, or under hydrogenation condition with a catalyst, such as Pd/C or Pd(OH)₂/C to give compound of formula (XI), which can be further coupled with compound of formula (XII) in the presence of a base, such as DIPEA or K₂CO₃, or under Buchwald-Hartwig amination conditions (ref: Acc. Chem. Res. 1998, 31, 805-818; Chem. Rev. 2016, 116, 12564-12649; Topics in Current Chemistry, 2002, 219, 131-209; and references cited therein) with a catalyst, such as tBuXPhos Pd G3 and Ruphos Pd G2, and a base, such as Cs₂CO₃, to provide compound of formula (XIII) The coupling of compound of formula (VI) with (XIII) can be achieved in the presence of a base, such as DIPEA and K₂CO₃, or under Buchwald-Hartwig amination conditions (ref: Acc. Chem. Res. 1998, 31, 805-818; Chem. Rev. 2016, 116, 12564-12649; Topics in Current Chemistry, 2002, 219, 131-209; and references cited therein) with a catalyst, such as tBuXPhos Pd G3, RuPhos Pd G2, BrettPhos Pd G3, XPhos Pd G3, Pd₂(dba)₃/BINAP and Pd₂(dba)₃/XantPhos, and a base, such as Cs₂CO₃ and t-BuONa, to provide compound of formula (I). In some embodiment, the coupling of compound of formula (VIII) and (IV), or formula (XIII) and (VI) may give a product containing a protecting group, e.g. Boc or Cbz, originated from (VIII) or (VI), which will be removed before affording the final compound of formula (I).

Compounds of this invention can be obtained as mixtures of diastereomers or enantiomers, which can be separated by methods well known in the art, e.g. (chiral) HPLC or SFC. In another embodiment, compound of formula (Ia) can be obtained according to above scheme by using corresponding chiral starting materials.

This invention also relates to a process for the preparation of a compound of formula (I) or (Ia) comprising any of the following steps:

a) the substitution of compound of formula (IV),

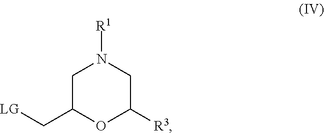

with compound of formula (VIII),

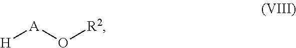

in the presence of a base;

b) the coupling of compound of formula (XIII),

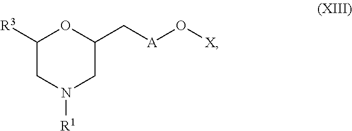

with compound of formula (VI) in the presence of a base or under Buchwald-Hartwig amination condition;

wherein

in step a) and b), the base can be, for example, K₂CO₃, DIPEA or Cs₂CO₃;

in step b), the Buchwald-Hartwig amination condition includes a catalyst and a base, wherein the catalyst can be, for example, such as tBuXPhos Pd G3, RuPhos Pd G2, BrettPhos Pd G3, XPhos Pd G3, Pd₂(dba)₃/BINAP and Pd₂(dba)₃/XantPhos; the base can be, for example, Cs₂CO₃ and t-BuONa;

A compound of formula (I) or (Ia) when manufactured according to the above process is also an object of the invention.

### Indications and Methods of Treatment

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I) or (Ia), a stereoisomer, tautomer, prodrug or pharmaceutically acceptable salt thereof.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN: acetonitrile
BINAP: (2,2′-bis(diphenylphosphino)-1,1′-binaphthyl)
BrettPhos Pd G3: [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate
t-BuXPhosPd G3: [(2-di-tert-butylphosphino-2′,4′,6′-triisopropyl-1,1′-biphenyl)-2-(2′-amino-1,1′-biphenyl)] palladium(II) methanesulfonate
DCM: dichloromethane
DIPEA: N,N-diisopropylethylamine
EtOAc or EA: ethyl acetate
HLM human liver microsome
IC₅₀: half inhibition concentration
IPA: isopropanol
LCMS liquid chromatography-mass spectrometry
MS: mass spectrometry
NMP: N-methylpyrrolidin-2-one
Boc₂O: di-tert butyl dicarbonate
Pd₂(dba)₃: tris(dibenzylideneacetone)dipalladium(0)
PE: petroleum ether
prep-HPLC: preparative high performance liquid chromatography
prep-TLC: preparative thin layer chromatography
Rf: retention factor
rt: room temperature
RuPhos Pd G2: chloro(2 dicyclohexylphosphino-2′,6′-diisopropoxy-1,1′-biphenyl) [2-(2′-amino-1,1′-biphenyl)]palladium(II)2nd generation
SFC: supercritical fluid chromatography
TEA: trimethylamine
TFA: trifluoroacetic acid
Tf₂O: trifluoromethanesulfonic anhydride
XantPhos: 4,5-bis(diphenylphosphino)-9,9-dimethylxanthene
XPhos: 2-dicyclohexylphosphino-2′,4′,6′-triisopropylbiphenyl

### GENERAL EXPERIMENTAL CONDITIONS

Intermediates and final compounds were purified by flash chromatography using one of the following instruments: i) Biotage SP1 system and the Quad 12/25 Cartridge module, ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃.H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):

Acidic condition I: A: 0.1% TFA in H₂O; B: 0.1% TFA in acetonitrile;

Acidic condition II: A: 0.0375% TFA in H₂O; B: 0.01875% TFA in acetonitrile;

Basic condition I: A: 0.1% NH₃.H₂O in H₂O; B: acetonitrile;

Basic condition II: A: 0.025% NH₃—H₂O in H₂O; B: acetonitrile;

Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Intermediate A

### [(2R,6R)-4-(8-cyano-5-quinolyl)-6-methyl-morpholin-2-yl]methyl trifluoromethanesulfonate

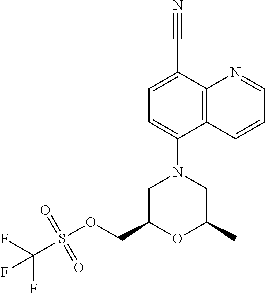 [SMILES: C[C@@H]1CN(c2ccc(C#N)c3ncccc23)C[C@H](COS(=O)(=O)C(F)(F)F)O1]

The title compound was prepared according to the following scheme:

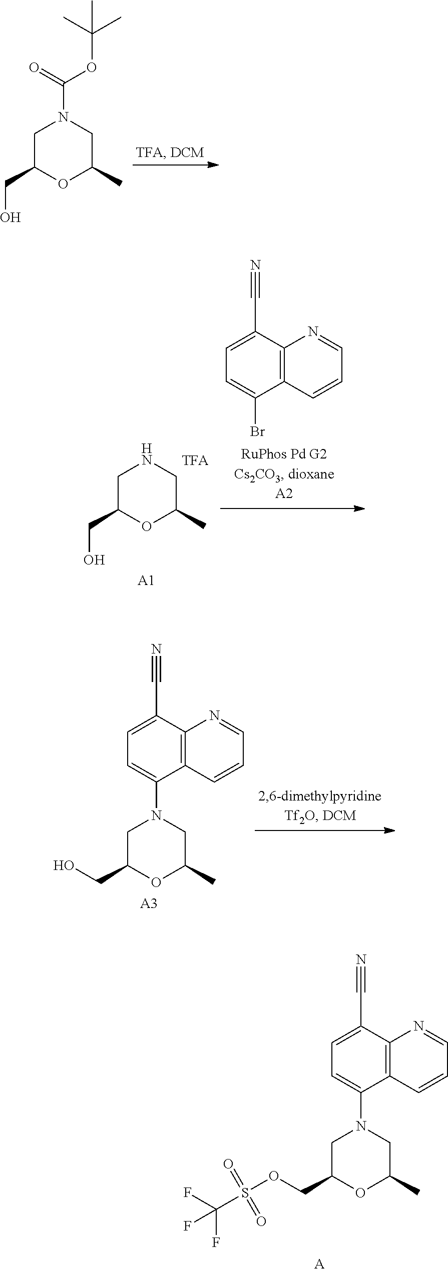

### Step 1: preparation of [(2R,6R)-6-methylmorpholin-2-yl]methanol;2,2,2-trifluoroacetic acid (Compound A1)

To a solution of tert-butyl (2R,6R)-2-(hydroxymethyl)-6-methylmorpholine-4-carboxylate (CAS: 1700609-48-8, Vendor: WuXi Apptec, 1.35 g, 5.84 mmol) in DCM (10 mL) was added TFA (2.66 g, 23.30 mmol). After being stirred at rt for 3 hrs, the reaction mixture was concentrated in vacuo to give the crude product compound A1 (1.43 g) which was used in next step directly. MS: calc'd 132 (MH⁺), measured 132 (MH⁺).

### Step 2: preparation of 5-[(2R,6R)-2-(hydroxymethyl)-6-methyl-morpholin-4-yl]quinoline-8-carbonitrile (Compound A3)

The mixture of 5-bromoquinoline-8-carbonitrile (compound A2, CAS: 507476-70-2, Vendor: BePharm, 1.50 g, 6.42 mmol), [(2R,6R)-6-methylmorpholin-2-yl]methanol; 2,2,2-trifluoroacetic acid (compound A1, 1.43 g, 5.83 mmol), RuPhos Pd G2 (136 mg, 175 μmol) and Cs₂CO₃ (5.70 g, 17.50 mmol) in 1,4-dioxane (10 mL) was charged with N₂, and heated to 90° C. overnight. After being cooled down, the solid was filtered off and washed with EA (10 mL) twice. The filtrate was concentrated and the residue was purified by silica gel chromatography (EA/PE=0 to 100%) to afford compound A3 (709 mg) as a light yellow solid. MS: calc'd 284 (MH⁺), measured 284 (MH⁺).

### Step 3: preparation of [(2R,6R)-4-(8-cyano-5-quinolyl)-6-methyl-morpholin-2-yl]methyl trifluoromethanesulfonate (Intermediate A)

To a flask was added 5-[(2R,6R)-2-(hydroxymethyl)-6-methyl-morpholin-4-yl]quinoline-8-carbonitrile (compound A3, 709 mg, 2.50 mmol), DCM (10 mL) and 2,6-dimethylpyridine (536 mg, 577 μL, 5.00 mmol). Then the reaction mixture was cooled with ice bath and trifluoromethanesulfonic anhydride (1.06 g, 634 μL, 3.75 mmol) was added dropwise. After being stirred for 2 hrs, the mixture was concentrated and purified by silica gel chromatography (EA/PE=0 to 40%) to give Intermediate A (720 mg) as a yellow solid. MS: calc'd 416 (MH⁺), measured 416 (MH⁺).

### Intermediate C

### [(2R,6R)-4-(8-cyanoquinoxalin-5-yl)-6-methyl-morpholin-2-yl]methyl trifluoromethanesulfonate

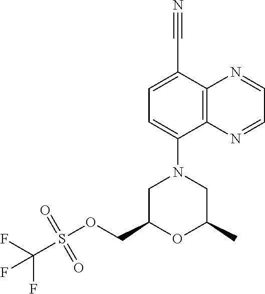 [SMILES: C[C@@H]1CN(c2ccc(C#N)c3nccnc23)C[C@H](COS(=O)(=O)C(F)(F)F)O1]

The title compound was prepared in analogy to the preparation of Intermediate A by using 8-bromoquinoxaline-5-carbonitrile (Synthesis refers to US 20170174653 A1) instead of 5-bromoquinoline-8-carbonitrile (compound A2). Intermediate C (825 mg) was obtained as an off-white solid. MS: calc'd 417 (MH⁺), measured 417 (MH⁺).

### Intermediate D

### [(2R,6R)-4-(7-cyanopyrazolo[1,5-a]pyridin-4-yl)-6-methyl-morpholin-2-yl]methyl trifluoromethanesulfonate

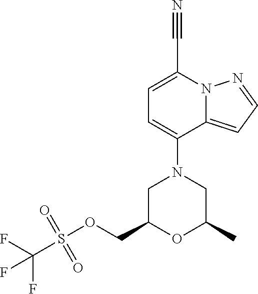 [SMILES: C[C@@H]1CN(c2ccc(C#N)n3nccc23)C[C@H](COS(=O)(=O)C(F)(F)F)O1]

The title compound was prepared in analogy to the preparation of Intermediate A by using 4-chloropyrazolo[1,5-a]pyridine-7-carbonitrile (CAS: 1268520-74-6, Vendor: PharmaBlock) instead of 5-bromoquinoline-8-carbonitrile (compound A2). Intermediate D (166 mg) was obtained as a white solid. MS: calc'd 405 (MH⁺), measured 405 (MH⁺).

### Intermediate E

### [(2R,6R)-4-(7-cyano-3-fluoro-pyrazolo[1,5-a]pyridin-4-yl)-6-methyl-morpholin-2-yl]methyl trifluoromethanesulfonate

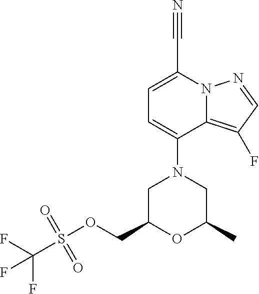 [SMILES: C[C@@H]1CN(c2ccc(C#N)n3ncc(F)c23)C[C@H](COS(=O)(=O)C(F)(F)F)O1]

The title compound was prepared according to the following scheme:

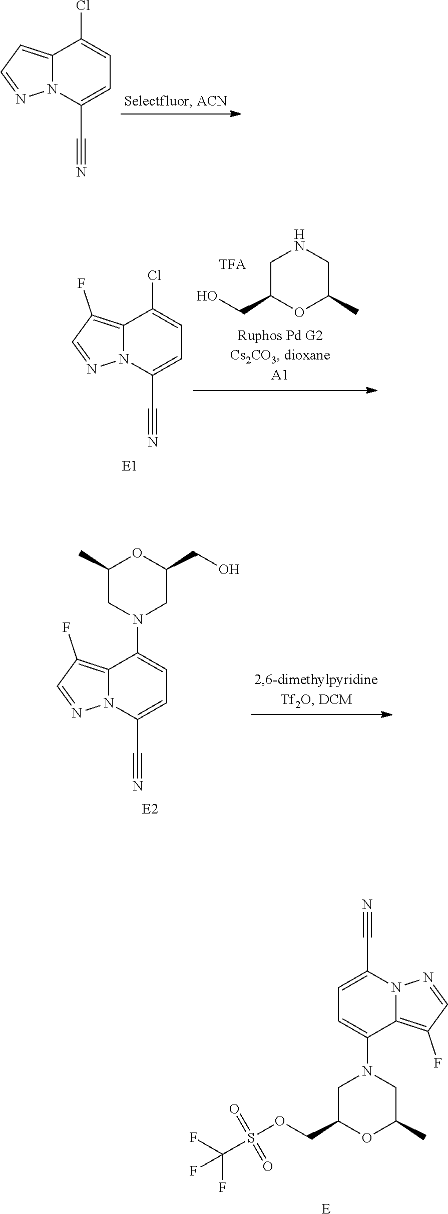

### Step 1: preparation of 4-chloro-3-fluoro-pyrazolo[1,5-a]pyridine-7-carbonitrile (Compound E1)

To a solution of 4-chloropyrazolo[1,5-a]pyridine-7-carbonitrile (CAS: 1268520-74-6, Vendor: Pharmablock, 600 mg, 3.38 mmol) in acetonitrile (50 mL) was added SelectFluor (2.39 g, 6.76 mmol). After being stirred at rt for 24 hrs, the mixture was concentrated and diluted with water (30 mL), extracted with DCM (30 mL) twice. The organic layer was washed with sat. NH₄Cl and brine, dried over Na₂SO₄, and concentrated to give a crude product which was purified by silica gel chromatography to give compound E1 (419 mg) as a light yellow powder. MS: calc'd 196 (MH⁺), measured 196 (MH⁺). ¹H NMR (400 MHz, METHANOL-d₄) δ=8.17 (d, J=3.5 Hz, 1H), 7.55 (d, J=7.6 Hz, 1H), 7.35 (d, J=7.7 Hz, 1H).

### Step 2: preparation of 3-fluoro-4-[(2R,6R)-2-(hydroxymethyl)-6-methyl-morpholin-4-yl]pyrazolo[1,5-a]pyridine-7-carbonitrile (Compound E2)

To a solution of 4-chloro-3-fluoropyrazolo[1,5-a]pyridine-7-carbonitrile (compound E1, 419 mg, 2.14 mmol), [(2R,6R)-6-methylmorpholin-2-yl]methanol;2,2,2-trifluoroacetic acid (compound A1, 526 mg, 2.14 mmol) and Cs₂CO₃ (2.79 g, 8.57 mmol) in 1,4-dioxane (10 mL) was added RuPhos Pd G2 (116 mg, 0.15 mmol) under N₂. The reaction mixture was heated at 90° C. for 2 hrs. After being cooled down, the mixture was diluted with EtOAc and filtered through celite. The filtrate was concentrated to give a brown oil which was purified by silica gel chromatography to give compound E2 (325 mg) as a yellow oil. MS: calc'd 291 (MH⁺), measured 291 (MH⁺).

### Step 3: preparation of (2R,6R)-4-(7-cyanopyrazolo[1,5-a]pyridin-4-yl)-6-methyl-morpholin-2-yl]methyl trifluoromethanesulfonate (Intermediate E)

To a solution of 3-fluoro-4-[(2R,6R)-2-(hydroxymethyl)-6-methyl-morpholin-4-yl]pyrazolo[1,5-a]pyridine-7-carbonitrile (compound E2, 325 mg, 1.12 mmol) in DCM (3 mL) was added 2,6-dimethylpyridine (240 mg, 258 μL, 2.24 mmol) and Tf₂O (474 mg, 284 μL, 1.68 mmol) at rt. After being stirred for 1.5 hrs, the mixture was diluted with DCM, washed with sat. NH₄Cl and brine. The organic layer was dried over Na₂SO₄ and concentrated to give the crude product which was purified by silica gel chromatography to give Intermediate E (180 mg) as a yellow solid. MS: calc'd 423 (MH⁺), measured 423 (MH⁺).

### Intermediate G

### [(2R,6R)-6-methyl-4-(8-methylquinoxalin-5-yl)morpholin-2-yl]methyl trifluoromethanesulfonate

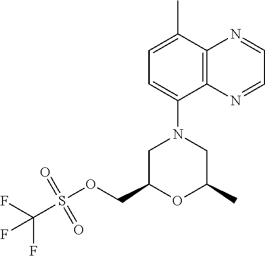 [SMILES: Cc1ccc(N2C[C@H](COS(=O)(=O)C(F)(F)F)O[C@H](C)C2)c2nccnc12]

The title compound was prepared in analogy to the preparation of Intermediate A by using 5-bromo-8-methyl-quinoxaline (CAS: 1360599-43-4, Vendor: Bepharm) instead of 5-bromoquinoline-8-carbonitrile (compound A2), replacing RuPhos Pd G2 and Cs₂CO₃ with Pd₂(dba)₃, BINAP and t-BuONa in the Buchwald-Hartwig amination reaction. Intermediate G (200 mg) was obtained as a brown solid. MS: calc'd 406 (MH⁺), measured 406 (MH⁺).

### Intermediate K

### [(2R,6R)-4-(8-cyano-2,3-dideuterio-quinoxalin-5-yl)-6-methyl-morpholin-2-yl]methyl trifluoromethanesulfonate

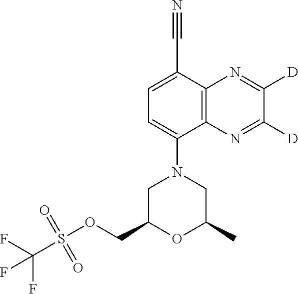 [SMILES: [2H]c1nc2c(C#N)ccc(N3C[C@H](COS(=O)(=O)C(F)(F)F)O[C@H](C)C3)c2nc1[2H]]

The title compound was prepared in analogy to the preparation of Intermediate A by using 8-bromo-2,3-dideuterio-quinoxaline-5-carbonitrile (compound K₈) instead of 5-bromoquinoline-8-carbonitrile (compound A2). Intermediate K (200 mg) was obtained as a yellow solid. MS: calc'd 419 (MH⁺), measured 419 (MH⁺).

The compound K8 was prepared according to the following scheme:

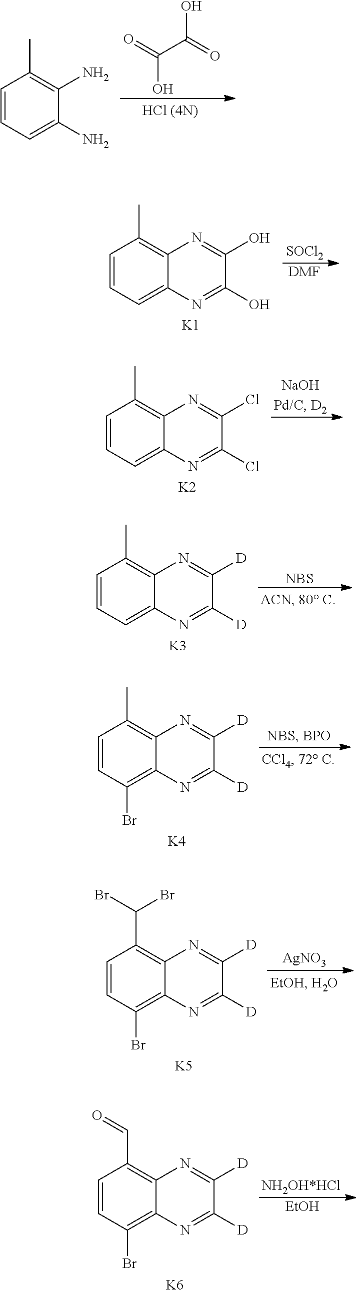

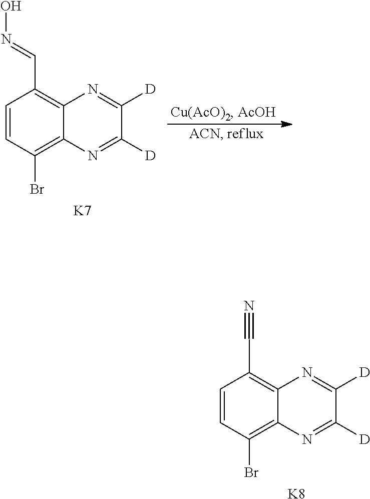

### Step 1: preparation of 5-methylquinoxaline-2,3-diol (Compound K1)

To the solution of 2,3-diaminotoluene (CAS: 2687-25-4, Vendor: Aldrich, 150 g, 1228 mmol) and hydrogenchloride (4N, 3750 mL) was added oxalic acid (221 g, 2456 mmol). The mixture was stirred at 100° C. for 3 hrs. After being cooled down, the mixture was poured into 3000 mL ice-water. A black solid was appeared. The mixture was filtered, and the wet-cake was washed with 500 mL water and dried under vacuum to give compound K1 (230 g) as a black solid. MS calc'd 177 (MH⁺), measured 177 (MH⁺).

### Step 2: preparation of 2,3-dichloro-5-methyl-quinoxaline (Compound K2)

To the solution of 5-methylquinoxaline-2,3-diol (compound K1, 220 g, 1249 mmol) in 1,2-dichloroethane (500 mL) was added thionyl chloride (500 mL, 2498 mmol) and DMF (0.97 mL, 12.49 mmol). The final mixture was stirred at 80° C. for 6 hrs. After being cooled down, the mixture was concentrated to give a crude product which was purified by silica gel chromatography (PE/EA=5/1) to give compound K2 (152 g) as a yellow solid. MS calc'd 213 (MH⁺), measured 213 (MH⁺).

### Step 3: preparation of 2,3-dideuterio-5-methyl-quinoxaline (Compound K3)

To the solution of 2,3-dichloro-5-methyl-quinoxaline (compound K2, 46 g, 215.90 mmol) in 1,4-dioxane (760 mL) and deuterium oxide (160 mL) was added 2N aqueous solution of NaOH in D₂O (215.9 mL, 431.80 mmol) and Pd/C (10 wt. %, 9200 mg). The mixture was stirred at r.t. under deuterium atmosphere for 3 hrs. Then the mixture was filtered and diluted with 800 mL water, extracted with 250 mL EA for three times. The combined organic layer was washed with 300 mL water twice and 200 mL brine once. After dried over Na₂SO₄, the organic layer was concentrated to give the crude product which was purified by silica gel chromatography (PE/EA=10/1) to give compound K3 (16 g) as a brown oil. MS calc'd 147 (MH⁺), measured 147 (MH⁺).

### Step 4: preparation of 5-bromo-2,3-dideuterio-8-methyl-quinoxaline (Compound K4)

A solution of 2,3-dideuterio-5-methyl-quinoxaline (compound K3, 10 g, 58.14 mmol), N-bromosuccinimide (20697 mg, 116.29 mmol) in ACN (250 mL) was stirred at 80° C. for 18 hrs. After being cooled down, the solution was concentrated to give a crude solid which was washed with 300 mL water. The crude product was purified by silica gel chromatography (PE/EA=5/1) to give compound K4 (8500 mg) as a light yellow solid. MS calc'd 225 (MH⁺), measured 225 (MH⁺).

### Step 5: preparation of 5-bromo-2,3-dideuterio-8-(dibromomethyl)quinoxaline (Compound K5)

A solution of 5-bromo-2,3-dideuterio-8-methyl-quinoxaline (compound K4, 8500 mg, 37.76 mmol), N-bromosuccinimide (26886 mg, 151.06 mmol), 2,2′-azobis(2-methylpropionitrile) (1240 mg, 7.55 mmol) in carbon tetrachloride (250 mL) was stirred at 80° C. for 18 hrs. After being cooled down, the mixture was concentrated to give an oil which was diluted with 300 mL water. The mixture was stirred for 1 h and filtered. The wet-cake was purified by silica gel chromatography (PE/EA=10/1) to give compound K5 (10 g) as a yellow solid. MS calc'd 381 (MH⁺), measured 381 (MH⁺).

### Step 6: Preparation of 8-bromo-2,3-dideuterio-quinoxaline-5-carbaldehyde (Compound K6)

A solution of silver nitrate (17.8 g, 104.47 mmol) in water (200 mL) was added to the solution of 5-bromo-2,3-dideuterio-8-(dibromomethyl)quinoxaline (compound K5, 10000 mg, 26.12 mmol) in ethanol (400 mL). After being stirred at rt for 2 hrs, the mixture was concentrated to about 300 mL and filtered. The filtrate was diluted with 500 mL water and extracted with 200 mL EA for three times. The combined organic layer was washed with 200 mL water twice and 100 mL brine once, dried over Na₂SO₄ and concentrated to give the crude product (batch 1). The wet-cake was stirred in 500 mL DCM for 1 h, filtered and the wet-cake was washed with 100 mL DCM twice. The filtrate was concentrated to give the crude product as an oil (batch 2). The two batches were combined to give compound K6 (5.5 g) as a yellow solid. MS calc'd 239 (MH⁺), measured 239 (MH⁺).

### Step 7: Preparation of (5E)-8-bromo-2,3-dideuterio-quinoxaline-5-carbaldehyde oxime (Compound K7)

A solution of 8-bromo-2,3-dideuterio-quinoxaline-5-carbaldehyde (compound K6, 5.5 g, 23.01 mmol), hydroxylamine hydrochloride (2.1 g, 29.91 mmol), sodium acetate (4.15 g, 50.61 mmol) in ethanol (250 mL) was stirred at 70° C. for 16 hrs. After being cooled down, the solution was concentrated to give a residue which was treated with 300 mL water and stirred for 0.5 h. The suspension was filtered, and the wet-cake was treated with 300 mL MeCN. After concentration, the compound K7 (4.5 g) was obtained as a yellow solid. MS calc'd 254 (MH⁺), measured 254 (MH⁺).

### Step 8: Preparation of 8-bromo-2,3-dideuterio-quinoxaline-5-carbonitrile (Compound K8)

A solution of (5E)-8-bromo-2,3-dideuterio-quinoxaline-5-carbaldehyde oxime (compound K7, 4.5 g, 17.71 mmol), copper(II) acetate (965 mg, 5.31 mmol), acetic acid (1.38 g, 23.02 mmol) in acetonitrile (120 mL) was stirred at 80° C. for 18 hrs. After being cooled down, the solution was concentrated to give an oil which was dissolved in 500 mL DCM. The suspension was stirred for 1 h, filtered, and the wet-cake was washed with 100 mL DCM twice. The filtrate was concentrated and purified by silica gel chromatography (PE/EA/DCM=3/1/1) to give compound K8 (2.6 g) as a yellow solid. MS calc'd 236 (MH⁺), measured 236 (MH⁺).

### Intermediate L

### [(2R,6R)-4-(8-cyano-2-deuterio-5-quinolyl)-6-methyl-morpholin-2-yl]methyl trifluoromethanesulfonate

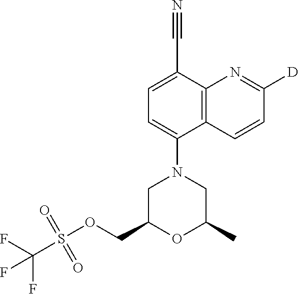 [SMILES: [2H]c1ccc2c(N3C[C@H](COS(=O)(=O)C(F)(F)F)O[C@H](C)C3)ccc(C#N)c2n1]

The compound L was prepared according to the following scheme:

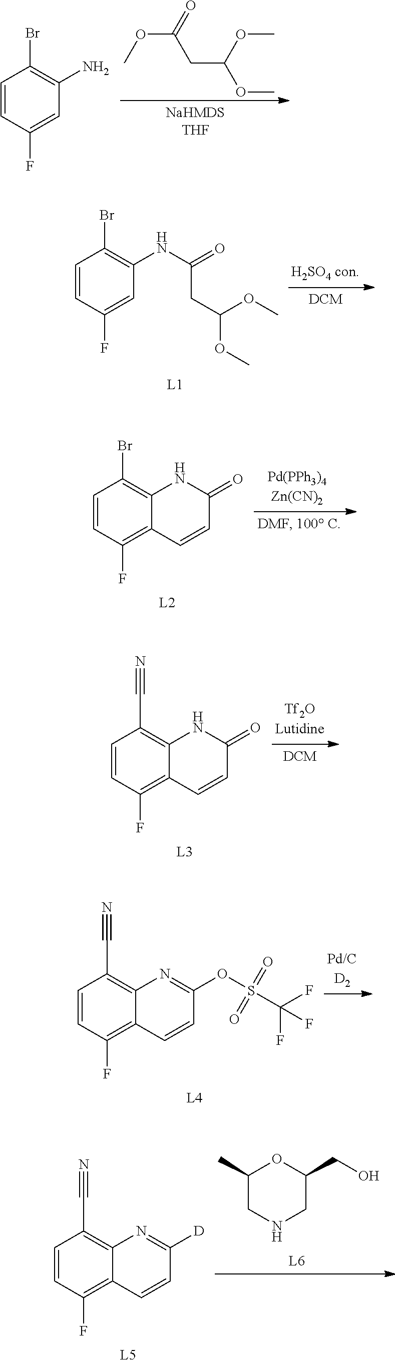

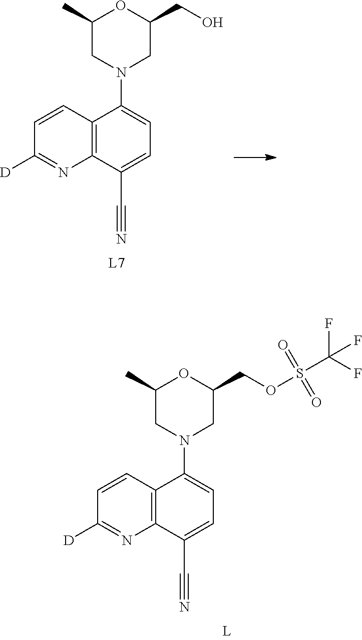 [SMILES: [2H]c1ccc2c(N3C[C@H](CO)O[C@H](C)C3)ccc(C#N)c2n1 ; [2H]c1ccc2c(N3C[C@H](COS(=O)(=O)C(F)(F)F)O[C@H](C)C3)ccc(C#N)c2n1]

Step 1: preparation of N-(2-bromo-5-fluoro-phenyl)-3,3-dimethoxy-propanamide (compound L1)

To the solution of 2-bromo-5-fluoroaniline (CAS: 1003-99-2, Vendor: TCI, 50 g, 263.14 mmol) and methyl 3,3-dimethoxypropionate (CAS: 7424-91-1, Vendor: Accela, 45 mL, 315.77 mmol) in THF (150 mL) was added NaHMDS in THF (1M, 394 mL, 394.72 mmol) dropwise at 0° C. After being stirred at 0° C. for 10 mins, the mixture was warmed up to 15° C. and stirred for 18 hrs. The reaction was quenched by addition of 100 mL sat. NH₄Cl and concentrated to about 300 mL. The solution was diluted with 500 mL water and extracted with 200 mL EA for three times. The combined organic layer was washed with 200 mL water twice and 100 mL brine, dried over Na₂SO₄ and concentrated to give the crude product L1 (100 g) as a brown oil. MS calc'd 321 (MH⁺), measured 321 (MH⁺).

### Step 2: Preparation of 8-bromo-5-fluoro-1H-quinolin-2-one (Compound L2)

A solution of N-(2-bromo-5-fluoro-phenyl)-3,3-dimethoxy-propanamide (compound L1, 100 g, 238.46 mmol) in DCM (500 mL) was added to concentrated sulfuric acid (300 mL) at 0° C. After being stirred at 15° C. for 2 hrs, the mixture was poured slowly into 2000 mL ice-water, a yellow precipitate was appeared. The mixture was filtered, and the wet-cake was washed with 500 mL water, 200 mL isopropyl alcohol and 300 mL PE. The solid was dried in vacuum to give compound L2 (50 g) as a yellow solid. MS calc'd 242 (MH⁺), measured 242 (MH⁺).

### Step 3: Preparation of 5-fluoro-2-oxo-1H-quinoline-8-carbonitrile (Compound L3)

A solution of 8-bromo-5-fluoro-1H-quinolin-2-one (compound L2, 50 g, 206.58 mmol), zinc cyanide (48.50 g, 413.15 mmol), Pd(PPh₃)₄ (24.29 g, 21.02 mmol) in DMF (1000 mL) was stirred at 120° C. for 5 hrs. After being cooled down, the reaction mixture was quenched with 300 mL saturated NH₄Cl, diluted with 2000 mL water and extracted with 500 mL DCM for three times. The combined organic layer was washed with 500 mL water twice and 200 mL brine once, dried over Na₂SO₄ and concentrated to give the crude product which was purified by silica gel chromatography (PE/EA=3/1) to give compound L3 (29 g) as a yellow solid. MS calc'd 189 (MH⁺), measured 189 (MH⁺).

### Step 4: Preparation of (8-cyano-5-fluoro-2-quinolyl) trifluoromethanesulfonate (Compound L4)

To the solution of 5-fluoro-2-oxo-1H-quinoline-8-carbonitrile (compound L3, 17000 mg, 90.35 mmol), 2,6-dimethylpyridine (38705 mg, 361.39 mmol) in DCM (500 mL) was added trifluoromethanesulfonic anhydride (50975 mg, 180.70 mmol) at 0° C. After being stirred at 0° C. for 1 h, the mixture was diluted with 500 mL water and extracted with 200 mL DCM for three times. The combined organic layer was washed with 200 mL water twice and 100 mL brine once, dried over Na₂SO₄ and concentrated to give the crude product which was purified by silica gel chromatography (PE/EA=5/1) to give compound L4 (23000 mg) as a yellow solid. MS calc'd 321 (MH⁺), measured 321 (MH⁺).

### Step 5: Preparation of 2-deuterio-5-fluoro-quinoline-8-carbonitrile (Compound L5)

To the solution of (8-cyano-5-fluoro-2-quinolyl) trifluoromethanesulfonate (compound L4, 23 g, 71.83 mmol) in THF (230 mL) and deuterium oxide (100 mL) was added potassium carbonate (19.8 g, 1.44 mol) and Pd/C (10 wt. %, 6 g). The mixture was stirred at 40° C. for 5 hrs under deuterium atmosphere. Then the mixture was filtered and the filtrate was concentrated and purified by silica gel chromatography (PE/EA=5/1) to give compound L5 (11.4 g) as a light yellow solid. MS calc'd 174 (MH⁺), measured 174 (MH⁺).

### Step 6: preparation of 5-((2R,6R)-2-(hydroxymethyl)-6-methylmorpholino)quinoline-8-carbonitrile-2-d (Compound L7)

A mixture of 5-fluoroquinoline-8-carbonitrile-2-d (compound L5, 611 mg, 3.53 mmol), ((2R,6R)-6-methylmorpholin-2-yl)methanol hydrochloride salt (compound L6, 710 mg, 4.23 mmol), N-ethyl-N-isopropylpropan-2-amine (1.37 g, 10.6 mmol) in DMSO (3 mL) was stirred at 120° C. for 16 hours. Then the solution was diluted with EA and washed with water and brine. The organic layer was dried and concentrated. The residue was purified by silica gel chromatography (EA/PE from 20% to 80%) to afford 5-((2R,6R)-2-(hydroxymethyl)-6-methylmorpholino)quinoline-8-carbonitrile-2-d (compound L7, 990 mg) as a yellow solid. MS: calc'd 285 (MH⁺), measured 285 (MH⁺).

### Step 7: preparation of ((2R,6R)-4-(8-cyanoquinolin-5-yl-2-d)-6-methylmorpholin-2-yl)methyl trifluoromethanesulfonate (Intermediate L)

To a solution of 5-((2R,6R)-2-(hydroxymethyl)-6-methylmorpholino) quinoline-8-carbonitrile-2-d (compound L7, 3 g) and 2,6-dimethylpyridine (3.39 g, 3.77 ml, 31.7 mmol) in CH₂Cl₂ (20 ml) was added trifluoromethanesulfonic anhydride (4.47 g, 2.67 ml, 15.8 mmol) dropwise at 0° C. The mixture was stirred at 0° C. for 1 h. The mixture was then diluted with DCM and washed with saturated NH₄Cl solution and brine. The organic layer was dried over Na₂SO₄ and concentrated. The residue was purified by silica gel chromatography (EA/PE from 20% to 60%) to afford ((2R,6R)-4-(8-cyanoquinolin-5-yl-2-d)-6-methylmorpholin-2-yl)methyl trifluoromethanesulfonate (Intermediate L 4.087 g) as a yellow solid. MS: calc'd 417 (MH⁺), measured 417 (MH⁺).

### Intermediate M

### [(2R,6R)-6-methyl-4-(1-methyl-2-oxo-1,8-naphthyridin-4-yl)morpholin-2-yl]methyl trifluoromethanesulfonate

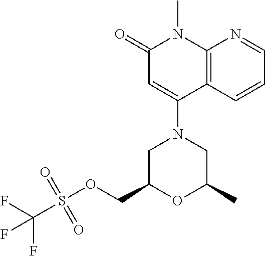 [SMILES: C[C@@H]1CN(c2cc(=O)n(C)c3ncccc23)C[C@H](COS(=O)(=O)C(F)(F)F)O1]

The title compound was prepared in analogy to the preparation of Intermediate A by using 4-bromo-1-methyl-1,8-naphthyridin-2-one (compound M1) instead of 5-bromoquinoline-8-carbonitrile (compound A2). Intermediate M (140 mg) was obtained as a yellow solid. MS: calc'd 422 (MH⁺), measured 422 (MH⁺).

The compound M1 was prepared according to the following scheme:

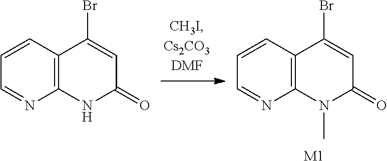

### Preparation of 4-bromo-1-methyl-1,8-naphthyridin-2-one (Compound M1)

To the solution of 4-bromo-1,8-naphthyridin-2(1H)-one (CAS: 72235-36-0, Vendor: Accela, 370 mg, 1.64 mmol) in DMF (15 mL) was added iodomethane (2.33 g, 16.40 mmol) and Cs₂CO₃ (1.07 g, 3.29 mmol). The reaction mixture was stirred at 80° C. overnight. After being cooled down, the reaction was quenched by ice-water (30 mL). The solid was collected by filtration to give crude compound M1 (419 mg) which was used directly for next step. MS calc'd 239 (MH⁺), measured 239 (MH⁺). ¹H NMR (400 MHz, METHANOL-d₄) δ=8.72 (dd, J=1.7, 4.7 Hz, 1H), 8.39 (dd, J=1.7, 7.9 Hz, 1H), 7.43 (dd, J=4.7, 8.0 Hz, 1H), 7.20 (s, 1H), 3.83 (s, 3H).

### Reference Compound R1

### 5-[(2S,6R)-2-[[4-(4-methoxyphenyl)piperazin-1-yl]methyl]-6-methyl-morpholin-4-yl]quinoline-8-carbonitrile

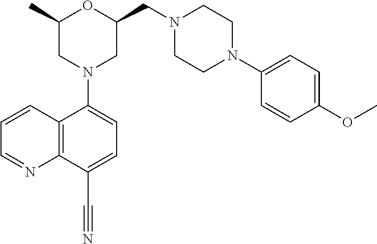 [SMILES: COc1ccc(N2CCN(C[C@H]3CN(c4ccc(C#N)c5ncccc45)C[C@@H](C)O3)CC2)cc1]

The title compound was prepared according to the following scheme:

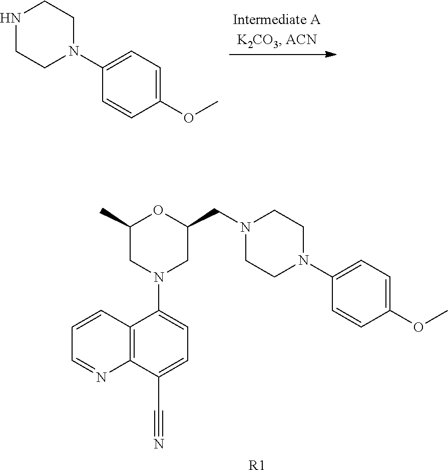

To a flask was added [(2R,6R)-4-(8-cyano-5-quinolyl)-6-methyl-morpholin-2-yl]methyl trifluoromethanesulfonate (Intermediate A, 30 mg, 72 μmol), 1-(4-methoxyphenyl)piperazine (CAS: 38212-30-5, Vendor: Accela, 21 mg, 108 μmol), potassium carbonate (30 mg, 217 μmol) and acetonitrile (4 mL). After being stirred at 85° C. for 2 hrs, the mixture was filtered through celite and purified by prep-HPLC to give compound R1 (22 mg) as a yellow solid. MS: calc'd 458 (MH⁺), measured 458 (MH⁺). ¹H NMR (400 MHz, METHANOL-d₄) δ=9.00 (dd, J=1.7, 4.2 Hz, 1H), 8.67 (dd, J=1.7, 8.6 Hz, 1H), 8.18 (d, J=8.1 Hz, 1H), 7.66 (dd, J=4.3, 8.6 Hz, 1H), 7.30 (d, J=7.9 Hz, 1H), 7.05-6.98 (m, 2H), 6.93-6.85 (m, 2H), 4.55-4.46 (m, 1H), 4.26-4.16 (m, 1H), 3.89-3.56 (m, 7H), 3.50-3.37 (m, 6H), 3.25-3.00 (m, 2H), 2.84-2.71 (m, 2H), 1.33 (d, J=6.2 Hz, 3H).

### Reference Compound R2

### 5-[(2S,6R)-2-[[4-(4-chlorophenyl)piperazin-1-yl]methyl]-6-methyl-morpholin-4-yl]quinoline-8-carbonitrile

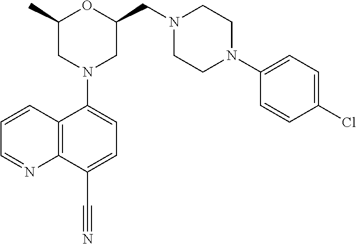 [SMILES: C[C@@H]1CN(c2ccc(C#N)c3ncccc23)C[C@H](CN2CCN(c3ccc(Cl)cc3)CC2)O1]

The title compound was prepared in analogy to the preparation of compound R1 by using 1-(4-cholophenyl)piperazine (CAS: 38212-33-8, Vendor: BePharm) instead of 1-(4-methoxyphenyl)piperazine. Compound R2 (24 mg) was obtained as a yellow solid. MS: calc'd 462 (MH⁺), measured 462 (MH⁺). ¹H NMR (400 MHz, METHANOL-d₄) δ=9.00 (dd, J=1.6, 4.3 Hz, 1H), 8.67 (dd, J=1.7, 8.6 Hz, 1H), 8.17 (d, J=8.1 Hz, 1H), 7.66 (dd, J=4.3, 8.6 Hz, 1H), 7.34-7.26 (m, 3H), 7.07-6.98 (m, 2H), 4.55-4.47 (m, 1H), 4.25-4.15 (m, 1H), 3.99-3.55 (m, 3H), 3.55-3.32 (m, 7H), 3.28-3.04 (m, 2H), 2.85-2.71 (m, 2H), 1.33 (d, J=6.2 Hz, 3H).

### Example 1

### 5-[(2R,6S)-2-methyl-6-[[3-(3-piperazin-1-ylphenyl)pyrrolidin-1-yl]methyl]morpholin-4-yl]quinoline-8-carbonitrile

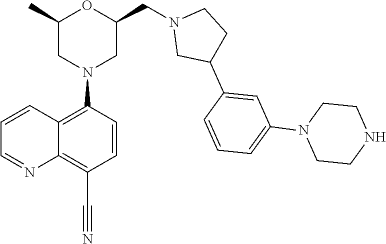

The title compound was prepared according to the following scheme:

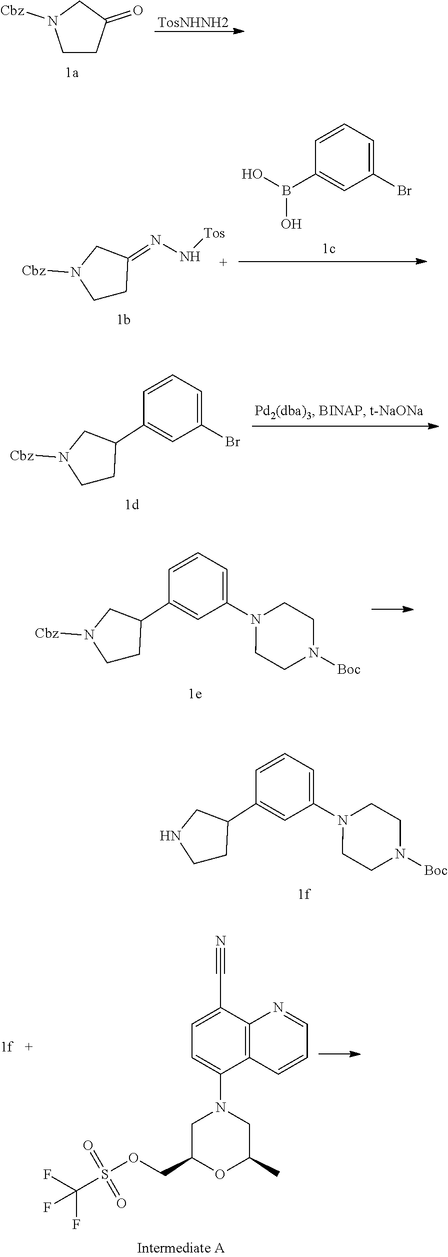

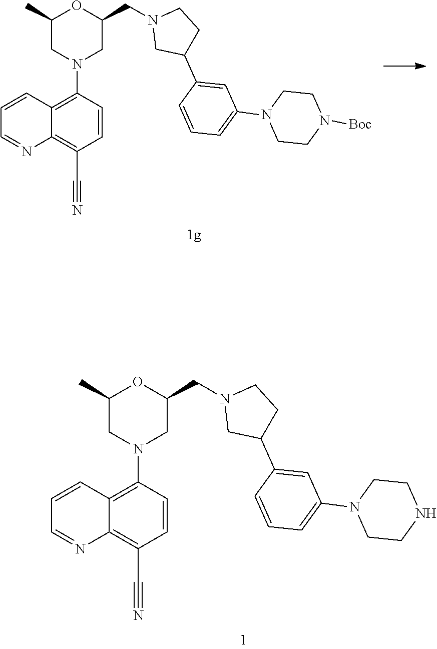 [SMILES: C[C@@H]1CN(c2ccc(C#N)c3ncccc23)C[C@H](CN2CCC(c3cccc(N4CCN(C(=O)OC(C)(C)C)CC4)c3)C2)O1 ; C[C@@H]1CN(c2ccc(C#N)c3ncccc23)C[C@H](CN2CCC(c3cccc(N4CCNCC4)c3)C2)O1]

### Step 1: Preparation of benzyl (3Z)-3-(p-tolylsulfonylhydrazono)pyrrolidine-1-carboxylate (Compound 1b)

To a solution of 4-methylbenzenesulfonhydrazide (2.8 g, 15.05 mmol) in ethanol (30 mL) was added benzyl 3-oxopyrrolidine-1-carboxylate (compound 1a, 2.56 mL, 13 mmol). The reaction was stirred at 25° C. for 3 hrs. The solid was filtrated, washed with EtOH (50 mL), and dried to give the desired product compound 1b (4.0 g) as a yellow solid. MS calc'd 388 (MH⁺); measured 388 (MH⁺).

### Step 2: preparation of benzyl 3-(3-bromophenyl)pyrrolidine-1-carboxylate (Compound 1d)

To a solution of 3-bromophenylboronic acid (compound 1c, 777 mg, 4 mmol) in 1,4-dioxane (10 mL) was added benzyl (3Z)-3-(p-tolylsulfonylhydrazono)pyrrolidine-1-carboxylate (compound 1b, 1.0 g, 2.58 mmol), and cesium carbonate (1.26 g, 3.87 mmol). The reaction mixture was stirred for 16 hours at 115° C. under N₂, then filtered and the filtrate was concentrated under reduced pressure. The residue was purified by column (PE/EA=3/1, Rf=0.6) to give compound 1d (300 mg) as yellow solid. MS calc'd 360 (MH⁺), measured 360 (MH⁺).

### Step 3: Preparation of tert-butyl 4-[3-(1-benzyloxycarbonylpyrrolidin-3-yl)phenyl]piperazine-1-carboxylate (Compound 1e)

To a solution of 1-Boc-piperazine (201.6 mg, 1.08 mmol) in toluene (5 mL) was added benzyl 3-(3-bromophenyl)pyrrolidine-1-carboxylate (compound 1d, 300 mg, 0.83 mmol), sodium tert-butoxide (200 mg, 2.08 mmol), (R)-binap (103 mg, 0.17 mmol) and bis(dibenzylideneacetone)palladium (48 mg, 0.08 mmol). The reaction was stirred at 110° C. for 18 hrs. The mixture was cooled to room temperature and filtered through celite. The filtrate was concentrated under reduced pressure and the residue was purified by silica gel chromatography (PE/EA=3/1, Rf=0.3) to give compound 1e (200 mg) as a yellow solid. MS calc'd 466.3 (MH⁺); measured 466.3 (MH⁺).

### Step 4: Preparation of tert-butyl 4-isoindolin-5-ylpiperazine-1-carboxylate (Compound 1f)

To a solution of tert-butyl 4-[3-(1-benzyloxycarbonylpyrrolidin-3-yl)phenyl]piperazine-1-carboxylate (compound 1e, 180 mg, 0.41 mmol) and Pd(OH)₂/C (18 mg, 0.41 mmol) in 2-propanol (2 mL) was stirred for 3 hrs at 25° C. under H₂ balloon atmosphere. The mixture was filtered and the filtrate was concentrated to give compound if (50 mg) as yellow oil. MS calc'd 332.2 (MH⁺), measured 332.2 (MH⁺).

### Step 5: preparation of tert-butyl 4-[3-[1-[[(2S,6R)-4-(8-cyano-5-quinolyl)-6-methyl-morpholin-2-yl]methyl]pyrrolidin-3-yl]phenyl]piperazine-1-carboxylate (Compound 1g)

To a solution of tert-butyl 4-(3-pyrrolidin-3-ylphenyl)piperazine-1-carboxylate (compound 1f, 50 mg, 0.15 mmol) in ACN (1 mL) was added potassium carbonate (52 mg, 0.38 mmol) and [(2R,6R)-4-(8-cyano-5-quinolyl)-6-methyl-morpholin-2-yl]methyl trifluoromethanesulfonate (intermediate A, 75 mg, 0.18 mmol). The mixture was stirred for 3 hrs at 55° C., then filtered and the filtrate was concentrated. The residue was purified by pre-TLC (EA:PE=1:1, Rf=0.5) to get compound 1g (50 mg) as yellow oil. MS calc'd 597.4 (MH⁺), measured 597.4 (MH⁺).

### Step 6: preparation of 5-[(2R,6S)-2-methyl-6-[[3-(3-piperazin-1-ylphenyl)pyrrolidin-1-yl]methyl]morpholin-4-yl]quinoline-8-carbonitrile (Example 1)

To a solution of tert-butyl 4-[3-[1-[[(2S,6R)-4-(8-cyano-5-quinolyl)-6-methyl-morpholin-2-yl]methyl]pyrrolidin-3-yl]phenyl]piperazine-1-carboxylate (compound 1g, 50 mg, 0.080 mmol) in DCM (2 mL) was added trifluoroacetic acid (1.0 mL, 12.98 mmol) under ice-bath. After being stirred at rt for 3 hrs, the mixture was concentrated in vacuo to give a yellow oil which was purified by prep-HPLC to give Example 1 (24 mg) as a yellow solid. MS calc'd 497.3 (MH⁺), measured 497.3 (MH⁺). ¹H NMR (400 MHz, METHANOL-d₄) δ: ppm 9.02 (br s, 1H), 8.64-8.73 (m, 1H), 8.19 (d, J=8.0 Hz, 1H), 7.67 (br d, J=4.1 Hz, 1H), 7.28-7.39 (m, 2H), 6.94-7.08 (m, 3H), 4.43 (br s, 1H), 4.20 (br s, 1H), 3.57-4.14 (m, 4H), 3.36-3.55 (m, 13H), 2.71-2.86 (m, 2H), 2.56 (br s, 1H), 2.17-2.38 (m, 1H), 1.34 (br d, J=6.1 Hz, 3H).

### Example 2

### 5-[(2R,6S)-2-methyl-6-[[4-[3-(4-piperidyl)phenyl]-1-piperidyl]methyl]morpholin-4-yl]quinoline-8-carbonitrile

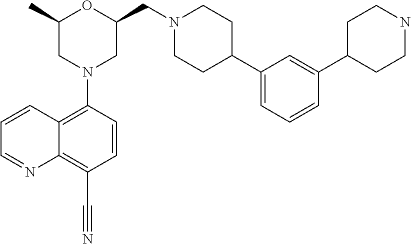 [SMILES: C[C@@H]1CN(c2ccc(C#N)c3ncccc23)C[C@H](CN2CCC(c3cccc(C4CCNCC4)c3)CC2)O1]

The title compound was prepared in analogy to the preparation of Example 1 by using benzyl 4-oxopiperidine-1-carboxylate and [3-(1-tert-butoxycarbonyl-4-piperidyl)phenyl]boronic acid instead of benzyl 3-oxopyrrolidine-1-carboxylate (compound 1a) and 3-bromophenylboronic acid (compound 1c). Example 2 (14 mg) was obtained as a yellow solid. MS: calc'd 510 (MH⁺), measured 510 (MH⁺). ¹H NMR (400 MHz, METHANOL-d4) δ 9.01 (dd, J=1.5, 4.1 Hz, 1H), 8.69 (dd, J=1.6, 8.6 Hz, 1H), 8.18 (d, J=7.9 Hz, 1H), 7.67 (dd, J=4.3, 8.5 Hz, 1H), 7.38-7.33 (m, 1H), 7.30 (d, J=8.0 Hz, 1H), 7.28-7.16 (m, 3H), 4.58-4.49 (m, 1H), 4.25-4.15 (m, 1H), 3.85 (br d, J=4.9 Hz, 2H), 3.53 (br d, J=12.7 Hz, 2H), 3.45 (br d, J=11.5 Hz, 2H), 3.39-3.35 (m, 2H), 3.29-3.11 (m, 4H), 3.00-2.88 (m, 2H), 2.84-2.71 (m, 2H), 2.22-1.89 (m, 8H), 1.34 (d, J=6.3 Hz, 3H).

### Example 3

### 5-[(2R,6S)-2-methyl-6-[[3-(4-piperazin-1-ylphenyl)azetidin-1-yl]methyl]morpholin-4-yl]quinoline-8-carbonitrile

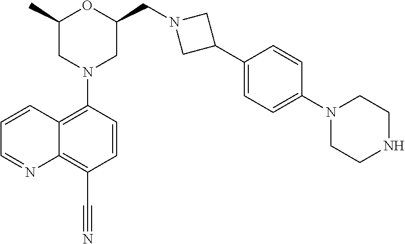 [SMILES: C[C@@H]1CN(c2ccc(C#N)c3ncccc23)C[C@H](CN2CC(c3ccc(N4CCNCC4)cc3)C2)O1]

The title compound was prepared in analogy to the preparation of Example 1 by using benzyl 2-(3-oxoazetidin-1-yl)acetate and 4-bromophenylboronic acid instead of benzyl 3-oxopyrrolidine-1-carboxylate (compound 1a) and 3-bromophenylboronic acid (compound 1c). Example 3 (5 mg) was obtained as a yellow solid. MS: calc'd 483 (MH⁺), measured 483 (MH⁺). ¹H NMR (400 MHz, METHANOL-d4) δ 9.01 (d, J=4.0 Hz, 1H), 8.68 (d, J=8.8 Hz, 1H), 8.18 (d, J=7.6 Hz, 1H), 7.67 (dd, J=4.3, 8.5 Hz, 1H), 7.38-7.36 (m, 2H), 7.29 (d, J=8 Hz, 1H), 7.10-7.08 (m, 2H), 4.62-4.16 (m, 7H), 3.52-3.39 (m, 12H), 2.82-2.70 (m, 2H), 1.33-1.31 (d, J=6.0 Hz, 3H).

### Example 4

### 5-[(2R,6S)-2-methyl-6-[[3-(3-piperazin-1-ylphenyl)azetidin-1-yl]methyl]morpholin-4-yl]quinoline-8-carbonitrile

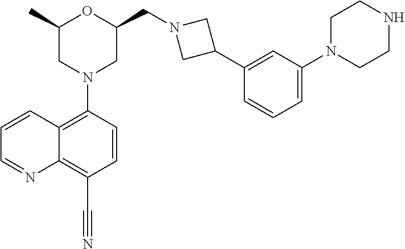 [SMILES: C[C@@H]1CN(c2ccc(C#N)c3ncccc23)C[C@H](CN2CC(c3cccc(N4CCNCC4)c3)C2)O1]

The title compound was prepared in analogy to the preparation of Example 1 by using benzyl 2-(3-oxoazetidin-1-yl)acetate instead of benzyl 3-oxopyrrolidine-1-carboxylate (compound 1a). Example 4 (26 mg) was obtained as a yellow solid. MS: calc'd 483 (MH⁺), measured 483 (MH⁺). ¹H NMR (400 MHz, METHANOL-d4) δ 9.01 (dd, J=4.3, 1.5 Hz, 1H), 8.67 (dd, J=8.7, 1.6 Hz, 1H), 8.19 (d, J=8.0 Hz, 1H), 7.67 (dd, J=8.5, 4.3 Hz, 1H), 7.37 (br t, J=8.3 Hz, 1H), 7.30 (d, J=8.0 Hz, 1H), 6.99-7.13 (m, 3H), 4.12-4.67 (m, 7H), 3.37-3.55 (m, 12H), 2.68-2.86 (m, 2H), 1.33 (br d, J=6.1 Hz, 3H).

### Example 5

### 5-[(2S,6R)-2-[[4-[4-(aminomethyl)phenyl]piperazin-1-yl]methyl]-6-methyl-morpholin-4-yl]quinoline-8-carbonitrile

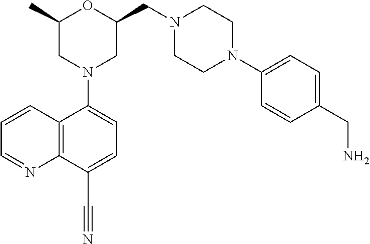 [SMILES: C[C@@H]1CN(c2ccc(C#N)c3ncccc23)C[C@H](CN2CCN(c3ccc(CN)cc3)CC2)O1]

The title compound was prepared according to the following scheme:

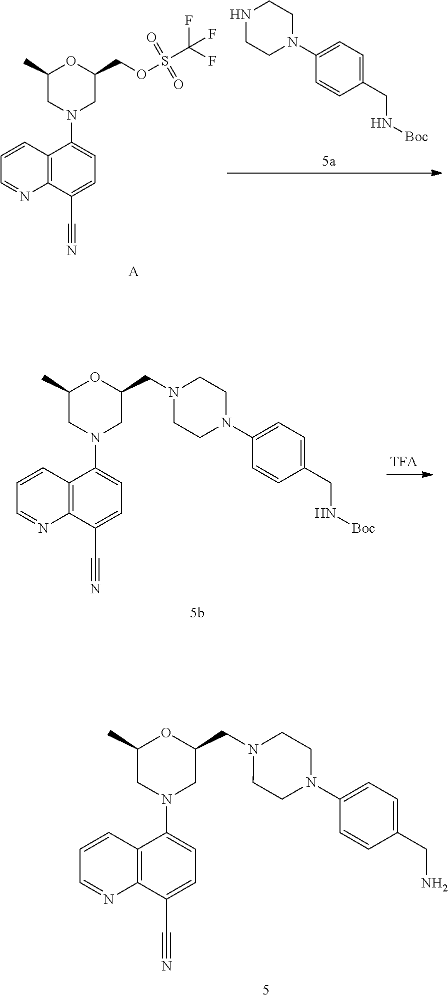

The solution of [(2R,6R)-4-(8-cyano-5-quinolyl)-6-methyl-morpholin-2-yl]methyl trifluoromethanesulfonate (intermediate A, 40 mg, 0.1 mmol), tert-butyl 4-(piperazin-1-yl)benzylcarbamate (compound 5a, 60 mg, 0.2 mmol) and K₂CO₃ (44 mg, 0.3 mmol) in ACN (3 mL) was stirred at 50° C. for 2 hrs. The reaction mixture was diluted with EA (20 mL) and washed with H₂O (20 mL). The organic layer was dried and concentrated under vacuum. The residue was purified by TLC (EA/PE=1/1, R_{f}=0.5) to give compound 5b (30 mg). MS calc'd 557 (MH⁺); measured 557 (MH⁺).

To a solution of tert-butyl N-[[4-[4-[[(2S,6R)-4-(8-cyano-5-quinolyl)-6-methyl-morpholin-2-yl]methyl]piperazin-1-yl]phenyl]methyl]carbamate (compound 5b, 30 mg) in DCM (2 mL) was added TFA (1 mL). After being stirred at 20° C. for 0.5 hour, the mixture was concentrated under vacuum. The residue was purified by prep-HPLC to give Example 5 (4 mg) as a yellow solid. MS calc'd 457 (MH⁺); measured 440 (MH⁺). ¹H NMR (400 MHz, METHANOL-d₄) δ ppm: 9.01 (d, J=2.8 Hz, 1H), 8.69 (d, J=8.4 Hz, 1H), 8.19 (d, J=7.9 Hz, 1H), 7.68 (dd, J=4.3, 8.5 Hz, 1H), 7.41 (d, J=8.5 Hz, 2H), 7.31 (d, J=8.0 Hz, 1H), 7.13 (d, J=8.7 Hz, 2H), 4.54 (br s, 1H), 4.32-4.18 (m, 1H), 4.06 (s, 2H), 3.88-3.34 (m, 12H), 2.93-2.69 (m, 2H), 1.34 (d, J=6.3 Hz, 3H).

### Example 6

### 5-[(2R,6S)-2-methyl-6-[[4-(5-piperazin-1-ylpyrazin-2-yl)piperazin-1-yl]methyl]morpholin-4-yl]quinoline-8-carbonitrile

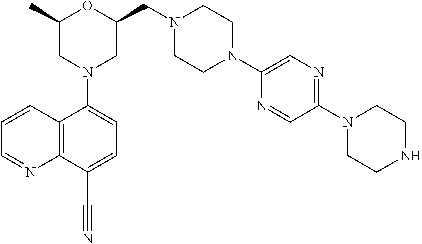 [SMILES: C[C@@H]1CN(c2ccc(C#N)c3ncccc23)C[C@H](CN2CCN(c3cnc(N4CCNCC4)cn3)CC2)O1]

The title compound was prepared according to the following scheme:

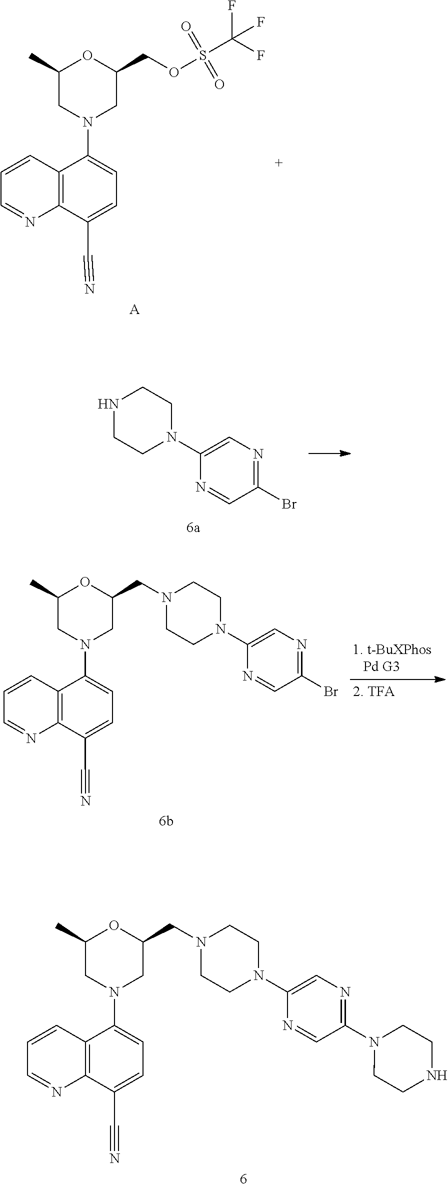

### Step 1: preparation of 5-[(2S,6R)-2-[[4-(5-bromopyrazin-2-yl)piperazin-1-yl]methyl]-6-methyl-morpholin-4-yl]quinoline-8-carbonitrile (Compound 6b)

To a tube was added 2-bromo-5-(piperazin-1-yl)pyrazine (compound 6a, 29 mg, 120 μmol), [(2R,6R)-4-(8-cyano-5-quinolyl)-6-methyl-morpholin-2-yl]methyl trifluoromethanesulfonate (intermediate A, 50 mg, 120 μmol), K₂CO₃ (166 mg, 1.2 mmol) and ACN (10 mL), a suspension was formed. The mixture was heated to reflux for 4 hrs, then diluted with some ACN and filtered through celite. The filtrate was concentrated and diluted with EA. The solution was washed with water. The organic layer was concentrated and the residue was purified by silica gel chromatography to give compound 6b (45 mg). MS calc'd 509 (MH⁺); measured 509 (MH⁺).

### Step 2: preparation of 5-[(2R,6S)-2-methyl-6-[[4-(5-piperazin-1-ylpyrazin-2-yl)piperazin-1-yl]methyl]morpholin-4-yl]quinoline-8-carbonitrile (Example 6)

A mixture of 5-[(2S,6R)-2-[[4-(5-bromopyrazin-2-yl)piperazin-1-yl]methyl]-6-methyl-morpholin-4-yl]quinoline-8-carbonitrile (compound 6b, 30 mg, 60 μmol), tert-butyl piperazine-1-carboxylate (12 mg, 60 μmol), t-BuXPhos Pd G3 (5 mg) and sodium tert-butoxide (17 mg, 180 μmol) in dioxane (10 mL) was heated at 90° C. overnight under N₂ atmosphere. After cooling, the solid was filtered off and washed with EA (20 mL). The filtrate was concentrated to crude intermediate.

The intermediate was dissolved in 5 mL DCM, followed by the addition of 0.5 mL TFA. After being stirred at rt for 3 hrs, the mixture was concentrated in vacuo to give a yellow oil which was purified by prep-HPLC. Example 6 (2 mg) was obtained as a yellow solid. MS: calc'd 514 (MH⁺), measured 514 (MH⁺). ¹H NMR (400 MHz, METHANOL-d4) δ 9.02 (dd, J=1.6, 4.3 Hz, 1H), 8.69 (dd, J=1.7, 8.6 Hz, 1H), 8.19 (d, J=7.9 Hz, 1H), 8.08-7.99 (m, 2H), 7.68 (dd, J=4.3, 8.6 Hz, 1H), 7.31 (d, J=8.1 Hz, 1H), 4.60-4.48 (m, 1H), 4.32-4.14 (m, 2H), 3.87-3.67 (m, 5H), 3.54-3.37 (m, 9H), 3.30-3.20 (m, 5H), 2.88-2.69 (m, 2H), 1.35 (d, J=6.2 Hz, 3H).

### Example 7

### 5-[(2R,6S)-2-methyl-6-[[4-(5-piperazin-1-yl-3-pyridyl)piperazin-1-yl]methyl]morpholin-4-yl]quinoline-8-carbonitrile

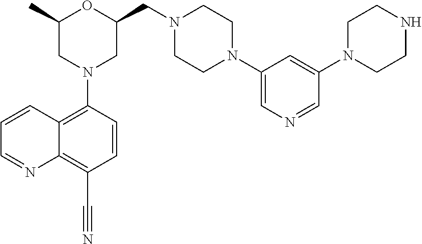 [SMILES: C[C@@H]1CN(c2ccc(C#N)c3ncccc23)C[C@H](CN2CCN(c3cncc(N4CCNCC4)c3)CC2)O1]

The title compound was prepared in analogy to the preparation of Example 6 by using 1-(5-bromo-3-pyridyl)piperazine instead of 2-bromo-5-(piperazin-1-yl)pyrazine (compound 6a). Example 7 (8 mg) was obtained as a yellow solid. MS: calc'd 513 (MH⁺), measured 513 (MH⁺). ¹H NMR (400 MHz, METHANOL-d4) δ 9.02 (dd, J=1.6, 4.3 Hz, 1H), 8.69 (dd, J=1.7, 8.6 Hz, 1H), 8.19 (d, J=7.9 Hz, 1H), 8.02 (t, J=2.7 Hz, 2H), 7.67 (dd, J=4.2, 8.6 Hz, 1H), 7.52 (t, J=2.1 Hz, 1H), 7.31 (d, J=8.1 Hz, 1H), 4.65-4.49 (m, 1H), 4.27-4.14 (m, 1H), 3.94-3.57 (m, 11H), 3.50-3.39 (m, 8H), 2.86-2.67 (m, 3H), 1.34 (d, J=6.2 Hz, 3H).

### Example 8

### 5-[(2R,6S)-2-methyl-6-[[4-(5-piperazin-1-yl-2-pyridyl)piperazin-1-yl]methyl]morpholin-4-yl]quinoline-8-carbonitrile

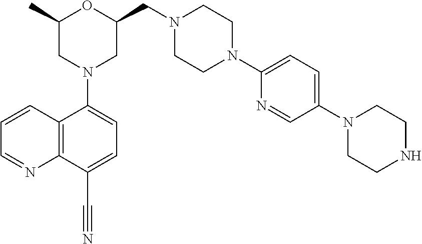 [SMILES: C[C@@H]1CN(c2ccc(C#N)c3ncccc23)C[C@H](CN2CCN(c3ccc(N4CCNCC4)cn3)CC2)O1]

The title compound was prepared in analogy to the preparation of Example 6 by using 1-(5-bromo-2-pyridyl)piperazine instead of 2-bromo-5-(piperazin-1-yl)pyrazine (compound 6a). Example 8 (11 mg) was obtained as a yellow solid. MS: calc'd 513 (MH⁺), measured 513 (MH⁺). ¹H NMR (400 MHz, METHANOL-d4) δ 9.01 (dd, J=1.7, 4.2 Hz, 1H), 8.68 (dd, J=1.6, 8.6 Hz, 1H), 8.18 (d, J=7.9 Hz, 1H), 7.93 (d, J=2.8 Hz, 1H), 7.72-7.61 (m, 2H), 7.30 (d, J=8.1 Hz, 1H), 7.12 (d, J=9.3 Hz, 1H), 4.61-4.49 (m, 1H), 4.29-4.17 (m, 1H), 3.97-3.76 (m, 4H), 3.70-3.53 (m, 4H), 3.49-3.36 (m, 12H), 2.87-2.70 (m, 2H), 1.34 (d, J=6.2 Hz, 3H).

### Example 9

### 5-[(2R,6S)-2-methyl-6-[[4-(4-piperazin-1-yl-2-pyridyl)piperazin-1-yl]methyl]morpholin-4-yl]quinoline-8-carbonitrile

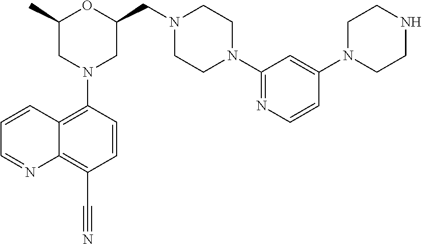 [SMILES: C[C@@H]1CN(c2ccc(C#N)c3ncccc23)C[C@H](CN2CCN(c3cc(N4CCNCC4)ccn3)CC2)O1]

The title compound was prepared in analogy to the preparation of Example 6 by using 1-(4-bromo-2-pyridyl)piperazine instead of 2-bromo-5-(piperazin-1-yl)pyrazine (compound 6a). Example 9 (8 mg) was obtained as a yellow solid. MS: calc'd 513 (MH⁺), measured 513 (MH⁺). ¹H NMR (400 MHz, METHANOL-d4) δ 9.01 (dd, J=1.7, 4.2 Hz, 1H), 8.68 (dd, J=1.6, 8.6 Hz, 1H), 8.19 (d, J=7.9 Hz, 1H), 7.84 (d, J=7.5 Hz, 1H), 7.67 (dd, J=4.3, 8.6 Hz, 1H), 7.30 (d, J=8.1 Hz, 1H), 6.84 (dd, J=2.4, 7.5 Hz, 1H), 6.46 (d, J=2.3 Hz, 1H), 4.61-4.46 (m, 1H), 4.25-4.15 (m, 1H), 4.03-3.85 (m, 8H), 3.72-3.56 (m, 4H), 3.52-3.39 (m, 8H), 2.84-2.71 (m, 2H), 1.33 (d, J=6.2 Hz, 3H).

### Example 10

### 5-[(2R,6S)-2-methyl-6-[[4-(6-piperazin-1-yl-2-pyridyl)piperazin-1-yl]methyl]morpholin-4-yl]quinoline-8-carbonitrile

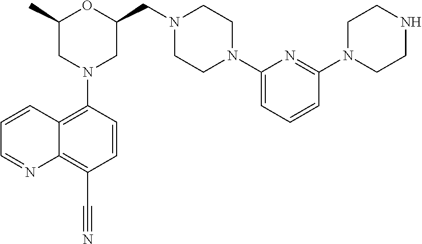 [SMILES: C[C@@H]1CN(c2ccc(C#N)c3ncccc23)C[C@H](CN2CCN(c3cccc(N4CCNCC4)n3)CC2)O1]

The title compound was prepared in analogy to the preparation of Example 6 by using 1-(6-bromo-2-pyridyl)piperazine instead of 2-bromo-5-(piperazin-1-yl)pyrazine (compound 6a). Example 10 (3 mg) was obtained as a yellow solid. MS: calc'd 513 (MH⁺), measured 513 (MH⁺). ¹H NMR (400 MHz, METHANOL-d4) δ 9.02 (dd, J=1.7, 4.2 Hz, 1H), 8.69 (dd, J=1.6, 8.6 Hz, 1H), 8.19 (d, J=7.9 Hz, 1H), 7.67 (dd, J=4.3, 8.6 Hz, 1H), 7.53 (t, J=8.1 Hz, 1H), 7.31 (d, J=8.1 Hz, 1H), 6.35 (dd, J=4.6, 8.1 Hz, 2H), 4.60-4.45 (m, 2H), 4.31-4.15 (m, 2H), 3.87-3.74 (m, 5H), 3.56-3.38 (m, 12H), 2.88-2.69 (m, 3H), 1.34 (d, J=6.4 Hz, 3H).

### Example 11

### 5-[(2S,6R)-2-[[4-cyano-4-(4-piperazin-1-ylphenyl)-1-piperidyl]methyl]-6-methyl-morpholin-4-yl]quinoline-8-carbonitrile

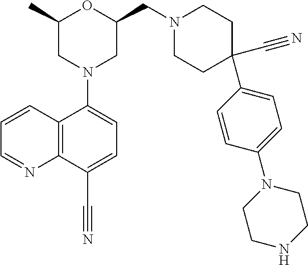 [SMILES: C[C@@H]1CN(c2ccc(C#N)c3ncccc23)C[C@H](CN2CCC(C#N)(c3ccc(N4CCNCC4)cc3)CC2)O1]

The title compound was prepared in analogy to the preparation of Example 6 by using 4-(4-bromophenyl)piperidine-4-carbonitrile instead of 2-bromo-5-(piperazin-1-yl)pyrazine (compound 6a). Example 11 (6 mg) was obtained as a yellow solid. MS: calc'd 536 (MH⁺), measured 536 (MH⁺). ¹H NMR (400 MHz, METHANOL-d4) δ 9.01 (dd, J=1.6, 4.3 Hz, 1H), 8.69 (dd, J=1.6, 8.6 Hz, 1H), 8.19 (d, J=7.9 Hz, 1H), 7.67 (dd, J=4.3, 8.6 Hz, 1H), 7.52 (d, J=8.8 Hz, 2H), 7.31 (d, J=7.9 Hz, 1H), 7.15 (d, J=8.9 Hz, 2H), 4.64-4.46 (m, 1H), 4.27-4.15 (m, 1H), 4.07-3.87 (m, 2H), 3.67-3.37 (m, 14H), 2.86-2.70 (m, 2H), 2.62-2.44 (m, 4H), 1.34 (d, J=6.2 Hz, 3H).

### Example 12

### 5-[(2R,6S)-2-methyl-6-[[4-(2-piperazin-1-ylpyrimidin-5-yl)piperazin-1-yl]methyl]morpholin-4-yl]quinoline-8-carbonitrile

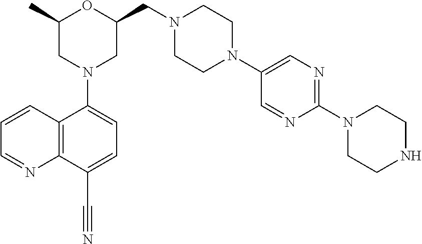 [SMILES: C[C@@H]1CN(c2ccc(C#N)c3ncccc23)C[C@H](CN2CCN(c3cnc(N4CCNCC4)nc3)CC2)O1]

The title compound was prepared in analogy to the preparation of Example 6 by using 2-chloro-5-piperazin-1-yl-pyrimidine instead of 2-bromo-5-(piperazin-1-yl)pyrazine (compound 6a). Example 12 (8 mg) was obtained as a yellow solid. MS: calc'd 514 (MH⁺), measured 514 (MH⁺). ¹H NMR (400 MHz, METHANOL-d4) δ 8.87 (dd, J=1.5, 4.2 Hz, 1H), 8.55 (dd, J=1.6, 8.6 Hz, 1H), 8.12 (s, 2H), 8.05 (d, J=7.9 Hz, 1H), 7.54 (dd, J=4.3, 8.6 Hz, 1H), 7.15 (d, J=8.1 Hz, 1H), 4.17-4.05 (m, 1H), 4.03-3.92 (m, 1H), 3.87-3.78 (m, 4H), 3.43-3.26 (m, 2H), 3.16-2.97 (m, 8H), 2.79-2.39 (m, 8H), 1.16 (d, J=6.2 Hz, 3H).

### Example 13

### 5-[(2R,6S)-2-methyl-6-[[4-(6-piperazin-1-yl-3-pyridyl)piperazin-1-yl]methyl]morpholin-4-yl]quinoline-8-carbonitrile

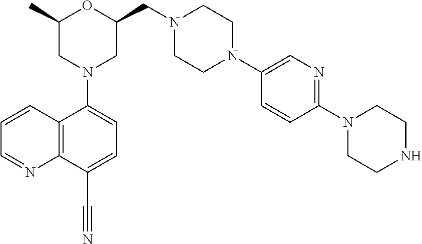 [SMILES: C[C@@H]1CN(c2ccc(C#N)c3ncccc23)C[C@H](CN2CCN(c3ccc(N4CCNCC4)nc3)CC2)O1]

The title compound was prepared in analogy to the preparation of Example 6 by using 1-(6-bromo-3-pyridyl)piperazine instead of 2-bromo-5-(piperazin-1-yl)pyrazine (compound 6a). Example 13 (10 mg) was obtained as a yellow solid. MS: calc'd 513 (MH⁺), measured 513 (MH⁺). ¹H NMR (400 MHz, METHANOL-d4) δ 9.02 (dd, J=1.6, 4.3 Hz, 1H), 8.69 (dd, J=1.7, 8.6 Hz, 1H), 8.19 (d, J=7.9 Hz, 1H), 7.99 (d, J=2.9 Hz, 1H), 7.68 (dd, J=4.3, 8.6 Hz, 1H), 7.54 (dd, J=2.9, 9.2 Hz, 1H), 7.31 (d, J=7.9 Hz, 1H), 7.00 (d, J=9.3 Hz, 1H), 4.62-4.45 (m, 1H), 4.29-4.16 (m, 1H), 3.90-3.41 (m, 14H), 3.30-3.20 (m, 5H), 2.90-2.65 (m, 3H), 1.34 (d, J=6.4 Hz, 3H).

### Example 14

### 5-[(2R,6S)-2-methyl-6-[[4-(4-piperazin-1-ylpyrimidin-2-yl)piperazin-1-yl]methyl]morpholin-4-yl]quinoline-8-carbonitrile

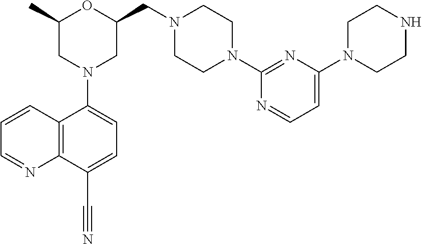 [SMILES: C[C@@H]1CN(c2ccc(C#N)c3ncccc23)C[C@H](CN2CCN(c3nccc(N4CCNCC4)n3)CC2)O1]

The title compound was prepared in analogy to the preparation of Example 6 by using 4-chloro-2-piperazin-1-yl-pyrimidine instead of 2-bromo-5-(piperazin-1-yl)pyrazine (compound 6a). Example 14 (26 mg) was obtained as a yellow solid. MS: calc'd 514 (MH⁺), measured 514 (MH⁺). ¹H NMR (400 MHz, METHANOL-d4) δ 9.02 (dd, J=1.7, 4.3 Hz, 1H), 8.68 (dd, J=1.6, 8.6 Hz, 1H), 8.19 (d, J=7.9 Hz, 1H), 8.01 (d, J=6.8 Hz, 1H), 7.67 (dd, J=4.3, 8.6 Hz, 1H), 7.31 (d, J=8.1 Hz, 1H), 6.53 (d, J=7.0 Hz, 1H), 4.59-4.46 (m, 1H), 4.32-4.15 (m, 2H), 4.15-3.95 (m, 6H), 3.64-3.36 (m, 13H), 2.88-2.70 (m, 2H), 1.34 (d, J=6.2 Hz, 3H).

### Example 15

### 5-[(2R,6S)-2-methyl-6-[[4-(2-piperazin-1-ylpyrimidin-4-yl)piperazin-1-yl]methyl]morpholin-4-yl]quinoline-8-carbonitrile

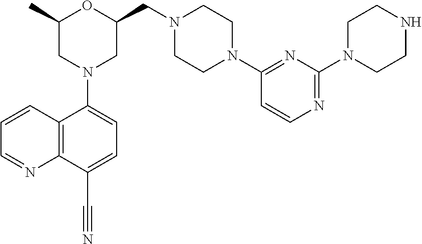 [SMILES: C[C@@H]1CN(c2ccc(C#N)c3ncccc23)C[C@H](CN2CCN(c3ccnc(N4CCNCC4)n3)CC2)O1]

The title compound was prepared in analogy to the preparation of Example 6 by using 2-chloro-4-piperazin-1-yl-pyrimidine instead of 2-bromo-5-(piperazin-1-yl)pyrazine (compound 6a). Example 15 (21 mg) was obtained as a yellow solid. MS: calc'd 514 (MH⁺), measured 514 (MH⁺). ¹H NMR (400 MHz, METHANOL-d4) δ 9.02 (dd, J=1.6, 4.3 Hz, 1H), 8.68 (dd, J=1.7, 8.6 Hz, 1H), 8.19 (d, J=8.1 Hz, 1H), 8.00 (d, J=6.8 Hz, 1H), 7.67 (dd, J=4.3, 8.6 Hz, 1H), 7.31 (d, J=8.1 Hz, 1H), 6.57 (d, J=7.0 Hz, 1H), 4.61-4.46 (m, 1H), 4.28-3.97 (m, 10H), 3.64-3.38 (m, 11H), 2.88-2.69 (m, 2H), 1.34 (d, J=6.2 Hz, 3H).

### Example 16

### 5-[(2R,6S)-2-methyl-6-[[4-(6-piperazin-1-ylpyridazin-3-yl)piperazin-1-yl]methyl]morpholin-4-yl]quinoline-8-carbonitrile

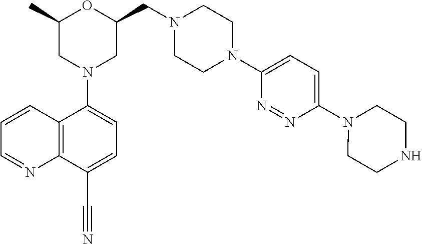 [SMILES: C[C@@H]1CN(c2ccc(C#N)c3ncccc23)C[C@H](CN2CCN(c3ccc(N4CCNCC4)nn3)CC2)O1]

The title compound was prepared in analogy to the preparation of Example 6 by using 3-bromo-6-piperazin-1-yl-pyridazine instead of 2-bromo-5-(piperazin-1-yl)pyrazine (compound 6a). Example 16 (2 mg) was obtained as a yellow solid. MS: calc'd 514 (MH⁺), measured 514 (MH⁺). ¹H NMR (400 MHz, METHANOL-d4) δ 9.08-8.97 (m, 1H), 8.69 (d, J=8.4 Hz, 1H), 8.19 (d, J=8.1 Hz, 1H), 7.74-7.61 (m, 3H), 7.31 (d, J=8.1 Hz, 1H), 4.61-4.47 (m, 1H), 4.30-4.16 (m, 1H), 4.07-3.75 (m, 8H), 3.73-3.37 (m, 12H), 2.87-2.70 (m, 2H), 1.34 (d, J=6.2 Hz, 3H).

### Example 17

### 5-[(2R,6S)-2-methyl-6-[[4-(5-piperazin-1-ylpyrimidin-2-yl)piperazin-1-yl]methyl]morpholin-4-yl]quinoline-8-carbonitrile

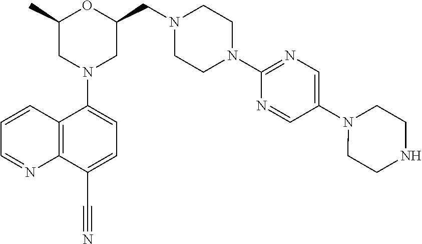 [SMILES: C[C@@H]1CN(c2ccc(C#N)c3ncccc23)C[C@H](CN2CCN(c3ncc(N4CCNCC4)cn3)CC2)O1]

The title compound was prepared in analogy to the preparation of Example 6 by using 5-chloro-2-piperazin-1-yl-pyrimidine instead of 2-bromo-5-(piperazin-1-yl)pyrazine (compound 6a). Example 17 (2 mg) was obtained as a yellow solid. MS: calc'd 514 (MH⁺), measured 514 (MH⁺). ¹H NMR (400 MHz, METHANOL-d4) δ 9.00 (dd, J=1.6, 4.3 Hz, 1H), 8.69 (dd, J=1.5, 8.6 Hz, 1H), 8.22-8.11 (m, 3H), 7.66 (dd, J=4.2, 8.5 Hz, 1H), 7.28 (d, J=7.9 Hz, 1H), 4.29-4.04 (m, 2H), 3.75 (t, J=5.0 Hz, 4H), 3.54-3.36 (m, 4H), 3.05 (br d, J=1.8 Hz, 6H), 2.80-2.40 (m, 8H), 1.36-1.19 (m, 3H).

### Example 18

### 5-[(2R,6S)-2-methyl-6-[[4-(6-piperazin-1-ylpyrimidin-4-yl)piperazin-1-yl]methyl]morpholin-4-yl]quinoline-8-carbonitrile

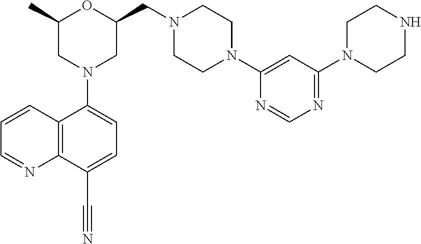 [SMILES: C[C@@H]1CN(c2ccc(C#N)c3ncccc23)C[C@H](CN2CCN(c3cc(N4CCNCC4)ncn3)CC2)O1]

The title compound was prepared in analogy to the preparation of Example 6 by using 4-chloro-6-piperazin-1-yl-pyrimidine instead of 2-bromo-5-(piperazin-1-yl)pyrazine (compound 6a). Example 18 (5 mg) was obtained as a yellow solid. MS: calc'd 514 (MH⁺), measured 514 (MH⁺). ¹H NMR (400 MHz, METHANOL-d4) δ 9.02 (dd, J=1.6, 4.3 Hz, 1H), 8.69 (dd, J=1.6, 8.6 Hz, 1H), 8.30-8.15 (m, 2H), 7.68 (dd, J=4.3, 8.6 Hz, 1H), 7.31 (d, J=8.1 Hz, 1H), 6.18 (s, 1H), 4.61-4.46 (m, 1H), 4.27-4.15 (m, 1H), 3.99-3.87 (m, 5H), 3.64-3.37 (m, 10H), 3.31-3.20 (m, 5H), 2.90-2.66 (m, 2H), 1.35 (d, J=6.2 Hz, 3H).

### Example 19

### 5-[(2R,6S)-2-methyl-6-[[4-[4-[(2R)-morpholin-2-yl]phenyl]piperazin-1-yl]methyl]morpholin-4-yl]quinoline-8-carbonitrile

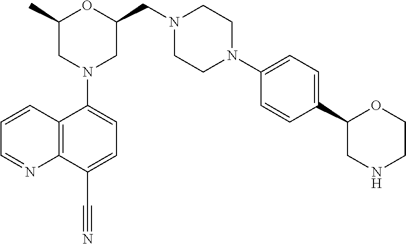 [SMILES: C[C@@H]1CN(c2ccc(C#N)c3ncccc23)C[C@H](CN2CCN(c3ccc([C@@H]4CNCCO4)cc3)CC2)O1]

The title compound was prepared according to the following scheme:

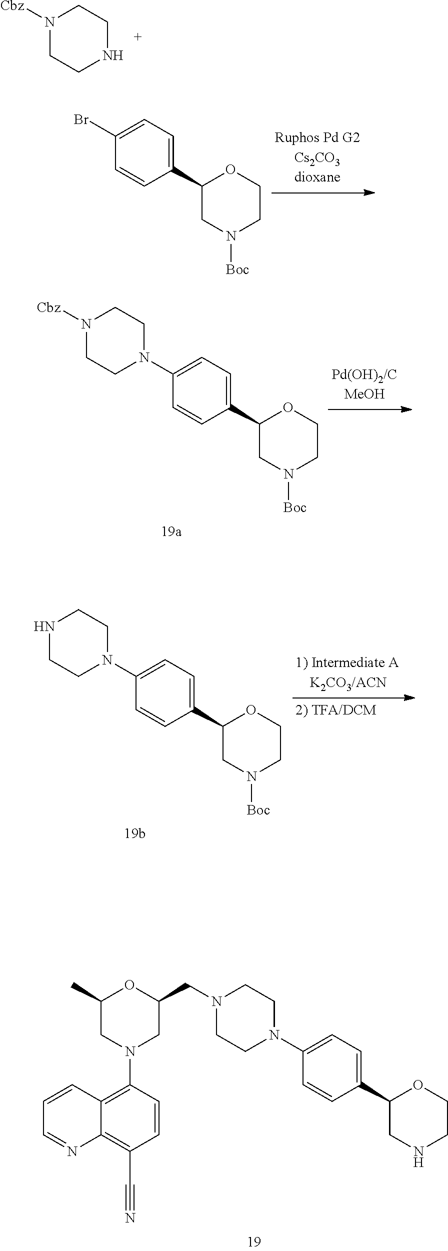 [SMILES: O=C(OCc1ccccc1)N1CCNCC1 ; CC(C)(C)OC(=O)N1CCO[C@H](c2ccc(Br)cc2)C1 ; CC(C)(C)OC(=O)N1CCO[C@H](c2ccc(N3CCN(C(=O)OCc4ccccc4)CC3)cc2)C1 ; CC(C)(C)OC(=O)N1CCO[C@H](c2ccc(N3CCNCC3)cc2)C1 ; C[C@@H]1CN(c2ccc(C#N)c3ncccc23)C[C@H](CN2CCN(c3ccc([C@@H]4CNCCO4)cc3)CC2)O1]

### Step 1: Preparation of tert-butyl (2R)-2-[4-(4-benzyloxycarbonylpiperazin-1-yl)phenyl]morpholine-4-carboxylate (Compound 19a)

To a tube was added benzyl piperazine-1-carboxylate (77.2 mg, 351 μmol), tert-butyl (R)-2-(4-bromophenyl)morpholine-4-carboxylate (WO 2012168260, 100 mg, 292 μmol), Cs₂CO₃ (190 mg, 584 μmol) and dioxane (4 mL). The suspension was bubbled with N₂ for 5 mins and Ruphos Pd G2 (22.7 mg, 29.2 μmol) was added. The mixture was heated at 90° C. and stirred for 2 hrs. The mixture was cooled and filtered. The filtrate was concentrated to give an oil. The crude was purified via silica gel chromatography, the elution was concentrated to give compound 19a (100 mg) as an oil. MS calc'd 482 (MH⁺); measured 482 (MH⁺).

### Step 2: preparation of tert-butyl (2R)-2-(4-piperazin-1-ylphenyl)morpholine-4-carboxylate (Compound 19b)

The oil (compound 19a, 100 mg, 207 μmol) was dissolved in MeOH (4 mL) followed by the addition of Pd(OH)₂ (20% on carbon with 50% H₂O) (10 mg, 71.2 μmol). After being stirred at rt for 2 hrs under H₂ atmosphere, the mixture was filtered, and the filtrate was concentrated to give compound 19b (70 mg) as an oil. MS calc'd 348 (MH⁺); measured 348 (MH⁺).

### Step 3: preparation of 5-[(2R,6S)-2-methyl-6-[[4-[4-[(2R)-morpholin-2-yl]phenyl]piperazin-1-yl]methyl]morpholin-4-yl]quinoline-8-carbonitrile (Compound 19)

To a flask was added ((2R,6R)-4-(8-cyanoquinolin-5-yl)-6-methylmorpholin-2-yl)methyl trifluoromethanesulfonate (Intermediate A, 30 mg, 72.2 μmol), tert-butyl (2R)-2-(4-(piperazin-1-yl)phenyl)morpholine-4-carboxylate (compound 19b, 32.6 mg, 93.9 μmol), potassium carbonate (29.9 mg, 217 μmol) and ACN (4 mL). The mixture was heated to 85° C. for 2 hrs, then filtered through celite. The filtrate was concentrated to give a crude oil, which was dissolved in DCM (2 mL) followed by the addition of TFA (1.48 g, 1 mL, 13 mmol) was added. The mixture was stirred at rt for 1 hour. The mixture was concentrated directly to give an oil which was purified by prep-HPLC. Example 19 (29.5 mg) was obtained as a light yellow powder. MS: calc'd 513 (MH⁺), measured 513 (MH⁺). ¹H NMR (400 MHz, METHANOL-d₄) δ=9.02 (dd, J=1.6, 4.3 Hz, 1H), 8.69 (dd, J=1.6, 8.6 Hz, 1H), 8.20 (d, J=8.1 Hz, 1H), 7.68 (dd, J=4.2, 8.6 Hz, 1H), 7.38 (d, J=8.8 Hz, 2H), 7.31 (d, J=8.1 Hz, 1H), 7.10 (d, J=8.8 Hz, 2H), 4.71 (dd, J=2.3, 11.2 Hz, 1H), 4.57-4.49 (m, 1H), 4.27-4.17 (m, 2H), 3.99 (dt, J=2.9, 12.5 Hz, 1H), 3.95-3.69 (m, 3H), 3.49-3.42 (m, 5H), 3.42-3.34 (m, 6H), 3.30-3.24 (m, 1H), 3.16-3.09 (m, 1H), 2.86-2.73 (m, 2H), 1.35 (d, J=6.4 Hz, 3H).

### Example 20

### 5-[(2R,6S)-2-methyl-6-[[4-[4-[(2S)-morpholin-2-yl]phenyl]piperazin-1-yl]methyl]morpholin-4-yl]quinoline-8-carbonitrile

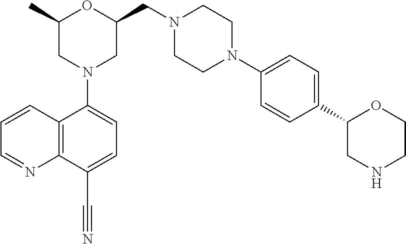 [SMILES: C[C@@H]1CN(c2ccc(C#N)c3ncccc23)C[C@H](CN2CCN(c3ccc([C@H]4CNCCO4)cc3)CC2)O1]

The title compound was prepared in analogy to the preparation of Example 19 by using tert-butyl (S)-2-(4-bromophenyl)morpholine-4-carboxylate) instead of tert-butyl (R)-2-(4-bromophenyl)morpholine-4-carboxylate. Example 20 (36.7 mg) was obtained as a pale yellow powder. MS: calc'd 513 (MH⁺), measured 513 (MH⁺). ¹H NMR (400 MHz, METHANOL-d₄) δ=9.01 (dd, J=1.6, 4.3 Hz, 1H), 8.68 (dd, J=1.6, 8.6 Hz, 1H), 8.18 (d, J=8.1 Hz, 1H), 7.67 (dd, J=4.2, 8.6 Hz, 1H), 7.37 (d, J=8.7 Hz, 2H), 7.30 (d, J=8.1 Hz, 1H), 7.09 (d, J=8.8 Hz, 2H), 4.71 (dd, J=2.3, 11.2 Hz, 1H), 4.58-4.50 (m, 1H), 4.27-4.17 (m, 2H), 4.04-3.95 (m, 1H), 3.94-3.56 (m, 4H), 3.49-3.42 (m, 5H), 3.41-3.34 (m, 4H), 3.32-3.18 (m, 2H), 3.12 (dd, J=11.4, 12.7 Hz, 1H), 2.86-2.72 (m, 2H), 1.34 (d, J=6.2 Hz, 3H).

### Example 21

### 5-[(2R,6S)-2-methyl-6-[[4-[6-(4-methylpiperazin-1-yl)pyrimidin-4-yl]piperazin-1-yl]methyl]morpholin-4-yl]quinoline-8-carbonitrile

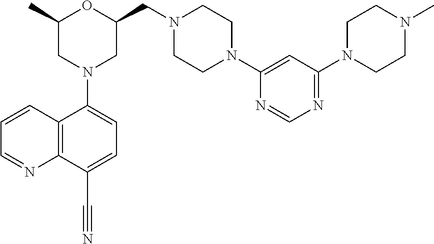 [SMILES: C[C@@H]1CN(c2ccc(C#N)c3ncccc23)C[C@H](CN2CCN(c3cc(N4CCN(C)CC4)ncn3)CC2)O1]

The title compound was prepared in analogy to the preparation of Example 6 by using 4-chloro-6-piperazin-1-yl-pyrimidine and 1-methylpiperazine instead of 2-bromo-5-(piperazin-1-yl)pyrazine (compound 6a) and tert-butyl piperazine-1-carboxylate. Example 21 (3 mg) was obtained as a yellow solid. MS: calc'd 528 (MH⁺), measured 528 (MH⁺). ¹H NMR (400 MHz, METHANOL-d4) δ 8.99 (dd, J=1.7, 4.2 Hz, 1H), 8.66 (dd, J=1.7, 8.6 Hz, 1H), 8.26 (s, 1H), 8.16 (d, J=7.9 Hz, 1H), 7.65 (dd, J=4.3, 8.6 Hz, 1H), 7.28 (d, J=8.1 Hz, 1H), 6.20 (s, 1H), 4.57-4.42 (m, 2H), 4.28-4.10 (m, 2H), 3.72-3.33 (m, 13H), 3.25-3.10 (m, 5H), 2.96 (s, 3H), 2.83-2.63 (m, 2H), 1.32 (d, J=6.2 Hz, 3H).

### Example 22

### 5-[(2S,6R)-2-[[4-[2-(3-aminoazetidin-1-yl)pyrimidin-4-yl]piperazin-1-yl]methyl]-6-methyl-morpholin-4-yl]quinoline-8-carbonitrile

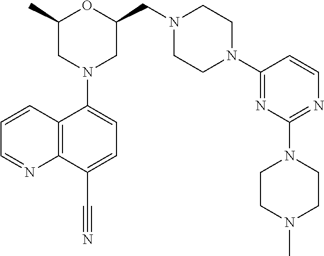 [SMILES: C[C@@H]1CN(c2ccc(C#N)c3ncccc23)C[C@H](CN2CCN(c3ccnc(N4CCN(C)CC4)n3)CC2)O1]

The title compound was prepared according to the following scheme:

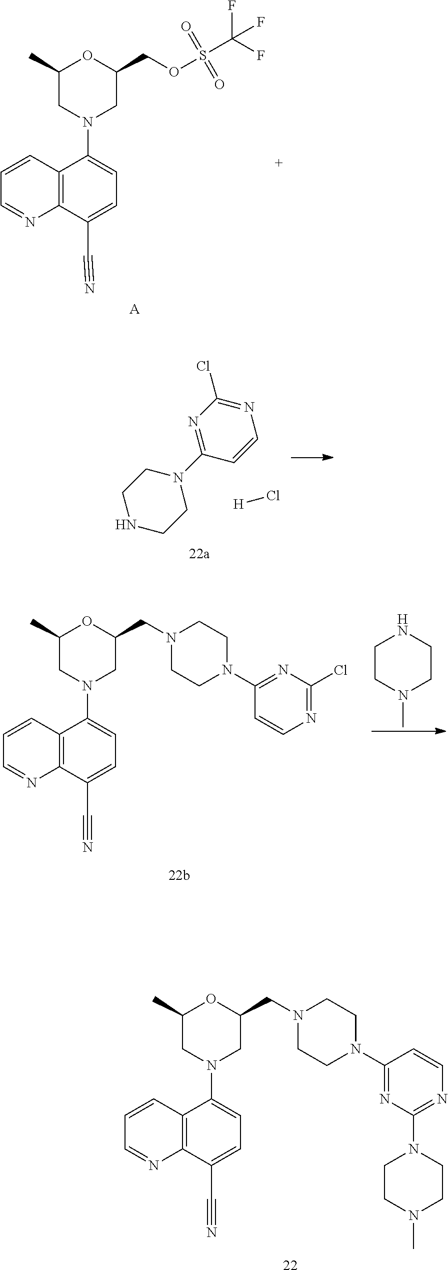

### Step 1: 5-((2S,6R)-2-((4-(2-chloropyrimidin-4-yl)piperazin-1-yl)methyl)-6-methylmorpholino)quinoline-8-carbonitrile (Compound 22b)

A suspension of 2-chloro-4-(piperazin-1-yl)pyrimidine hydrochloride (compound 22a, 420 mg, 1.78 mmol) and K₂CO₃ (710 mg, 5.1 mmol) in ACN (8 mL) was heated to 50° C. for 1 h. Then ((2R,6R)-4-(8-cyanoquinolin-5-yl)-6-methylmorpholin-2-yl)methyl trifluoromethanesulfonate (intermediate A, 706 mg, 1.7 mmol) was added. The reaction mixture was heat to 90° C. for 3 hrs. Then the mixture was filtered and the filtrate was concentrated and purified by silica gel chromatography to give compound 22b (245 mg, 528 μmol) as yellow solid. MS: calc'd 464 (MH⁺), measured 464 (MH⁺).

### Step 2: 5-((2R,6S)-2-methyl-6-((4-(2-(4-methylpiperazin-1-yl)pyrimidin-4-yl)piperazin-1-yl)methyl)morpholino)quinoline-8-carbonitrile (Example 22)

A suspension of 5-((2S,6R)-2-((4-(2-chloropyrimidin-4-yl)piperazin-1-yl)methyl)-6-methylmorpholino)quinoline-8-carbonitrile (compound 22b, 46 mg, 99.1 μmol), 1-methylpiperazine (29 mg, 297 μmol) and K₂CO₃ (41 mg, 297 μmol) in ACN (2 mL) was heated at 110° C. under microwave for 2 hrs. Then the mixture was cooled to room temperature and filtered. The filtrate was concentrated and purified by prep-HPLC to afford Example 22 (44 mg) as an orange solid. MS: calc'd 528 (MH⁺), measured 528 (MH⁺). ¹H NMR (METHANOL-d4, 400 MHz) δ 9.01 (dd, 1H, J=1.6, 4.3 Hz), 8.68 (dd, 1H, J=1.6, 8.6 Hz), 8.18 (d, 1H, J=7.9 Hz), 8.00 (d, 1H, J=7.2 Hz), 7.67 (dd, 1H, J=4.3, 8.6 Hz), 7.30 (d, 1H, J=8.1 Hz), 6.67 (d, 1H, J=7.2 Hz), 4.5-4.6 (m, 1H), 3.9-4.4 (m, 8H), 3.4-3.8 (m, 13H), 3.00 (s, 3H), 2.7-2.9 (m, 2H), 1.33 (d, 3H, J=6.2 Hz).

### Example 23

### 5-[(2S,6R)-2-[[4-[2-(3-aminoazetidin-1-yl)pyrimidin-4-yl]piperazin-1-yl]methyl]-6-methyl-morpholin-4-yl]quinoline-8-carbonitrile

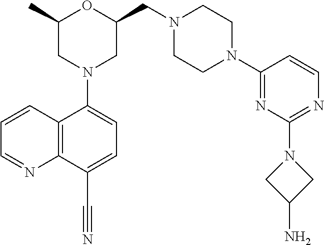 [SMILES: C[C@@H]1CN(c2ccc(C#N)c3ncccc23)C[C@H](CN2CCN(c3ccnc(N4CC(N)C4)n3)CC2)O1]

The title compound was prepared according to the following scheme:

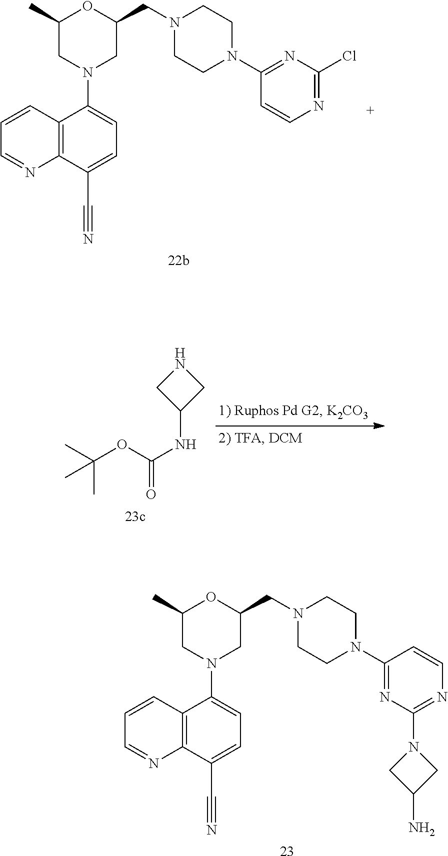 [SMILES: C[C@@H]1CN(c2ccc(C#N)c3ncccc23)C[C@H](CN2CCN(c3ccnc(Cl)n3)CC2)O1 ; "22b" ; CC(C)(C)OC(=O)NC1CNC1 ; C[C@@H]1CN(c2ccc(C#N)c3ncccc23)C[C@H](CN2CCN(c3ccnc(N4CC(N)C4)n3)CC2)O1 ; "23"]

A suspension of 5-((2S,6R)-2-((4-(2-chloropyrimidin-4-yl)piperazin-1-yl)methyl)-6-methylmorpholino)quinoline-8-carbonitrile (compound 22b, 22 mg, 47.4 μmol), tert-butyl azetidin-3-ylcarbamate (compound 23c, 25 mg, 142 μmol), Ruphos Pd G2 (7 mg, 8.62 μmol) and K₂CO₃ (20 mg, 142 μmol) in 1,4-Dioxane (2 mL) was heated to 100° C. for 12 hrs under nitrogen atmosphere. The reaction mixture was cooled to room temperature and filtered. The filtrate was concentrated to give an intermediate (MS: calc'd 600 (MH⁺), measured 600 (MH⁺)), which was dissolved in 5 mL DCM followed by the addition of 0.5 mL TFA. The reaction mixture was stirred at rt for 2 hours, then concentrated to give a crude product which was purified by prep-HPLC to afford Example 23 (4 mg) as light yellow solid. MS: calc'd 500 (MH⁺), measured 500 (MH⁺). ¹H NMR (METHANOL-d₄, 400 MHz) δ 8.89 (dd, 1H, J=1.6, 4.3 Hz), 8.56 (dd, 1H, J=1.7, 8.6 Hz), 8.07 (d, 1H, J=7.9 Hz), 7.78 (d, 1H, J=7.1 Hz), 7.55 (dd, 1H, J=4.3, 8.6 Hz), 7.18 (d, 1H, J=8.1 Hz), 6.39 (d, 1H, J=7.1 Hz), 4.43 (dd, 2H, J=6.7, 9.8 Hz), 4.30 (br s, 1H), 4.0-4.2 (m, 4H), 3.90 (br s, 3H), 3.3-3.4 (m, 2H), 3.1-3.1 (m, 2H), 2.9-3.0 (m, 2H), 2.6-2.7 (m, 2H), 1.93 (s, 3H), 1.20 (d, 3H, J=6.2 Hz).

### Example 24

### 5-[(2S,6R)-2-[[4-[2-[[(3S,4R)-4-fluoropyrrolidin-3-yl]amino]pyrimidin-4-yl]piperazin-1-yl]methyl]-6-methyl-morpholin-4-yl]quinoline-8-carbonitrile

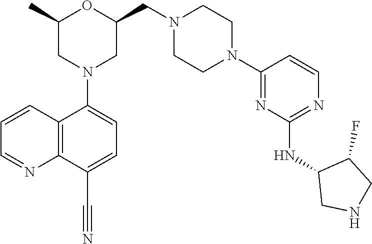 [SMILES: C[C@@H]1CN(c2ccc(C#N)c3ncccc23)C[C@H](CN2CCN(c3ccnc(N[C@H]4CNC[C@H]4F)n3)CC2)O1]

The title compound was prepared in analogy to the preparation of Example 23 by using tert-butyl (3S,4R)-3-amino-4-fluoropyrrolidine-1-carboxylate (Pharmablock, PB07374, CAS: 1174020-30-4) instead of tert-butyl azetidin-3-ylcarbamate (compound 23c). Example 24 (12 mg) was obtained as a light yellow solid. MS: calc'd 532 (MH⁺), measured 532 (MH⁺). ¹H NMR (METHANOL-d₄, 400 MHz) δ 9.02 (dd, 1H, J=1.7, 4.2 Hz), 8.68 (dd, 1H, J=1.7, 8.6 Hz), 8.19 (d, 1H, J=7.9 Hz), 7.92 (d, 1H, J=7.5 Hz), 7.67 (dd, 1H, J=4.3, 8.6 Hz), 7.31 (d, 1H, J=8.1 Hz), 6.63 (d, 1H, J=7.3 Hz), 5.3-5.5 (m, 1H), 4.9-5.2 (m, 2H), 4.48 (br s, 1H), 4.0-4.3 (m, 4H), 3.7-4.0 (m, 3H), 3.4-3.5 (m, 6H), 3.28 (br d, 3H, J=6.4 Hz), 2.7-2.8 (m, 2H), 1.33 (d, 3H, J=6.2 Hz).

### Example 25

### 5-[(2S,6R)-2-[[4-[2-(6-hydroxy-1,4-diazepan-1-yl)pyrimidin-4-yl]piperazin-1-yl]methyl]-6-methyl-morpholin-4-yl]quinoline-8-carbonitrile

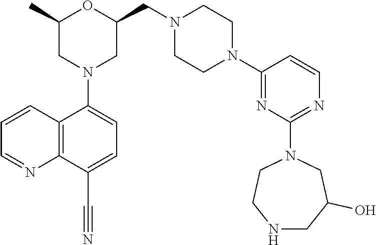 [SMILES: C[C@@H]1CN(c2ccc(C#N)c3ncccc23)C[C@H](CN2CCN(c3ccnc(N4CCNCC(O)C4)n3)CC2)O1]

The title compound was prepared in analogy to the preparation of Example 23 by using tert-butyl 6-hydroxy-1,4-diazepane-1-carboxylate (Wuxi Apptec, WX604354, CAS: 956317-40-1) instead of tert-butyl azetidin-3-ylcarbamate (compound 23c). Example 25 (13 mg) was obtained as a light yellow solid. MS: calc'd 544 (MH⁺), measured 544 (MH⁺). ¹H NMR (METHANOL-d₄, 400 MHz) δ 9.02 (dd, 1H, J=1.5, 4.2 Hz), 8.68 (dd, 1H, J=1.6, 8.7 Hz), 8.19 (d, 1H, J=7.9 Hz), 7.93 (d, 1H, J=7.2 Hz), 7.67 (dd, 1H, J=4.2, 8.6 Hz), 7.31 (d, 1H, J=7.9 Hz), 6.67 (d, 1H, J=7.5 Hz), 4.53 (br s, 1H), 4.4-4.5 (m, 1H), 3.9-4.3 (m, 8H), 3.78 (s, 1H), 3.47 (br s, 13H), 2.7-2.9 (m, 2H), 1.34 (d, 3H, J=6.4 Hz).

### Example 26

### 5-[(2S,6R)-2-[[4-[2-trans-(3-amino-4-methoxy-pyrrolidin-1-yl)pyrimidin-4-yl]piperazin-1-yl]methyl]-6-methyl-morpholin-4-yl]quinoline-8-carbonitrile

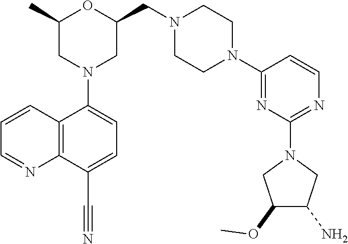 [SMILES: CO[C@H]1CN(c2nccc(N3CCN(C[C@H]4CN(c5ccc(C#N)c6ncccc56)C[C@@H](C)O4)CC3)n2)C[C@@H]1N]

The title compound was prepared in analogy to the preparation of Example 23 by using tert-butyl trans-(4-methoxypyrrolidin-3-yl)carbamate instead of tert-butyl azetidin-3-ylcarbamate (compound 23c). Example 26 (4.4 mg) was obtained as a light yellow solid. MS: calc'd 544 (MH⁺), measured 544 (MH⁺). ¹H NMR (METHANOL-d₄, 400 MHz) δ 8.88 (dd, 1H, J=1.6, 4.2 Hz), 8.56 (d, 1H, J=10.3 Hz), 8.05 (d, 1H, J=8.1 Hz), 7.70 (d, 1H, J=6.4 Hz), 7.54 (dd, 1H, J=4.2, 8.7 Hz), 7.16 (d, 1H, J=8.1 Hz), 5.97 (d, 1H, J=6.4 Hz), 3.9-4.2 (m, 2H), 3.5-3.8 (m, 7H), 3.3-3.5 (m, 9H), 3.0-3.1 (m, 1H), 2.4-2.7 (m, 8H), 1.16 (d, 3H, J=6.2 Hz).

### Example 27

### 5-[(2S,6R)-2-[[4-[2-(4-amino-1-piperidyl)pyrimidin-4-yl]piperazin-1-yl]methyl]-6-methyl-morpholin-4-yl]quinoline-8-carbonitrile

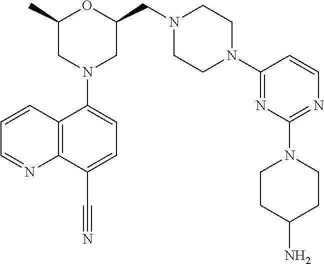 [SMILES: C[C@@H]1CN(c2ccc(C#N)c3ncccc23)C[C@H](CN2CCN(c3ccnc(N4CCC(N)CC4)n3)CC2)O1]

The title compound was prepared in analogy to the preparation of Example 23 by using tert-butyl piperidin-4-ylcarbamate instead of tert-butyl azetidin-3-ylcarbamate (compound 23c). Example 27 (15.5 mg) was obtained as a light yellow solid. MS: calc'd 528 (MH⁺), measured 528 (MH⁺). 1H NMR (METHANOL-d₄, 400 MHz) δ 9.02 (dd, 1H, J=1.7, 4.3 Hz), 8.68 (dd, 1H, J=1.7, 8.6 Hz), 8.19 (d, 1H, J=8.1 Hz), 7.90 (d, 1H, J=7.5 Hz), 7.67 (dd, 1H, J=4.3, 8.6 Hz), 7.31 (d, 1H, J=8.1 Hz), 6.62 (d, 1H, J=7.5 Hz), 4.5-4.6 (m, 3H), 4.0-4.3 (m, 4H), 3.4-3.6 (m, 8H), 3.4-3.4 (m, 2H), 3.1-3.2 (m, 2H), 2.7-2.9 (m, 2H), 2.1-2.2 (m, 2H), 1.6-1.8 (m, 2H), 1.34 (d, 3H, J=6.4 Hz).

### Example 28

### 5-[(2S,6R)-2-[[4-[2-(azetidin-3-ylamino)pyrimidin-4-yl]piperazin-1-yl]methyl]-6-methyl-morpholin-4-yl]quinoline-8-carbonitrile

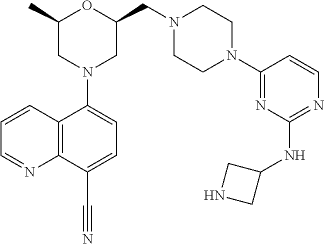 [SMILES: C[C@@H]1CN(c2ccc(C#N)c3ncccc23)C[C@H](CN2CCN(c3ccnc(NC4CNC4)n3)CC2)O1]

The title compound was prepared in analogy to the preparation of Example 23 by using tert-butyl 3-aminoazetidine-1-carboxylate instead of tert-butyl azetidin-3-ylcarbamate (compound 23c). Example 28 (9.0 mg) was obtained as light yellow solid. MS: calc'd 500 (MH⁺), measured 500 (MH⁺). ¹H NMR (METHANOL-d₄, 400 MHz) δ 9.02 (dd, 1H, J=1.5, 4.2 Hz), 8.68 (d, 1H, J=8.7 Hz), 8.19 (d, 1H, J=7.9 Hz), 7.92 (d, 1H, J=7.3 Hz), 7.67 (dd, 1H, J=4.3, 8.5 Hz), 7.31 (d, 1H, J=8.1 Hz), 6.64 (d, 1H, J=7.5 Hz), 4.6-4.7 (m, 1H), 4.4-4.6 (m, 2H), 4.3-4.4 (m, 2H), 4.2-4.2 (m, 2H), 3.5-3.6 (m, 3H), 3.4-3.5 (m, 2H), 3.4-3.4 (m, 1H), 2.7-2.9 (m, 2H), 2.05 (s, 6H), 1.33 (dd, 3H, J=1.0, 6.2 Hz).

### Example 29

### 5-[(2S,6R)-2-[[4-[2-[(3R)-3-(hydroxymethyl)piperazin-1-yl]pyrimidin-4-yl]piperazin-1-yl]methyl]-6-methyl-morpholin-4-yl]quinoline-8-carbonitrile

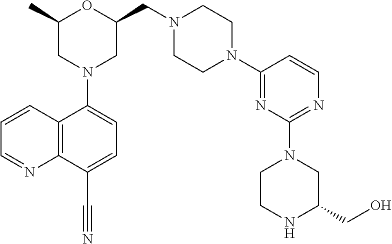 [SMILES: C[C@@H]1CN(c2ccc(C#N)c3ncccc23)C[C@H](CN2CCN(c3ccnc(N4CCN[C@@H](CO)C4)n3)CC2)O1]

The title compound was prepared in analogy to the preparation of Example 23 by using tert-butyl (R)-2-(hydroxymethyl)piperazine-1-carboxylate (Bepharm, B32231, CAS: 169448-87-7) instead of tert-butyl azetidin-3-ylcarbamate (compound 23c). Example 29 (20.9 mg) was obtained as light yellow solid. MS: calc'd 544 (MH⁺), measured 544 (MH⁺). ¹H NMR (METHANOL-d₄, 400 MHz) δ 9.02 (dd, 1H, J=1.7, 4.2 Hz), 8.68 (dd, 1H, J=1.6, 8.7 Hz), 8.2-8.2 (m, 1H), 8.02 (d, 1H, J=6.8 Hz), 7.67 (dd, 1H, J=4.2, 8.6 Hz), 7.31 (d, 1H, J=7.9 Hz), 6.51 (d, 1H, J=6.7 Hz), 4.5-4.7 (m, 4H), 4.20 (br d, 2H, J=10.3 Hz), 4.10 (br s, 3H), 3.7-3.9 (m, 3H), 3.4-3.6 (m, 12H), 2.7-2.9 (m, 2H), 1.34 (d, 3H, J=6.2 Hz).

### Example 30

### 5-[(2S,6R)-2-[[4-[2-[2-(dimethylamino)ethoxy]pyrimidin-4-yl]piperazin-1-yl]methyl]-6-methyl-morpholin-4-yl]quinoline-8-carbonitrile

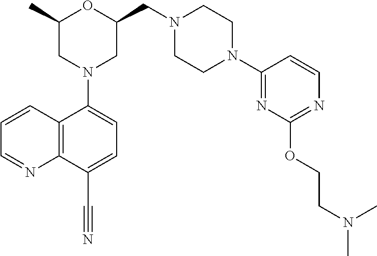 [SMILES: C[C@@H]1CN(c2ccc(C#N)c3ncccc23)C[C@H](CN2CCN(c3ccnc(OCCN(C)C)n3)CC2)O1]

The title compound was prepared in analogy to the preparation of Example 23 by using 2-(dimethylamino)ethan-1-ol instead of tert-butyl azetidin-3-ylcarbamate (compound 23c). Example 30 (17.8 mg) was obtained as light yellow solid. MS: calc'd 517 (MH⁺), measured 517 (MH⁺). ¹H NMR (METHANOL-d₄, 400 MHz) δ 8.9-8.9 (m, 1H), 8.82 (d, 1H, J=8.7 Hz), 8.0-8.0 (m, 2H), 7.4-7.6 (m, 2H), 7.3-7.4 (m, 2H), 6.97 (d, 1H, J=9.2 Hz), 6.88 (d, 1H, J=8.6 Hz), 4.05 (dd, 1H, J=7.0, 9.5 Hz), 3.8-3.9 (m, 3H), 3.7-3.8 (m, 4H), 3.4-3.6 (m, 7H), 3.1-3.3 (m, 5H), 2.4-2.6 (m, 1H), 2.2-2.3 (m, 1H), 1.3-1.3 (m, 3H).

### Example 31

### 5-[(2R,6S)-2-methyl-6-[[4-[2-[4-[(2S)-pyrrolidine-2-carbonyl]piperazin-1-yl]pyrimidin-4-yl]piperazin-1-yl]methyl]morpholin-4-yl]quinoline-8-carbonitrile

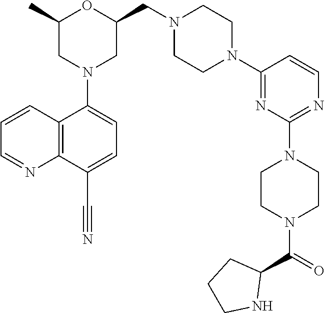 [SMILES: C[C@@H]1CN(c2ccc(C#N)c3ncccc23)C[C@H](CN2CCN(c3ccnc(N4CCN(C(=O)[C@@H]5CCCN5)CC4)n3)CC2)O1]

The title compound was prepared according to the following scheme:

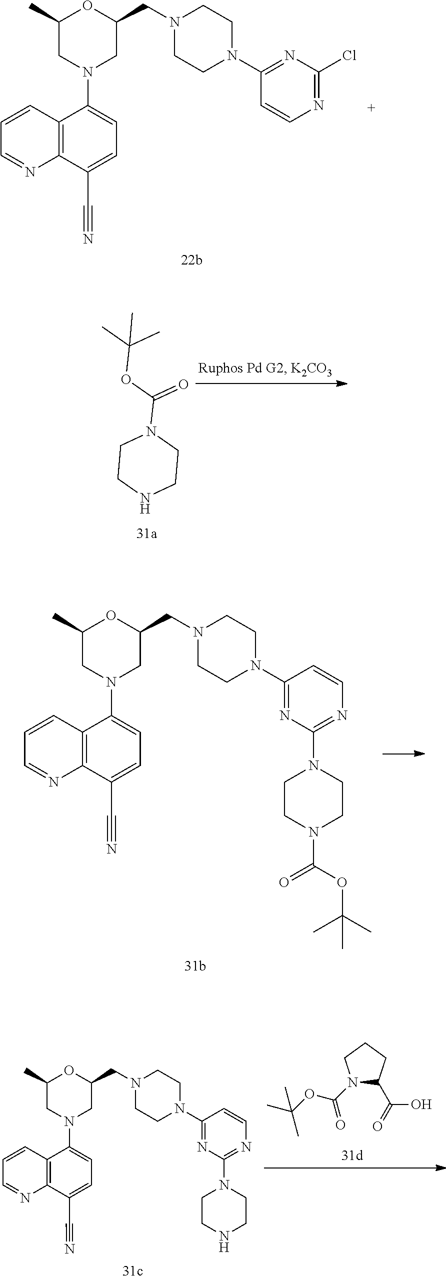

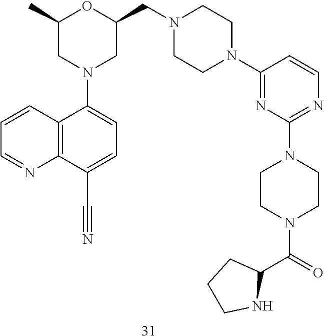

### Step 1: tert-butyl 4-(4-(4-(((2S,6R)-4-(8-cyanoquinolin-5-yl)-6-methylmorpholin-2-yl)methyl)piperazin-1-yl)pyrimidin-2-yl)piperazine-1-carboxylate (Compound 31b)

A suspension of 5-((2S,6R)-2-((4-(2-chloropyrimidin-4-yl)piperazin-1-yl)methyl)-6-methylmorpholino)quinoline-8-carbonitrile (compound 22b, 92 mg, 198 μmol), tert-butyl piperazine-1-carboxylate (compound 31a, 44 mg, 238 μmol) in ACN (2 mL) was heated at 110° C. under microwave for 2 hrs. The reaction mixture was cooled to room temperature and filtered. The filtrate was concentrated to afford Compound 31b (120 mg) as a yellow solid. MS: calc'd 614 (MH⁺), measured 614 (MH⁺).

### Step 2: 5-((2R,6S)-2-methyl-6-((4-(2-(piperazin-1-yl)pyrimidin-4-yl)piperazin-1-yl)methyl)morpholino)quinoline-8-carbonitrile (Compound 31c)

A solution of tert-butyl 4-(4-(4-(((2S,6R)-4-(8-cyanoquinolin-5-yl)-6-methylmorpholin-2-yl)methyl)piperazin-1-yl)pyrimidin-2-yl)piperazine-1-carboxylate (compound 31b, 120 mg, 196 μmol) in hexafluoroisopropanol (2 mL) was heated at 110° C. under microwave for 2 hrs. The reaction mixture was cooled to room temperature and filtered. The filtrate was concentrated to afford compound 31c (90 mg) as a yellow solid. MS: calc'd 514 (MH⁺), measured 514 (MH⁺).

### Step 3: 5-[(2R,6S)-2-methyl-6-[[4-[2-[4-[(2S)-pyrrolidine-2-carbonyl]piperazin-1-yl]pyrimidin-4-yl]piperazin-1-yl]methyl]morpholin-4-yl]quinoline-8-carbonitrile (Example 31)

A solution of 5-((2R,6S)-2-methyl-6-((4-(2-(piperazin-1-yl)pyrimidin-4-yl)piperazin-1-yl)methyl)morpholino)quinoline-8-carbonitrile (compound 31c, 30 mg, 58.4 μmol), (tert-butoxycarbonyl)-D-proline (compound 31d, 14 mg, 64.2 μmol) and Et₃N (18 mg, 24.4 μl, 175 μmol) in N,N-Dimethylformamide (1 mL) was stirred at rt for 2 hrs. The reaction mixture was diluted with EA (10 mL) and washed with water. The organic layer was concentrated and purified by silica gel chromatography to afford tert-butyl (R)-2-(4-(4-(4-(((2S,6R)-4-(8-cyanoquinolin-5-yl)-6-methylmorpholin-2-yl)methyl)piperazin-1-yl)pyrimidin-2-yl)piperazine-1-carbonyl)pyrrolidine-1-carboxylate (20 mg) as colorless oil. MS: calc'd 711 (MH⁺), measured 711 (MH⁺).

To a solution of tert-butyl (R)-2-(4-(4-(4-(((2S,6R)-4-(8-cyanoquinolin-5-yl)-6-methylmorpholin-2-yl)methyl)piperazin-1-yl)pyrimidin-2-yl)piperazine-1-carbonyl)pyrrolidine-1-carboxylate (20 mg, 28.1 μmol) in DCM (2 mL) was added TFA (9.62 mg, 84.4 μmol) at 0° C. The reaction mixture was stirred at rt for 2 hrs. Then concentrated and purified by prep-HPLC (TFA/ACN) to afford Example 31 (11.7 mg) as a light yellow solid. MS: calc'd 611 (MH⁺), measured 611 (MH⁺). ¹H NMR (METHANOL-d₄, 400 MHz) δ 9.02 (dd, 1H, J=1.7, 4.3 Hz), 8.68 (dd, 1H, J=1.6, 8.7 Hz), 8.19 (d, 1H, J=7.9 Hz), 7.94 (d, 1H, J=7.2 Hz), 7.67 (dd, 1H, J=4.3, 8.6 Hz), 7.31 (d, 1H, J=8.1 Hz), 6.62 (d, 1H, J=7.3 Hz), 4.7-4.8 (m, 1H), 4.52 (br s, 1H), 4.20 (br d, 5H, J=6.2 Hz), 3.7-4.0 (m, 9H), 3.4-3.6 (m, 9H), 2.7-2.9 (m, 2H), 2.5-2.6 (m, 1H), 2.0-2.2 (m, 3H), 1.34 (d, 3H, J=6.2 Hz).

### Example 32

### 5-[(2S,6R)-2-[[4-[2-[4-[2-(dimethylamino)acetyl]piperazin-1-yl]pyrimidin-4-yl]piperazin-1-yl]methyl]-6-methyl-morpholin-4-yl]quinoline-8-carbonitrile

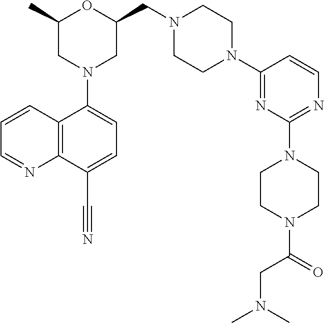 [SMILES: C[C@@H]1CN(c2ccc(C#N)c3ncccc23)C[C@H](CN2CCN(c3ccnc(N4CCN(C(=O)CN(C)C)CC4)n3)CC2)O1]

The title compound was prepared in analogy to the preparation of Example 31 by using dimethylglycine instead of (tert-butoxycarbonyl)-D-proline (compound 31d). Example 32 (8.8 mg) was obtained as a light yellow solid. MS: calc'd 599 (MH⁺), measured 599 (MH⁺). ¹H NMR (METHANOL-d₄, 400 MHz) δ 9.02 (dd, 1H, J=1.6, 4.3 Hz), 8.68 (dd, 1H, J=1.7, 8.6 Hz), 8.19 (d, 1H, J=8.1 Hz), 7.93 (d, 1H, J=7.2 Hz), 7.67 (dd, 1H, J=4.3, 8.6 Hz), 7.30 (d, 1H, J=8.1 Hz), 6.58 (d, 1H, J=7.2 Hz), 4.50 (br s, 1H), 4.34 (s, 2H), 4.0-4.3 (m, 5H), 3.8-3.9 (m, 6H), 3.6-3.7 (m, 2H), 3.4-3.6 (m, 7H), 2.99 (s, 6H), 2.7-2.8 (m, 2H), 1.33 (d, 3H, J=6.2 Hz).

### Example 33

### 5-[(2R,6S)-2-methyl-6-[[4-[6-(4-methylpiperazin-1-yl)-2-pyridyl]piperazin-1-yl]methyl]morpholin-4-yl]quinoline-8-carbonitrile

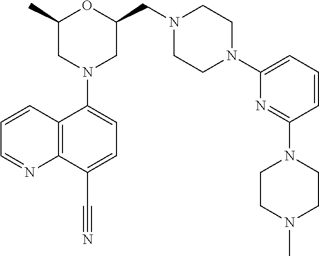 [SMILES: C[C@@H]1CN(c2ccc(C#N)c3ncccc23)C[C@H](CN2CCN(c3cccc(N4CCN(C)CC4)n3)CC2)O1]

The title compound was prepared according to the following scheme:

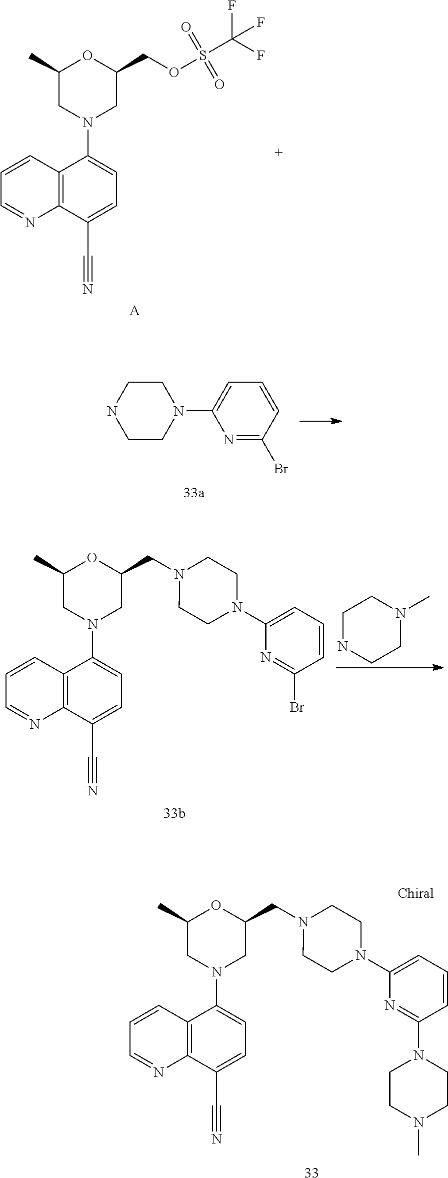

### Step 1: 5-((2S,6R)-2-((4-(6-bromopyridin-2-yl)piperazin-1-yl)methyl)-6-methylmorpholino)quinoline-8-carbonitrile (Compound 33b)

A suspension of ((2R,6R)-4-(8-cyanoquinolin-5-yl)-6-methylmorpholin-2-yl)methyl trifluoromethanesulfonate (intermediate A, 831 mg, 2 mmol) and 1-(6-bromopyridin-2-yl)piperazine (compound 33a, 489 mg, 2.02 mmol) and K₂CO₃ (553 mg, 4 mmol) in ACN (8 mL) was heat at 90° C. for 3 hrs. Then the mixture was filtered and the filtrate was concentrated and purified by silica gel chromatography to afford Compound 33b (652.6 mg) as a yellow solid. MS: calc'd 508 (MH⁺), measured 508 (MH⁺).

### Step 2: 5-[(2R,6S)-2-methyl-6-[[4-[6-(4-methylpiperazin-1-yl)-2-pyridyl]piperazin-1-yl]methyl]morpholin-4-yl]quinoline-8-carbonitrile (Example 33)

A suspension of 5-((2S,6R)-2-(4-(6-bromopyridin-2-yl)piperazin-1-yl)methyl)-6-methylmorpholino)quinoline-8-carbonitrile (compound 33b, 40 mg, 78.8 μmol), 1-methylpiperazine (10 mg, 94.6 μmol), XPhos Palladacycle Gen. 2 (7 mg, 7.88 μmol) and cesium carbonate (52 mg, 158 μmol) in 1,4-Dioxane (2 mL) was heated at 100° C. for 12 hrs under nitrogen atmosphere. Then the mixture was filtered. The filtrate was concentrated and purified by prep-HPLC to afford Example 33 (5.8 mg) as a light yellow solid. MS: calc'd 527 (MH⁺), measured 527 (MH⁺). ¹H NMR (METHANOL-d₄, 400 MHz) δ 9.01 (dd, 1H, J=1.6, 4.3 Hz), 8.68 (dd, 1H, J=1.7, 8.6 Hz), 8.18 (d, 1H, J=7.9 Hz), 7.67 (dd, 1H, J=4.3, 8.7 Hz), 7.53 (t, 1H, J=8.1 Hz), 7.30 (d, 1H, J=8.1 Hz), 6.35 (dd, 2H, J=3.6, 8.1 Hz), 4.48 (br d, 4H, J=9.7 Hz), 4.0-4.3 (m, 2H), 3.7-3.9 (m, 2H), 3.5-3.7 (m, 3H), 3.4-3.5 (m, 6H), 3.0-3.3 (m, 5H), 2.97 (s, 3H), 2.7-2.9 (m, 2H), 1.34 (d, 3H, J=6.2 Hz).

### Example 34

### 5-[(2S,6R)-2-[[4-[2-[4-(azetidine-2-carbonyl)piperazin-1-yl]pyrimidin-4-yl]piperazin-1-yl]methyl]-6-methyl-morpholin-4-yl]quinoline-8-carbonitrile

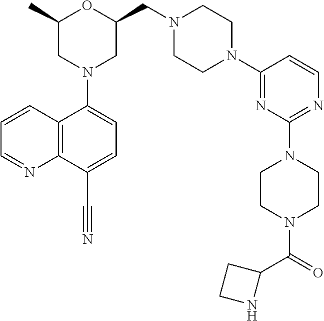 [SMILES: C[C@@H]1CN(c2ccc(C#N)c3ncccc23)C[C@H](CN2CCN(c3ccnc(N4CCN(C(=O)C5CCN5)CC4)n3)CC2)O1]

The title compound was prepared in analogy to the preparation of Example 31 by using azetidine-2-carboxylic acid instead of (tert-butoxycarbonyl)-D-proline (compound 31d). Example 34 (8.8 mg) was obtained as a light yellow solid. MS: calc'd 597 (MH⁺), measured 597 (MH⁺). ¹H NMR (METHANOL-d₄, 400 MHz) δ 8.90 (dd, 1H, J=1.6, 4.3 Hz), 8.56 (dd, 1H, J=1.7, 8.6 Hz), 8.07 (d, 1H, J=7.9 Hz), 7.81 (d, 1H, J=7.2 Hz), 7.55 (dd, 1H, J=4.2, 8.6 Hz), 7.19 (d, 1H, J=8.1 Hz), 6.47 (d, 1H, J=7.2 Hz), 5.36 (t, 1H, J=8.8 Hz), 4.39 (br s, 2H), 4.0-4.1 (m, 6H), 3.6-3.9 (m, 7H), 3.3-3.5 (m, 9H), 2.8-2.9 (m, 1H), 2.6-2.7 (m, 2H), 2.5-2.6 (m, 1H), 1.21 (d, 3H, J=6.2 Hz).

### Example 35

### 5-[(2S,6R)-2-[[4-[6-[[(3S,4S)-4-fluoropyrrolidin-3-yl]amino]-2-pyridyl]piperazin-1-yl]methyl]-6-methyl-morpholin-4-yl]quinoline-8-carbonitrile

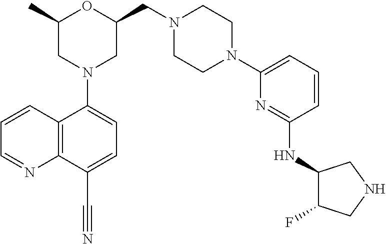 [SMILES: C[C@@H]1CN(c2ccc(C#N)c3ncccc23)C[C@H](CN2CCN(c3cccc(N[C@H]4CNC[C@@H]4F)n3)CC2)O1]

The title compound was prepared according to the following scheme:

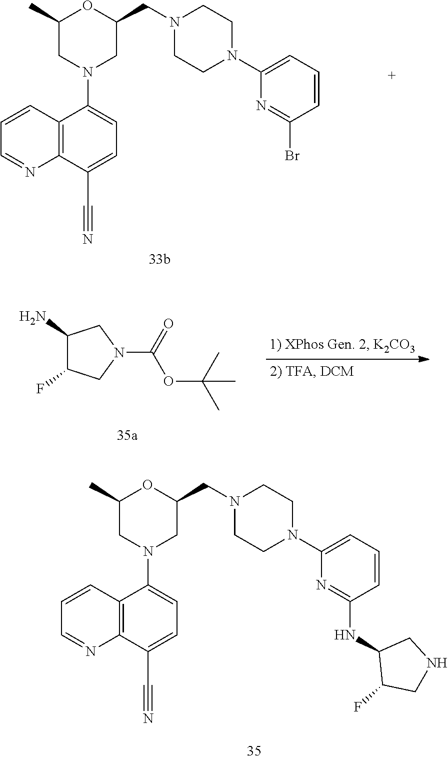 [SMILES: C[C@@H]1CN(c2ccc(C#N)c3ncccc23)C[C@H](CN2CCN(c3cccc(Br)n3)CC2)O1 ; CC(C)(C)OC(=O)N1C[C@H](N)[C@@H](F)C1 ; C[C@@H]1CN(c2ccc(C#N)c3ncccc23)C[C@H](CN2CCN(c3cccc(N[C@H]4CNC[C@@H]4F)n3)CC2)O1]

A suspension of 5-((2S,6R)-2-((4-(6-bromopyridin-2-yl)piperazin-1-yl)methyl)-6-methylmorpholino)quinoline-8-carbonitrile (compound 33b, 40 mg, 78.8 μmol), tert-butyl (3S,4S)-3-amino-4-fluoropyrrolidine-1-carboxylate (compound 35a, 18 mg, 86.7 μmol), cesium carbonate (52 mg, 158 μmol) and XPhos Palladacycle Gen. 2 (6.2 mg, 7.88 μmol) in 1,4-dioxane (2 mL) was heated at 100° C. for 12 hrs under nitrogen atmosphere. The reaction mixture was cooled to room temperature and filtered. The filtrate was concentrated to get crude intermediate. MS: calc'd 631 (MH⁺), measured 631 (MH⁺).

The intermediate was dissolved in 5 mL DCM. Then 0.5 mL TFA was added to the solution. After the mixture was stirred at rt for 3 hrs. The mixture was concentrated in vacuo to give a yellow oil which was purified by prep-HPLC. Example 35 (5.4 mg) was obtained as a yellow oil. MS: calc'd 531 (MH⁺), measured 531 (MH⁺). ¹H NMR (METHANOL-d₄, 400 MHz) δ 9.0-9.0 (m, 1H), 8.68 (dd, 1H, J=1.7, 8.6 Hz), 8.17 (d, 1H, J=7.9 Hz), 7.66 (dd, 1H, J=4.3, 8.6 Hz), 7.4-7.5 (m, 1H), 7.29 (d, 1H, J=8.1 Hz), 6.24 (d, 1H, J=8.1 Hz), 6.08 (d, 1H, J=7.9 Hz), 5.3-5.5 (m, 1H), 4.69 (br dd, 1H, J=4.1, 13.5 Hz), 4.53 (br dd, 1H, J=2.8, 7.4 Hz), 4.44 (br s, 1H), 4.1-4.3 (m, 1H), 3.43 (br s, 13H), 3.1-3.3 (m, 2H), 2.7-2.9 (m, 2H), 1.34 (d, 3H, J=6.4 Hz).

### Example 36

### 5-[(2S,6R)-2-[[4-[6-(4-amino-1-piperidyl)-2-pyridyl]piperazin-1-yl]methyl]-6-methyl-morpholin-4-yl]quinoline-8-carbonitrile

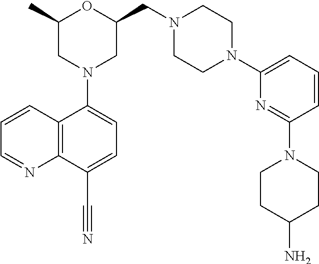 [SMILES: C[C@@H]1CN(c2ccc(C#N)c3ncccc23)C[C@H](CN2CCN(c3cccc(N4CCC(N)CC4)n3)CC2)O1]

The title compound was prepared in analogy to the preparation of Example 35 by using Boc-4-aminopiperidine instead of tert-butyl (3S,4S)-3-amino-4-fluoropyrrolidine-1-carboxylate (compound 35a). Example 36 (3.3 mg) was obtained as a light yellow solid. MS: calc'd 527 (MH⁺), measured 527 (MH⁺). ¹H NMR (METHANOL-d₄, 400 MHz) δ 9.01 (dd, 1H, J=1.6, 4.3 Hz), 8.68 (dd, 1H, J=1.6, 8.6 Hz), 8.18 (d, 1H, J=7.9 Hz), 7.67 (dd, 1H, J=4.2, 8.6 Hz), 7.4-7.5 (m, 1H), 7.30 (d, 1H, J=8.1 Hz), 6.30 (d, 1H, J=8.2 Hz), 6.24 (d, 1H, J=8.1 Hz), 4.3-4.6 (m, 4H), 4.2-4.3 (m, 1H), 3.4-4.0 (m, 10H), 3.1-3.3 (m, 2H), 2.9-3.0 (m, 2H), 2.7-2.9 (m, 2H), 2.0-2.1 (m, 2H), 1.62 (dq, 2H, J=4.2, 12.2 Hz), 1.34 (d, 3H, J=6.2 Hz).

### Example 37

### 5-[(2S,6R)-2-[[4-[6-[[(3S,4R)-4-fluoropyrrolidin-3-yl]amino]-2-pyridyl]piperazin-1-yl]methyl]-6-methyl-morpholin-4-yl]quinoline-8-carbonitrile

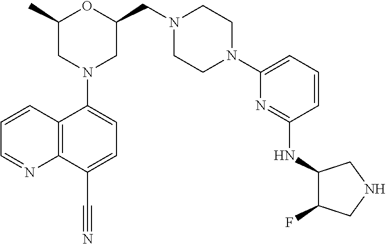 [SMILES: C[C@@H]1CN(c2ccc(C#N)c3ncccc23)C[C@H](CN2CCN(c3cccc(N[C@H]4CNC[C@H]4F)n3)CC2)O1]

The title compound was prepared in analogy to the preparation of Example 35 by using tert-butyl (3S,4R)-3-amino-4-fluoropyrrolidine-1-carboxylate (CAS: 1174020-30-4) instead of tert-butyl (3S,4S)-3-amino-4-fluoropyrrolidine-1-carboxylate (compound 35a). Example 37 (11.3 mg) was obtained as a light yellow solid. MS: calc'd 531 (MH⁺), measured 531 (MH⁺). ¹H NMR (METHANOL-d₄, 400 MHz) δ 9.00 (br d, 1H, J=2.8 Hz), 8.67 (br d, 1H, J=8.6 Hz), 8.17 (dd, 1H, J=3.7, 8.0 Hz), 7.66 (ddd, 1H, J=2.1, 4.2, 8.6 Hz), 7.38 (t, 1H, J=4.0 Hz), 7.29 (dd, 1H, J=3.3, 8.1 Hz), 6.19 (d, 1H, J=8.1 Hz), 6.12 (d, 1H, J=8.1 Hz), 5.41 (t, 1H, J=3.1 Hz), 5.28 (br s, 1H), 4.9-5.0 (m, 2H), 4.8-4.9 (m, 1H), 4.5-4.6 (m, 1H), 4.39 (br s, 1H), 4.2-4.3 (m, 1H), 3.7-3.9 (m, 4H), 3.3-3.5 (m, 6H), 3.19 (t, 2H, J=11.2 Hz), 2.7-2.9 (m, 2H), 1.34 (d, 3H, J=6.2 Hz).

### Example 38

### 5-[(2S,6R)-2-[[4-[6-(4-amino-1-piperidyl)-2-pyridyl]piperazin-1-yl]methyl]-6-methyl-morpholin-4-yl]quinoline-8-carbonitrile

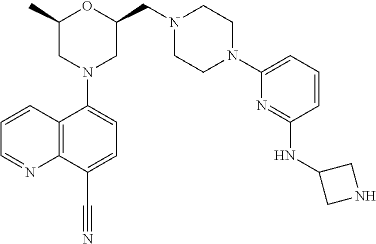 [SMILES: C[C@@H]1CN(c2ccc(C#N)c3ncccc23)C[C@H](CN2CCN(c3cccc(NC4CNC4)n3)CC2)O1]

The title compound was prepared in analogy to the preparation of Example 35 by using 1-BOC-3-(amino)azetidine instead of tert-butyl (3S,4S)-3-amino-4-fluoropyrrolidine-1-carboxylate (compound 35a). Example 38 (8.7 mg) was obtained as a light yellow solid. MS: calc'd 499 (MH⁺), measured 499 (MH⁺). ¹H NMR (METHANOL-d₄, 400 MHz) δ 9.01 (dd, 1H, J=1.7, 4.2 Hz), 8.68 (dd, 1H, J=1.6, 8.6 Hz), 8.18 (d, 1H, J=7.9 Hz), 7.67 (dd, 1H, J=4.2, 8.6 Hz), 7.47 (t, 1H, J=4.0 Hz), 7.30 (d, 1H, J=8.1 Hz), 6.28 (d, 1H, J=8.2 Hz), 5.93 (d, 1H, J=7.9 Hz), 4.4-4.7 (m, 3H), 4.1-4.4 (m, 5H), 3.99 (dd, 2H, J=4.3, 9.4 Hz), 3.4-3.9 (m, 9H), 2.7-2.9 (m, 2H), 1.34 (d, 3H, J=6.2 Hz).

### Example 39

### 5-[(2S,6R)-2-[[4-[6-[[(3R,4R)-4-methoxypyrrolidin-3-yl]amino]-2-pyridyl]piperazin-1-yl]methyl]-6-methyl-morpholin-4-yl]quinoline-8-carbonitrile

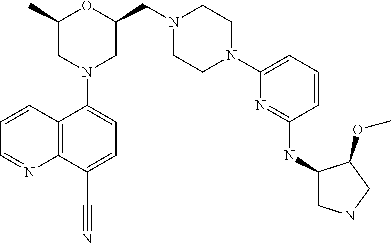 [SMILES: CO[C@H]1CNC[C@H]1Nc1cccc(N2CCN(C[C@H]3CN(c4ccc(C#N)c5ncccc45)C[C@@H](C)O3)CC2)n1]

The title compound was prepared in analogy to the preparation of Example 35 by using tert-butyl (3R,4R)-3-amino-4-methoxypyrrolidine-1-carboxylate (PharmaBlock, PBXA3109, CAS: 1400562-12-0) instead of tert-butyl (3S,4S)-3-amino-4-fluoropyrrolidine-1-carboxylate (compound 35a). Example 39 (16.2 mg) was obtained as a light yellow solid. MS: calc'd 543 (MH⁺), measured 543 (MH⁺). ¹H NMR (METHANOL-d₄, 400 MHz) δ 8.88 (dd, 1H, J=1.7, 4.2 Hz), 8.55 (dd, 1H, J=1.7, 8.6 Hz), 8.05 (d, 1H, J=7.9 Hz), 7.54 (dd, 1H, J=4.2, 8.6 Hz), 7.3-7.3 (m, 1H), 7.17 (d, 1H, J=8.1 Hz), 6.1-6.1 (m, 1H), 5.95 (d, 1H, J=8.1 Hz), 4.4-4.5 (m, 2H), 4.2-4.3 (m, 1H), 3.9-4.2 (m, 3H), 3.5-3.8 (m, 4H), 3.2-3.5 (m, 13H), 2.6-2.7 (m, 2H), 1.22 (d, 3H, J=6.2 Hz).

### Example 40

### 5-[(2S,6R)-2-[[4-[6-(3-amino-4-fluoro-pyrrolidin-1-yl)-2-pyridyl]piperazin-1-yl]methyl]-6-methyl-morpholin-4-yl]quinoline-8-carbonitrile

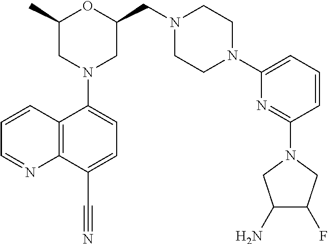 [SMILES: C[C@@H]1CN(c2ccc(C#N)c3ncccc23)C[C@H](CN2CCN(c3cccc(N4CC(N)C(F)C4)n3)CC2)O1]

The title compound was prepared in analogy to the preparation of Example 35 by using tert-butyl N-(4-fluoropyrrolidin-3-yl)carbamate instead of tert-butyl (3S,4S)-3-amino-4-fluoropyrrolidine-1-carboxylate (compound 35a). Example 40 (14.8 mg) was obtained as a light yellow solid. MS: calc'd 531 (MH⁺), measured 531 (MH⁺). ¹H NMR (METHANOL-d₄, 400 MHz) δ 8.90 (dd, 1H, J=1.7, 4.2 Hz), 8.57 (dd, 1H, J=1.6, 8.6 Hz), 8.07 (d, 1H, J=7.9 Hz), 7.55 (dd, 1H, J=4.2, 8.6 Hz), 7.3-7.4 (m, 1H), 7.19 (d, 1H, J=7.9 Hz), 6.15 (d, 1H, J=8.1 Hz), 5.91 (d, 1H, J=7.9 Hz), 5.2-5.4 (m, 1H), 4.2-4.5 (m, 3H), 4.0-4.2 (m, 3H), 3.7-3.9 (m, 3H), 3.5-3.7 (m, 4H), 3.2-3.4 (m, 6H), 2.66 (ddd, 2H, J=10.3, 12.0, 18.8 Hz), 1.22 (d, 3H, J=6.2 Hz).

### Example 41

### 5-[(2S,6R)-2-[[4-[6-(6-hydroxy-1,4-diazepan-1-yl)-2-pyridyl]piperazin-1-yl]methyl]-6-methyl-morpholin-4-yl]quinoline-8-carbonitrile

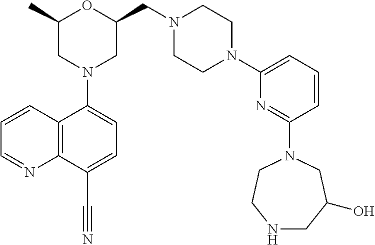 [SMILES: C[C@@H]1CN(c2ccc(C#N)c3ncccc23)C[C@H](CN2CCN(c3cccc(N4CCNCC(O)C4)n3)CC2)O1]

The title compound was prepared in analogy to the preparation of Example 35 by using tert-butyl 6-hydroxy-1,4-diazepane-1-carboxylate (Wuxi Apptec, WX604354, CAS: 956317-40-1) instead of tert-butyl (3S,4S)-3-amino-4-fluoropyrrolidine-1-carboxylate (compound 35a). Example 41 (0.9 mg) was obtained as a light yellow solid. MS: calc'd 543 (MH⁺), measured 543 (MH⁺). ¹H NMR (METHANOL-d₄, 400 MHz) δ 8.9-9.1 (m, 1H), 8.68 (dd, 1H, J=1.5, 8.7 Hz), 8.18 (d, 1H, J=7.9 Hz), 7.67 (dd, 1H, J=4.2, 8.6 Hz), 7.4-7.5 (m, 1H), 7.30 (d, 1H, J=7.9 Hz), 6.26 (d, 1H, J=8.3 Hz), 6.2-6.2 (m, 1H), 6.22 (s, 1H), 4.5-4.6 (m, 1H), 4.40 (q, 2H, J=4.2 Hz), 4.1-4.3 (m, 2H), 3.8-4.1 (m, 4H), 3.74 (br dd, 3H, J=4.2, 14.9 Hz), 3.5-3.6 (m, 2H), 3.4-3.5 (m, 5H), 3.1-3.3 (m, 3H), 2.7-2.9 (m, 2H), 1.34 (d, 3H, J=6.2 Hz).

### Example 42

### 5-[(2S,6R)-2-[[4-[6-[(3R,4S)-3-amino-4-methoxy-pyrrolidin-1-yl]-2-pyridyl]piperazin-1-yl]methyl]-6-methyl-morpholin-4-yl]quinoline-8-carbonitrile

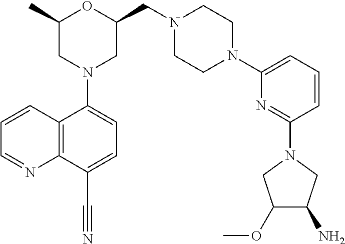 [SMILES: COC1CN(c2cccc(N3CCN(C[C@H]4CN(c5ccc(C#N)c6ncccc56)C[C@@H](C)O4)CC3)n2)C[C@H]1N]

The title compound was prepared in analogy to the preparation of Example 35 by using tert-butyl N-((3R,4S)-4-methoxypyrrolidin-3-yl)carbamate (PharmaBlock, PBZ4729, CAS: 1932508-77-4) instead of tert-butyl (3S,4S)-3-amino-4-fluoropyrrolidine-1-carboxylate (compound 35a). Example 42 (20.6 mg) was obtained as a light yellow solid. MS: calc'd 543 (MH⁺), measured 543 (MH⁺). ¹H NMR (METHANOL-d₄, 400 MHz) δ 9.01 (dd, 1H, J=1.6, 4.3 Hz), 8.68 (dd, 1H, J=1.7, 8.6 Hz), 8.19 (d, 1H, J=7.9 Hz), 7.67 (dd, 1H, J=4.2, 8.6 Hz), 7.46 (quin, 1H, J=4.0 Hz), 7.30 (d, 1H, J=8.1 Hz), 6.22 (d, 1H, J=8.1 Hz), 5.98 (d, 1H, J=8.1 Hz), 4.3-4.6 (m, 3H), 4.1-4.3 (m, 3H), 3.6-4.1 (m, 7H), 3.4-3.6 (m, 9H), 3.2-3.3 (m, 1H), 2.78 (ddd, 2H, J=10.3, 12.0, 19.3 Hz), 1.34 (d, 3H, J=6.2 Hz).

### Example 43

### 5-[(2S,6R)-2-[[4-[6-(3-amino-4-methoxy-pyrrolidin-1-yl)-3-pyridyl]piperazin-1-yl]methyl]-6-methyl-morpholin-4-yl]quinoline-8-carbonitrile

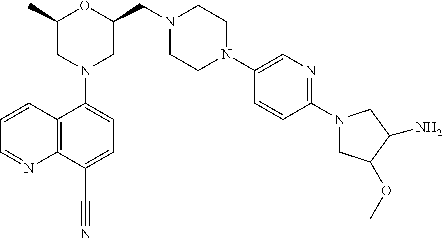 [SMILES: COC1CN(c2ccc(N3CCN(C[C@H]4CN(c5ccc(C#N)c6ncccc56)C[C@@H](C)O4)CC3)cn2)CC1N]

The title compound was prepared in analogy to the preparation of Example 33 by using 1-(6-bromopyridin-3-yl)piperazine and tert-butyl N-(4-methoxypyrrolidin-3-yl)carbamate instead of 1-(6-bromopyridin-2-yl)piperazine (compound 33a) and 1-methylpiperazine. Example 43 (4.1 mg) was obtained as a light yellow solid. MS: calc'd 543 (MH⁺), measured 543 (MH⁺). ¹H NMR (METHANOL-d₄, 400 MHz) δ 9.01 (dd, 1H, J=1.6, 4.3 Hz), 8.69 (dd, 1H, J=1.7, 8.6 Hz), 8.19 (d, 1H, J=7.9 Hz), 7.93 (dd, 1H, J=2.8, 9.7 Hz), 7.6-7.8 (m, 2H), 7.31 (d, 1H, J=8.1 Hz), 7.03 (d, 1H, J=9.5 Hz), 4.5-4.6 (m, 1H), 4.3-4.4 (m, 1H), 4.1-4.3 (m, 2H), 4.0-4.1 (m, 1H), 3.8-3.9 (m, 1H), 3.7-3.8 (m, 2H), 3.6-3.7 (m, 4H), 3.53 (s, 3H), 3.3-3.5 (m, 7H), 3.0-3.2 (m, 1H), 2.7-2.9 (m, 2H), 1.34 (d, 3H, J=6.2 Hz).

### Example 44

### 5-[(2S,6R)-2-[[4-[6-(3-amino-4-fluoro-pyrrolidin-1-yl)-3-pyridyl]piperazin-1-yl]methyl]-6-methyl-morpholin-4-yl]quinoline-8-carbonitrile

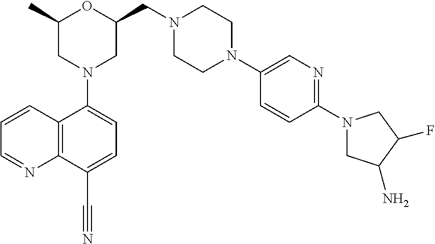 [SMILES: C[C@@H]1CN(c2ccc(C#N)c3ncccc23)C[C@H](CN2CCN(c3ccc(N4CC(N)C(F)C4)nc3)CC2)O1]

The title compound was prepared in analogy to the preparation of Example 33 by using 1-(6-bromopyridin-3-yl)piperazine and tert-butyl N-(4-fluoropyrrolidin-3-yl)carbamate and instead of 1-(6-bromopyridin-2-yl)piperazine (compound 33a) and 1-methylpiperazine. Example 44 (2.0 mg) was obtained as a light yellow solid. MS: calc'd 531 (MH⁺), measured 531 (MH⁺). ¹H NMR (METHANOL-d₄, 400 MHz) δ 8.88 (d, 1H, J=4.5 Hz), 8.56 (d, 1H, J=7.0 Hz), 8.05 (d, 1H, J=7.9 Hz), 7.67 (d, 1H, J=2.8 Hz), 7.5-7.5 (m, 1H), 7.5-7.5 (m, 1H), 7.53 (s, 1H), 7.31 (dd, 1H, J=2.8, 9.1 Hz), 7.1-7.2 (m, 1H), 6.44 (d, 1H, J=8.9 Hz), 4.1-4.1 (m, 1H), 3.9-4.0 (m, 1H), 3.6-3.8 (m, 2H), 3.5-3.6 (m, 3H), 3.3-3.4 (m, 4H), 2.97 (s, 3H), 2.8-2.8 (m, 1H), 2.7-2.7 (m, 1H), 2.5-2.7 (m, 6H), 1.16 (d, 3H, J=6.2 Hz).

### Example 45

### 5-[(2S,6R)-2-[[4-(5-fluoro-2-piperazin-1-yl-pyrimidin-4-yl)piperazin-1-yl]methyl]-6-methyl-morpholin-4-yl]quinoline-8-carbonitrile

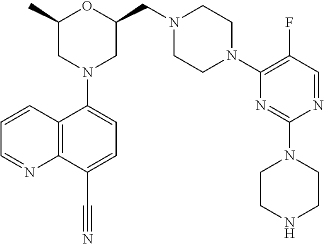 [SMILES: C[C@@H]1CN(c2ccc(C#N)c3ncccc23)C[C@H](CN2CCN(c3nc(N4CCNCC4)ncc3F)CC2)O1]

The title compound was prepared according to the following scheme:

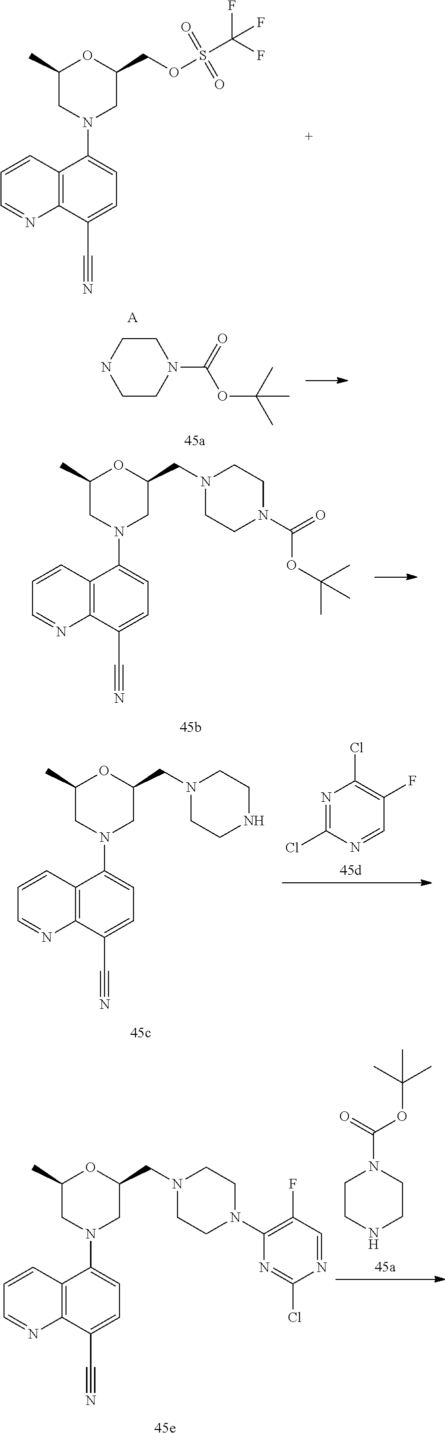

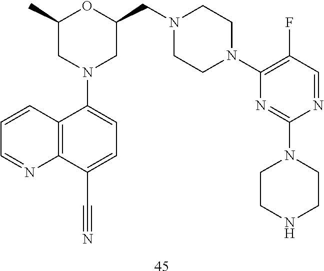 [SMILES: C[C@@H]1CN(c2ccc(C#N)c3ncccc23)C[C@H](CN2CCN(c3nc(N4CCNCC4)ncc3F)CC2)O1 ; "45"]

### Step 1: tert-butyl 4-(((2S,6R)-4-(8-cyanoquinolin-5-yl)-6-methylmorpholin-2-yl)methyl)piperazine-1-carboxylate (Compound 45b)

A suspension of ((2R,6R)-4-(8-cyanoquinolin-5-yl)-6-methylmorpholin-2-yl)methyl trifluoromethanesulfonate (Intermediate A, 800 mg, 1.93 mmol), tert-butyl piperazine-1-carboxylate (compound 45a, 430 mg, 2.31 mmol) and K₂CO₃ (532 mg, 3.85 mmol) in ACN (8 mL) was heated at 90° C. for 3 hrs. Then the mixture was filtered and the filtrate was concentrated and purified by silica gel chromatography to afford tert-butyl 4-(((2S,6R)-4-(8-cyanoquinolin-5-yl)-6-methylmorpholin-2-yl)methyl)piperazine-1-carboxylate (compound 45b, 800 mg) as yellow solid. MS: calc'd 452 (MH⁺), measured 452 (MH⁺).

### Step 2: 5-((2R,6S)-2-methyl-6-(piperazin-1-ylmethyl)morpholino)quinoline-8-carbonitrile 2,2,2-trifluoroacetate (Compound 45c)

A solution of tert-butyl 4-(((2S,6R)-4-(8-cyanoquinolin-5-yl)-6-methylmorpholin-2-yl)methyl)piperazine-1-carboxylate (compound 45b, 800 mg, 1.77 mmol) and added TFA (0.5 mL) in DCM (6 mL) was stirred at rt for 2 hrs. Then the mixture was concentrated to afford crude 5-((2R,6S)-2-methyl-6-(piperazin-1-ylmethyl)morpholino)quinoline-8-carbonitrile 2,2,2-trifluoroacetate (compound 45c, 800 mg) as yellow oil. MS: calc'd 352 (MH⁺), measured 352 (MH⁺).

### Step 3: 5-((2S,6R)-2-((4-(2-chloro-5-fluoropyrimidin-4-yl)piperazin-1-yl)methyl)-6-methylmorpholino)quinoline-8-carbonitrile (Compound 45e)

A solution of 2,4-dichloro-5-fluoropyrimidine (compound 45d, 63 mg, 376 μmol) and 5-((2R,6S)-2-methyl-6-(piperazin-1-ylmethyl)morpholino)quinoline-8-carbonitrile 2,2,2-trifluoroacetate (compound 45c, 250 mg, 376 μmol) in ethanol (6 mL) was stirred at room temperature for 12 hrs. Then the mixture was concentrated and purified by silica gel Chromatography to afford 5-((2S,6R)-2-((4-(2-chloro-5-fluoropyrimidin-4-yl)piperazin-1-yl)methyl)-6-methylmorpholino)quinoline-8-carbonitrile (compound 45e, 90 mg). MS: calc'd 482 (MH⁺), measured 482 (MH⁺).

### Step 4: 5-[(2S,6R)-2-[[4-(5-fluoro-2-piperazin-1-yl-pyrimidin-4-yl)piperazin-1-yl]methyl]-6-methyl-morpholin-4-yl]quinoline-8-carbonitrile (Compound 45)

A suspension of 5-((2S,6R)-2-((4-(2-chloro-5-fluoropyrimidin-4-yl)piperazin-1-yl)methyl)-6-methylmorpholino)quinoline-8-carbonitrile (compound 45e, 90 mg, 187 μmol), tert-butyl piperazine-1-carboxylate (compound 45a, 37 mg, 196 μmol), Ruphos Pd G2 (15 mg, 18.7 μmol) and k₂CO₃ (52 mg, 373 μmol) in dioxane (10 mL) was heated at 100° C. for 12 hrs under nitrogen atmosphere. The reaction mixture was cooled to room temperature and filtered. The filtrate was concentrated. Then the residue was purified by silica gel to get crude intermediate (tert-butyl 4-(4-(4-(((2S,6R)-4-(8-cyanoquinolin-5-yl)-6-methylmorpholin-2-yl)methyl)piperazin-1-yl)-5-fluoropyrimidin-2-yl)piperazine-1-carboxylate) (60 mg). MS: calc'd 632 (MH⁺), measured 632 (MH⁺).

To a solution of crude intermediate tert-butyl 4-(4-(4-(((2S,6R)-4-(8-cyanoquinolin-5-yl)-6-methylmorpholin-2-yl)methyl)piperazin-1-yl)-5-fluoropyrimidin-2-yl)piperazine-1-carboxylate (60 mg, 95 μmol) in DCM (2 mL) was added TFA (49 mg, 427 μmol) at 0° C. The reaction mixture was stirred at rt for 2 hrs. Then the mixture was concentrated and purified by prep-HPLC to afford Example 45 (27.1 mg) as a light yellow solid. MS: calc'd 532 (MH⁺), measured 532 (MH⁺). ¹H NMR (METHANOL-d₄, 400 MHz) δ 9.0-9.0 (m, 1H), 8.6-8.7 (m, 1H), 8.1-8.2 (m, 1H), 8.0-8.1 (m, 1H), 7.6-7.7 (m, 1H), 7.2-7.3 (m, 1H), 4.5-4.6 (m, 1H), 4.3-4.3 (m, 1H), 4.1-4.3 (m, 1H), 4.1-4.1 (m, 1H), 4.0-4.0 (m, 4H), 3.7-3.7 (m, 1H), 3.5-3.7 (m, 3H), 3.4-3.5 (m, 4H), 3.3-3.3 (m, 6H), 2.7-2.8 (m, 2H), 1.3-1.4 (m, 3H).

### Example 46

### 5-[(2S,6R)-2-[[4-(5,6-dimethyl-2-piperazin-1-yl-pyrimidin-4-yl)piperazin-1-yl]methyl]-6-methyl-morpholin-4-yl]quinoline-8-carbonitrile

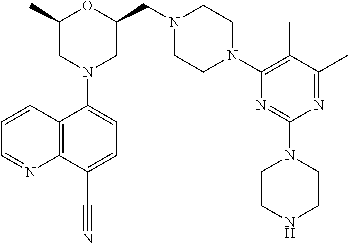 [SMILES: Cc1nc(N2CCNCC2)nc(N2CCN(C[C@H]3CN(c4ccc(C#N)c5ncccc45)C[C@@H](C)O3)CC2)c1C]

The title compound was prepared in analogy to the preparation of Example 45 by using 2,4-dichloro-5,6-dimethylpyrimidine instead of 2,4-dichloro-5-fluoropyrimidine (compound 45d). Example 46 (18.1 mg) was obtained as a light yellow solid. MS: calc'd 542 (MH⁺), measured 542 (MH⁺). ¹H NMR (METHANOL-d₄, 400 MHz) δ 8.9-9.0 (m, 1H), 8.6-8.7 (m, 1H), 8.1-8.2 (m, 1H), 7.6-7.7 (m, 1H), 7.2-7.3 (m, 1H), 4.5-4.6 (m, 1H), 4.11 (br d, 6H, J=4.3 Hz), 3.8-4.1 (m, 2H), 3.5-3.8 (m, 4H), 3.3-3.5 (m, 9H), 2.7-2.8 (m, 2H), 2.4-2.5 (m, 3H), 2.2-2.2 (m, 3H), 1.3-1.4 (m, 3H).

### Example 47

### 5-[(2R,6S)-2-methyl-6-[[4-(6-methyl-2-piperazin-1-yl-pyrimidin-4-yl)piperazin-1-yl]methyl]morpholin-4-yl]quinoline-8-carbonitrile

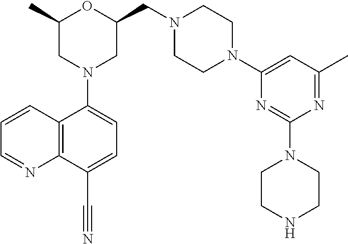 [SMILES: Cc1cc(N2CCN(C[C@H]3CN(c4ccc(C#N)c5ncccc45)C[C@@H](C)O3)CC2)nc(N2CCNCC2)n1]

The title compound was prepared in analogy to the preparation of Example 45 by using 2,4-dichloro-6-methylpyrimidine instead of 2,4-dichloro-5-fluoropyrimidine (compound 45d). Example 47 (196.3 mg) was obtained as a light yellow solid. MS: calc'd 528 (MH⁺), measured 528 (MH⁺). ¹H NMR (METHANOL-d₄, 400 MHz) δ 9.00 (dd, 1H, J=1.6, 4.3 Hz), 8.66 (dd, 1H, J=1.7, 8.6 Hz), 8.17 (d, 1H, J=7.9 Hz), 7.65 (dd, 1H, J=4.3, 8.6 Hz), 7.28 (d, 1H, J=8.1 Hz), 6.55 (s, 1H), 4.5-4.6 (m, 1H), 3.9-4.4 (m, 9H), 3.58 (br d, 4H, J=1.6 Hz), 3.3-3.5 (m, 8H), 2.7-2.8 (m, 2H), 2.43 (s, 3H), 1.31 (d, 3H, J=6.2 Hz).

### Example 48

### 5-[(2S,6R)-2-[[4-(5-methoxy-2-piperazin-1-yl-pyrimidin-4-yl)piperazin-1-yl]methyl]-6-methyl-morpholin-4-yl]quinoline-8-carbonitrile

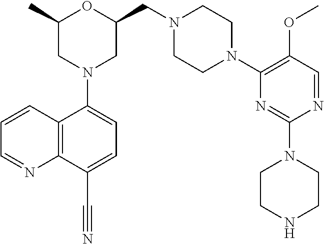 [SMILES: COc1cnc(N2CCNCC2)nc1N1CCN(C[C@H]2CN(c3ccc(C#N)c4ncccc34)C[C@@H](C)O2)CC1]

The title compound was prepared in analogy to the preparation of Example 45 by using 2,4-dichloro-5-methoxypyrimidine instead of 2,4-dichloro-5-fluoropyrimidine (compound 45d). Example 48 (54.3 mg) was obtained as a light yellow solid. MS: calc'd 544 (MH⁺), measured 544 (MH⁺). ¹H NMR (METHANOL-d₄, 400 MHz) δ 9.01 (dd, 1H, J=1.6, 4.3 Hz), 8.68 (dd, 1H, J=1.7, 8.6 Hz), 8.18 (d, 1H, J=8.1 Hz), 7.75 (s, 1H), 7.67 (dd, 1H, J=4.3, 8.6 Hz), 7.30 (d, 1H, J=8.1 Hz), 4.5-4.6 (m, 1H), 4.2-4.3 (m, 2H), 4.0-4.0 (m, 4H), 3.88 (s, 3H), 3.4-3.7 (m, 8H), 3.3-3.4 (m, 7H), 2.7-2.8 (m, 2H), 1.33 (d, 3H, J=6.2 Hz).

### Example 49

### 5-[(2R,6S)-2-methyl-6-[[4-(2-piperazin-1-ylquinazolin-4-yl)piperazin-1-yl]methyl]morpholin-4-yl]quinoline-8-carbonitrile

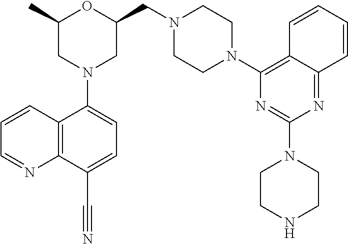 [SMILES: C[C@@H]1CN(c2ccc(C#N)c3ncccc23)C[C@H](CN2CCN(c3nc(N4CCNCC4)nc4ccccc34)CC2)O1]

The title compound was prepared in analogy to the preparation of Example 45 by using 2,4-dichloroquinazoline instead of 2,4-dichloro-5-fluoropyrimidine (compound 45d). Example 49 (10.3 mg) was obtained as a light yellow solid. MS: calc'd 564 (MH⁺), measured 564 (MH⁺). ¹H NMR (METHANOL-d₄, 400 MHz) δ 9.02 (dd, 1H, J=1.7, 4.3 Hz), 8.69 (dd, 1H, J=1.6, 8.6 Hz), 8.20 (d, 1H, J=8.1 Hz), 8.01 (d, 1H, J=8.2 Hz), 7.8-7.8 (m, 1H), 7.6-7.7 (m, 2H), 7.4-7.5 (m, 1H), 7.31 (d, 1H, J=8.1 Hz), 4.5-4.6 (m, 1H), 4.1-4.3 (m, 8H), 3.6-3.7 (m, 4H), 3.4-3.5 (m, 9H), 2.7-2.9 (m, 2H), 1.34 (d, 3H, J=6.4 Hz).

### Example 50

### 5-[(2S,6R)-2-[[4-(5-cyano-2-piperazin-1-yl-pyrimidin-4-yl)piperazin-1-yl]methyl]-6-methyl-morpholin-4-yl]quinoline-8-carbonitrile

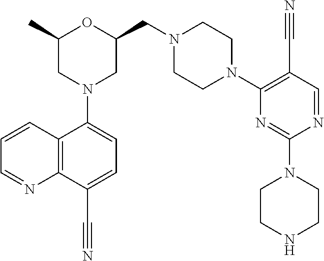 [SMILES: C[C@@H]1CN(c2ccc(C#N)c3ncccc23)C[C@H](CN2CCN(c3nc(N4CCNCC4)ncc3C#N)CC2)O1]

The title compound was prepared according to the following scheme:

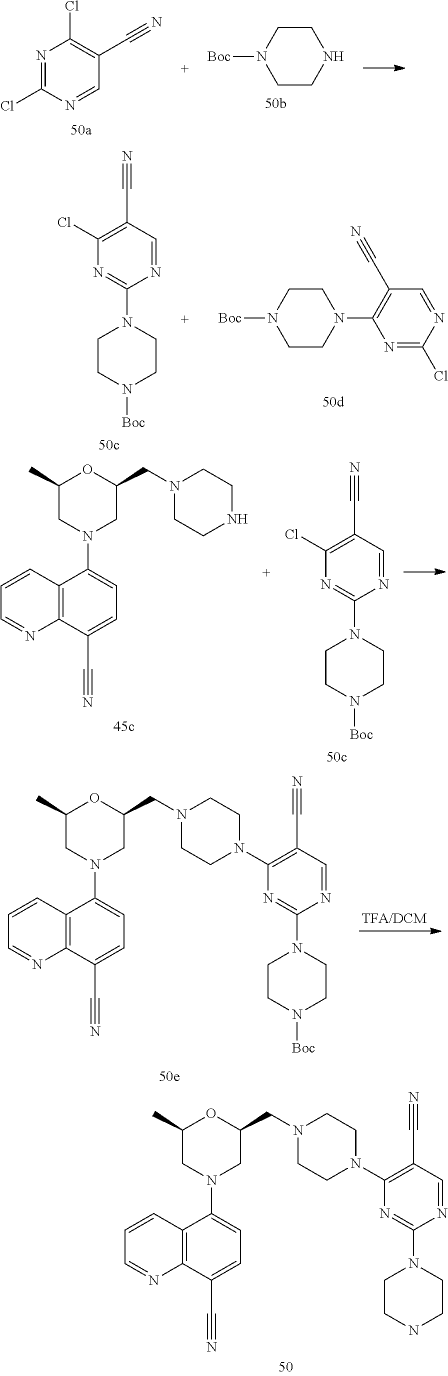

### Step 1: tert-butyl 4-(4-chloro-5-cyanopyrimidin-2-yl)piperazine-1-carboxylate (Compound 50c) and tert-butyl 4-(2-chloro-5-cyanopyrimidin-4-yl)piperazine-1-carboxylate (Compound 50d)

A solution of 2,4-dichloropyrimidine-5-carbonitrile (174 mg, 1 mmol) and tert-butyl piperazine-1-carboxylate (186 mg, 1 mmol) in ethanol (6 mL) was stirred at rt for 12 hrs. Then the mixture was concentrated and purified by silica gel to get tert-butyl 4-(4-chloro-5-cyanopyrimidin-2-yl)piperazine-1-carboxylate (compound 50c, 100 mg) and tert-butyl 4-(2-chloro-5-cyanopyrimidin-4-yl)piperazine-1-carboxylate (compound 50d, 120 mg).

Compound 50c, MS: calc'd 324 (MH⁺), measured 324 (MH⁺). ¹H NMR (CHLOROFORM-d, 400 MHz) δ 8.35 (s, 1H), 3.7-3.9 (m, 4H), 3.4-3.5 (m, 4H), 1.42 (s, 9H).

Compound 50d, MS: calc'd 324 (MH⁺), measured 324 (MH⁺). ¹H NMR (CHLOROFORM-d, 400 MHz) δ 8.33 (s, 1H), 3.9-4.0 (m, 4H), 3.5-3.6 (m, 4H), 1.42 (s, 9H).

### Step 2: tert-butyl 4-(5-cyano-4-(4-(((2S,6R)-4-(8-cyanoquinolin-5-yl)-6-methylmorpholin-2-yl)methyl)piperazin-1-yl)pyrimidin-2-yl)piperazine-1-carboxylate (Compound 50e)

A solution of 5-((2R,6S)-2-methyl-6-(piperazin-1-ylmethyl)morpholino)quinoline-8-carbonitrile 2,2,2-trifluoroacetate (compound 45c, 200 mg, 301 μmol), tert-butyl 4-(4-chloro-5-cyanopyrimidin-2-yl)piperazine-1-carboxylate (compound 50c, 100 mg, 309 μmol) and K₂CO₃ (83 mg, 602 μmol) in ACN (6 mL) was heated at 90° C. for 2 hrs. Then the reaction mixture was cooled to room temperature, diluted with EtOAc (10 mL) and washed with water (5 mL). The organic layer was dried and concentrated to afford tert-butyl 4-(5-cyano-4-(4-(((2S,6R)-4-(8-cyanoquinolin-5-yl)-6-methylmorpholin-2-yl)methyl)piperazin-1-yl)pyrimidin-2-yl)piperazine-1-carboxylate (compound 50e, 140 mg) which can be used for next step without purification. MS: calc'd 639 (MH⁺), measured 639 (MH⁺).

### Step 3: 5-[(2S,6R)-2-[[4-(5-cyano-2-piperazin-1-yl-pyrimidin-4-yl)piperazin-1-yl]methyl]-6-methyl-morpholin-4-yl]quinoline-8-carbonitrile (Example 50)

To a solution of tert-butyl 4-(5-cyano-4-(4-(((2S,6R)-4-(8-cyanoquinolin-5-yl)-6-methylmorpholin-2-yl)methyl)piperazin-1-yl)pyrimidin-2-yl)piperazine-1-carboxylate (compound 50e, 140 mg, 219 μmol) in DCM (2 mL) was added TFA (75 mg, 658 μmol) at 0° C. The reaction mixture was stirred at rt for 2 hrs. Then the mixture was concentrated and purified by prep-HPLC to afford Example 50 (88.8 mg) as a light yellow solid. MS: calc'd 539 (MH⁺), measured 539 (MH⁺). ¹H NMR (METHANOL-d₄, 400 MHz) δ 9.02 (dd, 1H, J=1.7, 4.3 Hz), 8.67 (d, 1H, J=1.6 Hz), 8.45 (s, 1H), 8.19 (d, 1H, J=7.9 Hz), 7.67 (dd, 1H, J=4.3, 8.6 Hz), 7.30 (d, 1H, J=8.1 Hz), 4.5-4.6 (m, 1H), 4.2-4.3 (m, 2H), 4.1-4.2 (m, 5H), 3.5-3.7 (m, 4H), 3.4-3.5 (m, 5H), 3.3-3.3 (m, 5H), 2.78 (ddd, 2H, J=10.1, 12.0, 18.5 Hz), 1.34 (d, 3H, J=6.2 Hz).

### Example 51

### 5-[(2S,6R)-2-[[4-(5-cyano-4-piperazin-1-yl-pyrimidin-2-yl)piperazin-1-yl]methyl]-6-methyl-morpholin-4-yl]quinoline-8-carbonitrile

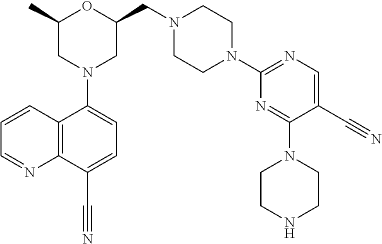 [SMILES: C[C@@H]1CN(c2ccc(C#N)c3ncccc23)C[C@H](CN2CCN(c3ncc(C#N)c(N4CCNCC4)n3)CC2)O1]

The title compound was prepared in analogy to the preparation of Example 50 by using tert-butyl 4-(2-chloro-5-cyanopyrimidin-4-yl)piperazine-1-carboxylate (compound 50d) instead of tert-butyl 4-(4-chloro-5-cyanopyrimidin-2-yl)piperazine-1-carboxylate (compound 50c). Example 51 (68 mg) was obtained as a light yellow solid. MS: calc'd 539 (MH⁺), measured 539 (MH⁺). ¹H NMR (METHANOL-d₄, 400 MHz) δ 9.02 (dd, 1H, J=1.7, 4.2 Hz), 8.68 (dd, 1H, J=1.7, 8.6 Hz), 8.45 (s, 1H), 8.19 (d, 1H, J=8.1 Hz), 7.67 (dd, 1H, J=4.2, 8.6 Hz), 7.31 (d, 1H, J=8.1 Hz), 4.5-4.6 (m, 1H), 4.1-4.3 (m, 6H), 3.3-3.9 (m, 15H), 2.7-2.8 (m, 2H), 1.34 (d, 3H, J=6.2 Hz).

### Example 52

### 5-[(2S,6R)-2-[[4-[2-[(3R,4R)-3-amino-4-methoxy-pyrrolidin-1-yl]pyrimidin-4-yl]piperazin-1-yl]methyl]-6-methyl-morpholin-4-yl]quinoline-8-carbonitrile

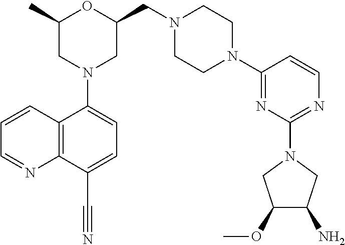 [SMILES: CO[C@H]1CN(c2nccc(N3CCN(C[C@H]4CN(c5ccc(C#N)c6ncccc56)C[C@@H](C)O4)CC3)n2)C[C@H]1N]

The title compound was prepared in analogy to the preparation of Example 23 by using tert-butyl N-[(3R,4R)-4-methoxypyrrolidin-3-yl]carbamate (PharmaBlock, PBZ4728, CAS: 1932066-52-8) instead of tert-butyl azetidin-3-ylcarbamate (compound 23c). Example 52 (33.8 mg) was obtained as a light yellow solid. MS: calc'd 544 (MH⁺), measured 544 (MH⁺). ¹H NMR (METHANOL-d₄, 400 MHz) δ 9.01 (br d, 1H, J=3.3 Hz), 8.67 (br d, 1H, J=7.8 Hz), 8.18 (d, 1H, J=7.9 Hz), 7.91 (d, 1H, J=7.3 Hz), 7.66 (dd, 1H, J=4.2, 8.6 Hz), 7.29 (d, 1H, J=7.9 Hz), 6.67 (d, 1H, J=7.5 Hz), 4.5-4.6 (m, 1H), 4.1-4.3 (m, 6H), 4.04 (br s, 4H), 3.5-3.7 (m, 4H), 3.50 (s, 3H), 3.4-3.5 (m, 5H), 2.7-2.9 (m, 2H), 1.33 (d, 3H, J=6.1 Hz).

### Example 53

### 5-[(2S,6R)-2-[[4-[2-[(3S,4S)-3-amino-4-methoxy-pyrrolidin-1-yl]pyrimidin-4-yl]piperazin-1-yl]methyl]-6-methyl-morpholin-4-yl]quinoline-8-carbonitrile

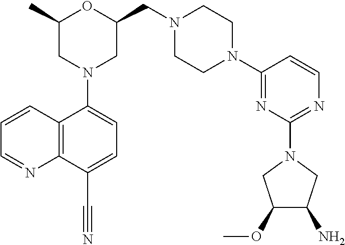 [SMILES: CO[C@H]1CN(c2nccc(N3CCN(C[C@H]4CN(c5ccc(C#N)c6ncccc56)C[C@@H](C)O4)CC3)n2)C[C@H]1N]

The title compound was prepared in analogy to the preparation of Example 23 by using tert-butyl N-[(3S,4S)-4-methoxypyrrolidin-3-yl]carbamate (PharmaBlock, PBZ4724, CAS: 1627185-88-9) instead of tert-butyl azetidin-3-ylcarbamate (compound 23c). Example 53 (33.6 mg) was obtained as a light yellow solid. MS: calc'd 544 (MH⁺), measured 544 (MH⁺). ¹H NMR (METHANOL-d₄, 400 MHz) δ 9.01 (dd, 1H, J=1.2, 4.2 Hz), 8.68 (dd, 1H, J=1.2, 8.4 Hz), 8.18 (d, 1H, J=7.9 Hz), 7.91 (d, 1H, J=7.5 Hz), 7.67 (dd, 1H, J=4.2, 8.6 Hz), 7.30 (d, 1H, J=8.1 Hz), 6.67 (d, 1H, J=7.6 Hz), 4.55 (br s, 1H), 4.1-4.4 (m, 6H), 4.0-4.1 (m, 4H), 3.5-3.7 (m, 4H), 3.50 (s, 3H), 3.4-3.5 (m, 5H), 2.7-2.9 (m, 2H), 1.33 (d, 3H, J=6.2 Hz).

### Example 54

### 5-[(2S,6R)-2-[[4-[2-[(3S,4R)-3-amino-4-fluoro-pyrrolidin-1-yl]-6-methyl-pyrimidin-4-yl]piperazin-1-yl]methyl]-6-methyl-morpholin-4-yl]quinoline-8-carbonitrile

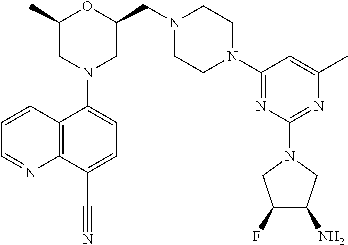 [SMILES: Cc1cc(N2CCN(C[C@H]3CN(c4ccc(C#N)c5ncccc45)C[C@@H](C)O3)CC2)nc(N2C[C@@H](N)[C@@H](F)C2)n1]

The title compound was prepared in analogy to the preparation of Example 45 by using tert-butyl N-((3S,4R)-4-fluoropyrrolidin-3-yl)carbamate (PharmaBlock, PB09204, CAS: 1033718-91-0) and 2,4-dichloro-6-methylpyrimidine instead of 2,4-dichloro-5-fluoropyrimidine (compound 45d) and tert-butyl piperazine-1-carboxylate (compound 45a). Example 54 (69 mg) was obtained as an orange solid. MS: calc'd 546 (MH⁺), measured 546 (MH⁺). ¹H NMR (METHANOL-d₄, 400 MHz) δ 8.98 (dd, 1H, J=1.6, 4.3 Hz), 8.65 (dd, 1H, J=1.6, 8.6 Hz), 8.15 (d, 1H, J=8.1 Hz), 7.64 (dd, 1H, J=4.3, 8.6 Hz), 7.27 (d, 1H, J=8.1 Hz), 6.55 (s, 1H), 5.4-5.6 (m, 1H), 4.5-4.6 (m, 1H), 3.9-4.4 (m, 9H), 3.7-3.8 (m, 1H), 3.5-3.7 (m, 4H), 3.4-3.5 (m, 4H), 2.7-2.8 (m, 2H), 2.44 (s, 3H), 1.31 (d, 3H, J=6.2 Hz).

### Example 55

### 5-[(2S,6R)-2-[[4-[2-[(3R,4S)-3-amino-4-fluoro-pyrrolidin-1-yl]-6-methyl-pyrimidin-4-yl]piperazin-1-yl]methyl]-6-methyl-morpholin-4-yl]quinoline-8-carbonitrile

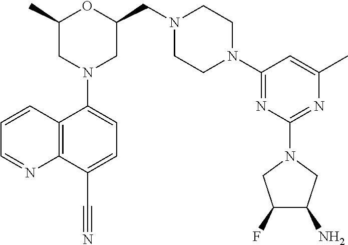 [SMILES: Cc1cc(N2CCN(C[C@H]3CN(c4ccc(C#N)c5ncccc45)C[C@@H](C)O3)CC2)nc(N2C[C@@H](N)[C@@H](F)C2)n1]

The title compound was prepared in analogy to the preparation of Example 45 by using tert-butyl N-((3R,4S)-4-fluoropyrrolidin-3-yl)carbamate and 2,4-dichloro-6-methylpyrimidine instead of 2,4-dichloro-5-fluoropyrimidine (compound 45d) and tert-butyl piperazine-1-carboxylate (compound 45a). Example 55 (66 mg) was obtained as an orange solid. MS: calc'd 546 (MH⁺), measured 546 (MH⁺). ¹H NMR (METHANOL-d₄, 400 MHz) δ 8.98 (dd, 1H, J=1.5, 4.3 Hz), 8.65 (dd, 1H, J=1.5, 8.6 Hz), 8.15 (d, 1H, J=7.9 Hz), 7.64 (dd, 1H, J=4.3, 8.6 Hz), 7.27 (d, 1H, J=8.1 Hz), 6.55 (s, 1H), 5.4-5.6 (m, 1H), 4.5-4.6 (m, 1H), 3.9-4.4 (m, 9H), 3.7-3.8 (m, 1H), 3.5-3.7 (m, 4H), 3.43 (br d, 4H, J=3.1 Hz), 2.7-2.8 (m, 2H), 2.44 (s, 3H), 1.31 (d, 3H, J=6.2 Hz).

### Example 56

### 5-[(2S,6R)-2-[[4-[2-(6-amino-1,4-oxazepan-4-yl)-6-methyl-pyrimidin-4-yl]piperazin-1-yl]methyl]-6-methyl-morpholin-4-yl]quinoline-8-carbonitrile

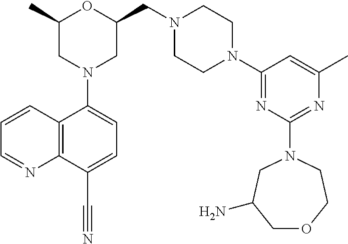 [SMILES: Cc1cc(N2CCN(C[C@H]3CN(c4ccc(C#N)c5ncccc45)C[C@@H](C)O3)CC2)nc(N2CCOCC(N)C2)n1]

The title compound was prepared in analogy to the preparation of Example 45 by using tert-butyl N-(1,4-oxazepan-6-yl)carbamate (CAS: 1782916-90-8) and 2,4-dichloro-6-methylpyrimidine instead of 2,4-dichloro-5-fluoropyrimidine (compound 45d) and tert-butyl piperazine-1-carboxylate (compound 45a). Example 56 (25.3 mg) was obtained as an orange solid. MS: calc'd 558 (MH⁺), measured 558 (MH⁺). ¹H NMR (METHANOL-d₄, 400 MHz) δ 8.99 (dd, 1H, J=1.6, 4.3 Hz), 8.65 (dd, 1H, J=1.7, 8.6 Hz), 8.16 (d, 1H, J=8.1 Hz), 7.64 (dd, 1H, J=4.3, 8.6 Hz), 7.28 (d, 1H, J=8.1 Hz), 6.54 (s, 1H), 4.4-4.6 (m, 2H), 3.7-4.3 (m, 13H), 3.59 (br d, 4H, J=3.4 Hz), 3.4-3.5 (m, 4H), 2.7-2.8 (m, 2H), 2.44 (s, 3H), 1.31 (d, 3H, J=6.2 Hz).

### Example 57

### 5-[(2S,6R)-2-[[4-[2-[(4-cyanopyrrolidin-3-yl)amino]-6-methyl-pyrimidin-4-yl]piperazin-1-yl]methyl]-6-methyl-morpholin-4-yl]quinoline-8-carbonitrile

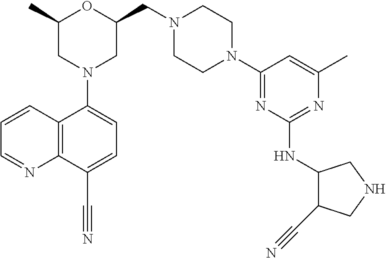 [SMILES: Cc1cc(N2CCN(C[C@H]3CN(c4ccc(C#N)c5ncccc45)C[C@@H](C)O3)CC2)nc(NC2CNCC2C#N)n1]

The title compound was prepared in analogy to the preparation of Example 45 by using tert-butyl 3-amino-4-cyanopyrrolidine-1-carboxylate and 2,4-dichloro-6-methylpyrimidine instead of 2,4-dichloro-5-fluoropyrimidine (compound 45d) and tert-butyl piperazine-1-carboxylate (compound 45a). Example 57 (53.7 mg) was obtained as an orange solid. MS: calc'd 558 (MH⁺), measured 558 (MH⁺). ¹H NMR (METHANOL-d₄, 400 MHz) δ 8.99 (dd, 1H, J=1.5, 4.2 Hz), 8.65 (dd, 1H, J=1.5, 8.6 Hz), 8.16 (d, 1H, J=8.1 Hz), 7.64 (dd, 1H, J=4.3, 8.6 Hz), 7.28 (d, 1H, J=8.1 Hz), 6.54 (d, 1H, J=5.3 Hz), 5.2-5.3 (m, 1H), 4.5-4.5 (m, 1H), 3.9-4.4 (m, 5H), 3.7-3.9 (m, 4H), 3.4-3.7 (m, 9H), 2.7-2.8 (m, 2H), 2.4-2.4 (m, 3H), 1.31 (d, 3H, J=6.2 Hz).

### Example 58

### 5-[(2R,6S)-2-methyl-6-[[4-[6-methyl-2-[(1R,5R)-3-oxa-7,9-diazabicyclo[3.3.1]nonan-7-yl]pyrimidin-4-yl]piperazin-1-yl]methyl]morpholin-4-yl]quinoline-8-carbonitrile

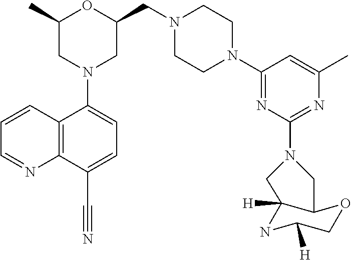

The title compound was prepared in analogy to the preparation of Example 45 by using tert-butyl (1R,5R)-3-oxa-7,9-diazabicyclo[3.3.1]nonane-9-carboxylate (PharmaBlock, PB07078, CAS: 1251010-45-3) and 2,4-dichloro-6-methylpyrimidine instead of 2,4-dichloro-5-fluoropyrimidine (compound 45d) and tert-butyl piperazine-1-carboxylate (compound 45a). Example 58 (9.9 mg) was obtained as an orange solid. MS: calc'd 570 (MH⁺), measured 570 (MH⁺). ¹H NMR (METHANOL-d₄, 400 MHz) δ 8.99 (dd, 1H, J=1.6, 4.3 Hz), 8.66 (dd, 1H, J=1.5, 8.6 Hz), 8.16 (d, 1H, J=8.1 Hz), 7.65 (dd, 1H, J=4.2, 8.6 Hz), 7.28 (d, 1H, J=7.9 Hz), 6.54 (s, 1H), 4.9-5.1 (m, 2H), 4.5-4.6 (m, 1H), 4.0-4.3 (m, 8H), 3.7-3.9 (m, 4H), 3.58 (br d, 4H, J=1.1 Hz), 3.4-3.5 (m, 4H), 2.7-2.8 (m, 2H), 2.44 (s, 3H), 1.3-1.3 (m, 3H).

### Example 59

### 5-[(2R,6S)-2-methyl-6-[[4-[6-methyl-2-[trans-3-amino-4-methoxy-pyrrolidin-1-yl]pyrimidin-4-yl]piperazin-1-yl]methyl]morpholin-4-yl]quinoline-8-carbonitrile

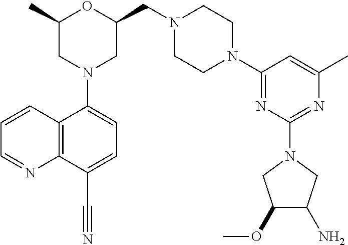 [SMILES: CO[C@H]1CN(c2nc(C)cc(N3CCN(C[C@H]4CN(c5ccc(C#N)c6ncccc56)C[C@@H](C)O4)CC3)n2)CC1N]

The title compound was prepared in analogy to the preparation of Example 45 by using tert-butyl N-(trans-4-methoxypyrrolidin-3-yl)carbamate and 2,4-dichloro-6-methylpyrimidine instead of 2,4-dichloro-5-fluoropyrimidine (compound 45d) and tert-butyl piperazine-1-carboxylate (compound 45a). Example 59 (4.5 mg) was obtained as an orange solid. MS: calc'd 558 (MH⁺), measured 558 (MH⁺). ¹H NMR (METHANOL-d₄, 400 MHz) δ 9.00 (dd, 1H, J=1.6, 4.3 Hz), 8.66 (dd, 1H, J=1.6, 8.7 Hz), 8.17 (d, 1H, J=7.9 Hz), 7.65 (dd, 1H, J=4.3, 8.6 Hz), 7.28 (d, 1H, J=8.1 Hz), 6.51 (s, 1H), 4.4-4.5 (m, 1H), 4.0-4.4 (m, 9H), 3.6-4.0 (m, 3H), 3.4-3.6 (m, 10H), 2.7-2.8 (m, 2H), 2.42 (s, 3H), 1.31 (d, 3H, J=6.2 Hz).

### Example 60

### 5-[(2R,6S)-2-methyl-6-[[4-[6-methyl-2-[cis-3-amino-4-methoxy-pyrrolidin-1-yl]pyrimidin-4-yl]piperazin-1-yl]methyl]morpholin-4-yl]quinoline-8-carbonitrile

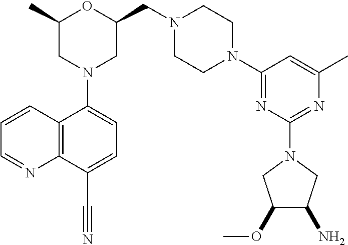 [SMILES: CO[C@H]1CN(c2nc(C)cc(N3CCN(C[C@H]4CN(c5ccc(C#N)c6ncccc56)C[C@@H](C)O4)CC3)n2)C[C@H]1N]

The title compound was prepared in analogy to the preparation of Example 45 by using tert-butyl N-(cis-4-methoxypyrrolidin-3-yl)carbamate and 2,4-dichloro-6-methylpyrimidine instead of 2,4-dichloro-5-fluoropyrimidine (compound 45d) and tert-butyl piperazine-1-carboxylate (compound 45a). Example 59 (6.2 mg) was obtained as an orange solid. MS: calc'd 558 (MH⁺), measured 558 (MH⁺). ¹H NMR (METHANOL-d₄, 400 MHz) δ 8.99 (dd, 1H, J=1.6, 4.3 Hz), 8.66 (dd, 1H, J=1.7, 8.6 Hz), 8.16 (d, 1H, J=7.9 Hz), 7.65 (dd, 1H, J=4.3, 8.6 Hz), 7.28 (d, 1H, J=8.1 Hz), 6.53 (s, 1H), 4.5-4.6 (m, 1H), 4.0-4.4 (m, 8H), 3.7-4.0 (m, 3H), 3.5-3.7 (m, 4H), 3.51 (s, 3H), 3.4-3.5 (m, 4H), 2.7-2.8 (m, 2H), 2.43 (s, 3H), 1.31 (d, 3H, J=6.2 Hz).

### Example 61

### 5-[(2R,6S)-2-methyl-6-[[4-[6-methyl-2-(5-oxa-2,8-diazaspiro[3.5]nonan-2-yl)pyrimidin-4-yl]piperazin-1-yl]methyl]morpholin-4-yl]quinoline-8-carbonitrile

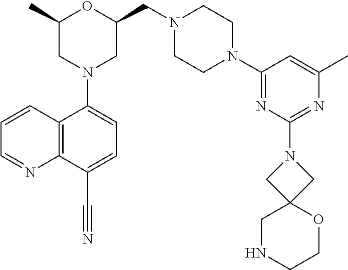 [SMILES: Cc1cc(N2CCN(C[C@H]3CN(c4ccc(C#N)c5ncccc45)C[C@@H](C)O3)CC2)nc(N2CC3(CNCCO3)C2)n1]

The title compound was prepared in analogy to the preparation of Example 45 by using tert-butyl 5-oxa-2,8-diazaspiro[3.5]nonane-8-carboxylate and 2,4-dichloro-6-methylpyrimidine instead of 2,4-dichloro-5-fluoropyrimidine (compound 45d) and tert-butyl piperazine-1-carboxylate (compound 45a). Example 61 (55 mg) was obtained as an orange solid. MS: calc'd 570 (MH⁺), measured 570 (MH⁺). ¹H NMR (METHANOL-d₄, 400 MHz) δ 8.98 (dd, 1H, J=1.6, 4.3 Hz), 8.64 (dd, 1H, J=1.5, 8.6 Hz), 8.14 (d, 1H, J=8.1 Hz), 7.64 (dd, 1H, J=4.2, 8.6 Hz), 7.26 (d, 1H, J=8.1 Hz), 6.49 (s, 1H), 4.5-4.6 (m, 1H), 4.3-4.4 (m, 2H), 4.1-4.3 (m, 6H), 4.0-4.0 (m, 2H), 3.56 (s, 6H), 3.41 (br s, 4H), 3.3-3.4 (m, 1H), 3.3-3.3 (m, 2H), 2.7-2.8 (m, 2H), 2.39 (s, 3H), 1.31 (d, 3H, J=6.2 Hz).

### Example 62

### 5-[(2R,6S)-2-methyl-6-[[4-(3-morpholin-2-ylphenyl)piperazin-1-yl]methyl]-morpholin-4-yl]quinoline-8-carbonitrile

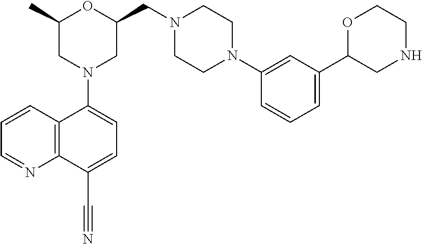 [SMILES: C[C@@H]1CN(c2ccc(C#N)c3ncccc23)C[C@H](CN2CCN(c3cccc(C4CNCCO4)c3)CC2)O1]

The title compound was prepared according to the following scheme:

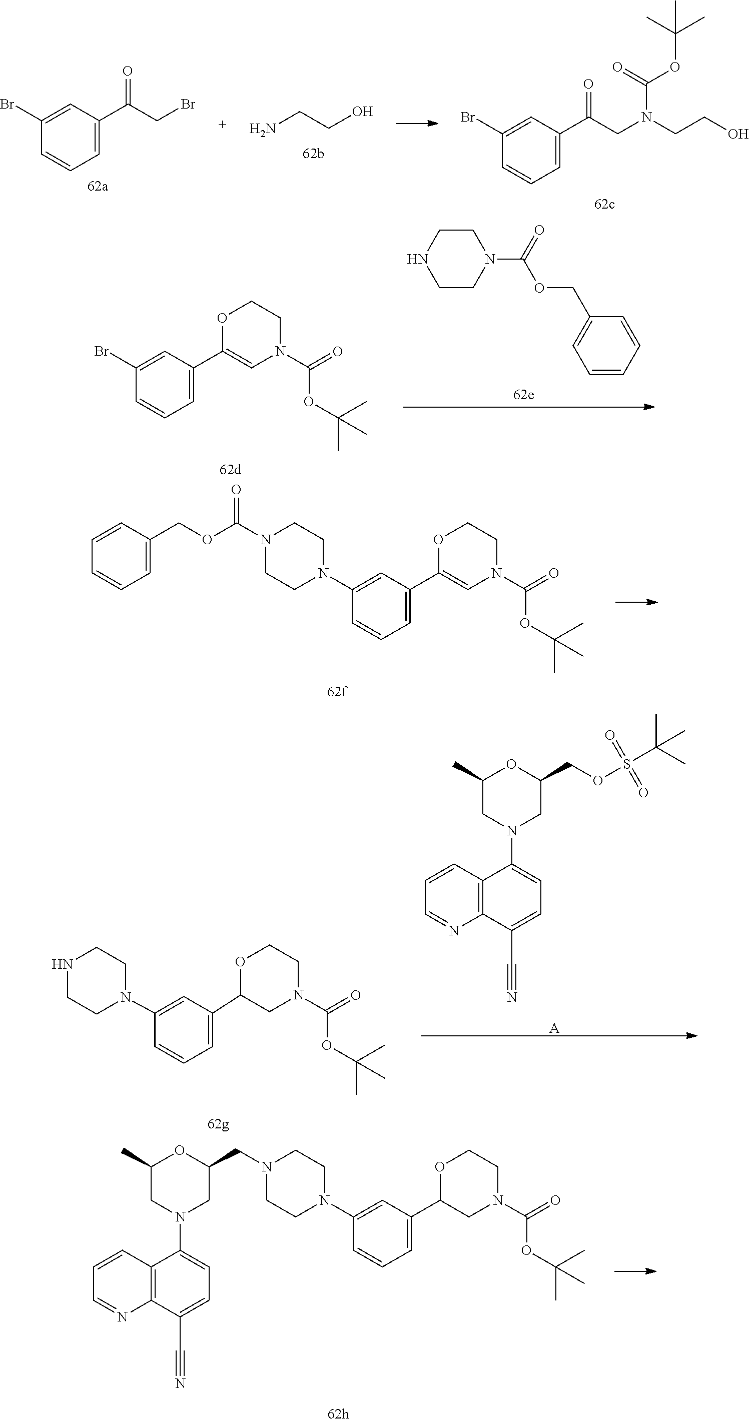

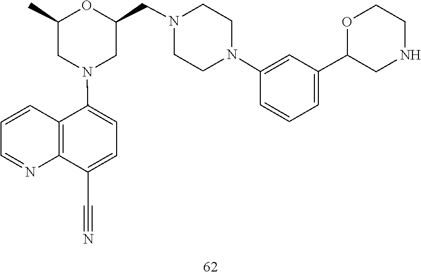 [SMILES: C[C@@H]1CN(c2ccc(C#N)c3ncccc23)C[C@H](CN2CCN(c3cccc(C4CNCCO4)c3)CC2)O1]

### Step 1: preparation of tert-butyl N-[2-(3-bromophenyl)-2-oxo-ethyl]-N-(2-hydroxyethyl)carbamate (Compound 62c)

To the solution of 2-aminoethan-1-ol (compound 62b, 1.424 mL, 23.8 mmol) in EtOH (60 mL) was added 2-bromo-1-(3-bromophenyl)ethan-1-one (compound 62a, 3.3 g, 11.9 mmol) at −10° C. Then N,N-Diisopropylethylamine (6.2 mL) was added to the mixture slowly at the same temperature. The mixture was stirred for 40 minutes at 20° C. Then Et₃N and (Boc)₂O were added to the mixture and the mixture was stirred for 30 minutes at 20° C. The mixture was concentrated and the crude was dissolved in DCM (100 mL). The mixture was washed with saturated NH₄Cl aqueous solution (80 mL) and H₂O (80 mL). The organic layer was dried and concentrated in vacuo. The residue was purified by column chromatography to give compound 62c (1.01 g). MS calc'd 358, 360 (MH⁺); measured 358, 360 (MH⁺).

### Step 2: preparation of tert-butyl 6-(3-bromophenyl)-2,3-dihydro-1,4-oxazine-4-carboxylate (Compound 62d)

To the solution of tert-butyl N-[2-(3-bromophenyl)-2-oxo-ethyl]-N-(2-hydroxyethyl)-carbamate (compound 62c, 358 mg, 1 mmol) and Et₃SiH (160 μL, 2 mmol) in dry DCM (10 mL) was added InBr₃ at 0° C. Then the mixture was stirred for 16 h at 20° C. The mixture was diluted with DCM (15 mL) and washed with H₂O (10 mL). The organic layer was dried and concentrated. The residue was purified by column chromatography to give compound 62d (270 mg). MS calc'd 341 (MH⁺); measured 341 (MH⁺).

### Step 3: preparation of tert-butyl 6-[3-(4-benzyloxycarbonylpiperazin-1-yl)phenyl]-2,3-dihydro-1,4-oxazine-4-carboxylate (Compound 621)

The solution of tert-butyl 6-(3-bromophenyl)-2,3-dihydro-1,4-oxazine-4-carboxylate (compound 62d, 135 mg, 0.397 mmol), benzyl piperazine-1-carboxylate (compound 63e, 131 mg, 0.595 mmol), tBuXPhos Pd G3 (31.5 mg, 0.040 mmol) and tBuONa (76.3 mg, 0.794 mmol) in dry dioxane (3 mL) was stirred at 90° C. for 2 hrs. The mixture was diluted with EA (20 mL) and filtered through Na₂SO₄. The filtrate was washed with saturated NH₄Cl aqueous solution (10 mL) and brine (10 mL). The organic layer was dried and concentrated under vacuum. The residue was purified by column chromatography to give compound 62f (96 mg). MS calc'd 480 (MH⁺); measured 480 (MH⁺).

### Step 4: preparation of tert-butyl 2-(3-piperazin-1-ylphenyl)morpholine-4-carboxylate (Compound 62g)

The solution of tert-butyl 6-[3-(4-benzyloxycarbonylpiperazin-1-yl)phenyl]-2,3-dihydro-1,4-oxazine-4-carboxylate (compound 62f, 96 mg, 0.2 mmol), Pd(OH)₂ (20% on carbon with 50% H₂O) (11.2 mg, 0.04 mmol) in MeOH (3.5 mL) was stirred at 20° C. under H₂ for 20 hours. The mixture was filtered and the filtrate was concentrated to give compound 62g (69 mg). MS calc'd 348 (MH⁺); measured 348 (MH⁺).

### Step 5: preparation of tert-butyl 2-[3-[4-[[(2S,6R)-4-(8-cyano-5-quinolyl)-6-methyl-morpholin-2-yl]methyl]piperazin-1-yl]phenyl]morpholine-4-carboxylate (Compound 62h)

To a tube was added tert-butyl 2-(3-(piperazin-1-yl)phenyl)morpholine-4-carboxylate (compound 62g, 69 mg, 200 μmol), [(2R,6R)-4-(8-cyano-5-quinolyl)-6-methyl-morpholin-2-yl]methyl trifluoromethanesulfonate (intermediate A, 100 mg, 240 μmol), K₂CO₃ (55 mg, 400 μmol) and ACN (6 mL), The mixture was stirred for 2 hrs at 80° C. The mixture was diluted with some ACN and filtered through celite. The filtrate was concentrated to give a yellow oil which was purified by prep-HPLC to give compound 62h (35 mg) as yellow solid. MS calc'd 613 (MH⁺); measured 613 (MH⁺).

### Step 6: preparation of 5-[(2R,6S)-2-methyl-6-[[4-(3-morpholin-2-ylphenyl)piperazin-1-yl]methyl]morpholin-4-yl]quinoline-8-carbonitrile (Example 62)

To a solution of tert-butyl 2-[3-[4-[[(2S,6R)-4-(8-cyano-5-quinolyl)-6-methyl-morpholin-2-yl]methyl]piperazin-1-yl]phenyl]morpholine-4-carboxylate (compound 62h, 7 mg, 0.011 mmol) in EA (3 mL) was added HCl in EA (1M) (1.0 mL, 1 mmol). After being stirred at rt overnight, the mixture was concentrated in vacuo to give Example 62 (7 mg) as a brown solid. MS calc'd 513 (MH⁺), measured 513 (MH⁺). ¹H NMR (400 MHz, METHANOL-d₄) δ: ppm 9.01 (d, J=3.8 Hz, 1H), 8.71 (br d, J=8.3 Hz, 1H), 8.19 (d, J=7.9 Hz, 1H), 7.68 (dd, J=4.3, 8.4 Hz, 1H), 7.38-7.28 (m, 2H), 7.13 (s, 1H), 7.06 (br d, J=8.1 Hz, 1H), 6.98 (d, J=7.6 Hz, 1H), 4.78-4.74 (m, 1H), 4.60-4.50 (m, 1H), 4.29-4.16 (m, 2H), 4.06-3.96 (m, 1H), 3.95-3.76 (m, 4H), 3.50-3.36 (m, 9H), 3.26-3.19 (m, 2H), 3.13-3.06 (m, 1H), 2.84-2.71 (m, 2H), 1.33 (d, J=6.0 Hz, 3H).

### Example 63

### 5-[(2S,6R)-2-[[4-[2-(4-amino-3,3-difluoro-pyrrolidin-1-yl)-6-methyl-pyrimidin-4-yl]piperazin-1-yl]methyl]-6-methyl-morpholin-4-yl]quinoline-8-carbonitrile

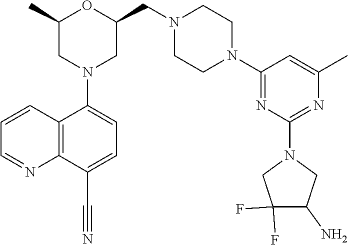 [SMILES: Cc1cc(N2CCN(C[C@H]3CN(c4ccc(C#N)c5ncccc45)C[C@@H](C)O3)CC2)nc(N2CC(N)C(F)(F)C2)n1]

The title compound was prepared in analogy to the preparation of Example 45 by using tert-butyl N-(4,4-difluoropyrrolidin-3-yl)carbamate (CAS: 1434141-95-3) and 2,4-dichloro-6-methylpyrimidine instead of 2,4-dichloro-5-fluoropyrimidine (compound 45d) and tert-butyl piperazine-1-carboxylate (compound 45a). Example 63 (17.8 mg) was obtained as an orange solid. MS: calc'd 564 (MH⁺), measured 564 (MH⁺). ¹H NMR (METHANOL-d₄, 400 MHz) δ 8.99 (dd, 1H, J=1.7, 4.2 Hz), 8.66 (dd, 1H, J=1.6, 8.6 Hz), 8.16 (d, 1H, J=7.9 Hz), 7.65 (dd, 1H, J=4.3, 8.7 Hz), 7.28 (d, 1H, J=8.1 Hz), 6.56 (s, 1H), 4.4-4.6 (m, 2H), 4.1-4.4 (m, 7H), 3.8-4.0 (m, 2H), 3.5-3.7 (m, 4H), 3.4-3.5 (m, 4H), 2.7-2.8 (m, 2H), 2.43 (s, 3H), 1.31 (d, 3H, J=6.2 Hz).

### Example 64

### 8-[(2R,6S)-2-methyl-6-[[4-(6-methyl-2-piperazin-1-yl-pyrimidin-4-yl)piperazin-1-yl]methyl]morpholin-4-yl]quinoxaline-5-carbonitrile

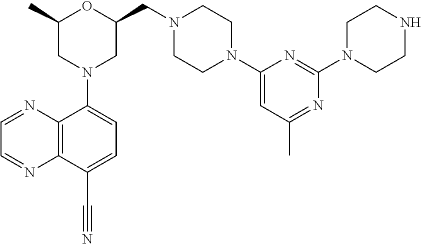 [SMILES: Cc1cc(N2CCN(C[C@H]3CN(c4ccc(C#N)c5nccnc45)C[C@@H](C)O3)CC2)nc(N2CCNCC2)n1]

The title compound was prepared in analogy to the preparation of Example 45 by using 2,4-dichloro-6-methylpyrimidine and ((2R,6R)-4-(8-cyanoquinoxalin-5-yl)-6-methylmorpholin-2-yl)methyl trifluoromethanesulfonate (Compound 64a) instead of 2,4-dichloro-5-fluoropyrimidine (compound 45d) and ((2R,6R)-4-(8-cyanoquinolin-5-yl)-6-methylmorpholin-2-yl)methyl trifluoromethanesulfonate (Intermediate A). Example 64 (18.8 mg) was obtained as an orange solid. MS: calc'd 529 (MH⁺), measured 529 (MH⁺). ¹H NMR (METHANOL-d₄, 400 MHz) δ 8.97 (d, 1H, J=1.7 Hz), 8.89 (d, 1H, J=1.7 Hz), 8.15 (d, 1H, J=8.4 Hz), 7.26 (d, 1H, J=8.3 Hz), 6.47 (s, 1H), 4.4-4.5 (m, 1H), 4.2-4.4 (m, 1H), 4.0-4.2 (m, 9H), 3.4-3.6 (m, 4H), 3.3-3.4 (m, 7H), 2.8-2.9 (m, 2H), 2.39 (s, 3H), 1.32 (d, 3H, J=6.1 Hz).

### Preparation of ((2R,6R)-4-(8-cyanoquinoxalin-5-yl)-6-methylmorpholin-2-yl)methyl trifluoromethanesulfonate (Compound 64a)

Compound 64a was prepared in analogy to the preparation of intermediate A by using 8-bromoquinoxaline-5-carbonitrile instead of 5-bromoquinoline-8-carbonitrile (compound A2)

### Example 65

### (2R,6S)-2-methyl-6-[[4-(6-methyl-2-piperazin-1-yl-pyrimidin-4-yl)piperazin-1-yl]methyl]-4-[8-(trifluoromethyl)quinoxalin-5-yl]morpholine

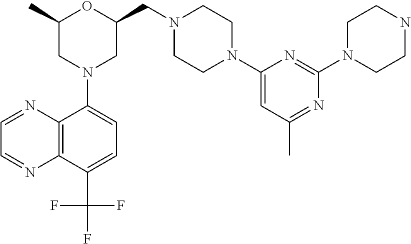 [SMILES: Cc1cc(N2CCN(C[C@H]3CN(c4ccc(C(F)(F)F)c5nccnc45)C[C@@H](C)O3)CC2)nc(N2CCNCC2)n1]

The title compound was prepared in analogy to the preparation of Example 45 by using 2,4-dichloro-6-methylpyrimidine and ((2R,6R)-4-(8-(trifluoromethyl)quinoxalin-5-yl)-6-methylmorpholin-2-yl)methyl trifluoromethanesulfonate (Compound 65a) instead of 2,4-dichloro-5-fluoropyrimidine (compound 45d) and ((2R,6R)-4-(8-cyanoquinolin-5-yl)-6-methylmorpholin-2-yl)methyl trifluoromethanesulfonate (Intermediate A). Example 65 (17.6 mg) was obtained as an orange solid. MS: calc'd 572 (MH⁺), measured 572 (MH⁺). ¹H NMR (METHANOL-d₄, 400 MHz) δ 8.95 (d, 1H, J=1.8 Hz), 8.88 (d, 1H, J=1.7 Hz), 8.1-8.1 (m, 1H), 7.27 (d, 1H, J=8.4 Hz), 6.44 (s, 1H), 4.4-4.5 (m, 1H), 4.0-4.2 (m, 10H), 3.4-3.6 (m, 4H), 3.3-3.4 (m, 7H), 2.7-2.8 (m, 2H), 2.38 (s, 3H), 1.33 (d, 3H, J=6.2 Hz).

### Preparation of ((2R,6R)-4-(8-(trifluoromethyl)quinoxalin-5-yl)-6-methylmorpholin-2-yl)methyl trifluoromethanesulfonate (Compound 65a)

Compound 65a was prepared in analogy to the preparation of intermediate A by using 5-bromo-8-(trifluoromethyl)quinoxaline instead of 5-bromoquinoline-8-carbonitrile (compound A2)

### Example 66

### 5-[(2S,6R)-2-[[4-[2-(6-hydroxy-1,4-diazepan-1-yl)-6-methyl-pyrimidin-4-yl]piperazin-1-yl]methyl]-6-methyl-morpholin-4-yl]quinoline-8-carbonitrile

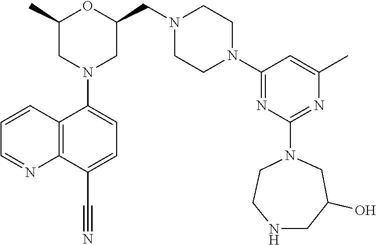 [SMILES: Cc1cc(N2CCN(C[C@H]3CN(c4ccc(C#N)c5ncccc45)C[C@@H](C)O3)CC2)nc(N2CCNCC(O)C2)n1]

The title compound was prepared in analogy to the preparation of Example 45 by using tert-butyl 6-hydroxy-1,4-diazepane-1-carboxylate (Wuxi Apptec, WX604354, CAS: 956317-40-1) and 2,4-dichloro-6-methylpyrimidine instead of 2,4-dichloro-5-fluoropyrimidine (compound 45d) and tert-butyl piperazine-1-carboxylate (compound 45a). Example 66 (24.1 mg) was obtained as an orange solid. MS: calc'd 558 (MH⁺), measured 558 (MH⁺). ¹H NMR (METHANOL-d₄, 400 MHz) δ 9.0-9.1 (m, 1H), 8.68 (br d, 1H, J=8.4 Hz), 8.19 (dd, 1H, J=1.7, 7.8 Hz), 7.6-7.8 (m, 1H), 7.31 (br d, 1H, J=8.2 Hz), 6.53 (s, 1H), 4.5-4.6 (m, 2H), 4.4-4.5 (m, 1H), 4.3-4.4 (m, 2H), 4.1-4.2 (m, 4H), 4.0-4.1 (m, 1H), 3.8-3.9 (m, 2H), 3.6-3.7 (m, 1H), 3.4-3.6 (m, 10H), 2.7-2.9 (m, 2H), 2.43 (s, 3H), 1.33 (br d, 3H, J=6.1 Hz).

### Example 67

### 3-Fluoro-4-[(2R,6S)-2-methyl-6-[[4-(6-methyl-2-piperazin-1-yl-pyrimidin-4-yl)piperazin-1-yl]methyl]morpholin-4-yl]pyrazolo[1,5-a]pyridine-7-carbonitrile

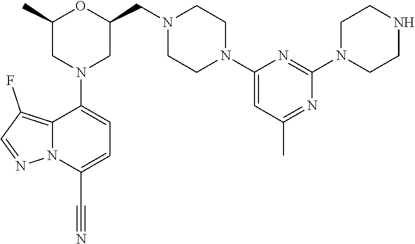 [SMILES: Cc1cc(N2CCN(C[C@H]3CN(c4ccc(C#N)n5ncc(F)c45)C[C@@H](C)O3)CC2)nc(N2CCNCC2)n1]

The title compound was prepared in analogy to the preparation of Example 45 by using 2,4-dichloro-6-methylpyrimidine and (2R,6R)-4-(7-cyano-3-fluoro-pyrazolo[1,5-a]pyridin-4-yl)-6-methyl-morpholin-2-yl]methyl trifluoromethanesulfonate (Compound 67a) instead of 2,4-dichloro-5-fluoropyrimidine (compound 45d) and ((2R,6R)-4-(8-cyanoquinolin-5-yl)-6-methylmorpholin-2-yl)methyl trifluoromethanesulfonate (Intermediate A). Example 67 (28.6 mg) was obtained as an orange solid. MS: calc'd 535 (MH⁺), measured 535 (MH⁺). ¹H NMR (METHANOL-d₄, 400 MHz) δ 8.03 (br d, 1H, J=2.1 Hz), 7.46 (dd, 1H, J=2.1, 7.9 Hz), 6.57 (dd, 1H, J=1.9, 7.9 Hz), 6.48 (s, 1H), 4.3-4.4 (m, 1H), 4.09 (br s, 8H), 3.6-3.7 (m, 3H), 3.4-3.5 (m, 4H), 3.38 (br d, 6H, J=3.5 Hz), 2.7-2.8 (m, 2H), 2.41 (d, 3H, J=1.7 Hz), 1.32 (dd, 3H, J=2.0, 6.2 Hz).

### Preparation of (2R,6R)-4-(7-cyano-3-fluoro-pyrazolo[1,5-a]pyridin-4-yl)-6-methyl-morpholin-2-yl]methyl trifluoromethanesulfonate (Compound 67a)

Compound 67a was prepared in analogy to the preparation of intermediate A by using 4-bromo-3-fluoro-pyrazolo[1,5-a]pyridine-7-carbonitrile instead of 5-bromoquinoline-8-carbonitrile (compound A2)

### Example 68

### 5-[(2R,6S)-2-methyl-6-[[4-(4-methyl-6-piperazin-1-yl-pyrimidin-2-yl)piperazin-1-yl]methyl]morpholin-4-yl]quinoline-8-carbonitrile

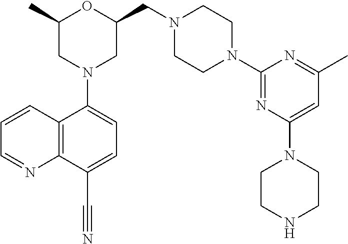 [SMILES: Cc1cc(N2CCNCC2)nc(N2CCN(C[C@H]3CN(c4ccc(C#N)c5ncccc45)C[C@@H](C)O3)CC2)n1]

The title compound was prepared in analogy to the preparation of Example 45 by using 2,4-dichloro-6-methylpyrimidine instead of 2,4-dichloro-5-fluoropyrimidine (compound 45d). Example 68 (5.2 mg) was obtained as an orange solid. MS: calc'd 558 (MH⁺), measured 558 (MH⁺). ¹H NMR (METHANOL-d₄, 400 MHz) δ 9.0-9.1 (m, 1H), 8.7-8.7 (m, 1H), 8.19 (d, 1H, J=7.9 Hz), 7.67 (dd, 1H, J=4.2, 8.6 Hz), 7.31 (d, 1H, J=7.9 Hz), 6.50 (s, 1H), 4.5-4.6 (m, 2H), 4.2-4.2 (m, 4H), 4.09 (br s, 4H), 3.57 (br d, 4H, J=2.0 Hz), 3.4-3.5 (m, 8H), 2.7-2.9 (m, 2H), 2.42 (s, 3H), 1.33 (br d, 3H, J=6.2 Hz).

### Example 69A and 69B

### 5-[(2S,6R)-2-[[4-[2-[(3S,4S)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile and 5-[(2S,6R)-2-[[4-[2-[(3R,4R)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile

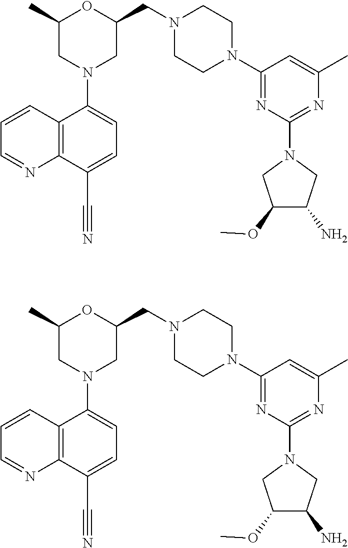 [SMILES: CO[C@H]1CN(c2nc(C)cc(N3CCN(C[C@H]4CN(c5ccc(C#N)c6ncccc56)C[C@@H](C)O4)CC3)n2)C[C@@H]1N ; CO[C@@H]1CN(c2nc(C)cc(N3CCN(C[C@H]4CN(c5ccc(C#N)c6ncccc56)C[C@@H](C)O4)CC3)n2)C[C@H]1N]

The title compound was prepared according to the following scheme:

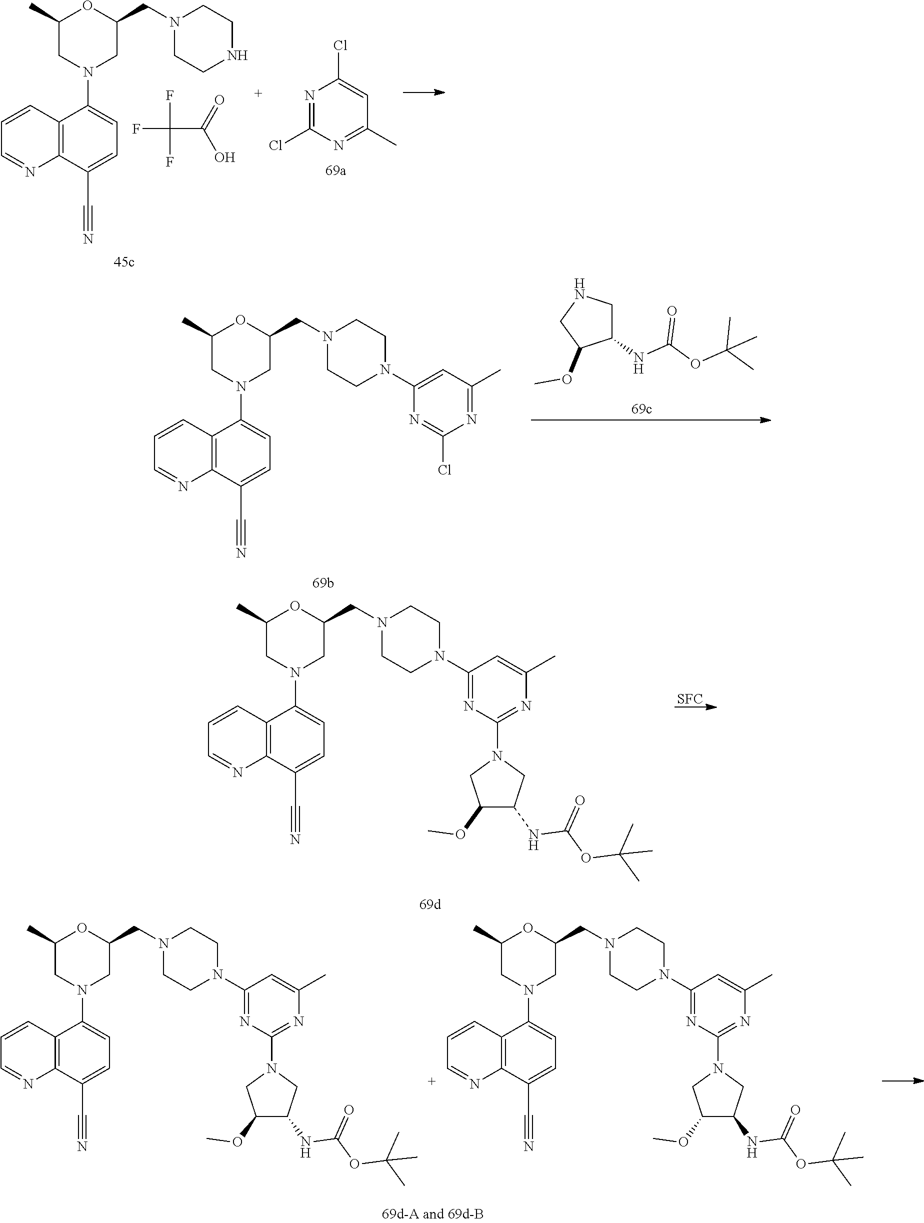

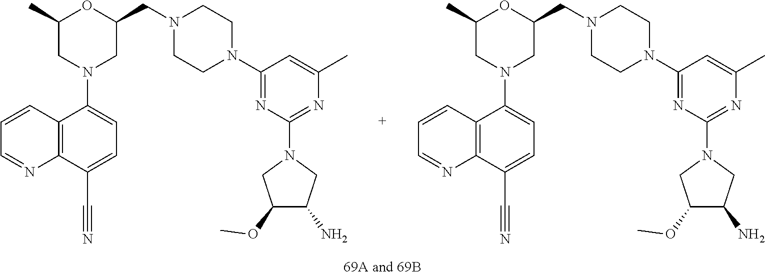 [SMILES: CO[C@H]1CN(c2nc(C)cc(N3CCN(C[C@H]4CN(c5ccc(C#N)c6ncccc56)C[C@@H](C)O4)CC3)n2)C[C@@H]1N ; CO[C@@H]1CN(c2nc(C)cc(N3CCN(C[C@H]4CN(c5ccc(C#N)c6ncccc56)C[C@@H](C)O4)CC3)n2)C[C@H]1N]

### Step 1: 5-((2S,6R)-2-((4-(2-chloro-6-methylpyrimidin-4-yl)piperazin-1-yl)methyl)-6-methylmorpholino)quinoline-8-carbonitrile (Compound 69b)

A solution of 2,4-dichloro-6-methylpyrimidine (compound 69a, 350 mg, 2.15 mmol) and 5-((2R,6S)-2-methyl-6-(piperazin-1-ylmethyl)morpholino)quinoline-8-carbonitrile 2,2,2-trifluoroacetate (compound 45c, 2 g, 2.15 mmol) in ethanol (10 mL) was stirred at room temperature for 12 hrs. Then reaction mixture was concentrated and purified by silica gel Chromatography to afford 5-((2S,6R)-2-((4-(2-chloro-6-methylpyrimidin-4-yl)piperazin-1-yl)methyl)-6-methylmorpholino)quinoline-8-carbonitrile (compound 69b, 1 g) as a light yellow solid. MS: calc'd 478 (MH⁺), measured 478 (MH⁺).

### Step 2: 5-((2S,6R)-2-((4-(2-chloro-6-methylpyrimidin-4-yl)piperazin-1-yl)methyl)-6-methylmorpholino)quinoline-8-carbonitrile (Compound 69d)

A suspension of 5-((2S,6R)-2-((4-(2-chloro-6-methylpyrimidin-4-yl)piperazin-1-yl)methyl)-6-methylmorpholino)quinoline-8-carbonitrile (compound 69b, 240 mg, 502 μmol), tert-butyl N-(trans-4-methoxypyrrolidin-3-yl)carbamate (compound 69c, 119 mg, 552 μmol), Ruphos Pd G2 (39 mg, 50.2 μmol) and K₂CO₃ (139 mg, 1 mmol) in 1,4-dioxane (4 mL) was heated to 100° C. for 12 hrs under nitrogen atmosphere. The mixture was cooled to rt and then diluted with water (5 mL) and the mixture was extracted with EtOAC (10 mL) three times. The combined organic layer was washed with brine and concentrated. The resulting residue was purified by prep-HPLC to afford tert-butyl (trans-1-(4-(4-(((2S,6R)-4-(8-cyanoquinolin-5-yl)-6-methylmorpholin-2-yl)methyl)piperazin-1-yl)-6-methylpyrimidin-2-yl)-4-methoxypyrrolidin-3-yl)carbamate product (160 mg). Then it was purified by SFC-HPLC (40% CO₂/0.5% NH₃ in methanol as eluent on Daicel AD-H Column) to afford tert-butyl ((3S,4R)-1-(4-(4-(((2S,6R)-4-(8-cyanoquinolin-5-yl)-6-methylmorpholin-2-yl)methyl)piperazin-1-yl)-6-methylpyrimidin-2-yl)-4-methoxypyrrolidin-3-yl)carbamate and tert-butyl((3R,4S)-1-(4-(4-(((2S,6R)-4-(8-cyanoquinolin-5-yl)-6-methylmorpholin-2-yl)methyl)piperazin-1-yl)-6-methylpyrimidin-2-yl)-4-methoxypyrrolidin-3-yl)carbamate (compound 69d-A, faster eluted, 76 mg and compound 69d-B, slower eluted, 77 mg). MS: calc'd 658 (MH⁺), measured 658 (MH⁺).

### Step 3: Preparation of Example 69A and 69B

To a solution of tert-butyl ((3S,4S)-1-(4-(4-(((2S,6R)-4-(8-cyanoquinolin-5-yl)-6-methylmorpholin-2-yl)methyl)piperazin-1-yl)-6-methylpyrimidin-2-yl)-4-methoxypyrrolidin-3-yl)carbamate or tert-butyl ((3R,4R)-1-(4-(4-(((2S,6R)-4-(8-cyanoquinolin-5-yl)-6-methylmorpholin-2-yl)methyl)piperazin-1-yl)-6-methylpyrimidin-2-yl)-4-methoxypyrrolidin-3-yl)carbamate (compound 69d-A, 76 mg or compound 69d-B, 77 mg) in DCM (2 mL) was added hydrogen chloride (4M in dioxane, 0.5 mL) at 0° C. The reaction mixture was stirred at rt for 2 hrs. Then the mixture was concentrated and purified by prep-HPLC to afford Example 69A and

### Example 69B: 5-[(2S,6R)-2-[[4-[2-[(3S,4S)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile and 5-[(2S,6R)-2-[[4-[2-[(3R,4R)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile

Example 69A (44.2 mg) was obtained as a light yellow solid. MS: calc'd 558 (MH⁺), measured 558 (MH⁺). ¹H NMR (METHANOL-d₄, 400 MHz) δ 9.04 (dd, 1H, J=1.7, 4.3 Hz), 8.77 (dd, 1H, J=1.7, 8.6 Hz), 8.22 (d, 1H, J=7.9 Hz), 7.72 (dd, 1H, J=4.3, 8.6 Hz), 7.33 (d, 1H, J=8.1 Hz), 6.59 (s, 1H), 5.0-5.2 (m, 1H), 4.6-4.7 (m, 1H), 4.3-4.5 (m, 1H), 4.2-4.3 (m, 2H), 3.8-4.2 (m, 8H), 3.6-3.7 (m, 1H), 3.52 (s, 9H), 2.7-2.9 (m, 2H), 2.48 (s, 3H), 1.35 (d, 3H, J=6.2 Hz).

Example 69B (48 mg) was obtained as a light yellow solid. MS: calc'd 558 (MH⁺), measured 558 (MH⁺). ¹H NMR (METHANOL-d₄, 400 MHz) δ 9.05 (dd, 1H, J=1.6, 4.4 Hz), 8.80 (dd, 1H, J=1.5, 8.6 Hz), 8.23 (d, 1H, J=7.9 Hz), 7.75 (dd, 1H, J=4.4, 8.6 Hz), 7.35 (d, 1H, J=8.1 Hz), 6.59 (s, 1H), 5.0-5.2 (m, 1H), 4.61 (br t, 1H, J=9.7 Hz), 4.3-4.5 (m, 1H), 4.2-4.3 (m, 2H), 4.0-4.2 (m, 3H), 3.7-4.0 (m, 5H), 3.4-3.5 (m, 9H), 2.81 (ddd, 2H, J=10.0, 12.1, 17.5 Hz), 2.48 (s, 3H), 1.35 (d, 4H, J=6.2 Hz).

### Example 70

### 5-[(2S,6R)-2-[[4-[2-[[(3R,4R)-4-methoxypyrrolidin-3-yl]amino]-6-methyl-pyrimidin-4-yl]piperazin-1-yl]methyl]-6-methyl-morpholin-4-yl]quinoline-8-carbonitrile

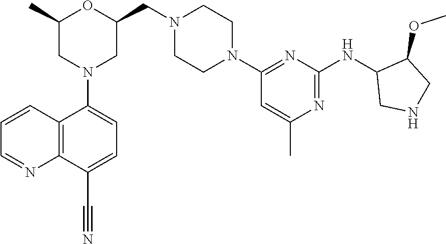 [SMILES: CO[C@H]1CNCC1Nc1nc(C)cc(N2CCN(C[C@H]3CN(c4ccc(C#N)c5ncccc45)C[C@@H](C)O3)CC2)n1]

The title compound was prepared in analogy to the preparation of Example 47 by using tert-butyl (3R,4R)-3-amino-4-methoxy-pyrrolidine-1-carboxylate (CAS: 1400562-12-0) and 2,4-dichloro-6-methylpyrimidine instead of 2,4-dichloro-5-fluoropyrimidine (compound 45d) and tert-butyl piperazine-1-carboxylate (compound 45a). Example 70 (19.5 mg) was obtained as an orange solid. MS: calc'd 558 (MH⁺), measured 558 (MH⁺). ¹H NMR (METHANOL-d₄, 400 MHz) δ 8.8-8.9 (m, 1H), 8.55 (dd, 1H, J=1.2, 8.6 Hz), 8.05 (td, 1H, J=2.1, 7.9 Hz), 7.54 (br dd, 1H, J=4.3, 8.6 Hz), 7.1-7.2 (m, 1H), 6.29 (s, 1H), 4.6-4.6 (m, 1H), 4.1-4.3 (m, 1H), 4.0-4.1 (m, 1H), 3.8-3.9 (m, 4H), 3.6-3.7 (m, 1H), 3.4-3.4 (m, 1H), 3.4-3.4 (m, 3H), 3.3-3.3 (m, 1H), 3.3-3.3 (m, 1H), 3.2-3.2 (m, 5H), 2.8-2.9 (m, 4H), 2.5-2.7 (m, 2H), 2.23 (s, 3H), 1.17 (d, 3H, J=6.2 Hz).

### Example 71

### 5-[(2S,6R)-2-[[4-[2-[[(3R,4S)-4-fluoropyrrolidin-3-yl]amino]-6-methyl-pyrimidin-4-yl]piperazin-1-yl]methyl]-6-methyl-morpholin-4-yl]quinoline-8-carbonitrile

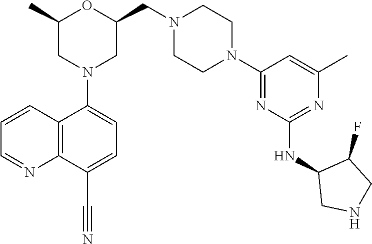 [SMILES: Cc1cc(N2CCN(C[C@H]3CN(c4ccc(C#N)c5ncccc45)C[C@@H](C)O3)CC2)nc(N[C@@H]2CNC[C@@H]2F)n1]

The title compound was prepared in analogy to the preparation of Example 45 by using tert-butyl (3R,4S)-3-amino-4-fluoro-pyrrolidine-1-carboxylate (Pharmablock, PB07375, CAS: 1009075-48-2) and 2,4-dichloro-6-methylpyrimidine instead of 2,4-dichloro-5-fluoropyrimidine (compound 45d) and tert-butyl piperazine-1-carboxylate (compound 45a). Example 71 (10.7 mg) was obtained as an orange solid. MS: calc'd 546 (MH⁺), measured 546 (MH⁺). ¹H NMR (METHANOL-d₄, 400 MHz) δ 8.99 (dd, 1H, J=1.6, 4.3 Hz), 8.66 (dd, 1H, J=1.7, 8.6 Hz), 8.17 (d, 1H, J=8.1 Hz), 7.65 (dd, 1H, J=4.2, 8.6 Hz), 7.28 (d, 1H, J=8.1 Hz), 6.49 (s, 1H), 5.3-5.5 (m, 1H), 5.0-5.1 (m, 3H), 4.4-4.5 (m, 1H), 4.0-4.2 (m, 4H), 3.79 (s, 3H), 3.3-3.5 (m, 6H), 3.2-3.3 (m, 2H), 2.7-2.8 (m, 2H), 2.38 (s, 3H), 1.30 (d, 3H, J=6.2 Hz).

### Example 72

### 5-[(2S,6R)-2-[[4-[2-[(3S,4R)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile

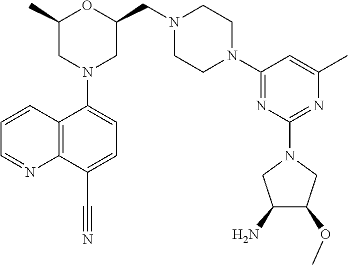 [SMILES: CO[C@@H]1CN(c2nc(C)cc(N3CCN(C[C@H]4CN(c5ccc(C#N)c6ncccc56)C[C@@H](C)O4)CC3)n2)C[C@@H]1N]

The title compound was prepared in analogy to the preparation of Example 45 by using tert-butyl N-[(3S,4R)-4-methoxypyrrolidin-3-yl]carbamate (PharmaBlock, PBZ4730, CAS: 1931911-57-7) and 2,4-dichloro-6-methylpyrimidine instead of 2,4-dichloro-5-fluoropyrimidine (compound 45d) and tert-butyl piperazine-1-carboxylate (compound 45a). Example 72 (31.3 mg) was obtained as an orange solid. MS: calc'd 558 (MH⁺), measured 558 (MH⁺). ¹H NMR (METHANOL-d₄, 400 MHz) δ 8.99 (dd, 1H, J=1.7, 4.2 Hz), 8.66 (dd, 1H, J=1.6, 8.6 Hz), 8.16 (d, 1H, J=8.1 Hz), 7.65 (dd, 1H, J=4.3, 8.6 Hz), 7.28 (d, 1H, J=8.1 Hz), 6.53 (s, 1H), 4.5-4.6 (m, 1H), 3.9-4.4 (m, 9H), 3.7-3.8 (m, 2H), 3.5-3.7 (m, 4H), 3.51 (s, 3H), 3.4-3.5 (m, 4H), 2.7-2.8 (m, 2H), 2.43 (s, 3H), 1.31 (d, 3H, J=6.2 Hz).

### Example 73

### 5-[(2S,6R)-2-[[4-[2-[[(3S,4R)-4-fluoropyrrolidin-3-yl]amino]-6-methyl-pyrimidin-4-yl]piperazin-1-yl]methyl]-6-methyl-morpholin-4-yl]quinoline-8-carbonitrile

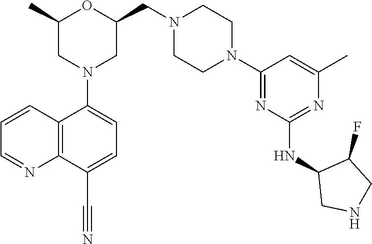 [SMILES: Cc1cc(N2CCN(C[C@H]3CN(c4ccc(C#N)c5ncccc45)C[C@@H](C)O3)CC2)nc(N[C@@H]2CNC[C@@H]2F)n1]

The title compound was prepared in analogy to the preparation of Example 45 by using tert-butyl (3S,4R)-3-amino-4-fluoro-pyrrolidine-1-carboxylate and 2,4-dichloro-6-methylpyrimidine instead of 2,4-dichloro-5-fluoropyrimidine (compound 45d) and tert-butyl piperazine-1-carboxylate (compound 45a). Example 73 (9.5 mg) was obtained as an orange solid. MS: calc'd 546 (MH⁺), measured 546 (MH⁺). ¹H NMR (METHANOL-d₄, 400 MHz) δ 8.99 (dd, 1H, J=1.7, 4.2 Hz), 8.65 (dd, 1H, J=1.7, 8.6 Hz), 8.16 (d, 1H, J=8.1 Hz), 7.65 (dd, 1H, J=4.3, 8.6 Hz), 7.28 (d, 1H, J=8.1 Hz), 6.49 (s, 1H), 5.3-5.5 (m, 1H), 4.4-4.5 (m, 1H), 4.0-4.2 (m, 4H), 3.6-3.9 (m, 4H), 3.3-3.5 (m, 8H), 3.23 (br d, 2H, J=5.6 Hz), 2.7-2.9 (m, 2H), 2.38 (s, 3H), 1.30 (d, 3H, J=6.2 Hz).

### Example 74

### 5-[(2R,6S)-2-methyl-6-[[4-[6-methyl-2-[cis-3-amino-5-fluoro-1-piperidyl]pyrimidin-4-yl]piperazin-1-yl]methyl]morpholin-4-yl]quinoline-8-carbonitrile

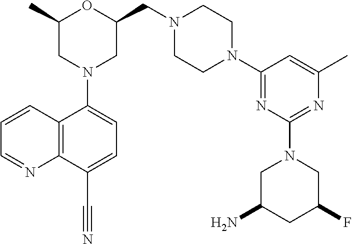 [SMILES: Cc1cc(N2CCN(C[C@H]3CN(c4ccc(C#N)c5ncccc45)C[C@@H](C)O3)CC2)nc(N2C[C@H](N)C[C@H](F)C2)n1]

The title compound was prepared in analogy to the preparation of Example 45 by using tert-butyl N-(cis-5-fluoropiperidin-3-yl)carbamate (CAS:1363378-08-8) and 2,4-dichloro-6-methylpyrimidine instead of 2,4-dichloro-5-fluoropyrimidine (compound 45d) and tert-butyl piperazine-1-carboxylate (compound 45a). Example 74 (21.5 mg) was obtained as a light yellow solid. MS: calc'd 560 (MH⁺), measured 560 (MH⁺). ¹H NMR (METHANOL-d₄, 400 MHz) δ 8.99 (dd, 1H, J=1.6, 4.3 Hz), 8.66 (dd, 1H, J=1.6, 8.6 Hz), 8.16 (d, 1H, J=7.9 Hz), 7.64 (dd, 1H, J=4.3, 8.6 Hz), 7.28 (d, 1H, J=8.1 Hz), 6.55 (s, 1H), 5.0-5.2 (m, 1H), 4.8-4.8 (m, 2H), 4.5-4.7 (m, 2H), 4.1-4.3 (m, 4H), 3.5-3.8 (m, 7H), 3.4-3.5 (m, 4H), 2.7-2.8 (m, 2H), 2.44 (s, 3H), 2.3-2.4 (m, 2H), 1.31 (d, 3H, J=6.4 Hz).

### Example 75

### 5-[(2R,6S)-2-methyl-6-[[4-[6-methyl-2-[trans-3-amino-5-fluoro-1-piperidyl]pyrimidin-4-yl]piperazin-1-yl]methyl]morpholin-4-yl]quinoline-8-carbonitrile

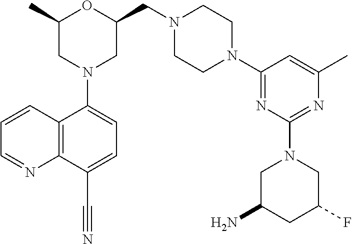 [SMILES: Cc1cc(N2CCN(C[C@H]3CN(c4ccc(C#N)c5ncccc45)C[C@@H](C)O3)CC2)nc(N2C[C@H](N)C[C@@H](F)C2)n1]

The title compound was prepared in analogy to the preparation of Example 45 by using tert-butyl N-(trans-5-fluoropiperidin-3-yl)carbamate and 2,4-dichloro-6-methylpyrimidine instead of 2,4-dichloro-5-fluoropyrimidine (compound 45d) and tert-butyl piperazine-1-carboxylate (compound 45a). Example 75 (9.8 mg) was obtained as a light yellow solid. MS: calc'd 560 (MH⁺), measured 560 (MH⁺). ¹H NMR (METHANOL-d₄, 400 MHz) δ 8.99 (dd, 1H, J=1.5, 4.2 Hz), 8.66 (dd, 1H, J=1.5, 8.6 Hz), 8.16 (d, 1H, J=8.1 Hz), 7.65 (dd, 1H, J=4.3, 8.6 Hz), 7.28 (d, 1H, J=8.1 Hz), 6.54 (s, 1H), 5.0-5.2 (m, 2H), 4.6-4.7 (m, 1H), 4.5-4.6 (m, 1H), 4.1-4.4 (m, 4H), 3.41 (s, 11H), 3.2-3.3 (m, 1H), 2.7-2.8 (m, 2H), 2.5-2.6 (m, 1H), 2.43 (s, 3H), 1.9-2.1 (m, 1H), 1.31 (d, 3H, J=6.2 Hz).

### Example 76

### 5-[(2S,6R)-2-[[4-[2-[[(3R,4R)-4-fluoropyrrolidin-3-yl]amino]-6-methyl-pyrimidin-4-yl]piperazin-1-yl]methyl]-6-methyl-morpholin-4-yl]quinoline-8-carbonitrile

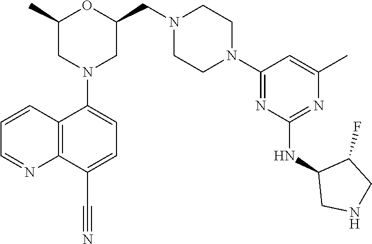 [SMILES: Cc1cc(N2CCN(C[C@H]3CN(c4ccc(C#N)c5ncccc45)C[C@@H](C)O3)CC2)nc(N[C@@H]2CNC[C@H]2F)n1]

The title compound was prepared in analogy to the preparation of Example 45 by using tert-butyl (3R,4R)-3-amino-4-fluoropyrrolidine-1-carboxylate (CAS: 1441392-27-3) and 2,4-dichloro-6-methylpyrimidine instead of 2,4-dichloro-5-fluoropyrimidine (compound 45d) and tert-butyl piperazine-1-carboxylate (compound 45a). Example 76 (14.6 mg) was obtained as a light yellow solid. MS: calc'd 546 (MH⁺, measured 546 (MH⁺). ¹H NMR (METHANOL-d₄, 400 MHz) δ 8.99 (dd, 1H, J=1.7, 4.2 Hz), 8.65 (dd, 1H, J=1.7, 8.6 Hz), 8.15 (d, 1H, J=7.9 Hz), 7.64 (dd, 1H, J=4.3, 8.6 Hz), 7.27 (d, 1H, J=8.1 Hz), 6.53 (s, 1H), 5.3-5.5 (m, 1H), 4.92 (br s, 1H), 4.5-4.6 (m, 1H), 4.0-4.4 (m, 4H), 3.9-3.9 (m, 1H), 3.5-3.8 (m, 7H), 3.4-3.5 (m, 4H), 3.2-3.3 (m, 1H), 2.7-2.9 (m, 2H), 2.40 (s, 3H), 1.3-1.3 (m, 3H).

### Example 77

### 5-[(2S,6R)-2-[[4-[2-[(3S,5R)-3-amino-5-fluoro-1-piperidyl]-6-methyl-pyrimidin-4-yl]piperazin-1-yl]methyl]-6-methyl-morpholin-4-yl]quinoline-8-carbonitrile

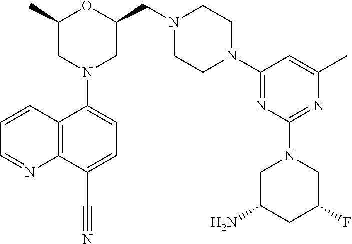 [SMILES: Cc1cc(N2CCN(C[C@H]3CN(c4ccc(C#N)c5ncccc45)C[C@@H](C)O3)CC2)nc(N2C[C@@H](N)C[C@@H](F)C2)n1]

The title compound was prepared in analogy to the preparation of Example 45 by using tert-butyl N-[(3S,5R)-5-fluoropiperidin-3-yl]carbamate (PharmaBlock, PBZS8178, CAS:1405128-38-2) and 2,4-dichloro-6-methylpyrimidine instead of 2,4-dichloro-5-fluoropyrimidine (compound 45d) and tert-butyl piperazine-1-carboxylate (compound 45a). Example 77 (12.8 mg) was obtained as a light yellow solid. MS: calc'd 560 (MH⁺), measured 560 (MH⁺). ¹H NMR (METHANOL-d₄, 400 MHz) δ 8.99 (dd, 1H, J=1.7, 4.2 Hz), 8.66 (dd, 1H, J=1.2, 8.6 Hz), 8.16 (d, 1H, J=8.1 Hz), 7.65 (dd, 1H, J=4.3, 8.7 Hz), 7.28 (d, 1H, J=7.9 Hz), 6.57 (d, 1H, J=19.6 Hz), 5.0-5.2 (m, 1H), 4.9-4.9 (m, 1H), 4.7-4.8 (m, 1H), 4.5-4.6 (m, 2H), 4.1-4.4 (m, 4H), 3.5-3.8 (m, 7H), 3.4-3.5 (m, 4H), 2.7-2.8 (m, 2H), 2.4-2.4 (m, 3H), 2.3-2.4 (m, 2H), 1.31 (d, 3H, J=6.2 Hz).

### Example 78

### 5-[(2S,6R)-2-[[4-[2-[(3S,4R)-4-amino-3-fluoro-1-piperidyl]-6-methyl-pyrimidin-4-yl]piperazin-1-yl]methyl]-6-methyl-morpholin-4-yl]quinoline-8-carbonitrile

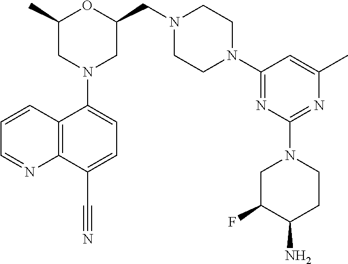 [SMILES: Cc1cc(N2CCN(C[C@H]3CN(c4ccc(C#N)c5ncccc45)C[C@@H](C)O3)CC2)nc(N2CC[C@@H](N)[C@@H](F)C2)n1]

The title compound was prepared in analogy to the preparation of Example 45 by using tert-butyl N-[(3S,4R)-3-fluoropiperidin-4-yl]carbamate and 2,4-dichloro-6-methylpyrimidine instead of 2,4-dichloro-5-fluoropyrimidine (compound 45d) and tert-butyl piperazine-1-carboxylate (compound 45a). Example 78 (17.4 mg) was obtained as a light yellow solid. MS: calc'd 560 (MH⁺), measured 560 (MH⁺). ¹H NMR (METHANOL-d₄, 400 MHz) δ 8.99 (dd, 1H, J=1.5, 4.2 Hz), 8.66 (dd, 1H, J=1.6, 8.6 Hz), 8.16 (d, 1H, J=7.9 Hz), 7.65 (dd, 1H, J=4.3, 8.7 Hz), 7.28 (d, 1H, J=8.1 Hz), 6.52 (s, 1H), 4.9-5.0 (m, 1H), 4.7-4.8 (m, 1H), 4.5-4.7 (m, 3H), 4.1-4.3 (m, 4H), 3.5-3.7 (m, 5H), 3.42 (br d, 4H, J=11.9 Hz), 3.2-3.3 (m, 2H), 2.7-2.8 (m, 2H), 2.41 (s, 3H), 2.2-2.3 (m, 1H), 1.82 (br s, 1H), 1.31 (d, 3H, J=6.2 Hz).

### Example 79

### 5-[(2S,6R)-2-[[4-[2-[(3R,4R)-3-amino-4-fluoro-pyrrolidin-1-yl]-6-methyl-pyrimidin-4-yl]piperazin-1-yl]methyl]-6-methyl-morpholin-4-yl]quinoline-8-carbonitrile

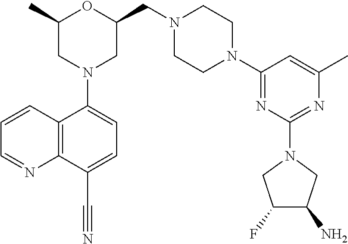 [SMILES: Cc1cc(N2CCN(C[C@H]3CN(c4ccc(C#N)c5ncccc45)C[C@@H](C)O3)CC2)nc(N2C[C@@H](N)[C@H](F)C2)n1]

The title compound was prepared in analogy to the preparation of Example 45 by using tert-butyl N-[(3R,4R)-4-fluoropyrrolidin-3-yl)]carbamate hydrochloride (CAS: 2097061-04-4) and 2,4-dichloro-6-methylpyrimidine instead of 2,4-dichloro-5-fluoropyrimidine (compound 45d) and tert-butyl piperazine-1-carboxylate (compound 45a). Example 79 (39.1 mg) was obtained as a light yellow solid. MS: calc'd 546 (MH⁺, measured 546 (MH⁺). ¹H NMR (METHANOL-d₄, 400 MHz) δ 9.01 (dd, 1H, J=1.6, 4.3 Hz), 8.68 (dd, 1H, J=1.6, 8.6 Hz), 8.18 (d, 1H, J=8.1 Hz), 7.67 (dd, 1H, J=4.3, 8.6 Hz), 7.30 (d, 1H, J=8.1 Hz), 6.57 (s, 1H), 5.4-5.6 (m, 1H), 4.5-4.6 (m, 1H), 4.0-4.3 (m, 10H), 3.5-3.6 (m, 4H), 3.4-3.5 (m, 4H), 2.7-2.9 (m, 2H), 2.45 (s, 3H), 1.33 (d, 3H, J=6.2 Hz).

### Example 80

### 5-[(2S,6R)-2-[[4-[2-(5-amino-3,3-difluoro-1-piperidyl)-6-methyl-pyrimidin-4-yl]piperazin-1-yl]methyl]-6-methyl-morpholin-4-yl]quinoline-8-carbonitrile

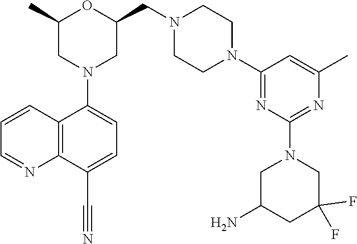 [SMILES: Cc1cc(N2CCN(C[C@H]3CN(c4ccc(C#N)c5ncccc45)C[C@@H](C)O3)CC2)nc(N2CC(N)CC(F)(F)C2)n1]

The title compound was prepared in analogy to the preparation of Example 45 by using tert-butyl N-(5,5-difluoropiperidin-3-yl)carbamate and 2,4-dichloro-6-methylpyrimidine instead of 2,4-dichloro-5-fluoropyrimidine (compound 45d) and tert-butyl piperazine-1-carboxylate (compound 45a). Example 80 (14.3 mg) was obtained as a light yellow solid. MS: calc'd 578 (MH⁺), measured 578 (MH⁺). ¹H NMR (METHANOL-d₄, 400 MHz) δ 9.00 (dd, 1H, J=1.7, 4.2 Hz), 8.66 (dd, 1H, J=1.6, 8.6 Hz), 8.17 (d, 1H, J=8.1 Hz), 7.65 (dd, 1H, J=4.2, 8.6 Hz), 7.29 (d, 1H, J=8.1 Hz), 6.39 (s, 1H), 4.4-4.6 (m, 3H), 4.3-4.4 (m, 1H), 4.1-4.2 (m, 2H), 4.0-4.1 (m, 3H), 3.88 (br dd, 1H, J=6.2, 7.9 Hz), 3.66 (br d, 1H, J=2.7 Hz), 3.39 (br d, 8H, J=7.2 Hz), 2.7-2.8 (m, 2H), 2.5-2.7 (m, 1H), 2.3-2.4 (m, 4H), 1.32 (d, 3H, J=6.2 Hz).

### Example 81

### 5-[(2S,6R)-2-[[4-[2-[3-(1-hydroxy-1-methyl-ethyl)piperazin-1-yl]-6-methyl-pyrimidin-4-yl]piperazin-1-yl]methyl]-6-methyl-morpholin-4-yl]quinoline-8-carbonitrile

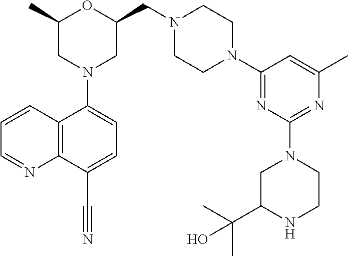 [SMILES: Cc1cc(N2CCN(C[C@H]3CN(c4ccc(C#N)c5ncccc45)C[C@@H](C)O3)CC2)nc(N2CCNC(C(C)(C)O)C2)n1]

The title compound was prepared in analogy to the preparation of Example 45 by using 2-(piperazin-2-yl)propan-2-ol and 2,4-dichloro-6-methylpyrimidine instead of 2,4-dichloro-5-fluoropyrimidine (compound 45d) and tert-butyl piperazine-1-carboxylate (compound 45a). Example 81 (15.2 mg) was obtained as a light yellow solid. MS: calc'd 586 (MH⁺), measured 586 (MH⁺). ¹H NMR (METHANOL-d₄, 400 MHz) δ 8.99 (dd, 1H, J=1.7, 4.2 Hz), 8.65 (dd, 1H, J=1.7, 8.6 Hz), 8.15 (d, 1H, J=7.9 Hz), 7.64 (dd, 1H, J=4.3, 8.6 Hz), 7.27 (d, 1H, J=8.1 Hz), 6.53 (s, 1H), 5.3-5.5 (m, 1H), 4.92 (br s, 1H), 4.5-4.6 (m, 1H), 4.0-4.4 (m, 4H), 3.9-3.9 (m, 1H), 3.5-3.8 (m, 7H), 3.4-3.5 (m, 4H), 3.2-3.3 (m, 1H), 2.7-2.9 (m, 2H), 2.40 (s, 3H), 1.3-1.3 (m, 3H).

### Example 82

### 5-[(2S,6R)-2-[[4-[2-[(3R,4R)-4-amino-3-fluoro-1-piperidyl]-6-methyl-pyrimidin-4-yl]piperazin-1-yl]methyl]-6-methyl-morpholin-4-yl]quinoline-8-carbonitrile

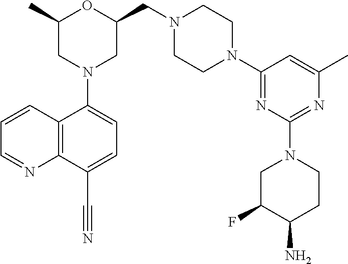 [SMILES: Cc1cc(N2CCN(C[C@H]3CN(c4ccc(C#N)c5ncccc45)C[C@@H](C)O3)CC2)nc(N2CC[C@@H](N)[C@@H](F)C2)n1]

The title compound was prepared in analogy to the preparation of Example 45 by using tert-butyl N-[(3R,4R)-3-fluoropiperidin-4-yl]carbamate (PharmaBlock, PB08067, CAS: 1523530-29-1) and 2,4-dichloro-6-methylpyrimidine instead of 2,4-dichloro-5-fluoropyrimidine (compound 45d) and tert-butyl piperazine-1-carboxylate (compound 45a). Example 82 (9.5 mg) was obtained as a light yellow solid. MS: calc'd 560 (MH⁺), measured 560 (MH⁺). ¹H NMR (METHANOL-d₄, 400 MHz) δ 9.0-9.0 (m, 1H), 8.68 (dd, 1H, J=1.2, 8.6 Hz), 8.18 (d, 1H, J=8.1 Hz), 7.67 (dd, 1H, J=4.2, 8.5 Hz), 7.30 (d, 1H, J=8.1 Hz), 6.54 (s, 1H), 5.0-5.2 (m, 3H), 4.7-4.8 (m, 2H), 4.5-4.6 (m, 1H), 4.19 (br d, 4H, J=6.6 Hz), 3.7-3.8 (m, 1H), 3.5-3.6 (m, 4H), 3.4-3.5 (m, 5H), 2.7-2.9 (m, 2H), 2.44 (s, 3H), 2.0-2.1 (m, 2H), 1.33 (d, 3H, J=6.2 Hz).

### Example 83

### 5-[(2R,6S)-2-methyl-6-[[4-[6-methyl-2-[(3S,4R)-3-amino-4-fluoro-1-piperidyl]-pyrimidin-4-yl]piperazin-1-yl]methyl]morpholin-4-yl]quinoline-8-carbonitrile

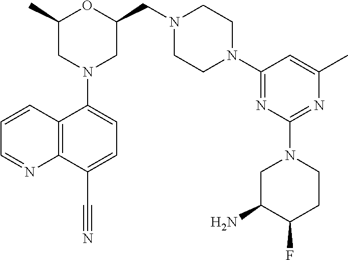 [SMILES: Cc1cc(N2CCN(C[C@H]3CN(c4ccc(C#N)c5ncccc45)C[C@@H](C)O3)CC2)nc(N2CC[C@@H](F)[C@@H](N)C2)n1]

The title compound was prepared in analogy to the preparation of Example 45 by using tert-butyl N-[cis-4-fluoropiperidin-3-yl]carbamate and 2,4-dichloro-6-methylpyrimidine instead of 2,4-dichloro-5-fluoropyrimidine (compound 45d) and tert-butyl piperazine-1-carboxylate (compound 45a). Example 83 (7.2 mg) was obtained as a light yellow solid. MS: calc'd 560 (MH⁺), measured 560 (MH⁺). ¹H NMR (METHANOL-d₄, 400 MHz) δ 9.01 (dd, 1H, J=1.7, 4.2 Hz), 8.68 (dd, 1H, J=1.5, 8.6 Hz), 8.18 (d, 1H, J=8.1 Hz), 7.67 (dd, 1H, J=4.3, 8.6 Hz), 7.30 (d, 1H, J=7.9 Hz), 6.53 (s, 1H), 5.1-5.3 (m, 1H), 4.72 (br d, 1H, J=9.4 Hz), 4.5-4.6 (m, 1H), 4.0-4.4 (m, 6H), 3.5-3.8 (m, 7H), 3.4-3.5 (m, 4H), 2.7-2.9 (m, 2H), 2.4-2.5 (m, 3H), 2.2-2.3 (m, 1H), 2.0-2.2 (m, 1H), 1.34 (d, 3H, J=6.2 Hz).

### Example 84

### 5-[(2R,6S)-2-methyl-6-[[4-[6-methyl-2-[(3R,4R)-3-amino-4-fluoro-1-piperidyl]pyrimidin-4-yl]piperazin-1-yl]methyl]morpholin-4-yl]quinoline-8-carbonitrile

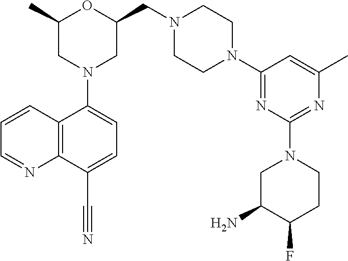 [SMILES: Cc1cc(N2CCN(C[C@H]3CN(c4ccc(C#N)c5ncccc45)C[C@@H](C)O3)CC2)nc(N2CC[C@@H](F)[C@@H](N)C2)n1]

The title compound was prepared in analogy to the preparation of Example 45 by using tert-butyl N-[trans-4-fluoropiperidin-3-yl]carbamate (CAS: 1052713-46-8) and 2,4-dichloro-6-methylpyrimidine instead of 2,4-dichloro-5-fluoropyrimidine (compound 45d) and tert-butyl piperazine-1-carboxylate (compound 45a). Example 84 (21.8 mg) was obtained as a light yellow solid. MS: calc'd 560 (MH⁺), measured 560 (MH⁺). ¹H NMR (METHANOL-d₄, 400 MHz) δ 9.01 (dd, 1H, J=1.6, 4.3 Hz), 8.68 (dd, 1H, J=1.5, 8.6 Hz), 8.18 (d, 1H, J=8.1 Hz), 7.67 (dd, 1H, J=4.3, 8.6 Hz), 7.30 (d, 1H, J=8.1 Hz), 6.53 (s, 1H), 4.9-5.0 (m, 1H), 4.7-4.8 (m, 1H), 4.5-4.6 (m, 1H), 4.4-4.5 (m, 1H), 3.9-4.3 (m, 5H), 3.5-3.7 (m, 5H), 3.4-3.5 (m, 6H), 2.7-2.9 (m, 2H), 2.44 (s, 3H), 2.3-2.4 (m, 1H), 1.8-2.0 (m, 1H), 1.34 (d, 3H, J=6.2 Hz).

### Example 85

### 5-[(2S,6R)-2-[[4-[2-(azetidin-3-ylamino)-6-methyl-pyrimidin-4-yl]piperazin-1-yl]methyl]-6-methyl-morpholin-4-yl]quinoline-8-carbonitrile

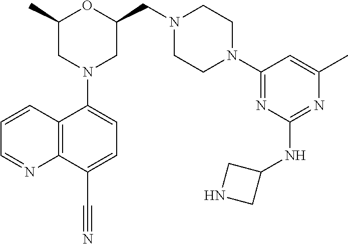 [SMILES: Cc1cc(N2CCN(C[C@H]3CN(c4ccc(C#N)c5ncccc45)C[C@@H](C)O3)CC2)nc(NC2CNC2)n1]

The title compound was prepared in analogy to the preparation of Example 45 by using 1-BOC-3-(amino)azetidine and 2,4-dichloro-6-methylpyrimidine instead of 2,4-dichloro-5-fluoropyrimidine (compound 45d) and tert-butyl piperazine-1-carboxylate (compound 45a). Example 85 (19.2 mg) was obtained as a light yellow solid. MS: calc'd 514 (MH⁺), measured 514 (MH⁺). ¹H NMR (METHANOL-d₄, 400 MHz) δ 9.01 (dd, 1H, J=1.6, 4.2 Hz), 8.67 (dd, 1H, J=1.6, 8.6 Hz), 8.17 (d, 1H, J=7.9 Hz), 7.66 (dd, 1H, J=4.3, 8.6 Hz), 7.29 (d, 1H, J=8.1 Hz), 6.52 (s, 1H), 5.08 (quin, 1H, J=7.6 Hz), 4.5-4.6 (m, 1H), 4.4-4.5 (m, 2H), 3.8-4.4 (m, 7H), 3.60 (br s, 4H), 3.4-3.5 (m, 4H), 2.7-2.8 (m, 2H), 2.41 (s, 3H), 1.33 (d, 3H, J=6.2 Hz).

### Example 86

### 5-[(2S,6R)-2-[[4-[2-(4-amino-3,3-difluoro-1-piperidyl)-6-methyl-pyrimidin-4-yl]piperazin-1-yl]methyl]-6-methyl-morpholin-4-yl]quinoline-8-carbonitrile

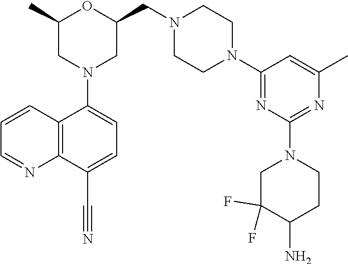 [SMILES: Cc1cc(N2CCN(C[C@H]3CN(c4ccc(C#N)c5ncccc45)C[C@@H](C)O3)CC2)nc(N2CCC(N)C(F)(F)C2)n1]

The title compound was prepared in analogy to the preparation of Example 45 by using 4-(Boc-amino)-3,3-difluoropiperidine (CAS: 1263180-22-8) and 2,4-dichloro-6-methylpyrimidine instead of 2,4-dichloro-5-fluoropyrimidine (compound 45d) and tert-butyl piperazine-1-carboxylate (compound 45a). Example 86 (8.3 mg) was obtained as a light yellow solid. MS: calc'd 578 (MH⁺), measured 578 (MH⁺). ¹H NMR (METHANOL-d₄, 400 MHz) δ9.01 (br d, 1H, J=3.2 Hz), 8.6-8.7 (m, 1H), 8.19 (d, 1H, J=8.1 Hz), 7.67 (dd, 1H, J=4.1, 8.5 Hz), 7.30 (d, 1H, J=8.1 Hz), 6.51 (s, 1H), 5.1-5.2 (m, 1H), 4.7-4.8 (m, 1H), 4.53 (br s, 1H), 4.0-4.3 (m, 6H), 3.5-3.7 (m, 5H), 3.4-3.5 (m, 5H), 2.78 (td, 2H, J=11.1, 19.1 Hz), 2.42 (s, 3H), 2.3-2.3 (m, 1H), 1.9-2.0 (m, 1H), 1.33 (d, 3H, J=6.1 Hz).

### Example 87

### 5-[(2S,6R)-2-[[4-[2-(3-aminoazetidin-1-yl)-6-methyl-pyrimidin-4-yl]piperazin-1-yl]methyl]-6-methyl-morpholin-4-yl]quinoline-8-carbonitrile

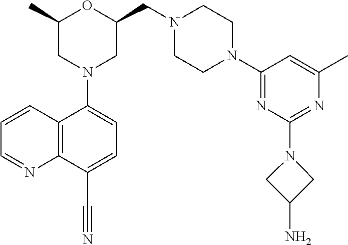 [SMILES: Cc1cc(N2CCN(C[C@H]3CN(c4ccc(C#N)c5ncccc45)C[C@@H](C)O3)CC2)nc(N2CC(N)C2)n1]

The title compound was prepared in analogy to the preparation of Example 45 by using 3-N-boc-amino-azetidine and 2,4-dichloro-6-methylpyrimidine instead of 2,4-dichloro-5-fluoropyrimidine (compound 45d) and tert-butyl piperazine-1-carboxylate (compound 45a). Example 87 (31 mg) was obtained as a light yellow solid. MS: calc'd 514 (MH⁺), measured 514 (MH⁺). ¹H NMR (METHANOL-d₄, 400 MHz) δ 9.00 (dd, 1H, J=1.3, 4.2 Hz), 8.67 (dd, 1H, J=1.2, 8.6 Hz), 8.17 (d, 1H, J=8.1 Hz), 7.66 (dd, 1H, J=4.3, 8.6 Hz), 7.29 (d, 1H, J=8.1 Hz), 6.53 (s, 1H), 4.64 (br dd, 2H, J=7.2, 10.0 Hz), 4.54 (br t, 1H, J=9.2 Hz), 4.34 (s, 4H), 4.0-4.2 (m, 4H), 3.5-3.7 (m, 4H), 3.4-3.5 (m, 4H), 2.7-2.9 (m, 2H), 2.41 (s, 3H), 1.33 (d, 3H, J=6.2 Hz).

### Example 88

### 5-[(2S,6R)-2-[[4-[2-[(3R,4S)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile

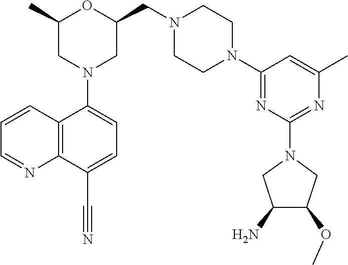 [SMILES: CO[C@@H]1CN(c2nc(C)cc(N3CCN(C[C@H]4CN(c5ccc(C#N)c6ncccc56)C[C@@H](C)O4)CC3)n2)C[C@@H]1N]

The title compound was prepared in analogy to the preparation of Example 45 by using tert-butyl N-[(3R,4S)-4-methoxypyrrolidin-3-yl]carbamate (CAS: 1932508-77-4) and 2,4-dichloro-6-methylpyrimidine instead of 2,4-dichloro-5-fluoropyrimidine (compound 45d) and tert-butyl piperazine-1-carboxylate (compound 45a). Example 88 (106.9 mg) was obtained as a light yellow solid. MS: calc'd 558 (MH⁺), measured 558 (MH⁺). ¹H NMR (METHANOL-d₄, 400 MHz) δ 9.01 (dd, 1H, J=1.5, 4.2 Hz), 8.67 (dd, 1H, J=1.4, 8.6 Hz), 8.18 (d, 1H, J=8.1 Hz), 7.67 (dd, 1H, J=4.3, 8.6 Hz), 7.30 (d, 1H, J=8.1 Hz), 6.55 (s, 1H), 4.5-4.6 (m, 1H), 4.13 (br s, 8H), 3.9-4.0 (m, 1H), 3.7-3.9 (m, 2H), 3.6-3.7 (m, 4H), 3.53 (s, 3H), 3.43 (br s, 4H), 2.7-2.9 (m, 2H), 2.45 (s, 3H), 1.33 (d, 3H, J=6.2 Hz).

### Example 89

### 5-[(2R,6S)-2-methyl-6-[[4-(2-morpholin-2-ylphenyl)piperazin-1-yl]methyl]morpholin-4-yl]quinoline-8-carbonitrile

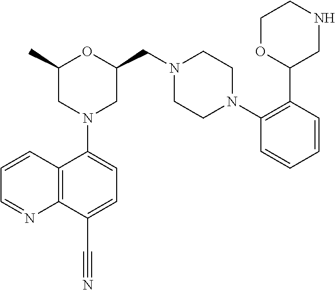 [SMILES: C[C@@H]1CN(c2ccc(C#N)c3ncccc23)C[C@H](CN2CCN(c3ccccc3C3CNCCO3)CC2)O1]

The title compound was prepared in analogy to the preparation of Example 62 by using 2-bromo-1-(2-bromophenyl)ethanone instead of 2-bromo-1-(3-bromophenyl)ethanone (compound 62a). Example 89 (14 mg) was obtained as a yellow solid. MS: calc'd 513 (MH⁺), measured 513 (MH⁺). ¹H NMR (METHANOL-d₄, 400 MHz) δ 9.1-9.2 (m, 2H), 8.37 (d, 1H, J=8.1 Hz), 7.98 (ddd, 1H, J=2.1, 5.0, 8.3 Hz), 7.57 (dd, 1H, J=1.2, 7.7 Hz), 7.47 (d, 1H, J=8.2 Hz), 7.4-7.4 (m, 1H), 7.4-7.4 (m, 1H), 7.3-7.3 (m, 1H), 5.4-5.5 (m, 1H), 4.6-4.7 (m, 1H), 4.2-4.3 (m, 3H), 3.9-4.0 (m, 1H), 3.7-3.9 (m, 4H), 3.3-3.6 (m, 9H), 3.2-3.3 (m, 1H), 3.1-3.2 (m, 1H), 2.8-3.0 (m, 2H), 1.36 (dd, 3H, J=1.5, 6.2 Hz).

### Example 90

### 5-[(2S,6R)-2-[[4-[2-[(3S,4S)-4-amino-3-methoxy-1-piperidyl]-6-methyl-pyrimidin-4-yl]piperazin-1-yl]methyl]-6-methyl-morpholin-4-yl]quinoline-8-carbonitrile

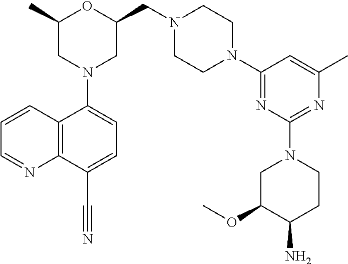 [SMILES: CO[C@H]1CN(c2nc(C)cc(N3CCN(C[C@H]4CN(c5ccc(C#N)c6ncccc56)C[C@@H](C)O4)CC3)n2)CC[C@H]1N]

The title compound was prepared in analogy to the preparation of Example 45 by using tert-butyl N-[(3R,4R)-3-methoxypiperidin-4-yl]carbamate hemioxalate (PharmaBlock, PB07428-1, CAS:907544-19-8) and 2,4-dichloro-6-methylpyrimidine instead of 2,4-dichloro-5-fluoropyrimidine (compound 45d) and tert-butyl piperazine-1-carboxylate (compound 45a). Example 90 (7 mg) was obtained as a light yellow solid. MS: calc'd 572 (MH⁺), measured 572 (MH⁺). ¹H NMR (METHANOL-d₄, 400 MHz) δ 8.90 (dd, 1H, J=1.7, 4.2 Hz), 8.56 (dd, 1H, J=1.7, 8.6 Hz), 8.07 (d, 1H, J=8.1 Hz), 7.55 (dd, 1H, J=4.3, 8.6 Hz), 7.18 (d, 1H, J=8.1 Hz), 6.39 (s, 1H), 4.8-4.9 (m, 1H), 4.8-4.8 (m, 1H), 4.5-4.6 (m, 1H), 4.3-4.4 (m, 1H), 3.9-4.2 (m, 4H), 3.46 (s, 3H), 3.3-3.4 (m, 6H), 3.2-3.3 (m, 3H), 3.1-3.2 (m, 1H), 3.0-3.1 (m, 1H), 2.7-2.9 (m, 1H), 2.6-2.7 (m, 2H), 2.31 (s, 3H), 2.0-2.1 (m, 1H), 1.6-1.7 (m, 1H), 1.21 (d, 3H, J=6.2 Hz).

### Example 91

### 5-[(2S,6R)-2-[[4-(6-methoxy-2-piperazin-1-yl-pyrimidin-4-yl)piperazin-1-yl]methyl]-6-methyl-morpholin-4-yl]quinoline-8-carbonitrile

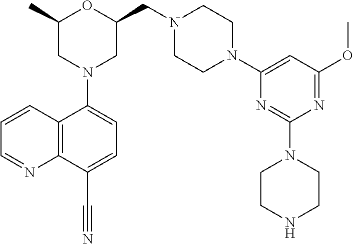 [SMILES: COc1cc(N2CCN(C[C@H]3CN(c4ccc(C#N)c5ncccc45)C[C@@H](C)O3)CC2)nc(N2CCNCC2)n1]

The title compound was prepared in analogy to the preparation of Example 45 by using 2,4-dichloro-6-methoxy-pyrimidine instead of 2,4-dichloro-5-fluoropyrimidine (compound 45d). Example 91 (58 mg) was obtained as a light yellow solid. MS: calc'd 544 (MH⁺), measured 544 (MH⁺). ¹H NMR (METHANOL-d₄, 400 MHz) δ 9.00 (dd, 1H, J=1.6, 4.3 Hz), 8.67 (dd, 1H, J=1.7, 8.6 Hz), 8.17 (d, 1H, J=7.9 Hz), 7.66 (dd, 1H, J=4.3, 8.6 Hz), 7.29 (d, 1H, J=8.1 Hz), 4.3-4.8 (m, 3H), 4.1-4.2 (m, 1H), 3.9-4.1 (m, 5H), 3.89 (s, 3H), 3.5-3.8 (m, 3H), 3.4-3.5 (m, 5H), 3.3-3.4 (m, 2H), 3.1-3.3 (m, 4H), 2.77 (ddd, 2H, J=10.3, 12.0, 18.6 Hz), 1.33 (d, 3H, J=6.2 Hz).

### Example 92

### 5-[(2S,6R)-2-[[4-[2-[(3S,4R)-3-amino-4-methoxy-pyrrolidin-1-yl]pyrimidin-4-yl]piperazin-1-yl]methyl]-6-methyl-morpholin-4-yl]quinoline-8-carbonitrile

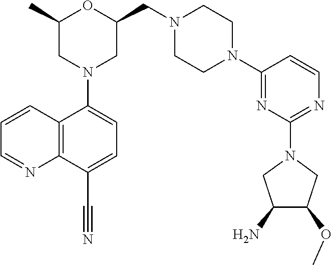 [SMILES: CO[C@@H]1CN(c2nccc(N3CCN(C[C@H]4CN(c5ccc(C#N)c6ncccc56)C[C@@H](C)O4)CC3)n2)C[C@@H]1N]

The title compound was prepared in analogy to the preparation of Example 23 by using tert-butyl N-[(3S,4R)-4-methoxypyrrolidin-3-yl]carbamate instead of tert-butyl azetidin-3-ylcarbamate (compound 23c). Example 92 (13 mg) was obtained as a light yellow solid. MS: calc'd 544 (MH⁺), measured 544 (MH⁺). ¹H NMR (METHANOL-d₄, 400 MHz) δ 9.02 (dd, 1H, J=1.6, 4.3 Hz), 8.68 (dd, 1H, J=1.7, 8.6 Hz), 8.19 (d, 1H, J=8.1 Hz), 7.91 (d, 1H, J=7.5 Hz), 7.67 (dd, 1H, J=4.3, 8.6 Hz), 7.31 (d, 1H, J=8.1 Hz), 6.63 (d, 1H, J=7.5 Hz), 4.4-4.5 (m, 1H), 3.9-4.4 (m, 9H), 3.8-3.8 (m, 1H), 3.6-3.8 (m, 1H), 3.53 (s, 3H), 3.4-3.5 (m, 6H), 3.37 (br s, 2H), 2.7-2.8 (m, 2H), 1.33 (d, 3H, J=6.4 Hz).

### Example 93

### 5-[(2S,6R)-2-[[4-[2-[(3R,4S)-3-amino-4-methoxy-pyrrolidin-1-yl]pyrimidin-4-yl]piperazin-1-yl]methyl]-6-methyl-morpholin-4-yl]quinoline-8-carbonitrile

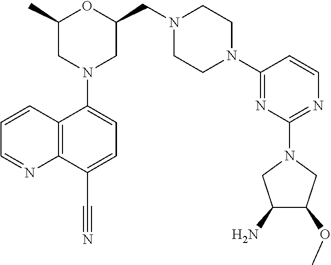 [SMILES: CO[C@@H]1CN(c2nccc(N3CCN(C[C@H]4CN(c5ccc(C#N)c6ncccc56)C[C@@H](C)O4)CC3)n2)C[C@@H]1N]

The title compound was prepared in analogy to the preparation of Example 23 by using tert-butyl N-[(3R,4S)-4-methoxypyrrolidin-3-yl]carbamate instead of tert-butyl azetidin-3-ylcarbamate (compound 23c). Example 93 (10 mg) was obtained as a light yellow solid. MS: calc'd 544 (MH⁺), measured 544 (MH⁺). ¹H NMR (METHANOL-d₄, 400 MHz) δ 9.01 (dd, 1H, J=1.6, 4.3 Hz), 8.68 (dd, 1H, J=1.7, 8.6 Hz), 8.18 (d, 1H, J=8.1 Hz), 7.91 (d, 1H, J=7.5 Hz), 7.67 (dd, 1H, J=4.2, 8.6 Hz), 7.30 (d, 1H, J=8.1 Hz), 6.67 (d, 1H, J=7.6 Hz), 4.5-4.6 (m, 1H), 3.9-4.5 (m, 9H), 3.8-3.9 (m, 2H), 3.5-3.7 (m, 4H), 3.5-3.5 (m, 3H), 3.4-3.5 (m, 4H), 2.7-2.8 (m, 2H), 1.33 (d, 3H, J=6.2 Hz).

### Example 94

### 5-[(2S,6R)-2-[[4-(5-fluoro-4-piperazin-1-yl-pyrimidin-2-yl)piperazin-1-yl]methyl]-6-methyl-morpholin-4-yl]quinoline-8-carbonitrile

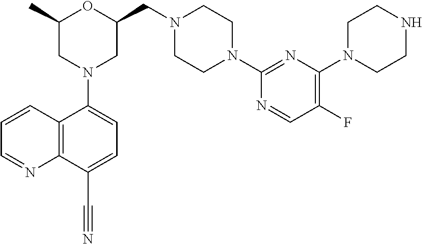 [SMILES: C[C@@H]1CN(c2ccc(C#N)c3ncccc23)C[C@H](CN2CCN(c3ncc(F)c(N4CCNCC4)n3)CC2)O1]

The title compound was prepared according to the following scheme:

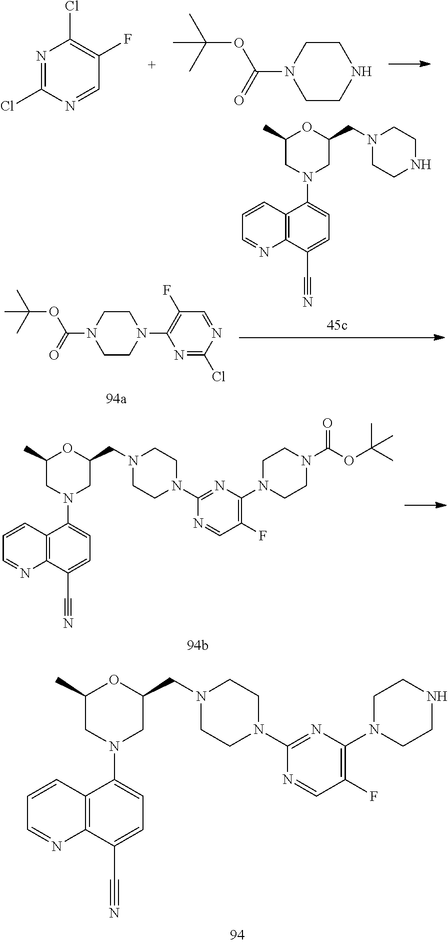

### Step 1: Preparation of tert-butyl 4-(2-chloro-5-fluoropyrimidin-4-yl)piperazine-1-carboxylate (Compound 94a)

A solution of 2,4-dichloro-5-fluoropyrimidine (CAS: 2927-71-1, 334 mg, 2 mmol) and tert-butyl piperazine-1-carboxylate (373 mg, 2 mmol) in ethanol (6 mL) was stirred at room temperature for 12 hrs. Then the mixture was concentrated and purified by silica gel chromatography to afford compound 94a (500 mg) as a white solid. MS: calc'd 317 (MH⁺), measured 317 (MH⁺).

### Step 2: Preparation of tert-butyl 4-(2-(4-(((2S,6R)-4-(8-cyanoquinolin-5-yl)-6-methylmorpholin-2-yl)methyl)piperazin-1-yl)-5-fluoropyrimidin-4-yl)piperazine-1-carboxylate (Compound 94b)

A suspension of 5-((2R,6S)-2-methyl-6-(piperazin-1-ylmethyl)morpholino)quinoline-8-carbonitrile (compound 45c, 80 mg, 228 μmol), tert-butyl 4-(2-chloro-5-fluoropyrimidin-4-yl)piperazine-1-carboxylate (compound 94a, 72 mg, 228 μmol), Ruphos Pd G2 (17.7 mg, 22.8 μmol) and K₂CO₃ (62 mg, 455 μmol) in 1,4-Dioxane (4 ml) was heated at 100° C. for 12 hrs under nitrogen atmosphere. The mixture was then cooled to room temperature and diluted with water (5 mL) and extracted with EtOAC (10 mL) three times. The combined organic layer was washed with brine and dried over Na₂SO₄. The solution was concentrated and the residue was purified by silica gel chromatography (EA/PE from 20% to 80%) to afford compound 94b (35 mg) as a pale yellow solid. MS: calc'd 632 (MH⁺), measured 632 (MH⁺).

### Step 3: Preparation of 5-[(2S,6R)-2-[[4-(5-fluoro-4-piperazin-1-yl-pyrimidin-2-yl)piperazin-1-yl]methyl]-6-methyl-morpholin-4-yl]quinoline-8-carbonitrile (Example 94)

To a solution of tert-butyl 4-(2-(4-(((2S,6R)-4-(8-cyanoquinolin-5-yl)-6-methylmorpholin-2-yl)methyl)piperazin-1-yl)-5-fluoropyrimidin-4-yl)piperazine-1-carboxylate (compound 94b, 35 mg, 55.4 μmol) in DCM (2 ml) was added 2,2,2-trifluoroacetic acid (740 mg, 0.5 ml, 6.49 mmol) at 0° C. The reaction mixture was stirred at r.t. for 2 hrs. Then the mixture was concentrated and purified by prep-HPLC to get 5-[(2S,6R)-2-[[4-(5-fluoro-4-piperazin-1-yl-pyrimidin-2-yl)piperazin-1-yl]methyl]-6-methyl-morpholin-4-yl]quinoline-8-carbonitrile (Example 94, 10 mg) as an orange solid. MS calc'd 532 (MH⁺), measured 532 (MH⁺). ¹H NMR (METHANOL-d₄, 400 MHz) δ 9.01 (dd, 1H, J=1.7, 4.2 Hz), 8.68 (dd, 1H, J=1.6, 8.6 Hz), 8.18 (d, 1H, J=8.1 Hz), 8.02 (d, 1H, J=6.2 Hz), 7.67 (dd, 1H, J=4.2, 8.6 Hz), 7.30 (d, 1H, J=8.1 Hz), 4.53 (dtd, 1H, J=2.7, 5.0, 10.0 Hz), 4.2-4.3 (m, 1H), 4.0-4.1 (m, 4H), 3.4-3.9 (m, 9H), 3.3-3.4 (m, 5H), 3.2-3.3 (m, 1H), 2.78 (ddd, 2H, J=10.3, 12.1, 18.9 Hz), 1.34 (d, 3H, J=6.2 Hz).

### Example 95

### 5-[(2S,6R)-2-[[4-[2-[(3S,4R)-3-amino-4-fluoro-pyrrolidin-1-yl]pyrimidin-4-yl]piperazin-1-yl]methyl]-6-methyl-morpholin-4-yl]quinoline-8-carbonitrile

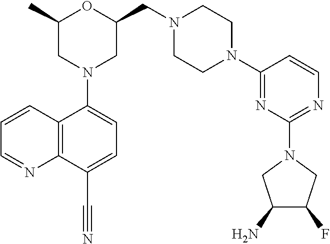 [SMILES: C[C@@H]1CN(c2ccc(C#N)c3ncccc23)C[C@H](CN2CCN(c3ccnc(N4C[C@@H](F)[C@@H](N)C4)n3)CC2)O1]

The title compound was prepared in analogy to the preparation of Example 23 by using tert-butyl N-[(3S,4R)-4-fluoropyrrolidin-3-yl]carbamate instead of tert-butyl azetidin-3-ylcarbamate (compound 23c). Example 95 (14 mg) was obtained as a light yellow solid. MS: calc'd 532 (MH⁺), measured 532 (MH⁺). ¹H NMR (METHANOL-d₄, 400 MHz) δ 9.02 (dd, 1H, J=1.7, 4.2 Hz), 8.68 (dd, 1H, J=1.7, 8.6 Hz), 8.19 (d, 1H, J=7.9 Hz), 7.94 (d, 1H, J=7.5 Hz), 7.67 (dd, 1H, J=4.2, 8.6 Hz), 7.30 (d, 1H, J=8.1 Hz), 6.67 (d, 1H, J=7.5 Hz), 5.4-5.7 (m, 1H), 4.5-4.6 (m, 1H), 3.9-4.4 (m, 9H), 3.72 (br t, 1H, J=10.5 Hz), 3.5-3.7 (m, 4H), 3.43 (br d, 4H, J=6.2 Hz), 2.7-2.9 (m, 2H), 1.34 (d, 3H, J=6.4 Hz).

### Example 96

### 5-[(2S,6R)-2-[[4-[2-[(3R,4R)-3-amino-4-fluoro-pyrrolidin-1-yl]pyrimidin-4-yl]piperazin-1-yl]methyl]-6-methyl-morpholin-4-yl]quinoline-8-carbonitrile

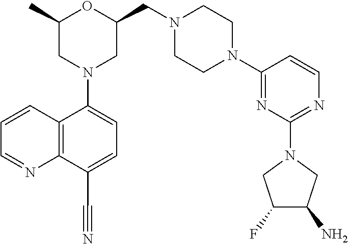 [SMILES: C[C@@H]1CN(c2ccc(C#N)c3ncccc23)C[C@H](CN2CCN(c3ccnc(N4C[C@@H](N)[C@H](F)C4)n3)CC2)O1]

The title compound was prepared in analogy to the preparation of Example 23 by using tert-butyl N-[(3R,4R)-4-fluoropyrrolidin-3-yl]carbamate instead of tert-butyl azetidin-3-ylcarbamate (compound 23c). Example 96 (14 mg) was obtained as a light yellow solid. MS: calc'd 532 (MH⁺), measured 532 (MH⁺). ¹H NMR (METHANOL-d₄, 400 MHz) δ 8.99 (dd, 1H, J=1.6, 4.3 Hz), 8.65 (dd, 1H, J=1.6, 8.6 Hz), 8.16 (d, 1H, J=7.9 Hz), 7.91 (d, 1H, J=7.5 Hz), 7.64 (dd, 1H, J=4.2, 8.6 Hz), 7.28 (d, 1H, J=8.1 Hz), 6.66 (d, 1H, J=7.6 Hz), 5.4-5.6 (m, 1H), 4.5-4.6 (m, 1H), 3.8-4.4 (m, 10H), 3.5-3.7 (m, 4H), 3.4-3.5 (m, 4H), 2.7-2.8 (m, 2H), 1.31 (d, 3H, J=6.2 Hz).

### Example 97

### 5-[(2S,6R)-2-[[4-[4-[(3S,4S)-3-amino-4-methoxy-pyrrolidin-1-yl]pyrimidin-2-yl]piperazin-1-yl]methyl]-6-methyl-morpholin-4-yl]quinoline-8-carbonitrile

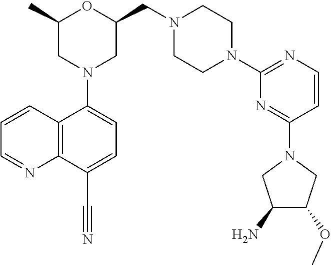 [SMILES: CO[C@H]1CN(c2ccnc(N3CCN(C[C@H]4CN(c5ccc(C#N)c6ncccc56)C[C@@H](C)O4)CC3)n2)C[C@@H]1N]

The title compound was prepared according to the following scheme:

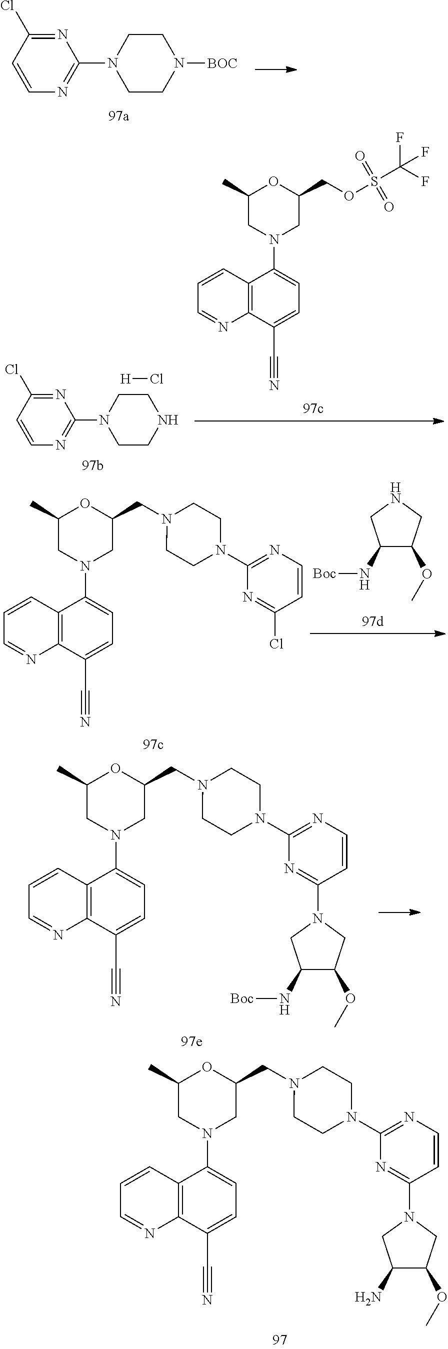

### Step 1: Preparation of 4-chloro-2-(piperazin-1-yl)pyrimidine hydrochloride (Compound 97b)

A solution of tert-butyl 4-(4-chloropyrimidin-2-yl)piperazine-1-carboxylate (compound 97^{a}, 600 mg, 2.01 mmol) in DCM (4 mL) was added a solution of hydrogen chloride in dioxane (2 mL, 4M). After the mixture was stirred at room temperature for 12 hrs, it was concentrated to afford compound 97b (383 mg). MS: calc'd 199 (MH⁺), measured 199 (MH⁺).

### Step 2: Preparation of 5-((2S,6R)-2-((4-(4-chloropyrimidin-2-yl)piperazin-1-yl)methyl)-6-methylmorpholino)quinoline-8-carbonitrile (Compound 97c)

A suspension of 4-chloro-2-(piperazin-1-yl)pyrimidine hydrochloride (compound 97b, 383 mg, 1.63 mmol), ((2R,6R)-4-(8-cyanoquinolin-5-yl)-6-methylmorpholin-2-yl)methyl trifluoromethanesulfonate (Intermediate A, 582 mg, 1.4 mmol) and K₂CO₃ (581 mg, 4.2 mmol) in MeCN (8 ml) was stirred at room temperature for 3 hrs. The mixture was cooled to room temperature and then diluted with water (5 mL) and extracted with EtOAc (10 mL) three times. The combined organic layer was washed with brine (5 mL), dried over Na₂SO₄, and concentrated. Then the residue was purified by silica gel chromatography (EA/PE from 20% to 80%) to afford compound 97c (492 mg) as yellow solid. MS: calc'd 464 (MH⁺), measured 464 (MH⁺).

### Step 3: Preparation of tert-butyl ((3S,4S)-1-(2-(4-(((2S,6R)-4-(8-cyanoquinolin-5-yl)-6-methylmorpholin-2-yl)methyl)piperazin-1-yl)pyrimidin-4-yl)-4-methoxypyrrolidin-3-yl)carbamate (Compound 97e)

A suspension of 5-((2S,6R)-2-((4-(4-chloropyrimidin-2-yl)piperazin-1-yl)methyl)-6-methylmorpholino)quinoline-8-carbonitrile (compound 97c, 46 mg, 99.1 μmol), tert-butyl N-[(3S,4S)-4-methoxypyrrolidin-3-yl]carbamate (compound 97d, 23.6 mg, 109 μmol) and K₂CO₃ (41.1 mg, 297 μmol) in MeCN (8 ml) was stirred at 120° C. for 12 hrs. The mixture was cooled to room temperature and then diluted with water (5 mL) and extracted with EtOAc (10 mL) three times. The combined organic layer was washed with brine (5 mL), dried over Na₂SO₄ and concentrated. Then the residue was purified by silica gel chromatography (EA/PE from 20% to 80%) to afford compound 97e (60 mg) as a yellow solid. MS: calc'd 644 (MH⁺), measured 644 (MH⁺).

### Step 4: preparation of 5-[(2S,6R)-2-[[4-[4-[(3S,4S)-3-amino-4-methoxy-pyrrolidin-1-yl]pyrimidin-2-yl]piperazin-1-yl]methyl]-6-methyl-morpholin-4-yl]quinoline-8-carbonitrile

### Example 97

To a solution of tert-butyl ((3S,4S)-1-(2-(4-(((2S,6R)-4-(8-cyanoquinolin-5-yl)-6-methylmorpholin-2-yl)methyl)piperazin-1-yl)pyrimidin-4-yl)-4-methoxypyrrolidin-3-yl)carbamate (compound 97e, 60 mg, 93.2 μmol) in DCM (4 ml) was added 2,2,2-trifluoroacetic acid (740 mg, 0.5 ml, 6.49 mmol) at 0° C. The reaction mixture was stirred at r.t. for 2 hrs. Then the mixture was concentrated and purified by prep-HPLC to afford 5-[(2S,6R)-2-[[4-[2-[(3R,4R)-3-amino-4-fluoro-pyrrolidin-1-yl]pyrimidin-4-yl]piperazin-1-yl]methyl]-6-methyl-morpholin-4-yl]quinoline-8-carbonitrile (Example 97, 54 mg) as a light yellow solid. MS calc'd 544 (MH⁺), measured 544 (MH⁺). ¹H NMR (METHANOL-d₄, 400 MHz) δ 9.01 (dd, 1H, J=1.7, 4.2 Hz), 8.67 (dd, 1H, J=1.6, 8.6 Hz), 8.18 (d, 1H, J=7.9 Hz), 7.91 (d, 1H, J=7.2 Hz), 7.66 (dd, 1H, J=4.3, 8.6 Hz), 7.30 (d, 1H, J=8.1 Hz), 6.39 (d, 1H, J=7.2 Hz), 4.5-4.6 (m, 1H), 4.0-4.3 (m, 9H), 3.7-3.9 (m, 2H), 3.5-3.7 (m, 4H), 3.50 (s, 3H), 3.4-3.5 (m, 4H), 2.7-2.8 (m, 2H), 1.33 (d, 3H, J=6.2 Hz).

### Example 98

### 8-[(2S,6R)-2-[[4-[2-[(3R,4R)-3-amino-4-methoxy-pyrrolidin-1-yl]pyrimidin-4-yl]piperazin-1-yl]methyl]-6-methyl-morpholin-4-yl]quinoxaline-5-carbonitrile

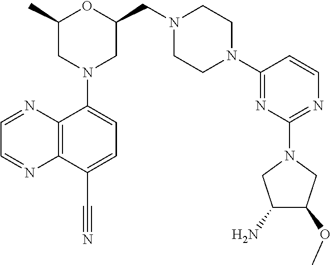 [SMILES: CO[C@@H]1CN(c2nccc(N3CCN(C[C@H]4CN(c5ccc(C#N)c6nccnc56)C[C@@H](C)O4)CC3)n2)C[C@H]1N]

The title compound was prepared in analogy to the preparation of Example 23 by using ((2R,6R)-4-(8-cyanoquinoxalin-5-yl)-6-methylmorpholin-2-yl)methyl trifluoromethanesulfonate (Intermediate C) and tert-butyl N-[(3R,4R)-4-methoxypyrrolidin-3-yl]carbamate instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 98 (31 mg) was obtained as a light yellow solid. MS: calc'd 545 (MH⁺), measured 545 (MH⁺). ¹H NMR (METHANOL-d₄, 400 MHz) δ 8.99 (d, 1H, J=1.7 Hz), 8.91 (d, 1H, J=1.7 Hz), 8.17 (d, 1H, J=8.3 Hz), 7.91 (d, 1H, J=7.5 Hz), 7.28 (d, 1H, J=8.3 Hz), 6.65 (d, 1H, J=7.5 Hz), 4.4-4.5 (m, 1H), 4.0-4.4 (m, 11H), 3.7-4.0 (m, 2H), 3.55 (br d, 4H, J=1.6 Hz), 3.5-3.5 (m, 3H), 3.4-3.5 (m, 2H), 2.8-2.9 (m, 2H), 1.34 (d, 3H, J=6.1 Hz).

### Example 99

### 8-[(2S,6R)-2-[[4-[2-[(3S,4S)-3-amino-4-methoxy-pyrrolidin-1-yl]pyrimidin-4-yl]piperazin-1-yl]methyl]-6-methyl-morpholin-4-yl]quinoxaline-5-carbonitrile

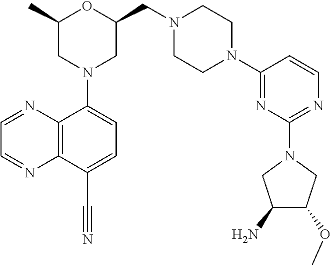 [SMILES: CO[C@H]1CN(c2nccc(N3CCN(C[C@H]4CN(c5ccc(C#N)c6nccnc56)C[C@@H](C)O4)CC3)n2)C[C@@H]1N]

The title compound was prepared in analogy to the preparation of Example 23 by using ((2R,6R)-4-(8-cyanoquinoxalin-5-yl)-6-methylmorpholin-2-yl)methyl trifluoromethanesulfonate (Intermediate C) and tert-butyl N-[(3S,4S)-4-methoxypyrrolidin-3-yl]carbamate instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 99 (18 mg) was obtained as a light yellow solid. MS: calc'd 545 (MH⁺), measured 545 (MH⁺). ¹H NMR (METHANOL-d₄, 400 MHz) δ 8.99 (d, 1H, J=1.8 Hz), 8.91 (d, 1H, J=1.8 Hz), 8.17 (d, 1H, J=8.3 Hz), 7.91 (d, 1H, J=7.5 Hz), 7.28 (d, 1H, J=8.3 Hz), 6.65 (d, 1H, J=7.6 Hz), 4.4-4.5 (m, 1H), 4.1-4.4 (m, 8H), 4.0-4.1 (m, 3H), 3.8-4.0 (m, 1H), 3.7-3.8 (m, 1H), 3.5-3.7 (m, 4H), 3.50 (s, 3H), 3.4-3.4 (m, 2H), 2.8-2.9 (m, 2H), 1.35 (d, 3H, J=6.1 Hz).

### Example 101

### 5-[(2S,6R)-2-[[4-[2-[(3R,4S)-3-amino-4-fluoro-pyrrolidin-1-yl]pyrimidin-4-yl]piperazin-1-yl]methyl]-6-methyl-morpholin-4-yl]quinoline-8-carbonitrile

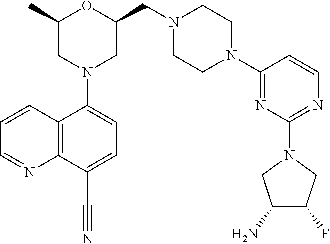 [SMILES: C[C@@H]1CN(c2ccc(C#N)c3ncccc23)C[C@H](CN2CCN(c3ccnc(N4C[C@@H](N)[C@@H](F)C4)n3)CC2)O1]

The title compound was prepared in analogy to the preparation of Example 23 by using tert-butyl N-[(3R,4S)-4-fluoropyrrolidin-3-yl]carbamate instead of tert-butyl azetidin-3-ylcarbamate (compound 23c). Example 101 (9 mg) was obtained as a light yellow solid. MS: calc'd 532 (MH⁺), measured 532 (MH⁺). ¹H NMR (METHANOL-d₄, 400 MHz) δ 8.88 (dd, 1H, J=1.7, 4.2 Hz), 8.55 (dd, 1H, J=1.7, 8.6 Hz), 8.05 (d, 1H, J=8.1 Hz), 7.82 (d, 1H, J=7.5 Hz), 7.54 (dd, 1H, J=4.3, 8.6 Hz), 7.17 (d, 1H, J=7.9 Hz), 6.56 (d, 1H, J=7.5 Hz), 5.3-5.5 (m, 1H), 4.4-4.5 (m, 1H), 3.8-4.3 (m, 9H), 3.61 (br t, 1H, J=10.3 Hz), 3.4-3.6 (m, 4H), 3.31 (br s, 4H), 2.6-2.7 (m, 2H), 1.21 (d, 3H, J=6.2 Hz).

### Example 102

### 5-[(2R,6S)-2-methyl-6-[[4-(5-methyl-4-piperazin-1-yl-pyrimidin-2-yl)piperazin-1-yl]methyl]morpholin-4-yl]quinoline-8-carbonitrile

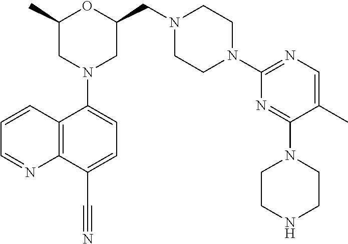 [SMILES: Cc1cnc(N2CCN(C[C@H]3CN(c4ccc(C#N)c5ncccc45)C[C@@H](C)O3)CC2)nc1N1CCNCC1]

The title compound was prepared according to the following scheme:

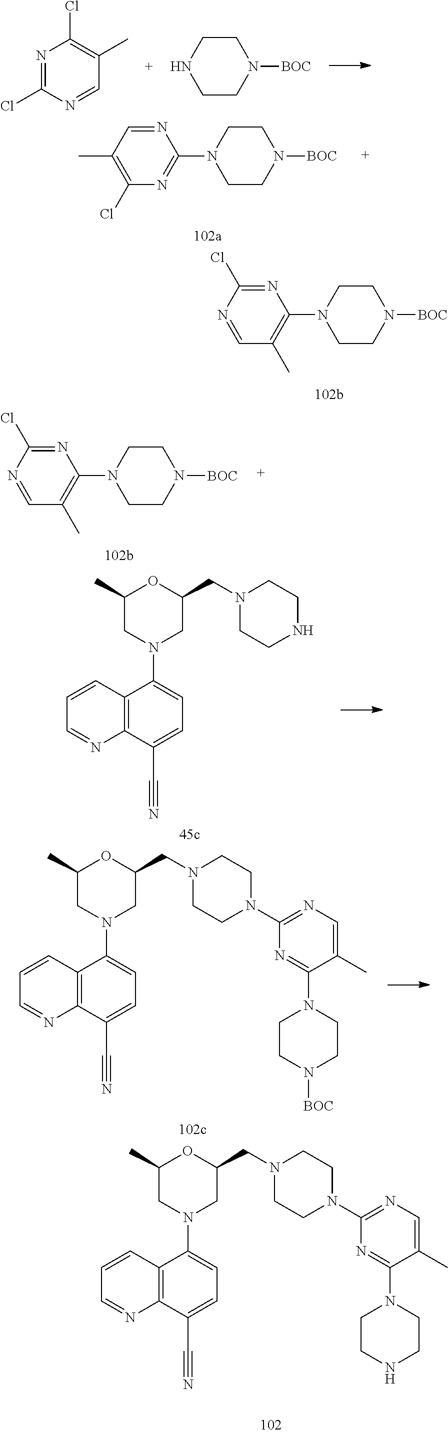

### Step 1: preparation of tert-butyl 4-(4-chloro-5-methylpyrimidin-2-yl)piperazine-1-carboxylate (Compound 102a) and tert-butyl 4-(2-chloro-5-methylpyrimidin-4-yl)piperazine-1-carboxylate (Compound 102b)

A solution of 2,4-dichloro-5-methylpyrimidine (326 mg, 2 mmol) and 2,4-dichloro-5-methylpyrimidine (326 mg, 2 mmol) in ethanol (6 mL) was stirred at room temperature for 12 hrs. Then the mixture was concentrated and purified by silica gel chromatography (EA/PE from 20% to 80%) to afford compound 102a (100 mg) as a white solid. MS: calc'd 313 (MH⁺), measured 313 (MH⁺).

### Step 2: Preparation of tert-butyl 4-(2-(4-(((2S,6R)-4-(8-cyanoquinolin-5-yl)-6-methylmorpholin-2-yl)methyl)piperazin-1-yl)-5-methylpyrimidin-4-yl)piperazine-1-carboxylate (Compound 102c)

A suspension of 5-((2R,6S)-2-methyl-6-(piperazin-1-ylmethyl)morpholino)quinoline-8-carbonitrile (compound 45c, 35 mg, 99.6 μmol), tert-butyl 4-(2-chloro-5-methylpyrimidin-4-yl)piperazine-1-carboxylate (compound 102b, 31 mg, 99.6 μmol), Ruphos Pd G2 (7 mg, 9.96 μmol) and k₂CO₃ (27 mg, 199 μmol) in 1,4-Dioxane (4 mL) was heated to 100° C. for 12 hrs under nitrogen atmosphere. The mixture was cooled to room temperature and then diluted with water (5 mL) and extracted with EtOAC (10 mL) three times. The combined organic layer was washed with brine, dried over Na₂SO₄, and concentrated. The resulting residue was purified by silica gel chromatography (EA/PE from 20% to 80%) to afford tert-butyl 4-(2-(4-(((2S,6R)-4-(8-cyanoquinolin-5-yl)-6-methylmorpholin-2-yl)methyl)piperazin-1-yl)-5-methylpyrimidin-4-yl)piperazine-1-carboxylate (compound 102c, 19 mg) as a pale yellow solid. MS: calc'd 628 (MH⁺), measured 628 (MH⁺).

### Step 3: Preparation of 5-[(2S,6R)-2-[[4-(5-fluoro-4-piperazin-1-yl-pyrimidin-2-yl)piperazin-1-yl]methyl]-6-methyl-morpholin-4-yl]quinoline-8-carbonitrile (Example 102)

To a solution of tert-butyl 4-(2-(4-(((2S,6R)-4-(8-cyanoquinolin-5-yl)-6-methylmorpholin-2-yl)methyl)piperazin-1-yl)-5-methylpyrimidin-4-yl)piperazine-1-carboxylate (compound 102c, 19 mg, 30.3 μmol) in DCM (4 mL) was added 2,2,2-trifluoroacetic acid (0.5 mL) at 0° C. The reaction mixture was stirred at r.t. for 2 hrs. Then the mixture was concentrated and purified by prep-HPLC (TFA/MeCN) to afford Example 102 (27 mg) as a light yellow solid. MS calc'd 528 (MH⁺), measured 528 (MH⁺). ¹H NMR (METHANOL-d₄, 400 MHz) δ 8.99 (dd, 1H, J=1.7, 4.2 Hz), 8.66 (dd, 1H, J=1.7, 8.6 Hz), 8.16 (d, 1H, J=8.1 Hz), 7.88 (d, 1H, J=0.9 Hz), 7.65 (dd, 1H, J=4.2, 8.6 Hz), 7.28 (d, 1H, J=8.1 Hz), 4.5-4.6 (m, 1H), 4.1-4.3 (m, 4H), 4.0-4.0 (m, 4H), 3.5-3.7 (m, 4H), 3.4-3.5 (m, 8H), 2.7-2.8 (m, 2H), 2.28 (s, 3H), 1.33 (d, 3H, J=6.2 Hz).

### Example 103

### 5-[(2S,6R)-2-[[4-[2-[(4aR,7aR)-3,4,4a,5,7,7a-hexahydro-2H-pyrrolo[3,4-b][1,4]oxazin-6-yl]pyrimidin-4-yl]piperazin-1-yl]methyl]-6-methyl-morpholin-4-yl]quinoline-8-carbonitrile

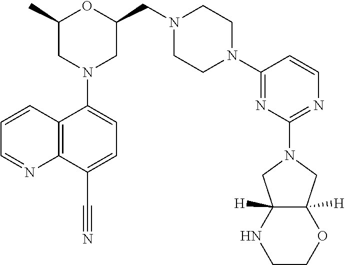 [SMILES: C[C@@H]1CN(c2ccc(C#N)c3ncccc23)C[C@H](CN2CCN(c3ccnc(N4C[C@H]5NCCO[C@@H]5C4)n3)CC2)O1]

The title compound was prepared in analogy to the preparation of Example 23 by using tert-butyl (4aR,7aR)-3,4a,5,6,7,7a-hexahydro-2H-pyrrolo[3,4-b][1,4]oxazine-4-carboxylate (PharmaBlock, PBXA8123, CAS: 1932337-68-2) instead of tert-butyl azetidin-3-ylcarbamate (compound 23c). Example 101 (2 mg) was obtained as a light yellow solid. MS: calc'd 556 (MH⁺), measured 556 (MH⁺). ¹H NMR (METHANOL-d₄, 400 MHz) δ 9.0-9.0 (m, 1H), 8.6-8.7 (m, 1H), 8.17 (d, 1H, J=8.1 Hz), 7.90 (d, 1H, J=7.1 Hz), 7.65 (dd, 1H, J=4.4, 8.6 Hz), 7.28 (d, 1H, J=8.1 Hz), 6.6-6.7 (m, 1H), 4.5-4.5 (m, 2H), 3.9-4.3 (m, 10H), 3.4-3.6 (m, 12H), 2.7-2.8 (m, 2H), 1.31 (d, 3H, J=6.2 Hz).

### Example 104

### 8-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile

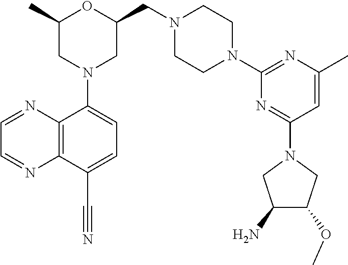 [SMILES: CO[C@H]1CN(c2cc(C)nc(N3CCN(C[C@H]4CN(c5ccc(C#N)c6nccnc56)C[C@@H](C)O4)CC3)n2)C[C@@H]1N]

The title compound was prepared according to the following scheme:

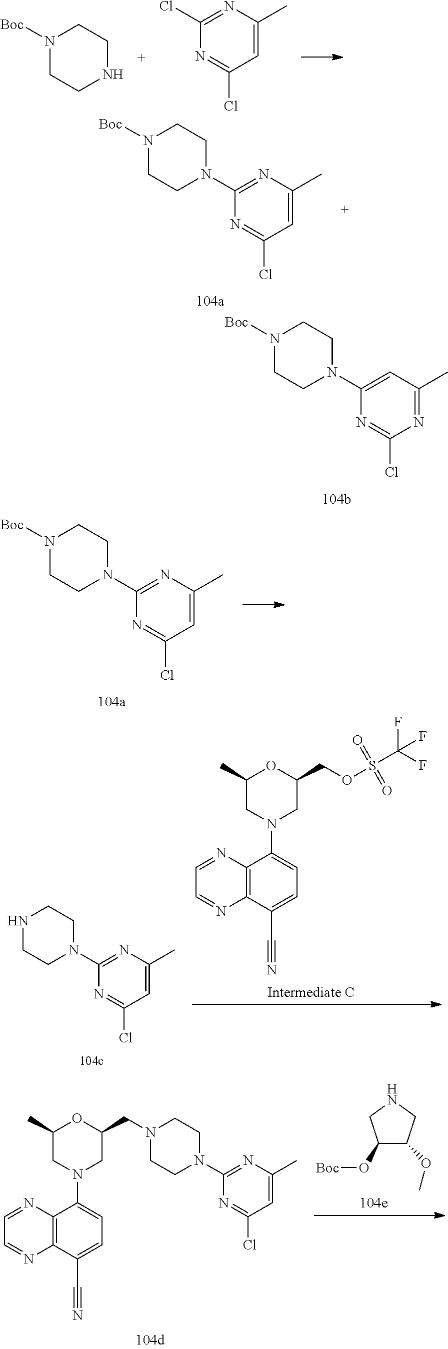

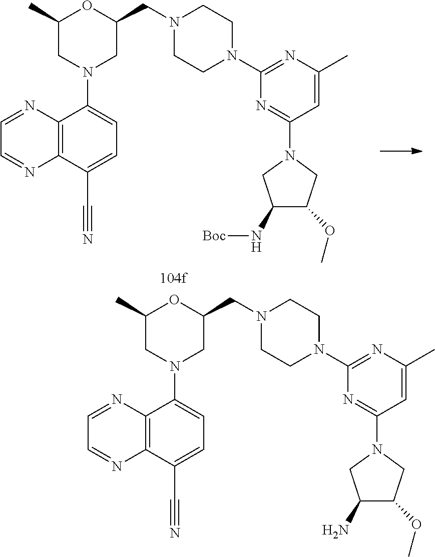 [SMILES: CO[C@H]1CN(c2cc(C)nc(N3CCN(C[C@H]4CN(c5ccc(C#N)c6nccnc56)C[C@@H](C)O4)CC3)n2)C[C@@H]1NC(=O)OC(C)(C)C ; CO[C@H]1CN(c2cc(C)nc(N3CCN(C[C@H]4CN(c5ccc(C#N)c6nccnc56)C[C@@H](C)O4)CC3)n2)C[C@@H]1N]

### Step 1: preparation of tert-butyl 4-(4-chloro-6-methylpyrimidin-2-yl)piperazine-1-carboxylate (Compound 104a) and tert-butyl 4-(2-chloro-6-methylpyrimidin-4-yl)piperazine-1-carboxylate (Compound 104b)

A solution of 2,4-dichloro-6-methylpyrimidine (3.26 g, 20 mmol), Et₃N (2.79 mL, 20 mmol) and tert-butyl piperazine-1-carboxylate (4.10 g, 22 mmol) in ethanol (40 mL) was stirred at room temperature for 12 hrs. Then the mixture was concentrated and diluted with DCM (20 mL) and washed with water (10 mL) three times, brine (10 mL) and dried over Na₂SO₄. The resulting residue was purified by silica gel chromatography (PE/EtOAc from 10% to 60%) to afford tert-butyl 4-(4-chloro-6-methylpyrimidin-2-yl)piperazine-1-carboxylate (compound 104a, 1 g) and tert-butyl 4-(2-chloro-6-methylpyrimidin-4-yl)piperazine-1-carboxylate (compound 104b, 4.35 g). MS: calc'd 313 (MH⁺), measured 313 (MH⁺).

### Step 2: preparation of 4-chloro-6-methyl-2-(piperazin-1-yl)pyrimidine (Compound 104c)

A solution of tert-butyl 4-(4-chloro-6-methylpyrimidin-2-yl)piperazine-1-carboxylate (compound 104a, 1 g) in DCM (4 mL) was added hydrogen chloride/dioxane (4 mL, 4M in dioxane). After the reaction mixture was stirred at room temperature for 12 hrs, it was concentrated to afford 4-chloro-6-methyl-2-(piperazin-1-yl)pyrimidine (compound 104c, 756 mg). MS: calc'd 213 (MH⁺), measured 213 (MH⁺).

### Step 3: preparation of 8-[(2S,6R)-2-[[4-(4-chloro-6-methyl-pyrimidin-2-yl)piperazin-1-yl]methyl]-6-methyl-morpholin-4-yl]quinoxaline-5-carbonitrile (Compound 104d)

A suspension of 4-chloro-6-methyl-2-(piperazin-1-yl)pyrimidine (compound 104c, 479 mg, 1.92 mmol), [(2R,6R)-4-(8-cyanoquinoxalin-5-yl)-6-methyl-morpholin-2-yl]methyl trifluoromethanesulfonate (intermediate C, 800 mg, 1.92 mmol) and K₂CO₃ (797 mg, 5.76 mmol) in MeCN (8 ml) was stirred at room temperature for 3 hrs. The mixture was cooled to room temperature and then diluted with water (5 mL) and extracted with EtOAc (10 mL) three times. The combined organic layer was washed with brine (5 mL), dried over Na₂SO₄, and concentrated. Then the residue was purified by silica gel chromatography (PE/EtOAc 20% to 100%) to afford 8-[(2S,6R)-2-[[4-(4-chloro-6-methyl-pyrimidin-2-yl)piperazin-1-yl]methyl]-6-methyl-morpholin-4-yl]quinoxaline-5-carbonitrile (compound 104d, 560 mg) as a yellow solid. MS: calc'd 479 (MH⁺), measured 479 (MH⁺).

### Step 4: Preparation of tert-butyl ((3S,4S)-1-(2-(4-(((2S,6R)-4-(8-cyanoquinoxalin-5-yl)-6-methylmorpholin-2-yl)methyl)piperazin-1-yl)-6-methylpyrimidin-4-yl)-4-methoxypyrrolidin-3-yl)carbamate (Compound 104f)

A suspension of 8-[(2S,6R)-2-[[4-(4-chloro-6-methyl-pyrimidin-2-yl)piperazin-1-yl]methyl]-6-methyl-morpholin-4-yl]quinoxaline-5-carbonitrile (compound 104d, 21 mg, 43.8 μmol), tert-butyl N-[(3S,4S)-4-methoxypyrrolidin-3-yl]carbamate (compound 104e, 10.4 mg, 48.2 μmol) and K₂CO₃ (18.2 mg, 132 μmol) in MeCN (8 mL) was stirred at 120° C. for 12 hrs. The mixture was cooled to room temperature and then diluted with water (5 mL) and extracted with EtOAc (10 mL) three times. The combined organic layer was washed with brine (5 mL), dried over Na₂SO₄ and concentrated. Then the residue was purified by silica gel chromatography (EA/PE from 20% to 80%) to afford compound tert-butyl ((3S,4S)-1-(2-(4-(((2S,6R)-4-(8-cyanoquinoxalin-5-yl)-6-methylmorpholin-2-yl)methyl)piperazin-1-yl)-6-methylpyrimidin-4-yl)-4-methoxypyrrolidin-3-yl)carbamate (compound 104f, 20 mg) as a yellow solid. MS: calc'd 659 (MH⁺), measured 659 (MH⁺).

### Step 5: preparation of 8-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile (Compound 104)

To a solution of tert-butyl ((3S,4S)-1-(2-(4-(((2S,6R)-4-(8-cyanoquinoxalin-5-yl)-6-methylmorpholin-2-yl)methyl)piperazin-1-yl)-6-methylpyrimidin-4-yl)-4-methoxypyrrolidin-3-yl)carbamate (compound 104f, 20 mg, 30.4 μmol) in DCM (4 mL) was added HCl (0.2 mL, 4M in dioxane) at 0° C. The reaction mixture was stirred at rt for 2 hrs. Then the mixture was concentrated to afford Example 104 (13.1 mg) as a light yellow solid. MS calc'd 559 (MH⁺), measured 559 (MH⁺). ¹H NMR (METHANOL-d₄, 400 MHz) δ 8.98 (d, 1H, J=1.8 Hz), 8.90 (d, 1H, J=1.7 Hz), 8.16 (d, 1H, J=8.3 Hz), 7.27 (d, 1H, J=8.3 Hz), 6.31 (s, 1H), 4.6-4.8 (m, 1H), 4.5-4.5 (m, 1H), 4.3-4.3 (m, 1H), 4.15 (br d, 8H, J=2.0 Hz), 3.6-3.9 (m, 6H), 3.5-3.5 (m, 1H), 3.5-3.5 (m, 3H), 3.4-3.5 (m, 2H), 2.8-2.9 (m, 2H), 2.46 (s, 3H), 1.34 (d, 3H, J=6.1 Hz).

### Example 105

### 8-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]-2,3-dideuterio-quinoxaline-5-carbonitrile

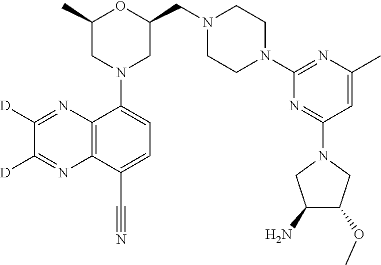 [SMILES: [2H]c1nc2c(C#N)ccc(N3C[C@H](CN4CCN(c5nc(C)cc(N6C[C@H](OC)[C@@H](N)C6)n5)CC4)O[C@H](C)C3)c2nc1[2H]]

The title compound was prepared in analogy to the preparation of Example 104 by using [(2R,6R)-4-(8-cyano-2,3-dideuterio-quinoxalin-5-yl)-6-methyl-morpholin-2-yl]methyl trifluoromethanesulfonate (Intermediate K) instead of [(2R,6R)-4-(8-cyanoquinoxalin-5-yl)-6-methyl-morpholin-2-yl]methyl trifluoromethanesulfonate (Intermediate C). Example 105 (21 mg) was obtained as a light yellow solid. MS: calc'd 561 (MH⁺), measured 561 (MH⁺). ¹H NMR (METHANOL-d₄, 400 MHz) δ 8.1-8.2 (m, 1H), 7.26 (d, 1H, J=8.3 Hz), 6.24 (s, 1H), 4.4-4.5 (m, 1H), 4.29 (br d, 1H, J=12.1 Hz), 4.1-4.2 (m, 6H), 3.9-4.1 (m, 4H), 3.7-3.8 (m, 1H), 3.5-3.6 (m, 4H), 3.5-3.5 (m, 3H), 3.4-3.4 (m, 2H), 2.8-2.9 (m, 2H), 2.41 (s, 3H), 1.4-1.4 (m, 1H), 1.3-1.3 (m, 3H).

### Example 106

### 8-[(2S,6R)-2-[[4-[2-[(3S,4S)-3-amino-4-methoxy-pyrrolidin-1-yl]-6-methyl-pyrimidin-4-yl]piperazin-1-yl]methyl]-6-methyl-morpholin-4-yl]quinoxaline-5-carbonitrile

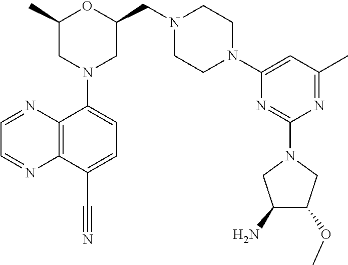 [SMILES: CO[C@H]1CN(c2nc(C)cc(N3CCN(C[C@H]4CN(c5ccc(C#N)c6nccnc56)C[C@@H](C)O4)CC3)n2)C[C@@H]1N]

The title compound was prepared in analogy to the preparation of Example 45 by using 2,4-dichloro-6-methyl-pyrimidine and ((2R,6R)-4-(8-cyanoquinoxalin-5-yl)-6-methylmorpholin-2-yl)methyl trifluoromethanesulfonate (Intermediate C) and tert-butyl N-[(3S,4S)-4-methoxypyrrolidin-3-yl]carbamate instead of 2,4-dichloro-5-fluoropyrimidine (compound 45d) and ((2R,6R)-4-(8-cyanoquinolin-5-yl)-6-methylmorpholin-2-yl)methyl trifluoromethanesulfonate (Intermediate A) and tert-butyl piperazine-1-carboxylate (compound 45a). Example 106 (41 mg) was obtained as a light yellow solid. MS: calc'd 559 (MH⁺), measured 559 (MH⁺). ¹H NMR (METHANOL-d₄, 400 MHz) δ 8.98 (d, 1H, J=1.8 Hz), 8.89 (d, 1H, J=1.7 Hz), 8.16 (d, 1H, J=8.3 Hz), 7.27 (d, 1H, J=8.6 Hz), 6.54 (s, 1H), 4.4-4.5 (m, 1H), 4.0-4.3 (m, 10H), 3.6-3.9 (m, 6H), 3.5-3.6 (m, 2H), 3.5-3.5 (m, 3H), 3.5-3.5 (m, 1H), 2.8-2.9 (m, 2H), 2.44 (s, 3H), 1.33 (d, 3H, J=5.9 Hz).

### Example 107

### 8-[(2S,6R)-2-[[4-[2-[(3S,4S)-3-amino-4-fluoro-pyrrolidin-1-yl]pyrimidin-4-yl]piperazin-1-yl]methyl]-6-methyl-morpholin-4-yl]quinoxaline-5-carbonitrile

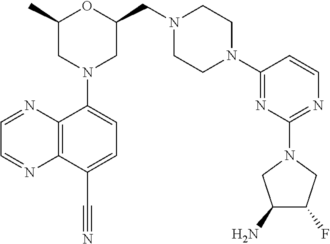 [SMILES: C[C@@H]1CN(c2ccc(C#N)c3nccnc23)C[C@H](CN2CCN(c3ccnc(N4C[C@H](N)[C@@H](F)C4)n3)CC2)O1]

The title compound was prepared in analogy to the preparation of Example 23 by using ((2R,6R)-4-(8-cyanoquinoxalin-5-yl)-6-methylmorpholin-2-yl)methyl trifluoromethanesulfonate (Intermediate C) and tert-butyl N-[(3S,4S)-4-fluoroprrolidin-3-yl]carbamate instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23^{c}). Example 107 (50 mg) was obtained as a light yellow solid. MS: calc'd 533 (MH⁺), measured 533 (MH⁺). ¹H NMR (METHANOL-d₄, 400 MHz) δ 9.00 (d, 1H, J=1.7 Hz), 8.18 (d, 1H, J=8.3 Hz), 7.94 (d, 1H, J=7.5 Hz), 7.30 (d, 1H, J=8.4 Hz), 6.74 (d, 1H, J=7.6 Hz), 5.5-5.7 (m, 1H), 5.16 (br s, 1H), 4.4-4.6 (m, 2H), 4.1-4.4 (m, 7H), 3.6-4.1 (m, 6H), 3.4-3.6 (m, 4H), 2.8-2.9 (m, 2H), 1.36 (d, 3H, J=6.1 Hz).

### Example 108

### 8-[(2S,6R)-2-[[4-[2-[(3R,4S)-3-amino-4-fluoro-pyrrolidin-1-yl]pyrimidin-4-yl]piperazin-1-yl]methyl]-6-methyl-morpholin-4-yl]quinoxaline-5-carbonitrile

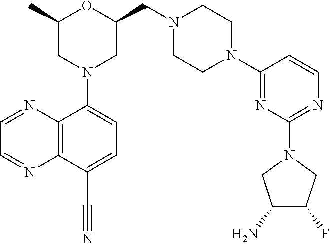 [SMILES: C[C@@H]1CN(c2ccc(C#N)c3nccnc23)C[C@H](CN2CCN(c3ccnc(N4C[C@@H](N)[C@@H](F)C4)n3)CC2)O1]

The title compound was prepared in analogy to the preparation of Example 23 by using ((2R,6R)-4-(8-cyanoquinoxalin-5-yl)-6-methylmorpholin-2-yl)methyl trifluoromethanesulfonate (Intermediate C) and tert-butyl N-[(3R,4S)-4-fluoroprrolidin-3-yl]carbamate instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 108 (27 mg) was obtained as a light yellow solid. MS: calc'd 533 (MH⁺), measured 533 (MH⁺). ¹H NMR (METHANOL-d₄, 400 MHz) δ 8.84 (d, 1H, J=1.7 Hz), 8.78 (d, 1H, J=1.7 Hz), 8.01 (d, 1H, J=8.3 Hz), 7.71 (d, 1H, J=6.2 Hz), 7.10 (d, 1H, J=8.4 Hz), 5.97 (d, 1H, J=6.2 Hz), 4.8-5.0 (m, 1H), 4.21 (br d, 1H, J=12.2 Hz), 4.0-4.1 (m, 2H), 3.8-4.0 (m, 2H), 3.6-3.8 (m, 6H), 3.3-3.5 (m, 1H), 3.0-3.1 (m, 1H), 2.6-2.7 (m, 4H), 2.49 (br d, 4H, J=7.0 Hz), 1.16 (d, 3H, J=6.2 Hz).

### Example 109

### 5-[(2S,6R)-2-[[4-[2-(3-amino-4-methoxy-1-piperidyl)pyrimidin-4-yl]piperazin-1-yl]methyl]-6-methyl-morpholin-4-yl]quinoline-8-carbonitrile

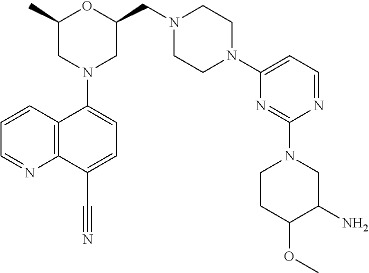 [SMILES: COC1CCN(c2nccc(N3CCN(C[C@H]4CN(c5ccc(C#N)c6ncccc56)C[C@@H](C)O4)CC3)n2)CC1N]

The title compound was prepared in analogy to the preparation of Example 23 by using tert-butyl N-(4-methoxy-3-piperidyl)carbamate (PharmaBlock, PBCS1406262, CAS: 1262407-41-9) instead of tert-butyl azetidin-3-ylcarbamate (compound 23c). Example 109 (28 mg) was obtained as a light yellow solid. MS: calc'd 558 (MH⁺), measured 558 (MH⁺). ¹H NMR (METHANOL-d₄, 400 MHz) δ 9.02 (dd, 1H, J=1.7, 4.2 Hz), 8.68 (dd, 1H, J=1.6, 8.6 Hz), 8.19 (d, 1H, J=7.9 Hz), 7.93 (d, 1H, J=7.3 Hz), 7.67 (dd, 1H, J=4.3, 8.6 Hz), 7.31 (d, 1H, J=8.1 Hz), 6.64 (d, 1H, J=7.3 Hz), 4.5-4.6 (m, 1H), 4.41 (br dd, 1H, J=3.4, 13.2 Hz), 4.1-4.3 (m, 4H), 3.84 (br s, 3H), 3.5-3.7 (m, 6H), 3.5-3.5 (m, 3H), 3.4-3.5 (m, 5H), 2.7-2.9 (m, 2H), 2.1-2.2 (m, 1H), 1.9-2.0 (m, 1H), 1.34 (d, 3H, J=6.2 Hz).

### Example 110

### 5-[(2S,6R)-2-[[4-[2-[(3S,4S)-3-amino-4-fluoro-pyrrolidin-1-yl]pyrimidin-4-yl]piperazin-1-yl]methyl]-6-methyl-morpholin-4-yl]quinoline-8-carbonitrile

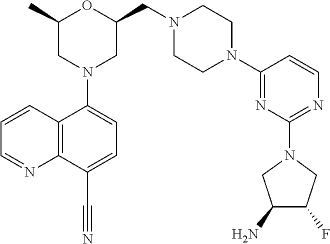 [SMILES: C[C@@H]1CN(c2ccc(C#N)c3ncccc23)C[C@H](CN2CCN(c3ccnc(N4C[C@H](N)[C@@H](F)C4)n3)CC2)O1]

The title compound was prepared in analogy to the preparation of Example 23 by using tert-butyl N-[(3S,4S)-4-fluoropyrrolidin-3-yl]carbamate instead of tert-butyl azetidin-3-ylcarbamate (compound 23c). Example 110 (14 mg) was obtained as a light yellow solid. MS: calc'd 532 (MH⁺), measured 532 (MH⁺). ¹H NMR (METHANOL-d₄, 400 MHz) δ 8.98 (dd, 1H, J=1.6, 4.2 Hz), 8.66 (dd, 1H, J=1.6, 8.6 Hz), 8.15 (d, 1H, J=7.9 Hz), 7.81 (d, 1H, J=6.2 Hz), 7.64 (dd, 1H, J=4.3, 8.6 Hz), 7.25 (d, 1H, J=7.9 Hz), 6.07 (d, 1H, J=6.4 Hz), 4.1-4.2 (m, 1H), 4.1-4.1 (m, 1H), 3.6-3.9 (m, 8H), 3.4-3.6 (m, 6H), 2.6-2.7 (m, 6H), 1.25 (d, 3H, J=6.2 Hz).

### Example 111

### 8-[(2S,6R)-2-[[4-[2-[(3R,4S)-4-amino-3-fluoro-1-piperidyl]pyrimidin-4-yl]piperazin-1-yl]methyl]-6-methyl-morpholin-4-yl]quinoxaline-5-carbonitrile

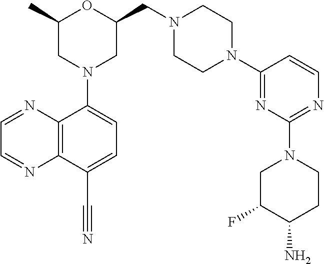 [SMILES: C[C@@H]1CN(c2ccc(C#N)c3nccnc23)C[C@H](CN2CCN(c3ccnc(N4CC[C@H](N)[C@H](F)C4)n3)CC2)O1]

The title compound was prepared in analogy to the preparation of Example 23 by using ((2R,6R)-4-(8-cyanoquinoxalin-5-yl)-6-methylmorpholin-2-yl)methyl trifluoromethanesulfonate (Intermediate C) and tert-butyl N-[(3R,4S)-3-fluoro-4-piperidyl]carbamate (PharmaBlock, PBZS1007, CAS:1630815-57-4) instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 111 (21 mg) was obtained as a light yellow solid. MS: calc'd 547 (MH⁺), measured 547 (MH⁺). ¹H NMR (METHANOL-d₄, 400 MHz) δ 8.99 (d, 1H, J=1.8 Hz), 8.92 (d, 1H, J=1.7 Hz), 8.17 (d, 1H, J=8.3 Hz), 7.92 (d, 1H, J=7.5 Hz), 7.29 (d, 1H, J=8.3 Hz), 6.72 (d, 1H, J=7.6 Hz), 5.1-5.3 (m, 2H), 4.9-5.0 (m, 1H), 4.6-4.7 (m, 1H), 4.4-4.6 (m, 2H), 4.3-4.3 (m, 1H), 4.1-4.2 (m, 2H), 3.54 (s, 11H), 2.8-3.0 (m, 2H), 2.0-2.2 (m, 2H), 1.3-1.4 (m, 3H).

### Example 112

### 8-[(2S,6R)-2-[[4-[2-(3-aminoazetidin-1-yl)-6-methyl-pyrimidin-4-yl]piperazin-1-yl]methyl]-6-methyl-morpholin-4-yl]quinoxaline-5-carbonitrile

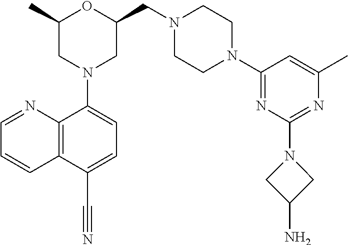 [SMILES: Cc1cc(N2CCN(C[C@H]3CN(c4ccc(C#N)c5cccnc45)C[C@@H](C)O3)CC2)nc(N2CC(N)C2)n1]

The title compound was prepared in analogy to the preparation of Example 45 by using 2,4-dichloro-6-methyl-pyrimidine and ((2R,6R)-4-(8-cyanoquinoxalin-5-yl)-6-methylmorpholin-2-yl)methyl trifluoromethanesulfonate (Intermediate C) and tert-butyl N-(azetidin-3-yl)carbamate instead of 2,4-dichloro-5-fluoropyrimidine (compound 45d) and ((2R,6R)-4-(8-cyanoquinolin-5-yl)-6-methylmorpholin-2-yl)methyl trifluoromethanesulfonate (Intermediate A) and tert-butyl piperazine-1-carboxylate (compound 45a). Example 112 (20 mg) was obtained as a light yellow solid. MS: calc'd 515 (MH⁺), measured 515 (MH⁺). ¹H NMR (METHANOL-d₄, 400 MHz) δ 8.99 (d, 1H, J=1.7 Hz), 8.92 (d, 1H, J=1.8 Hz), 8.17 (d, 1H, J=8.3 Hz), 7.29 (d, 1H, J=8.4 Hz), 6.56 (s, 1H), 4.68 (dd, 2H, J=7.6, 10.3 Hz), 4.5-4.6 (m, 1H), 4.3-4.4 (m, 4H), 4.1-4.2 (m, 2H), 3.4-4.0 (m, 10H), 2.8-2.9 (m, 2H), 2.44 (s, 3H), 1.36 (d, 3H, J=6.2 Hz).

### Example 113

### 8-[(2S,6R)-2-[[4-[2-[(3R,4R)-4-amino-3-fluoro-1-piperidyl]pyrimidin-4-yl]piperazin-1-yl]methyl]-6-methyl-morpholin-4-yl]quinoxaline-5-carbonitrile

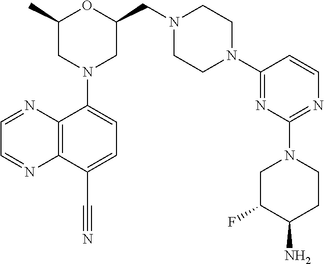 [SMILES: C[C@@H]1CN(c2ccc(C#N)c3nccnc23)C[C@H](CN2CCN(c3ccnc(N4CC[C@@H](N)[C@H](F)C4)n3)CC2)O1]

The title compound was prepared in analogy to the preparation of Example 23 by using ((2R,6R)-4-(8-cyanoquinoxalin-5-yl)-6-methylmorpholin-2-yl)methyl trifluoromethanesulfonate (Intermediate C) and tert-butyl N-[(3R,4R)-3-fluoro-4-piperidyl]carbamate (PharmaBlock, PB08067, CAS:1523530-29-1) instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 113 (17 mg) was obtained as a light yellow solid. MS: calc'd 547 (MH⁺), measured 547 (MH⁺). ¹H NMR (METHANOL-d₄, 400 MHz) δ 8.87 (d, 1H, J=1.8 Hz), 8.79 (d, 1H, J=1.7 Hz), 8.05 (d, 1H, J=8.3 Hz), 7.83 (d, 1H, J=7.3 Hz), 7.16 (d, 1H, J=8.4 Hz), 6.54 (d, 1H, J=7.3 Hz), 4.66 (dt, 1H, J=5.0, 9.7 Hz), 4.54 (dt, 1H, J=5.3, 9.8 Hz), 4.3-4.5 (m, 2H), 4.2-4.2 (m, 1H), 4.0-4.2 (m, 5H), 3.4-3.6 (m, 5H), 3.3-3.4 (m, 3H), 3.1-3.2 (m, 2H), 2.7-2.8 (m, 2H), 2.1-2.2 (m, 1H), 1.6-1.8 (m, 1H), 1.23 (d, 3H, J=6.1 Hz).

### Example 114

### 8-[(2R,6S)-2-methyl-6-[[4-(4-methyl-6-piperazin-1-yl-pyrimidin-2-yl)piperazin-1-yl]methyl]morpholin-4-yl]quinoxaline-5-carbonitrile

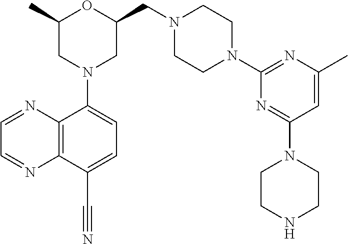 [SMILES: Cc1cc(N2CCNCC2)nc(N2CCN(C[C@H]3CN(c4ccc(C#N)c5nccnc45)C[C@@H](C)O3)CC2)n1]

The title compound was prepared in analogy to the preparation of Example 104 by using tert-butyl piperazine-1-carboxylate instead of tert-butyl N-[(3S,4S)-4-methoxypyrrolidin-3-yl]carbamate (compound 104e). Example 114 (13 mg) was obtained as a light yellow solid. MS: calc'd 529 (MH⁺), measured 529 (MH⁺). ¹H NMR (METHANOL-d₄, 400 MHz) δ 8.98 (d, 1H, J=1.8 Hz), 8.90 (d, 1H, J=1.7 Hz), 8.16 (d, 1H, J=8.3 Hz), 7.27 (d, 1H, J=8.3 Hz), 6.31 (s, 1H), 4.6-4.8 (m, 1H), 4.5-4.5 (m, 1H), 4.3-4.3 (m, 1H), 4.15 (br d, 8H, J=2.0 Hz), 3.6-3.9 (m, 6H), 3.5-3.5 (m, 1H), 3.5-3.5 (m, 3H), 3.4-3.5 (m, 2H), 2.8-2.9 (m, 2H), 2.46 (s, 3H), 1.34 (d, 3H, J=6.1 Hz).

### Example 115

### 8-[(2S,6R)-2-[[4-[4-[(3R,4S)-3-amino-4-fluoro-pyrrolidin-1-yl]-6-methyl-pyrimidin-2-yl]piperazin-1-yl]methyl]-6-methyl-morpholin-4-yl]quinoxaline-5-carbonitrile

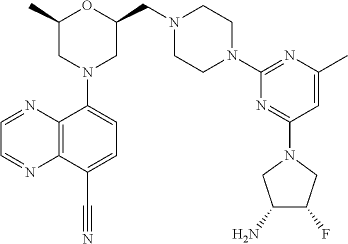 [SMILES: Cc1cc(N2C[C@@H](N)[C@@H](F)C2)nc(N2CCN(C[C@H]3CN(c4ccc(C#N)c5nccnc45)C[C@@H](C)O3)CC2)n1]

The title compound was prepared in analogy to the preparation of Example 104 by using tert-butyl N-[(3R,4S)-4-fluoropyrrolidin-3-yl]carbamate instead of tert-butyl N-[(3S,4S)-4-methoxypyrrolidin-3-yl]carbamate (compound 104e). Example 115 (22 mg) was obtained as a light yellow solid. MS: calc'd 547 (MH⁺), measured 547 (MH⁺). ¹H NMR (METHANOL-d₄, 400 MHz) δ 9.00 (d, 1H, J=1.8 Hz), 8.92 (d, 1H, J=1.7 Hz), 8.18 (d, 1H, J=8.3 Hz), 7.30 (d, 1H, J=8.3 Hz), 6.3-6.4 (m, 1H), 5.5-5.7 (m, 1H), 4.4-4.6 (m, 2H), 4.19 (br d, 9H, J=11.7 Hz), 3.6-3.8 (m, 4H), 3.4-3.6 (m, 4H), 2.8-2.9 (m, 2H), 2.48 (s, 3H), 1.36 (d, 3H, J=6.1 Hz).

### Example 116

### (2R,6S)-2-methyl-4-(8-methylquinoxalin-5-yl)-6-[[4-(4-piperazin-1-ylpyrimidin-2-yl)piperazin-1-yl]methyl]morpholine

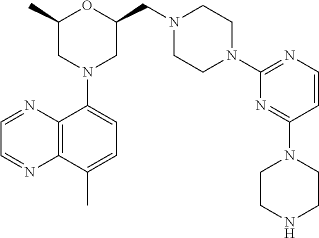 [SMILES: Cc1ccc(N2C[C@H](CN3CCN(c4nccc(N5CCNCC5)n4)CC3)O[C@H](C)C2)c2nccnc12]

The title compound was prepared in analogy to the preparation of Example 6 by using [(2R,6R)-6-methyl-4-(8-methylquinoxalin-5-yl)morpholin-2-yl]methyl trifluoromethanesulfonate (Intermediate G) and 4-chloro-2-piperazin-1-yl-pyrimidine and instead of [(2R,6R)-4-(8-cyano-5-quinolyl)-6-methyl-morpholin-2-yl]methyl trifluoromethanesulfonate (Intermediate A) and 2-bromo-5-(piperazin-1-yl)pyrazine (compound 6a). Example 116 (16 mg) was obtained as a light yellow solid. MS: calc'd 504 (MH⁺), measured 504 (MH⁺). ¹H NMR (METHANOL-d₄, 400 MHz) δ 8.87 (d, 1H, J=1.7 Hz), 8.81 (d, 1H, J=1.7 Hz), 7.84 (d, 1H, J=7.5 Hz), 7.6-7.6 (m, 1H), 7.40 (d, 1H, J=7.8 Hz), 6.62 (d, 1H, J=7.5 Hz), 4.5-4.6 (m, 2H), 3.9-4.3 (m, 6H), 3.5-3.9 (m, 6H), 3.3-3.5 (m, 8H), 2.8-2.9 (m, 2H), 2.64 (s, 3H), 1.25 (d, 3H, J=6.2 Hz).

### Example 117

### (2R,6S)-2-methyl-4-(8-methylquinoxalin-5-yl)-6-[[4-(2-piperazin-1-ylpyrimidin-4-yl)piperazin-1-yl]methyl]morpholine

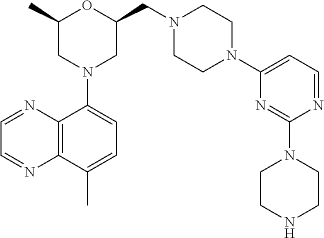 [SMILES: Cc1ccc(N2C[C@H](CN3CCN(c4ccnc(N5CCNCC5)n4)CC3)O[C@H](C)C2)c2nccnc12]

The title compound was prepared in analogy to the preparation of Example 23 by using [(2R,6R)-6-methyl-4-(8-methylquinoxalin-5-yl)morpholin-2-yl]methyl trifluoromethanesulfonate (Intermediate G) and piperazine-1-carboxylate instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 117 (9 mg) was obtained as a light yellow solid. MS: calc'd 504 (MH⁺), measured 504 (MH⁺). ¹H NMR (METHANOL-d₄, 400 MHz) δ 8.96 (d, 1H, J=1.6 Hz), 8.90 (d, 1H, J=1.6 Hz), 7.96 (d, 1H, J=7.5 Hz), 7.7-7.7 (m, 1H), 7.43 (d, 1H, J=7.8 Hz), 6.75 (d, 1H, J=7.5 Hz), 4.6-4.7 (m, 2H), 4.2-4.3 (m, 1H), 4.11 (br s, 5H), 3.7-3.9 (m, 4H), 3.4-3.6 (m, 9H), 2.8-2.9 (m, 2H), 2.75 (s, 3H), 1.3-1.4 (m, 4H).

### Example 118

### 8-[(2S,6R)-2-[[4-[4-(3-aminoazetidin-1-yl)pyrimidin-2-yl]piperazin-1-yl]methyl]-6-methyl-morpholin-4-yl]quinoxaline-5-carbonitrile

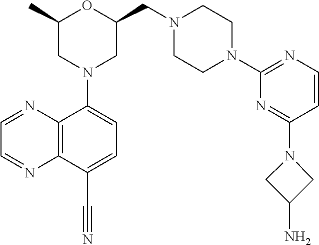 [SMILES: C[C@@H]1CN(c2ccc(C#N)c3nccnc23)C[C@H](CN2CCN(c3nccc(N4CC(N)C4)n3)CC2)O1]

The title compound was prepared in analogy to the preparation of Example 97 by using ((2R,6R)-4-(8-cyanoquinoxalin-5-yl)-6-methylmorpholin-2-yl)methyl trifluoromethanesulfonate (Intermediate C) and tert-butyl N-(azetidin-3-yl)carbamate instead of ((2R,6R)-4-(8-cyanoquinolin-5-yl)-6-methylmorpholin-2-yl)methyl trifluoromethanesulfonate (Intermediate A) and tert-butyl N-[(3S,4S)-4-methoxypyrrolidin-3-yl]carbamate (compound 97d). Example 118 (30 mg) was obtained as a light yellow solid. MS: calc'd 501 (MH⁺), measured 501 (MH⁺). ¹H NMR (METHANOL-d₄, 400 MHz) δ 8.98 (d, 1H, J=1.8 Hz), 8.90 (d, 1H, J=1.8 Hz), 8.15 (d, 1H, J=8.3 Hz), 7.87 (d, 1H, J=7.1 Hz), 7.26 (d, 1H, J=8.3 Hz), 6.22 (d, 1H, J=7.1 Hz), 4.6-4.7 (m, 2H), 4.4-4.5 (m, 1H), 4.3-4.4 (m, 4H), 4.0-4.2 (m, 6H), 3.5-3.7 (m, 4H), 3.4-3.5 (m, 2H), 2.8-2.9 (m, 2H), 1.34 (d, 3H, J=6.1 Hz).

### Example 119

### 8-[(2S,6R)-2-[[4-[4-[(3R,4R)-3-amino-4-fluoro-pyrrolidin-1-yl]pyrimidin-2-yl]piperazin-1-yl]methyl]-6-methyl-morpholin-4-yl]quinoxaline-5-carbonitrile

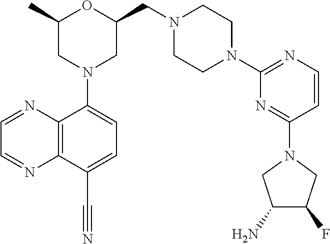 [SMILES: C[C@@H]1CN(c2ccc(C#N)c3nccnc23)C[C@H](CN2CCN(c3nccc(N4C[C@@H](N)[C@H](F)C4)n3)CC2)O1]

The title compound was prepared in analogy to the preparation of Example 97 by using ((2R,6R)-4-(8-cyanoquinoxalin-5-yl)-6-methylmorpholin-2-yl)methyl trifluoromethanesulfonate (Intermediate C) and tert-butyl N-[(3R,4R)-4-fluoropyrrolidin-3-yl]carbamate instead of ((2R,6R)-4-(8-cyanoquinolin-5-yl)-6-methylmorpholin-2-yl)methyl trifluoromethanesulfonate (Intermediate A) and tert-butyl N-[(3S,4S)-4-methoxypyrrolidin-3-yl]carbamate (compound 97d). Example 119 (29 mg) was obtained as a light yellow solid. MS: calc'd 533 (MH⁺), measured 533 (MH⁺). ¹H NMR (METHANOL-d₄, 400 MHz) δ 9.00 (d, 1H, J=1.8 Hz), 8.91 (d, 1H, J=1.7 Hz), 8.18 (d, 1H, J=8.3 Hz), 7.97 (d, 1H, J=6.8 Hz), 7.28 (d, 1H, J=8.3 Hz), 6.31 (d, 1H, J=6.8 Hz), 5.4-5.6 (m, 1H), 4.4-4.5 (m, 1H), 4.2-4.3 (m, 3H), 3.9-4.2 (m, 9H), 3.5-3.6 (m, 4H), 3.4-3.4 (m, 2H), 2.8-2.9 (m, 2H), 1.35 (d, 3H, J=6.1 Hz).

### Example 120

### 8-[(2S,6R)-2-[[4-[4-[(3S,4R)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile

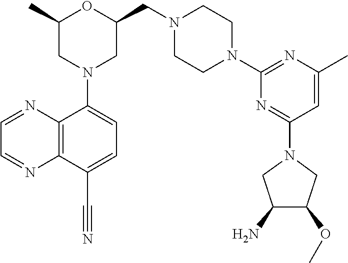 [SMILES: CO[C@@H]1CN(c2cc(C)nc(N3CCN(C[C@H]4CN(c5ccc(C#N)c6nccnc56)C[C@@H](C)O4)CC3)n2)C[C@@H]1N]

The title compound was prepared in analogy to the preparation of Example 104 by using tert-butyl N-[(3S,4R)-4-methoxypyrrolidin-3-yl]carbamate instead of tert-butyl N-[(3S,4S)-4-methoxypyrrolidin-3-yl]carbamate (compound 104e). Example 120 (18 mg) was obtained as a light yellow solid. MS: calc'd 559 (MH⁺), measured 559 (MH⁺). ¹H NMR (METHANOL-d₄, 400 MHz) δ 8.99 (d, 1H, J=1.8 Hz), 8.91 (d, 1H, J=1.7 Hz), 8.17 (d, 1H, J=8.3 Hz), 7.28 (d, 1H, J=8.3 Hz), 6.28 (br s, 1H), 4.4-4.5 (m, 1H), 4.31 (br d, 2H, J=11.9 Hz), 4.0-4.3 (m, 8H), 3.8-4.0 (m, 1H), 3.79 (dd, 1H, J=4.3, 13.1 Hz), 3.60 (br s, 5H), 3.52 (s, 3H), 3.4-3.5 (m, 2H), 2.86 (dt, 2H, J=10.8, 12.8 Hz), 2.44 (s, 3H), 1.34 (d, 3H, J=6.1 Hz).

### Example 121

### 8-[(2S,6R)-2-[[4-[4-[(3R,4R)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile

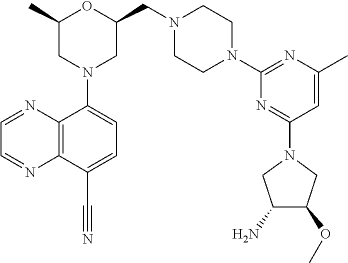 [SMILES: CO[C@@H]1CN(c2cc(C)nc(N3CCN(C[C@H]4CN(c5ccc(C#N)c6nccnc56)C[C@@H](C)O4)CC3)n2)C[C@H]1N]

The title compound was prepared in analogy to the preparation of Example 104 by using tert-butyl N-[(3R,4R)-4-methoxypyrrolidin-3-yl]carbamate instead of tert-butyl N-[(3S,4S)-4-methoxypyrrolidin-3-yl]carbamate (compound 104e). Example 121 (19 mg) was obtained as a light yellow solid. MS: calc'd 559 (MH⁺), measured 559 (MH⁺). ¹H NMR (METHANOL-d₄, 400 MHz) δ 9.00 (d, 1H, J=1.7 Hz), 8.91 (d, 1H, J=1.7 Hz), 8.17 (d, 1H, J=8.3 Hz), 7.28 (d, 1H, J=8.3 Hz), 6.27 (s, 1H), 4.4-4.5 (m, 1H), 4.31 (br d, 1H, J=12.2 Hz), 4.1-4.3 (m, 6H), 4.0-4.1 (m, 4H), 3.7-3.9 (m, 2H), 3.5-3.7 (m, 4H), 3.50 (s, 3H), 3.4-3.4 (m, 2H), 2.8-2.9 (m, 2H), 2.43 (s, 3H), 1.35 (d, 3H, J=6.2 Hz).

### Example 122

### 8-[(2S,6R)-2-[[4-[4-[(3R,4S)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile

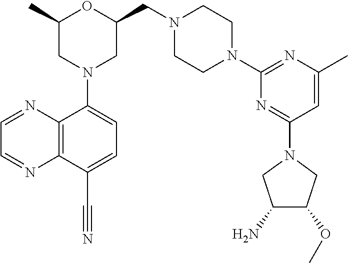 [SMILES: CO[C@H]1CN(c2cc(C)nc(N3CCN(C[C@H]4CN(c5ccc(C#N)c6nccnc56)C[C@@H](C)O4)CC3)n2)C[C@H]1N]

The title compound was prepared in analogy to the preparation of Example 104 by using tert-butyl N-[(3R,4S)-4-methoxypyrrolidin-3-yl]carbamate instead of tert-butyl N-[(3S,4S)-4-methoxypyrrolidin-3-yl]carbamate (compound 104e). Example 122 (19 mg) was obtained as a light yellow solid. MS: calc'd 559 (MH⁺), measured 559 (MH⁺). ¹H NMR (METHANOL-d₄, 400 MHz) δ 8.98 (d, 1H, J=1.5 Hz), 8.9-8.9 (m, 1H), 8.15 (dd, 1H, J=2.2, 8.3 Hz), 7.26 (d, 1H, J=8.3 Hz), 6.29 (br s, 1H), 4.48 (br t, 1H, J=9.8 Hz), 4.31 (br d, 2H, J=12.0 Hz), 4.0-4.3 (m, 8H), 3.91 (br d, 1H, J=11.4 Hz), 3.7-3.8 (m, 2H), 3.6-3.7 (m, 4H), 3.52 (s, 3H), 3.4-3.5 (m, 2H), 2.8-2.9 (m, 2H), 2.44 (s, 3H), 1.34 (d, 3H, J=6.2 Hz).

### Example 123

### 8-[(2S,6R)-2-[[4-[4-(3-aminoazetidin-1-yl)-6-methyl-pyrimidin-2-yl]piperazin-1-yl]methyl]-6-methyl-morpholin-4-yl]quinoxaline-5-carbonitrile

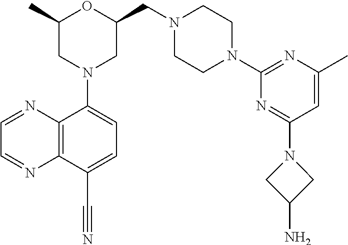 [SMILES: Cc1cc(N2CC(N)C2)nc(N2CCN(C[C@H]3CN(c4ccc(C#N)c5nccnc45)C[C@@H](C)O3)CC2)n1]

The title compound was prepared in analogy to the preparation of Example 104 by using tert-butyl N-(azetidin-3-yl)carbamate instead of tert-butyl N-[(3S,4S)-4-methoxypyrrolidin-3-yl]carbamate (compound 104e). Example 123 (15 mg) was obtained as a light yellow solid. MS: calc'd 515 (MH⁺), measured 515 (MH⁺). ¹H NMR (METHANOL-d₄, 400 MHz) δ 9.00 (d, 1H, J=1.7 Hz), 8.91 (d, 1H, J=1.8 Hz), 8.17 (d, 1H, J=8.2 Hz), 7.28 (d, 1H, J=8.3 Hz), 6.07 (s, 1H), 4.6-4.6 (m, 2H), 4.4-4.5 (m, 1H), 4.3-4.4 (m, 4H), 3.9-4.2 (m, 6H), 3.5-3.6 (m, 4H), 3.4-3.4 (m, 2H), 2.8-2.9 (m, 2H), 2.39 (s, 3H), 1.34 (d, 3H, J=6.1 Hz).

### Example 124

### 8-[(2S,6R)-2-[[4-[4-(3-aminoazetidin-1-yl)-6-methyl-pyrimidin-2-yl]piperazin-1-yl]methyl]-6-methyl-morpholin-4-yl]-2,3-dideuterio-quinoxaline-5-carbonitrile

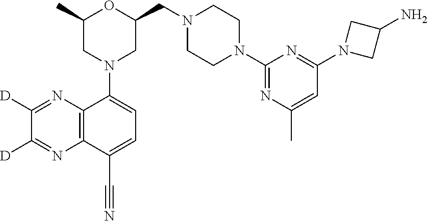 [SMILES: [2H]c1nc2c(C#N)ccc(N3C[C@H](CN4CCN(c5nc(C)cc(N6CC(N)C6)n5)CC4)O[C@H](C)C3)c2nc1[2H]]

The title compound was prepared in analogy to the preparation of Example 104 by using [(2R,6R)-4-(8-cyano-2,3-dideuterio-quinoxalin-5-yl)-6-methyl-morpholin-2-yl]methyl trifluoromethanesulfonate (Intermediate K) and tert-butyl N-(azetidin-3-yl)carbamate instead of ((2R,6R)-4-(8-cyanoquinoxalin-5-yl)-6-methylmorpholin-2-yl)methyl trifluoromethanesulfonate (Intermediate C) and tert-butyl N-[(3S,4S)-4-methoxypyrrolidin-3-yl]carbamate (compound 104e). Example 124 (18 mg) was obtained as a light yellow solid. MS: calc'd 517 (MH⁺), measured 517 (MH⁺). ¹H NMR (METHANOL-d₄, 400 MHz) δ 8.12 (d, 1H, J=8.4 Hz), 7.21 (d, 1H, J=8.4 Hz), 5.57 (s, 1H), 4.5-4.6 (m, 1H), 4.1-4.4 (m, 5H), 3.8-4.1 (m, 2H), 3.6-3.8 (m, 6H), 2.5-2.8 (m, 6H), 2.17 (s, 3H), 1.2-1.4 (m, 4H).

### Example 125

### 8-[(2S,6R)-2-[[4-[4-[(3R,4R)-4-amino-3-hydroxy-3-methyl-pyrrolidin-1-yl]-6-methyl-pyrimidin-2-yl]piperazin-1-yl]methyl]-6-methyl-morpholin-4-yl]quinoxaline-5-carbonitrile

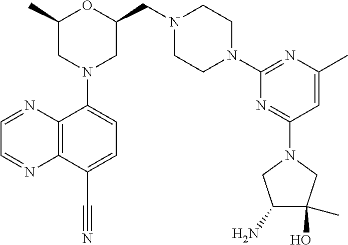 [SMILES: Cc1cc(N2C[C@@H](N)[C@](C)(O)C2)nc(N2CCN(C[C@H]3CN(c4ccc(C#N)c5nccnc45)C[C@@H](C)O3)CC2)n1]

The title compound was prepared in analogy to the preparation of Example 104 by using tert-butyl N-[(3R,4R)-4-hydroxy-4-methyl-pyrrolidin-3-yl]carbamate instead of tert-butyl N-[(3S,4S)-4-methoxypyrrolidin-3-yl]carbamate (compound 104e). Example 125 (11 mg) was obtained as a light yellow solid. MS: calc'd 559 (MH⁺), measured 559 (MH⁺). ¹H NMR (METHANOL-d₄, 400 MHz) δ 8.99 (d, 1H, J=1.8 Hz), 8.91 (d, 1H, J=1.8 Hz), 8.17 (d, 1H, J=8.3 Hz), 7.28 (d, 1H, J=8.4 Hz), 6.2-6.3 (m, 1H), 4.4-4.5 (m, 1H), 4.3-4.3 (m, 1H), 4.1-4.2 (m, 6H), 3.6-3.8 (m, 3H), 3.5-3.6 (m, 4H), 3.4-3.5 (m, 3H), 2.8-2.9 (m, 2H), 2.4-2.4 (m, 3H), 1.53 (s, 2H), 1.4-1.4 (m, 2H), 1.3-1.4 (m, 3H).

### Example 126

### 2-[4-[4-[[(2S,6R)-6-methyl-4-(8-methylquinoxalin-5-yl)morpholin-2-yl]methyl]piperazin-1-yl]pyrimidin-2-yl]-5-oxa-2,8-diazaspiro[3.5]nonane

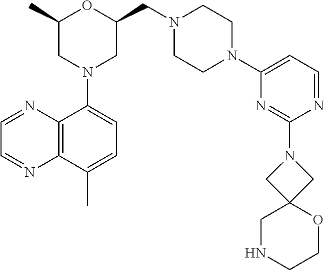 [SMILES: Cc1ccc(N2C[C@H](CN3CCN(c4ccnc(N5CC6(CNCCO6)C5)n4)CC3)O[C@H](C)C2)c2nccnc12]

The title compound was prepared in analogy to the preparation of Example 23 by using [(2R,6R)-6-methyl-4-(8-methylquinoxalin-5-yl)morpholin-2-yl]methyl trifluoromethanesulfonate (Intermediate G) and tert-butyl 5-oxa-2,8-diazaspiro[3.5]nonane-8-carboxylate (PharmaBlock, PBN20111065, CAS: 1251005-61-4) instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 126 (11 mg) was obtained as a light yellow solid. MS: calc'd 546 (MH⁺), measured 546 (MH⁺). ¹H NMR (METHANOL-d₄, 400 MHz) δ 8.89 (d, 1H, J=1.7 Hz), 8.83 (d, 1H, J=1.8 Hz), 7.89 (d, 1H, J=7.5 Hz), 7.6-7.6 (m, 1H), 7.23 (d, 1H, J=7.9 Hz), 6.62 (d, 1H, J=7.5 Hz), 4.4-4.5 (m, 1H), 4.37 (d, 2H, J=10.3 Hz), 4.2-4.3 (m, 3H), 4.1-4.2 (m, 3H), 3.9-4.0 (m, 3H), 3.78 (br d, 1H, J=11.2 Hz), 3.69 (br d, 1H, J=11.5 Hz), 3.5-3.6 (m, 6H), 3.4-3.4 (m, 2H), 3.3-3.3 (m, 2H), 2.69 (s, 3H), 2.63 (dd, 2H, J=10.5, 11.1 Hz), 1.31 (d, 3H, J=6.4 Hz).

### Example 127

### (2R,6S)-2-methyl-6-[[4-[2-[(3S)-3-methylpiperazin-1-yl]pyrimidin-4-yl]piperazin-1-yl]methyl]-4-(8-methylquinoxalin-5-yl)morpholine

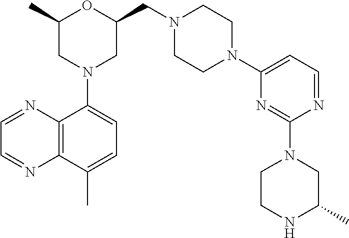 [SMILES: Cc1ccc(N2C[C@H](CN3CCN(c4ccnc(N5CCN[C@@H](C)C5)n4)CC3)O[C@H](C)C2)c2nccnc12]

The title compound was prepared in analogy to the preparation of Example 23 by using [(2R,6R)-6-methyl-4-(8-methylquinoxalin-5-yl)morpholin-2-yl]methyl trifluoromethanesulfonate (Intermediate G) and tert-butyl (2S)-2-methylpiperazine-1-carboxylate (Bidepharm, BD12408, CAS:169447-70-5) instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 127 (9 mg) was obtained as a light yellow solid. MS: calc'd 518 (MH⁺), measured 518 (MH⁺). ¹H NMR (METHANOL-d₄, 400 MHz) δ 8.89 (d, 1H, J=1.7 Hz), 8.83 (d, 1H, J=1.7 Hz), 7.98 (d, 1H, J=7.1 Hz), 7.6-7.6 (m, 1H), 7.23 (d, 1H, J=7.8 Hz), 6.57 (d, 1H, J=7.1 Hz), 4.5-4.7 (m, 3H), 4.0-4.3 (m, 4H), 3.78 (br d, 1H, J=11.4 Hz), 3.69 (br d, 1H, J=11.6 Hz), 3.3-3.7 (m, 10H), 3.2-3.3 (m, 2H), 2.69 (s, 3H), 2.6-2.7 (m, 2H), 1.41 (d, 3H, J=6.6 Hz), 1.31 (d, 3H, J=6.2 Hz).

### Example 128

### (2R,6S)-2-methyl-6-[[4-[2-[(3R)-3-methylpiperazin-1-yl]pyrimidin-4-yl]piperazin-1-yl]methyl]-4-(8-methylquinoxalin-5-yl)morpholine

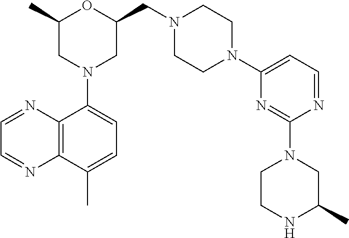 [SMILES: Cc1ccc(N2C[C@H](CN3CCN(c4ccnc(N5CCN[C@H](C)C5)n4)CC3)O[C@H](C)C2)c2nccnc12]

The title compound was prepared in analogy to the preparation of Example 23 by using [(2R,6R)-6-methyl-4-(8-methylquinoxalin-5-yl)morpholin-2-yl]methyl trifluoromethanesulfonate (Intermediate G) and tert-butyl (2R)-2-methylpiperazine-1-carboxylate (Accela ChemBio Inc, SY007943, CAS:170033-47-3) instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 128 (12 mg) was obtained as a light yellow solid. MS: calc'd 518 (MH⁺), measured 518 (MH⁺). ¹H NMR (METHANOL-d₄, 400 MHz) δ 8.89 (d, 1H, J=1.8 Hz), 8.83 (d, 1H, J=1.8 Hz), 7.97 (d, 1H, J=7.2 Hz), 7.6-7.6 (m, 1H), 7.23 (d, 1H, J=7.8 Hz), 6.62 (d, 1H, J=7.2 Hz), 4.5-4.6 (m, 3H), 3.9-4.4 (m, 5H), 3.78 (br d, 1H, J=11.4 Hz), 3.69 (br d, 1H, J=11.5 Hz), 3.4-3.7 (m, 9H), 3.2-3.3 (m, 2H), 2.69 (s, 3H), 2.6-2.7 (m, 2H), 1.41 (d, 3H, J=6.5 Hz), 1.31 (d, 3H, J=6.2 Hz).

### Example 129

### (4aR,7aR)-6-[4-[4-[[(2S,6R)-6-methyl-4-(8-methylquinoxalin-5-yl)morpholin-2-yl]methyl]piperazin-1-yl]pyrimidin-2-yl]-3,4,4a,5,7,7a-hexahydro-2H-pyrrolo[3,4-b][1,4]oxazine

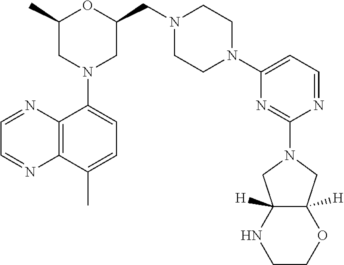 [SMILES: Cc1ccc(N2C[C@H](CN3CCN(c4ccnc(N5C[C@H]6NCCO[C@@H]6C5)n4)CC3)O[C@H](C)C2)c2nccnc12]

The title compound was prepared in analogy to the preparation of Example 23 by using [(2R,6R)-6-methyl-4-(8-methylquinoxalin-5-yl)morpholin-2-yl]methyl trifluoromethanesulfonate (Intermediate G) and tert-butyl (4aR,7aR)-3,4a,5,6,7,7a-hexahydro-2H-pyrrolo[3,4-b][1,4]oxazine-4-carboxylate (Pharmablock, PBXA8123, CAS: 1932337-68-2) instead of ((2R,6R)-4-(8-cyanoquinolin-5-yl)-6-methylmorpholin-2-yl)methyl trifluoromethanesulfonate (Intermediate A) and tert-butyl azetidin-3-ylcarbamate (compound 23c). Example 129 (10 mg) was obtained as a light yellow solid. MS: calc'd 546 (MH⁺), measured 546 (MH⁺). ¹H NMR (METHANOL-d₄, 400 MHz) δ 8.89 (d, 1H, J=1.7 Hz), 8.83 (d, 1H, J=1.8 Hz), 7.91 (d, 1H, J=7.5 Hz), 7.6-7.6 (m, 1H), 7.23 (d, 1H, J=7.8 Hz), 6.67 (d, 1H, J=7.6 Hz), 4.5-4.5 (m, 1H), 4.27 (br dd, 2H, J=3.7, 13.2 Hz), 4.1-4.2 (m, 5H), 4.01 (dt, 2H, J=2.5, 12.7 Hz), 3.78 (br d, 1H, J=11.4 Hz), 3.4-3.7 (m, 13H), 2.69 (s, 3H), 2.63 (t, 2H, J=10.8 Hz), 1.31 (d, 3H, J=6.2 Hz).

### Example 130

### 1-[4-[4-[[(2S,6R)-6-methyl-4-(8-methylquinoxalin-5-yl)morpholin-2-yl]methyl]piperazin-1-yl]pyrimidin-2-yl]azetidin-3-amine

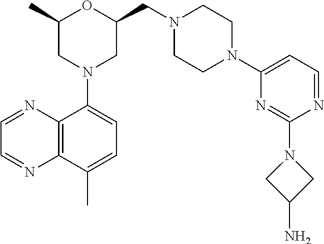 [SMILES: Cc1ccc(N2C[C@H](CN3CCN(c4ccnc(N5CC(N)C5)n4)CC3)O[C@H](C)C2)c2nccnc12]

The title compound was prepared in analogy to the preparation of Example 23 by using [(2R,6R)-6-methyl-4-(8-methylquinoxalin-5-yl)morpholin-2-yl]methyl trifluoromethanesulfonate (Intermediate G) instead of ((2R,6R)-4-(8-cyanoquinolin-5-yl)-6-methylmorpholin-2-yl)methyl trifluoromethanesulfonate (Intermediate A). Example 130 (10 mg) was obtained as a light yellow solid. MS: calc'd 490 (MH⁺), measured 490 (MH⁺). ¹H NMR (METHANOL-d₄, 400 MHz) δ 8.89 (d, 1H, J=1.8 Hz), 8.83 (d, 1H, J=1.8 Hz), 7.90 (d, 1H, J=7.6 Hz), 7.60 (d, 1H, J=7.8 Hz), 7.23 (d, 1H, J=7.8 Hz), 6.63 (d, 1H, J=7.5 Hz), 4.6-4.7 (m, 2H), 4.5-4.5 (m, 1H), 4.3-4.4 (m, 4H), 4.0-4.3 (m, 4H), 3.8-3.8 (m, 1H), 3.7-3.7 (m, 1H), 3.5-3.7 (m, 4H), 3.4-3.4 (m, 2H), 2.69 (s, 3H), 2.63 (t, 2H, J=10.8 Hz), 1.30 (d, 3H, J=6.2 Hz).

### Example 131

### 5-[(2S,6R)-2-[[4-[4-(3-amino-3-methyl-azetidin-1-yl)-6-methoxy-pyrimidin-2-yl]piperazin-1-yl]methyl]-6-methyl-morpholin-4-yl]-2-deuterio-quinoline-8-carbonitrile

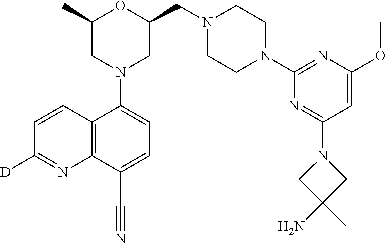 [SMILES: [2H]c1ccc2c(N3C[C@H](CN4CCN(c5nc(OC)cc(N6CC(C)(N)C6)n5)CC4)O[C@H](C)C3)ccc(C#N)c2n1]

The title compound was prepared in analogy to the preparation of Example 104 by using [(2R,6R)-4-(8-cyano-2-deuterio-5-quinolyl)-6-methyl-morpholin-2-yl]methyl trifluoromethanesulfonate (intermediate L) and 2,4-dichloro-6-methoxy-pyrimidine and tert-butyl N-(3-methylazetidin-3-yl)carbamate instead of [(2R,6R)-4-(8-cyanoquinoxalin-5-yl)-6-methyl-morpholin-2-yl]methyl trifluoromethanesulfonate (intermediate C) and 2,4-dichloro-6-methylpyrimidine and tert-butyl N-[(3S,4S)-4-methoxypyrrolidin-3-yl]carbamate (compound 104e). Example 131 (29 mg) was obtained as a light yellow solid. MS: calc'd 545 (MH⁺), measured 545 (MH⁺). ¹H NMR (METHANOL-d₄, 400 MHz) δ 8.66 (d, 1H, J=8.7 Hz), 8.16 (d, 1H, J=7.9 Hz), 7.65 (d, 1H, J=8.7 Hz), 7.28 (d, 1H, J=8.1 Hz), 4.5-4.5 (m, 1H), 4.2-4.2 (m, 1H), 4.1-4.1 (m, 2H), 4.0-4.0 (m, 2H), 3.85 (s, 3H), 3.5-3.8 (m, 2H), 3.4-3.5 (m, 5H), 3.3-3.3 (m, 5H), 3.2-3.3 (m, 1H), 2.7-2.8 (m, 2H), 1.66 (s, 3H), 1.32 (d, 3H, J=6.2 Hz).

### Example 132

### 5-[(2S,6R)-2-[[4-[6-(3-amino-3-methyl-azetidin-1-yl)-3-pyridyl]piperazin-1-yl]methyl]-6-methyl-morpholin-4-yl]-2-deuterio-quinoline-8-carbonitrile

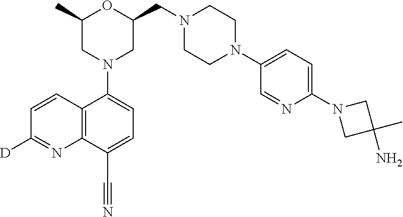 [SMILES: [2H]c1ccc2c(N3C[C@H](CN4CCN(c5ccc(N6CC(C)(N)C6)nc5)CC4)O[C@H](C)C3)ccc(C#N)c2n1]

The title compound was prepared according to the following scheme:

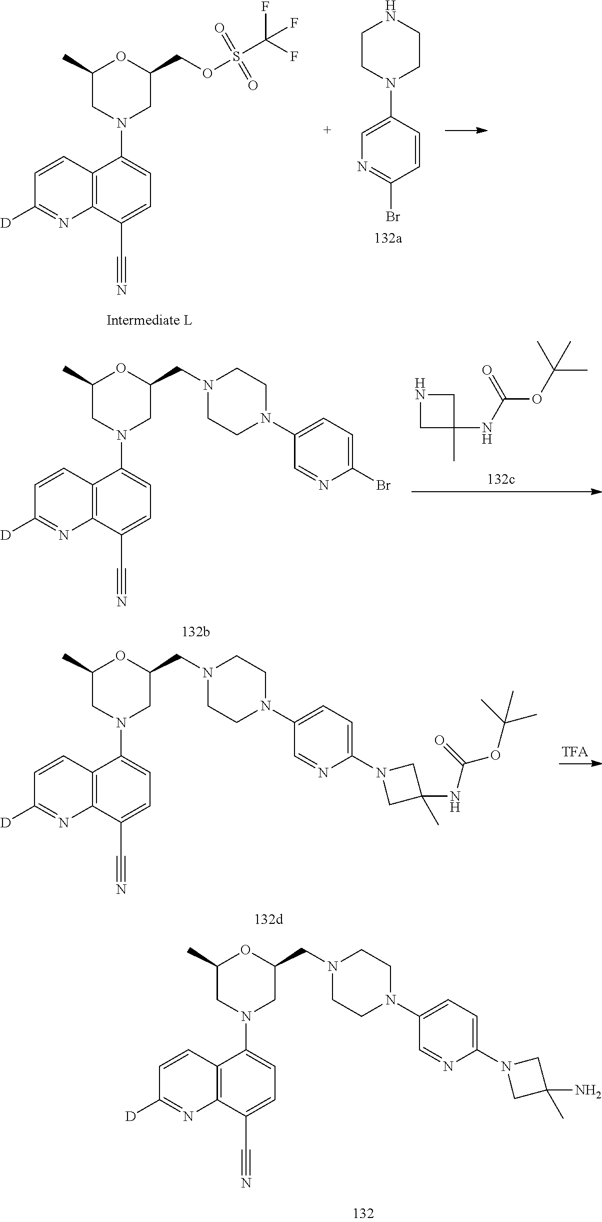 [SMILES: [2H]c1ccc2c(N3C[C@H](COS(=O)(=O)C(F)(F)F)O[C@H](C)C3)ccc(C#N)c2n1 ; Brc1ccc(N2CCNCC2)cn1 ; [2H]c1ccc2c(N3C[C@H](CN4CCN(c5ccc(Br)nc5)CC4)O[C@H](C)C3)ccc(C#N)c2n1 ; CC1(NC(=O)OC(C)(C)C)CNC1 ; [2H]c1ccc2c(N3C[C@H](CN4CCN(c5ccc(N6CC(C)(NC(=O)OC(C)(C)C)C6)nc5)CC4)O[C@H](C)C3)ccc(C#N)c2n1 ; [2H]c1ccc2c(N3C[C@H](CN4CCN(c5ccc(N6CC(C)(N)C6)nc5)CC4)O[C@H](C)C3)ccc(C#N)c2n1]

### Step 1: 2-deuterio-5-[(2S,6R)-2-[[4-(6-bromo-3-pyridyl)piperazin-1-yl]methyl]-6-methyl-morpholin-4-yl]quinoline-8-carbonitrile (Compound 132b)

A solution of 1-(6-bromopyridin-3-yl)piperazine (compound 132a, 1.5 g, 6.2 mmol) and ((2R,6R)-4-(8-cyanoquinolin-5-yl-2-d)-6-methylmorpholin-2-yl)methyl trifluoromethanesulfonate (Intermediate L, 2.58 g, 6.2 mmol) and DIPEA (2.4 g, 3.25 mL) in CH₂Cl₂ (20 mL) was stirred at room temperature for 2 hrs. The mixture was then diluted with DCM and washed with brine. The organic layer was dried over Na₂SO₄ and concentrated. The residue was purified by silica gel chromatography (EA/PE from 0% to 100%) to get 2-deuterio-5-[(2S,6R)-2-[[4-(6-bromo-3-pyridyl)piperazin-1-yl]methyl]-6-methyl-morpholin-4-yl]quinoline-8-carbonitrile (compound 132b, 2.96 g) as a yellow solid. MS: calc'd 508 (MH⁺), measured 508 (MH⁺).

### Step 2: tert-butyl N-[3-methyl-1-[5-[4-[[(2S,6R)-4-(8-cyano-2-deuterio-5-quinolyl)-6-methyl-morpholin-2-yl]methyl]piperazin-1-yl]-2-pyridyl]azetidin-3-yl]carbamate (Compound 132d)

To a solution of 2-deuterio-5-[(2S,6R)-2-[[4-(6-bromo-3-pyridyl)piperazin-1-yl]methyl]-6-methyl-morpholin-4-yl]quinoline-8-carbonitrile (compound 132b, 2.4 g, 4.72 mmol) in dioxane (60 mL) was added RuPhos-Pd-G2 (367 mg, 472 μmol), tert-butyl (3-methylazetidin-3-yl)carbamate (compound 132c, 1.32 g, 7.08 mmol) and Cs₂CO₃ (4.61 g, 14.2 mmol). The reaction mixture was stirred for 16 hours at 120° C. under N₂. Then the mixture was filtered and the filtrate was concentrated under reduced pressure to afford crude product, which was purified by silica gel chromatography (MeOH/DCM from 2% to 12%) to afford tert-butyl N-[3-methyl-1-[5-[4-[[(2S,6R)-4-(8-cyano-2-deuterio-5-quinolyl)-6-methyl-morpholin-2-yl]methyl]piperazin-1-yl]-2-pyridyl]azetidin-3-yl]carbamate (compound 132d, 2.4 g) as a light yellow solid. MS: calc'd 614 (MH⁺), measured 614 (MH⁺).

### Step 3: 5-[(2S,6R)-2-[[4-[6-(3-amino-3-methyl-azetidin-1-yl)-3-pyridyl]piperazin-1-yl]methyl]-6-methyl-morpholin-4-yl]-2-deuterio-quinoline-8-carbonitrile (Example 132)

To a solution of tert-butyl N-[3-methyl-1-[5-[4-[[(2S,6R)-4-(8-cyano-2-deuterio-5-quinolyl)-6-methyl-morpholin-2-yl]methyl]piperazin-1-yl]-2-pyridyl]azetidin-3-yl]carbamate (compound 132d, 360 mg) in DCM (10 mL) was added TFA (2 mL) at 0° C. The reaction mixture was stirred at r.t. for 2 hrs. Then the mixture was concentrated to crude product, which was purified by prep-HPLC to afford Example 132 (102 mg) as a yellow solid. MS calc'd 514 (MH⁺), measured 514 (MH⁺). ¹H NMR (METHANOL-d₄, 400 MHz) δ 8.68 (d, 1H, J=8.6 Hz), 8.18 (d, 1H, J=8.1 Hz), 7.90 (dd, 1H, J=2.7, 9.4 Hz), 7.6-7.7 (m, 2H), 7.30 (d, 1H, J=8.1 Hz), 6.86 (d, 1H, J=9.4 Hz), 4.5-4.6 (m, 1H), 4.3-4.4 (m, 2H), 4.2-4.3 (m, 3H), 3.5-3.8 (m, 5H), 3.4-3.5 (m, 6H), 2.7-2.9 (m, 2H), 1.73 (s, 3H), 1.4-1.5 (m, 1H), 1.33 (d, 3H, J=6.2 Hz).

### Example 133

### 5-[(2S,6R)-2-[[4-[6-(3-amino-3-methyl-azetidin-1-yl)-4-methyl-3-pyridyl]piperazin-1-yl]methyl]-6-methyl-morpholin-4-yl]-2-deuterio-quinoline-8-carbonitrile

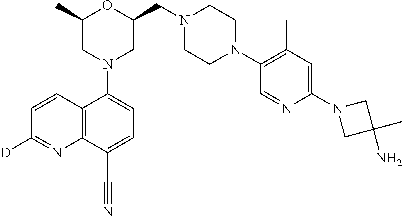 [SMILES: [2H]c1ccc2c(N3C[C@H](CN4CCN(c5cnc(N6CC(C)(N)C6)cc5C)CC4)O[C@H](C)C3)ccc(C#N)c2n1]

The title compound was prepared in analogy to the preparation of Example 132 by using 1-(6-bromo-4-methyl-3-pyridyl)piperazine instead of 1-(6-bromopyridin-3-yl)piperazine (compound 132a). Example 133 (3 mg) was obtained as a light yellow solid. MS: calc'd 528 (MH⁺), measured 528 (MH⁺). ¹H NMR (METHANOL-d₄, 400 MHz) δ 8.67 (d, 1H, J=8.6 Hz), 8.17 (d, 1H, J=8.1 Hz), 7.75 (s, 1H), 7.65 (d, 1H, J=8.6 Hz), 7.29 (d, 1H, J=8.1 Hz), 6.81 (s, 1H), 4.52 (dt, 1H, J=2.9, 8.2 Hz), 4.40 (d, 2H, J=9.9 Hz), 4.3-4.3 (m, 2H), 4.1-4.2 (m, 1H), 3.7-4.0 (m, 2H), 3.4-3.5 (m, 6H), 3.1-3.3 (m, 4H), 2.7-2.8 (m, 2H), 2.47 (s, 3H), 1.72 (s, 3H), 1.32 (d, 3H, J=6.2 Hz).

### Example 134

### 5-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]-2-deuterio-quinoline-8-carbonitrile

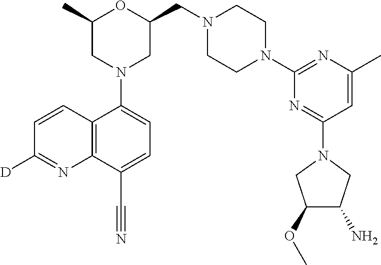 [SMILES: [2H]c1ccc2c(N3C[C@H](CN4CCN(c5nc(C)cc(N6C[C@H](OC)[C@@H](N)C6)n5)CC4)O[C@H](C)C3)ccc(C#N)c2n1]

The title compound was prepared in analogy to the preparation of Example 104 by using [(2R,6R)-4-(8-cyano-2-deuterio-5-quinolyl)-6-methyl-morpholin-2-yl]methyl trifluoromethanesulfonate (intermediate L) instead of [(2R,6R)-4-(8-cyanoquinoxalin-5-yl)-6-methyl-morpholin-2-yl]methyl trifluoromethanesulfonate (intermediate C). Example 134 (100 mg) was obtained as a light yellow solid. MS: calc'd 559 (MH⁺), measured 559 (MH⁺). ¹H NMR (METHANOL-d₄, 400 MHz) δ 8.67 (d, 1H, J=8.6 Hz), 8.15 (d, 1H, J=7.9 Hz), 7.64 (d, 1H, J=8.7 Hz), 7.25 (d, 1H, J=8.1 Hz), 5.70 (s, 1H), 4.2-4.3 (m, 1H), 4.0-4.1 (m, 1H), 3.7-3.8 (m, 6H), 3.6-3.7 (m, 1H), 3.4-3.5 (m, 3H), 3.40 (s, 3H), 3.4-3.4 (m, 1H), 2.5-2.7 (m, 9H), 2.19 (s, 3H), 1.26 (d, 3H, J=6.4 Hz).

### Example 135

### 5-[(2S,6R)-2-[[4-[4-(3-amino-3-methyl-azetidin-1-yl)-6-methyl-pyrimidin-2-yl]piperazin-1-yl]methyl]-6-methyl-morpholin-4-yl]-2-deuterio-quinoline-8-carbonitrile

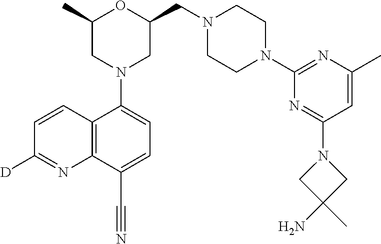 [SMILES: [2H]c1ccc2c(N3C[C@H](CN4CCN(c5nc(C)cc(N6CC(C)(N)C6)n5)CC4)O[C@H](C)C3)ccc(C#N)c2n1]

The title compound was prepared in analogy to the preparation of Example 104 by using [(2R,6R)-4-(8-cyano-2-deuterio-5-quinolyl)-6-methyl-morpholin-2-yl]methyl trifluoromethanesulfonate (intermediate L) and tert-butyl N-(3-methylazetidin-3-yl)carbamate instead of [(2R,6R)-4-(8-cyanoquinoxalin-5-yl)-6-methyl-morpholin-2-yl]methyl trifluoromethanesulfonate (intermediate C) and tert-butyl N-[(3S,4S)-4-methoxypyrrolidin-3-yl]carbamate (compound 104e). Example 135 (80 mg) was obtained as a light yellow solid. MS: calc'd 529 (MH⁺), measured 529 (MH⁺). ¹H NMR (METHANOL-d₄, 400 MHz) δ 8.66 (d, 1H, J=8.6 Hz), 8.15 (d, 1H, J=7.9 Hz), 7.64 (d, 1H, J=8.6 Hz), 7.25 (d, 1H, J=8.1 Hz), 5.57 (s, 1H), 4.2-4.2 (m, 1H), 4.0-4.1 (m, 1H), 3.8-3.9 (m, 2H), 3.7-3.8 (m, 6H), 3.46 (br d, 1H, J=12.5 Hz), 3.38 (br d, 1H, J=11.9 Hz), 2.5-2.7 (m, 8H), 2.16 (s, 3H), 1.46 (s, 3H), 1.25 (d, 3H, J=6.2 Hz).

### Example 136

### 5-[(2S,6R)-2-[[4-[4-(3-aminoazetidin-1-yl)-6-methyl-pyrimidin-2-yl]piperazin-1-yl]methyl]-6-methyl-morpholin-4-yl]-2-deuterio-quinoline-8-carbonitrile

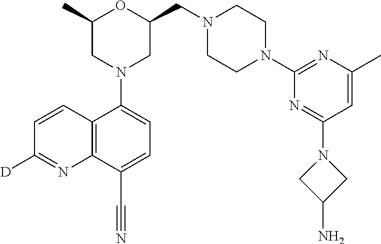 [SMILES: [2H]c1ccc2c(N3C[C@H](CN4CCN(c5nc(C)cc(N6CC(N)C6)n5)CC4)O[C@H](C)C3)ccc(C#N)c2n1]

The title compound was prepared in analogy to the preparation of Example 104 by using [(2R,6R)-4-(8-cyano-2-deuterio-5-quinolyl)-6-methyl-morpholin-2-yl]methyl trifluoromethanesulfonate (intermediate L) and tert-butyl N-(azetidin-3-yl)carbamate instead of [(2R,6R)-4-(8-cyanoquinoxalin-5-yl)-6-methyl-morpholin-2-yl]methyl trifluoromethanesulfonate (intermediate C) and tert-butyl N-[(3S,4S)-4-methoxypyrrolidin-3-yl]carbamate (compound 104e). Example 136 (73 mg) was obtained as a light yellow solid. MS: calc'd 515 (MH⁺), measured 515 (MH⁺). ¹H NMR (METHANOL-d₄, 400 MHz) δ 8.66 (d, 1H, J=8.6 Hz), 8.15 (d, 1H, J=7.9 Hz), 7.64 (d, 1H, J=8.7 Hz), 7.25 (d, 1H, J=8.1 Hz), 5.56 (s, 1H), 4.2-4.2 (m, 3H), 4.0-4.1 (m, 1H), 3.88 (tt, 1H, J=5.3, 7.3 Hz), 3.74 (t, 4H, J=5.0 Hz), 3.67 (dd, 2H, J=5.3, 9.2 Hz), 3.4-3.5 (m, 1H), 3.4-3.4 (m, 1H), 2.6-2.7 (m, 4H), 2.5-2.6 (m, 4H), 2.16 (s, 3H), 1.25 (d, 3H, J=6.4 Hz).

### Example 137

### 5-[(2S,6R)-2-[[4-[6-(3-amino-3-methyl-azetidin-1-yl)-2-methyl-3-pyridyl]piperazin-1-yl]methyl]-6-methyl-morpholin-4-yl]-2-deuterio-quinoline-8-carbonitrile

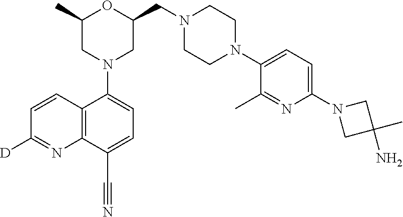 [SMILES: [2H]c1ccc2c(N3C[C@H](CN4CCN(c5ccc(N6CC(C)(N)C6)nc5C)CC4)O[C@H](C)C3)ccc(C#N)c2n1]

The title compound was prepared in analogy to the preparation of Example 132 by using 1-(6-bromo-2-methyl-3-pyridyl)piperazine instead of 1-(6-bromopyridin-3-yl)piperazine (compound 132a). Example 132 (33 mg) was obtained as a light yellow solid. MS: calc'd 528 (MH⁺), measured 528 (MH⁺). ¹H NMR (METHANOL-d₄, 400 MHz) δ 8.66 (d, 1H, J=8.6 Hz), 8.15 (d, 1H, J=7.9 Hz), 7.97 (d, 1H, J=9.3 Hz), 7.64 (d, 1H, J=8.7 Hz), 7.28 (d, 1H, J=8.1 Hz), 6.73 (d, 1H, J=9.2 Hz), 4.5-4.6 (m, 1H), 4.43 (d, 2H, J=9.9 Hz), 4.3-4.4 (m, 2H), 4.1-4.2 (m, 1H), 3.7-3.9 (m, 2H), 3.4-3.5 (m, 6H), 3.1-3.3 (m, 4H), 2.7-2.8 (m, 2H), 2.57 (s, 3H), 1.72 (s, 3H), 1.31 (d, 3H, J=6.4 Hz).

### Example 138

### 5-[(2S,6R)-2-[[3-[6-(3-amino-3-methyl-azetidin-1-yl)-3-pyridyl]azetidin-1-yl]methyl]-6-methyl-morpholin-4-yl]-2-deuterio-quinoline-8-carbonitrile

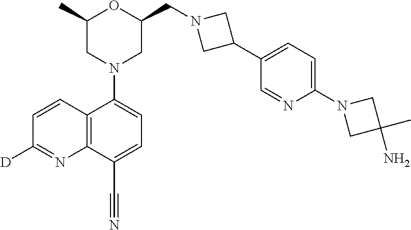 [SMILES: [2H]c1ccc2c(N3C[C@H](CN4CC(c5ccc(N6CC(C)(N)C6)nc5)C4)O[C@H](C)C3)ccc(C#N)c2n1]

The title compound was prepared according to the following scheme:

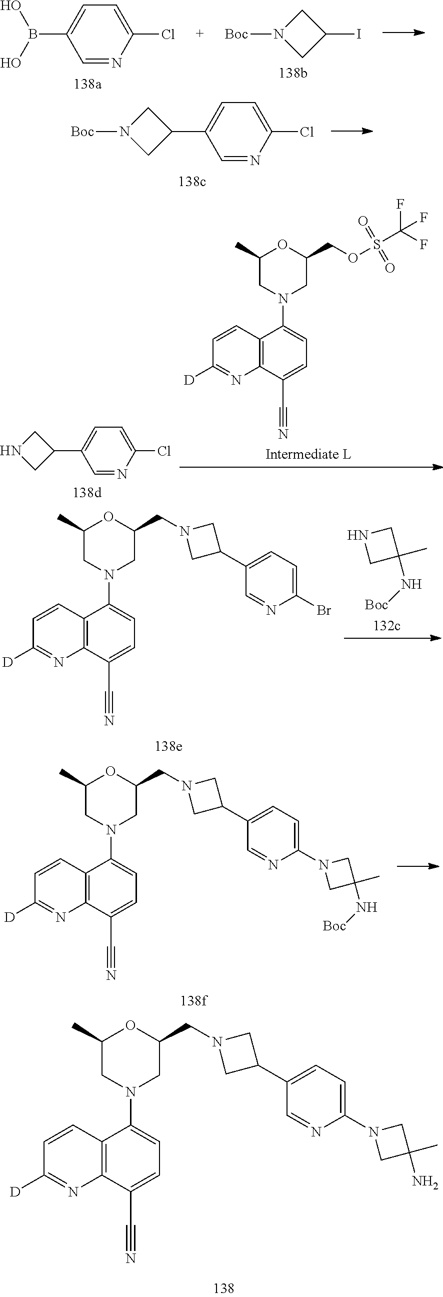

### Step 1: preparation of tert-butyl 3-(6-chloropyridin-3-yl)azetidine-1-carboxylate (Compound 138c)

To a stirred suspension of (6-chloropyridin-3-yl)boronic acid (compound 138a, 1.4 g, 8.9 mmol) in 2-propanol (4 mL) was added a solution of tert-butyl 3-iodoazetidine-1-carboxylate (compound 138b, 1.4 g, 4.95 mmol) in 2-propanol (7 mL) at room temperature. To the mixture was added rac-(1R,2R)-2-aminocyclohexan-1-ol hydrochloride (45 mg, 297 μmol), nickel (II) iodide (92.7 mg, 297 μmol) and sodium bis(trimethylsilyl)amide in THF (4.95 mL, 9.89 mmol) under argon. After the resulting mixture was stirred for 10 mins at r.t., the mixture was heated at 80° C. under microwave for 1 hour. The reaction mixture was poured to a solution of water and ethyl acetate. The aqueous layer was extracted twice with ethyl acetate. The combined organic layer was dried over MgSO₄ and concentrated. The residue was purified by silica gel chromatography (PE/EtOAc from 10% to 60%) to afford tert-butyl 3-(6-chloropyridin-3-yl)azetidine-1-carboxylate (compound 138c, 842 mg) as a colorless solid. MS: calc'd 269 (MH⁺), measured 269 (MH⁺).

### Step 2: preparation of 5-(azetidin-3-yl)-2-chloropyridine 2,2,2-trifluoroacetate (Compound 138d)

To a solution of tert-butyl 3-(6-chloropyridin-3-yl)azetidine-1-carboxylate (compound 138c, 842 mg, 2.69 mmol) in DCM (4 mL) was added 2,2,2-trifluoroacetic acid (4 mL) at 0° C. The reaction mixture was stirred at r.t. for 2 hrs. Then it was concentrated to get crude 5-(azetidin-3-yl)-2-chloropyridine 2,2,2-trifluoroacetate (compound 138d, 750 mg) as a light yellow oil which was used in next step without purification. MS: calc'd 169 (MH⁺), measured 169 (MH⁺).

### Step 3: preparation of 5-((2S,6R)-2-((3-(6-chloropyridin-3-yl)azetidin-1-yl)methyl)-6-methylmorpholino)quinoline-8-carbonitrile-2-d (Compound 138e)

A solution of 5-(azetidin-3-yl)-2-chloropyridine 2,2,2-trifluoroacetate (compound 138d, 377 mg, 1.15 mmol) and [(2R,6R)-4-(8-cyano-2-deuterio-5-quinolyl)-6-methyl-morpholin-2-yl]methyl trifluoromethanesulfonate (Intermediate L, 400 mg, 961 μmol) and TEA (1 mL) in CH₂Cl₂ (20 mL) was stirred at r.t. for 2 hrs. Then the mixture was concentrated and purified by silica gel chromatography (PE/EtOAC from 40% to 100%) to afford 5-((2S,6R)-2-((3-(6-chloropyridin-3-yl)azetidin-1-yl)methyl)-6-methylmorpholino)quinoline-8-carbonitrile-2-d (compound 138e, 320 mg) as a yellow solid. MS: calc'd 435 (MH⁺), measured 435 (MH⁺).

### Step 4: preparation of tert-butyl (1-(5-(1-(((2S,6R)-4-(8-cyanoquinolin-5-yl-2-d)-6-methylmorpholin-2-yl)methyl)azetidin-3-yl)pyridin-2-yl)-3-methylazetidin-3-yl)carbamate (Compound 138d)

To a solution of 5-((2S,6R)-2-((3-(6-chloropyridin-3-yl)azetidin-1-yl)methyl)-6-methylmorpholino)quinoline-8-carbonitrile-2-d (compound 138e, 80 mg, 184 μmol) in dioxane (4 mL) was added tert-butyl (3-methylazetidin-3-yl)carbamate (compound 132^{c}, 41.1 mg, 221 μmol), RuPhos-Pd-G2 (14.3 mg, 18.4 μmol) and Cs₂CO₃ (120 mg, 368 μmol). The reaction mixture was stirred for 16 hours at 120° C. under N₂. Then the mixture was filtered and the filtrate was concentrated and the crude oil was purified by silica gel chromatography (MeOH/DCM from 2% to 12%) to afford tert-butyl (1-(5-(1-(((2S,6R)-4-(8-cyanoquinolin-5-yl-2-d)-6-methylmorpholin-2-yl)methyl)azetidin-3-yl)pyridin-2-yl)-3-methylazetidin-3-yl)carbamate (compound 138f, 79 mg). MS: calc'd 585 (MH⁺), measured 585 (MH⁺).

### Step 5: preparation of 5-[(2S,6R)-2-[[3-[6-(3-amino-3-methyl-azetidin-1-yl)-3-pyridyl]azetidin-1-yl]methyl]-6-methyl-morpholin-4-yl]-2-deuterio-quinoline-8-carbonitrile

### Example 138

To a solution of tert-butyl (1-(5-(1-(((2S,6R)-4-(8-cyanoquinolin-5-yl-2-d)-6-methylmorpholin-2-yl)methyl)azetidin-3-yl)pyridin-2-yl)-3-methylazetidin-3-yl)carbamate (compound 138f, 79 mg, 135 μmol) in DCM (2 mL) was added 2,2,2-trifluoroacetic acid (740 mg, 0.5 ml, 6.49 mmol) at 0° C. The reaction mixture was stirred at rt for 2 hrs. Then the mixture was concentrated and the residue was purified by prep-HPLC to afford 5-[(2S,6R)-2-[[3-[6-(3-amino-3-methyl-azetidin-1-yl)-3-pyridyl]azetidin-1-yl]methyl]-6-methyl-morpholin-4-yl]-2-deuterio-quinoline-8-carbonitrile (Example 138, 22 mg) as a yellow solid. MS calc'd 485 (MH⁺), measured 485 (MH⁺). ¹H NMR (METHANOL-d₄, 400 MHz) δ 8.64 (d, 1H, J=8.7 Hz), 8.14 (d, 1H, J=8.1 Hz), 7.91 (d, 1H, J=2.2 Hz), 7.63 (br d, 2H, J=8.6 Hz), 7.24 (d, 1H, J=8.1 Hz), 6.43 (d, 1H, J=8.6 Hz), 4.0-4.1 (m, 2H), 3.8-3.9 (m, 2H), 3.7-3.8 (m, 4H), 3.6-3.7 (m, 1H), 3.3-3.4 (m, 2H), 3.2-3.3 (m, 2H), 2.6-2.8 (m, 4H), 1.49 (s, 3H), 1.24 (d, 3H, J=6.2 Hz).

### Example 139

### 5-[(2S,6R)-2-[[4-[5-(3-amino-3-methyl-azetidin-1-yl)-3-methyl-2-pyridyl]piperazin-1-yl]methyl]-6-methyl-morpholin-4-yl]-2-deuterio-quinoline-8-carbonitrile

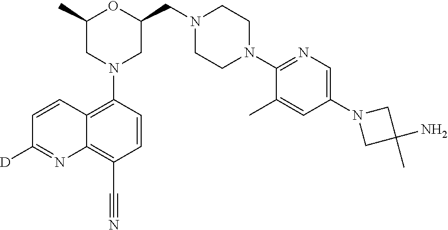 [SMILES: [2H]c1ccc2c(N3C[C@H](CN4CCN(c5ncc(N6CC(C)(N)C6)cc5C)CC4)O[C@H](C)C3)ccc(C#N)c2n1]

The title compound was prepared in analogy to the preparation of Example 132 by using 1-(5-bromo-3-methyl-2-pyridyl)piperazine instead of 1-(6-bromopyridin-3-yl)piperazine (compound 132a). Example 139 (8 mg) was obtained as a light yellow solid. MS: calc'd 528 (MH⁺), measured 528 (MH⁺). ¹H NMR (METHANOL-d₄, 400 MHz) δ 8.69 (d, 1H, J=8.6 Hz), 8.19 (d, 1H, J=8.1 Hz), 7.68 (d, 1H, J=8.6 Hz), 7.50 (d, 1H, J=2.8 Hz), 7.31 (d, 1H, J=8.1 Hz), 6.96 (d, 1H, J=2.4 Hz), 4.4-4.6 (m, 1H), 4.2-4.3 (m, 1H), 4.0-4.1 (m, 2H), 3.9-4.0 (m, 2H), 3.7-3.9 (m, 2H), 3.2-3.6 (m, 10H), 2.7-2.9 (m, 2H), 2.35 (s, 3H), 1.69 (s, 3H), 1.34 (d, 3H, J=6.2 Hz).

### Example 140

### 5-[(2S,6R)-2-[[3-[4-[(3S,4S)-3-amino-4-methoxy-pyrrolidin-1-yl]phenyl]azetidin-1-yl]methyl]-6-methyl-morpholin-4-yl]-2-deuterio-quinoline-8-carbonitrile

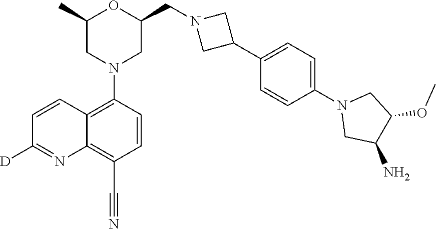 [SMILES: [2H]c1ccc2c(N3C[C@H](CN4CC(c5ccc(N6C[C@H](OC)[C@@H](N)C6)cc5)C4)O[C@H](C)C3)ccc(C#N)c2n1]

The title compound was prepared in analogy to the preparation of Example 132 by using 3-(4-bromophenyl)azetidine and tert-butyl N-[(3S,4S)-4-methoxypyrrolidin-3-yl]carbamate instead of 1-(6-bromopyridin-3-yl)piperazine (compound 132a) and tert-butyl (3-methylazetidin-3-yl)carbamate (compound 132c). Example 140 (1 mg) was obtained as a light yellow solid. MS: calc'd 514 (MH⁺), measured 514 (MH⁺). ¹H NMR (METHANOL-d₄, 400 MHz) δ 8.68 (d, 1H, J=8.7 Hz), 8.19 (d, 1H, J=8.1 Hz), 7.6-7.8 (m, 1H), 7.2-7.4 (m, 3H), 6.73 (d, 2H, J=8.8 Hz), 4.4-4.7 (m, 3H), 4.1-4.4 (m, 6H), 3.6-3.9 (m, 3H), 3.4-3.5 (m, 7H), 2.6-2.9 (m, 3H), 1.2-1.4 (m, 3H).

### Example 141

### 5-[(2S,6R)-2-[[3-[4-(3-amino-3-methyl-azetidin-1-yl)phenyl]azetidin-1-yl]methyl]-6-methyl-morpholin-4-yl]-2-deuterio-quinoline-8-carbonitrile

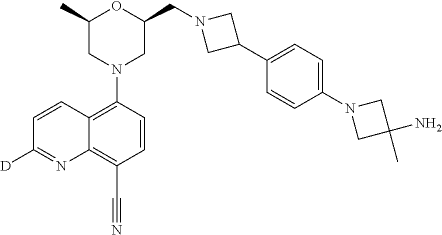 [SMILES: [2H]c1ccc2c(N3C[C@H](CN4CC(c5ccc(N6CC(C)(N)C6)cc5)C4)O[C@H](C)C3)ccc(C#N)c2n1]

The title compound was prepared in analogy to the preparation of Example 132 by using 3-(4-bromophenyl)azetidine instead of 1-(6-bromopyridin-3-yl)piperazine (compound 132a). Example 141 (13 mg) was obtained as a light yellow solid. MS: calc'd 484 (MH⁺), measured 484 (MH⁺). ¹H NMR (METHANOL-d₄, 400 MHz) δ 8.56 (d, 1H, J=8.7 Hz), 8.06 (d, 1H, J=7.9 Hz), 7.55 (d, 1H, J=8.6 Hz), 7.18 (t, 3H, J=7.7 Hz), 6.48 (br d, 2H, J=8.3 Hz), 4.3-4.6 (m, 3H), 4.0-4.3 (m, 4H), 3.8-3.9 (m, 2H), 3.7-3.8 (m, 2H), 3.3-3.5 (m, 4H), 2.5-2.8 (m, 2H), 1.57 (s, 3H), 1.20 (d, 3H, J=6.1 Hz).

### Example 142

### 5-[(2S,6R)-2-[[4-[5-[(3R,4R)-3-amino-4-methoxy-pyrrolidin-1-yl]-3-methyl-2-pyridyl]piperazin-1-yl]methyl]-6-methyl-morpholin-4-yl]-2-deuterio-quinoline-8-carbonitrile

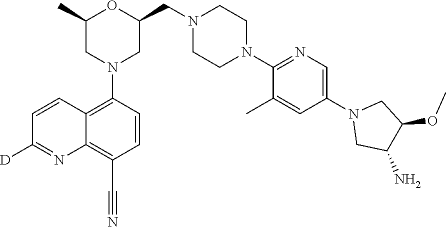 [SMILES: [2H]c1ccc2c(N3C[C@H](CN4CCN(c5ncc(N6C[C@@H](N)[C@H](OC)C6)cc5C)CC4)O[C@H](C)C3)ccc(C#N)c2n1]

The title compound was prepared in analogy to the preparation of Example 132 by using 1-(5-bromo-3-methyl-2-pyridyl)piperazine and tert-butyl N-[(3R,4R)-4-methoxypyrrolidin-3-yl]carbamate instead of 1-(6-bromopyridin-3-yl)piperazine (compound 132a) and tert-butyl (3-methylazetidin-3-yl)carbamate (compound 132c). Example 142 (8 mg) was obtained as a light yellow solid. MS: calc'd 558 (MH⁺), measured 558 (MH⁺). ¹H NMR (METHANOL-d₄, 400 MHz) δ 8.69 (d, 1H, J=8.7 Hz), 8.20 (d, 1H, J=7.9 Hz), 7.68 (d, 1H, J=8.7 Hz), 7.61 (d, 1H, J=2.8 Hz), 7.32 (d, 1H, J=8.1 Hz), 7.13 (d, 1H, J=2.7 Hz), 4.63 (br d, 1H, J=7.5 Hz), 4.1-4.3 (m, 2H), 3.8-4.0 (m, 3H), 3.6-3.7 (m, 2H), 3.2-3.5 (m, 15H), 2.6-3.0 (m, 2H), 2.38 (s, 3H), 1.35 (d, 3H, J=6.2 Hz).

### Example 143

### 5-[(2S,6R)-2-[[4-[6-[(3R,4R)-3-amino-4-methoxy-pyrrolidin-1-yl]-3-pyridyl]piperazin-1-yl]methyl]-6-methyl-morpholin-4-yl]-2-deuterio-quinoline-8-carbonitrile

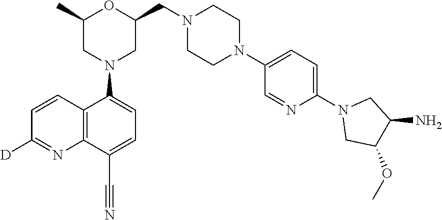

The title compound was prepared in analogy to the preparation of Example 132 by using tert-butyl N-[(3R,4R)-4-methoxypyrrolidin-3-yl]carbamate instead of tert-butyl (3-methylazetidin-3-yl)carbamate (compound 132c). Example 143 (52 mg) was obtained as a light yellow solid. MS: calc'd 544 (MH⁺), measured 544 (MH⁺). ¹H NMR (METHANOL-d₄, 400 MHz) δ 8.64 (d, 1H, J=8.7 Hz), 8.13 (d, 1H, J=8.1 Hz), 8.06 (dd, 1H, J=2.8, 9.8 Hz), 7.63 (d, 1H, J=8.7 Hz), 7.57 (d, 1H, J=2.7 Hz), 7.25 (d, 1H, J=8.1 Hz), 7.16 (d, 1H, J=9.8 Hz), 4.5-4.6 (m, 1H), 4.3-4.3 (m, 1H), 4.1-4.2 (m, 1H), 4.0-4.1 (m, 3H), 3.5-3.9 (m, 7H), 3.49 (s, 4H), 3.3-3.5 (m, 5H), 3.2-3.3 (m, 1H), 2.7-2.8 (m, 2H), 1.31 (d, 3H, J=6.4 Hz).

### Example 144

### 5-[(2S,6R)-2-[[4-[4-[(3R,4R)-3-amino-4-methoxy-pyrrolidin-1-yl]-6-methyl-pyrimidin-2-yl]piperazin-1-yl]methyl]-6-methyl-morpholin-4-yl]-2-deuterio-quinoline-8-carbonitrile

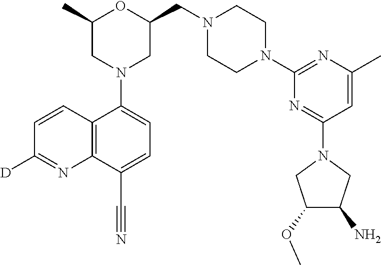 [SMILES: [2H]c1ccc2c(N3C[C@H](CN4CCN(c5nc(C)cc(N6C[C@@H](N)[C@H](OC)C6)n5)CC4)O[C@H](C)C3)ccc(C#N)c2n1]

The title compound was prepared in analogy to the preparation of Example 104 by using [(2R,6R)-4-(8-cyano-2-deuterio-5-quinolyl)-6-methyl-morpholin-2-yl]methyl trifluoromethanesulfonate (intermediate L) and tert-butyl N-[(3R,4R)-4-methoxypyrrolidin-3-yl]carbamate instead of [(2R,6R)-4-(8-cyanoquinoxalin-5-yl)-6-methyl-morpholin-2-yl]methyl trifluoromethanesulfonate (intermediate C) and tert-butyl N-[(3S,4S)-4-methoxypyrrolidin-3-yl]carbamate (compound 104e). Example 144 (43 mg) was obtained as a light yellow solid. MS: calc'd 559 (MH⁺), measured 559 (MH⁺). ¹H NMR (METHANOL-d₄, 400 MHz) δ 8.55 (d, 1H, J=8.6 Hz), 8.03 (d, 1H, J=8.1 Hz), 7.53 (d, 1H, J=8.6 Hz), 7.13 (d, 1H, J=8.1 Hz), 5.59 (s, 1H), 4.1-4.1 (m, 1H), 3.9-4.0 (m, 1H), 3.6-3.7 (m, 6H), 3.4-3.6 (m, 2H), 3.3-3.4 (m, 3H), 3.3-3.3 (m, 3H), 3.3-3.3 (m, 1H), 2.5-2.6 (m, 4H), 2.4-2.5 (m, 4H), 2.09 (s, 3H), 1.16 (d, 3H, J=6.2 Hz).

### Example 145

### 5-[(2S,6R)-2-[[3-[6-(3-amino-3-methyl-azetidin-1-yl)-4-methyl-3-pyridyl]azetidin-1-yl]methyl]-6-methyl-morpholin-4-yl]-2-deuterio-quinoline-8-carbonitrile

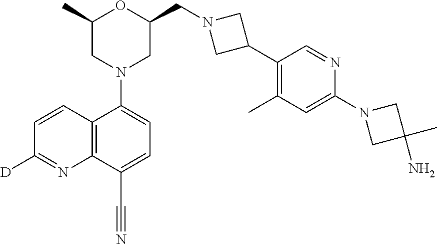 [SMILES: [2H]c1ccc2c(N3C[C@H](CN4CC(c5cnc(N6CC(C)(N)C6)cc5C)C4)O[C@H](C)C3)ccc(C#N)c2n1]

The title compound was prepared in analogy to the preparation of Example 138 by using (6-fluoro-4-methylpyridin-3-yl)boronic acid instead of (6-chloropyridin-3-yl)boronic acid (compound 138a). Example 145 (1 mg) was obtained as a light yellow solid. MS: calc'd 499 (MH⁺), measured 499 (MH⁺). ¹H NMR (METHANOL-d₄, 400 MHz) δ 8.65 (d, 1H, J=8.6 Hz), 8.15 (d, 1H, J=7.9 Hz), 7.81 (s, 1H), 7.64 (d, 1H, J=8.6 Hz), 7.24 (d, 1H, J=7.9 Hz), 6.27 (s, 1H), 4.0-4.1 (m, 2H), 3.8-4.0 (m, 7H), 3.3-3.4 (m, 4H), 2.6-2.8 (m, 4H), 2.16 (s, 3H), 1.51 (s, 3H), 1.2-1.3 (m, 3H).

### Example 146

### 5-[(2S,6R)-2-[[3-[6-[(3R,4R)-3-amino-4-methoxy-pyrrolidin-1-yl]-4-methyl-3-pyridyl]azetidin-1-yl]methyl]-6-methyl-morpholin-4-yl]-2-deuterio-quinoline-8-carbonitrile

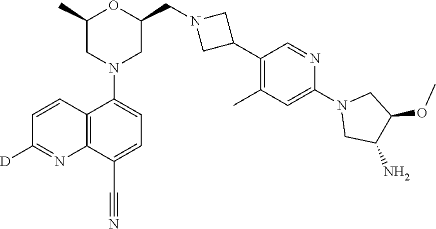 [SMILES: [2H]c1ccc2c(N3C[C@H](CN4CC(c5cnc(N6C[C@@H](N)[C@H](OC)C6)cc5C)C4)O[C@H](C)C3)ccc(C#N)c2n1]

The title compound was prepared in analogy to the preparation of Example 138 by using (6-fluoro-4-methylpyridin-3-yl)boronic acid and tert-butyl ((3R,4R)-4-methoxypyrrolidin-3-yl)carbamate instead of (6-chloropyridin-3-yl)boronic acid ((compound 138a) and tert-butyl (3-methylazetidin-3-yl)carbamate (compound 132c). Example 146 (1 mg) was obtained as a light yellow solid. MS: calc'd 529 (MH⁺), measured 529 (MH⁺). ¹H NMR (METHANOL-d₄, 400 MHz) δ 8.65 (d, 1H, J=8.6 Hz), 8.15 (d, 1H, J=7.9 Hz), 7.81 (s, 1H), 7.64 (d, 1H, J=8.6 Hz), 7.25 (d, 1H, J=8.1 Hz), 6.3-6.4 (m, 1H), 4.0-4.1 (m, 2H), 3.9-4.0 (m, 2H), 3.7-3.8 (m, 3H), 3.6-3.7 (m, 1H), 3.5-3.6 (m, 1H), 3.3-3.4 (m, 7H), 3.2-3.3 (m, 2H), 2.6-2.8 (m, 4H), 2.17 (s, 3H), 1.25 (d, 3H, J=6.2 Hz).

### Example 147

### 5-[(2S,6R)-2-[[4-[5-(3-amino-3-methyl-azetidin-1-yl)-6-methyl-2-pyridyl]piperazin-1-yl]methyl]-6-methyl-morpholin-4-yl]-2-deuterio-quinoline-8-carbonitrile

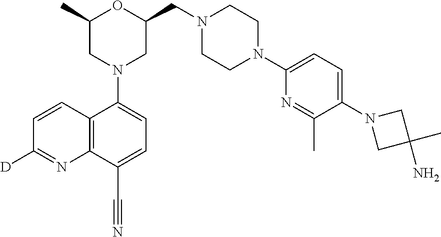 [SMILES: [2H]c1ccc2c(N3C[C@H](CN4CCN(c5ccc(N6CC(C)(N)C6)c(C)n5)CC4)O[C@H](C)C3)ccc(C#N)c2n1]

The title compound was prepared in analogy to the preparation of Example 132 by using 1-(5-bromo-6-methyl-2-pyridyl)piperazine instead of 1-(6-bromopyridin-3-yl)piperazine (compound 132a). Example 147 (4 mg) was obtained as a light yellow solid. MS: calc'd 528 (MH⁺), measured 528 (MH⁺). ¹H NMR (METHANOL-d₄, 400 MHz) δ 8.57 (d, 1H, J=8.7 Hz), 8.08 (d, 1H, J=7.9 Hz), 7.56 (d, 1H, J=8.7 Hz), 7.19 (d, 1H, J=7.9 Hz), 6.96 (d, 1H, J=8.8 Hz), 6.67 (d, 1H, J=8.8 Hz), 4.3-4.5 (m, 1H), 4.0-4.2 (m, 2H), 3.7-3.9 (m, 4H), 3.3-3.5 (m, 9H), 2.8-3.0 (m, 1H), 2.5-2.8 (m, 3H), 2.26 (s, 3H), 1.56 (s, 3H), 1.22 (d, 3H, J=6.4 Hz)

### Example 148

### 4-[(2S,6R)-2-[[4-[5-(3-amino-3-methyl-azetidin-1-yl)-3-methyl-2-pyridyl]piperazin-1-yl]methyl]-6-methyl-morpholin-4-yl]-3-fluoro-pyrazolo[1,5-a]pyridine-7-carbonitrile

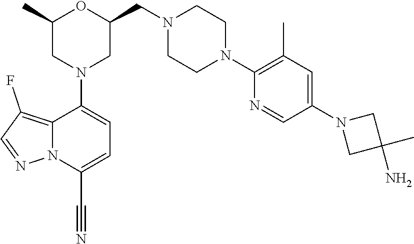 [SMILES: Cc1cc(N2CC(C)(N)C2)cnc1N1CCN(C[C@H]2CN(c3ccc(C#N)n4ncc(F)c34)C[C@@H](C)O2)CC1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and 1-(5-bromo-3-methyl-2-pyridyl)piperazine instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a). Example 148 (17 mg) was obtained as a light yellow solid. MS: calc'd 534 (MH⁺), measured 534 (MH⁺). ¹H NMR (METHANOL-d₄, 400 MHz) δ 8.00 (d, 1H, J=3.5 Hz), 7.47 (d, 1H, J=2.8 Hz), 7.43 (d, 1H, J=7.9 Hz), 7.06 (d, 1H, J=2.4 Hz), 6.55 (d, 1H, J=7.9 Hz), 4.3-4.4 (m, 1H), 4.0-4.1 (m, 3H), 3.95 (d, 2H, J=8.4 Hz), 3.5-3.7 (m, 5H), 3.3-3.5 (m, 7H), 2.7-2.8 (m, 2H), 2.34 (s, 3H), 1.68 (s, 3H), 1.3-1.3 (m, 3H).

### Example 149

### 5-[(2S,6R)-2-[[3-[6-[(3R,4R)-3-amino-4-methoxy-pyrrolidin-1-yl]-3-pyridyl]azetidin-1-yl]methyl]-6-methyl-morpholin-4-yl]-2-deuterio-quinoline-8-carbonitrile

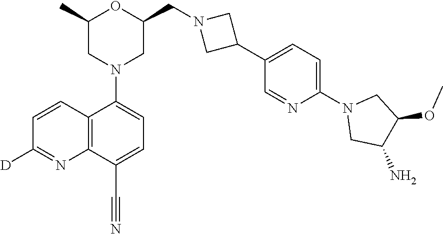 [SMILES: [2H]c1ccc2c(N3C[C@H](CN4CC(c5ccc(N6C[C@@H](N)[C@H](OC)C6)nc5)C4)O[C@H](C)C3)ccc(C#N)c2n1]

The title compound was prepared in analogy to the preparation of Example 138 by using tert-butyl ((3R,4R)-4-methoxypyrrolidin-3-yl)carbamate instead of tert-butyl (3-methylazetidin-3-yl)carbamate (compound 132c). Example 149 (4 mg) was obtained as a light yellow solid. MS: calc'd 515 (MH⁺), measured 515 (MH⁺). ¹H NMR (METHANOL-d₄, 400 MHz) δ 8.65 (d, 1H, J=8.6 Hz), 8.3-8.6 (m, 1H), 8.16 (d, 1H, J=7.9 Hz), 8.05 (d, 1H, J=2.1 Hz), 7.73 (br d, 1H, J=2.3 Hz), 7.64 (d, 1H, J=8.6 Hz), 7.27 (d, 1H, J=8.1 Hz), 6.63 (d, 1H, J=8.7 Hz), 4.3-4.4 (m, 2H), 4.2-4.3 (m, 1H), 4.0-4.2 (m, 5H), 3.8-3.9 (m, 3H), 3.5-3.6 (m, 1H), 3.5-3.5 (m, 1H), 3.46 (s, 3H), 3.41 (br d, 2H, J=12.2 Hz), 3.2-3.3 (m, 1H), 2.6-2.8 (m, 2H), 1.28 (d, 3H, J=6.2 Hz).

### Example 150

### 5-[(2S,6R)-2-[[4-[6-[(3S,4S)-3-amino-4-fluoro-pyrrolidin-1-yl]-3-pyridyl]piperazin-1-yl]methyl]-6-methyl-morpholin-4-yl]-2-deuterio-quinoline-8-carbonitrile

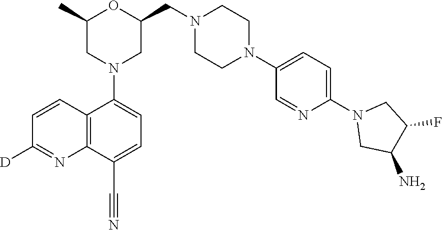 [SMILES: [2H]c1ccc2c(N3C[C@H](CN4CCN(c5ccc(N6C[C@H](N)[C@@H](F)C6)nc5)CC4)O[C@H](C)C3)ccc(C#N)c2n1]

The title compound was prepared in analogy to the preparation of Example 132 by using tert-butyl N-[(3S,4S)-4-fluoropyrrolidin-3-yl]carbamate instead of tert-butyl (3-methylazetidin-3-yl)carbamate (compound 132c). Example 150 (4 mg) was obtained as a light yellow solid. MS: calc'd 532 (MH⁺), measured 532 (MH⁺). ¹H NMR (METHANOL-d₄, 400 MHz) δ 8.66 (d, 1H, J=8.6 Hz), 8.3-8.4 (m, 1H), 8.15 (d, 1H, J=7.9 Hz), 7.82 (d, 1H, J=2.8 Hz), 7.64 (d, 1H, J=8.6 Hz), 7.45 (dd, 1H, J=2.9, 9.1 Hz), 7.26 (d, 1H, J=8.1 Hz), 6.60 (d, 1H, J=9.0 Hz), 5.1-5.3 (m, 1H), 4.2-4.4 (m, 1H), 4.1-4.2 (m, 1H), 3.9-4.0 (m, 1H), 3.8-3.9 (m, 1H), 3.6-3.7 (m, 1H), 3.5-3.6 (m, 1H), 3.4-3.5 (m, 2H), 3.1-3.2 (m, 4H), 2.9-3.1 (m, 4H), 2.6-2.9 (m, 4H), 1.27 (d, 3H, J=6.2 Hz).

### Example 151

### 2-deuterio-5-[(2S,6R)-2-[[4-[6-[(1S,4S)-2,5-diazabicyclo[2.2.1]heptan-2-yl]-3-pyridyl]piperazin-1-yl]methyl]-6-methyl-morpholin-4-yl]quinoline-8-carbonitrile

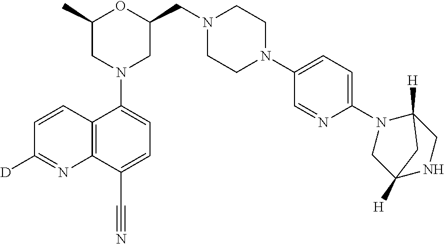 [SMILES: [2H]c1ccc2c(N3C[C@H](CN4CCN(c5ccc(N6C[C@@H]7C[C@H]6CN7)nc5)CC4)O[C@H](C)C3)ccc(C#N)c2n1]

The title compound was prepared in analogy to the preparation of Example 132 by using tert-butyl (1S,4S)-2,5-diazabicyclo[2.2.1]heptane-2-carboxylate (Bidepharm, BD30269, CAS: 113451-59-5) instead of tert-butyl (3-methylazetidin-3-yl)carbamate (compound 132c). Example 151 (5 mg) was obtained as a light yellow solid. MS: calc'd 526 (MH⁺), measured 526 (MH⁺). ¹H NMR (METHANOL-d₄, 400 MHz) δ 8.67 (d, 1H, J=8.7 Hz), 8.17 (d, 1H, J=8.1 Hz), 7.77 (d, 1H, J=2.7 Hz), 7.72 (dd, 1H, J=2.9, 9.4 Hz), 7.66 (d, 1H, J=8.6 Hz), 7.29 (d, 1H, J=8.1 Hz), 6.89 (d, 1H, J=9.4 Hz), 4.97 (s, 1H), 4.58 (s, 1H), 4.5-4.5 (m, 1H), 4.1-4.2 (m, 1H), 3.7-3.8 (m, 3H), 3.5-3.7 (m, 4H), 3.4-3.5 (m, 8H), 3.3-3.4 (m, 1H), 2.7-2.8 (m, 2H), 2.31 (br d, 1H, J=10.9 Hz), 2.11 (br d, 1H, J=11.5 Hz), 1.3-1.3 (m, 3H).

### Example 152

### 2-deuterio-5-[(2R,6S)-2-methyl-6-[[4-[6-(5-oxa-2,8-diazaspiro[3.5]nonan-2-yl)-3-pyridyl]piperazin-1-yl]methyl]morpholin-4-yl]quinoline-8-carbonitrile

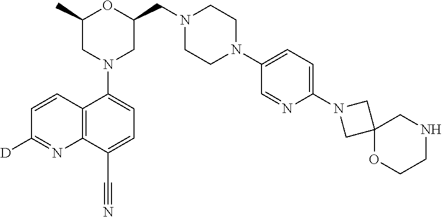 [SMILES: [2H]c1ccc2c(N3C[C@H](CN4CCN(c5ccc(N6CC7(CNCCO7)C6)nc5)CC4)O[C@H](C)C3)ccc(C#N)c2n1]

The title compound was prepared in analogy to the preparation of Example 132 by using tert-butyl 5-oxa-2,8-diazaspiro[3.5]nonane-8-carboxylate instead of tert-butyl (3-methylazetidin-3-yl)carbamate (compound 132c). Example 152 (10 mg) was obtained as a light yellow solid. MS: calc'd 556 (MH⁺), measured 556 (MH⁺). ¹H NMR (METHANOL-d₄, 400 MHz) δ 8.65 (d, 1H, J=8.6 Hz), 8.14 (d, 1H, J=7.9 Hz), 7.99 (dd, 1H, J=2.7, 9.7 Hz), 7.64 (d, 1H, J=8.6 Hz), 7.56 (d, 1H, J=2.6 Hz), 7.27 (d, 1H, J=8.1 Hz), 6.92 (d, 1H, J=9.7 Hz), 4.5-4.7 (m, 2H), 4.39 (d, 2H, J=9.8 Hz), 4.27 (d, 2H, J=9.7 Hz), 4.18 (ddd, 1H, J=2.1, 6.3, 10.0 Hz), 4.0-4.1 (m, 2H), 3.4-3.7 (m, 13H), 3.3-3.3 (m, 2H), 2.75 (ddd, 2H, J=10.4, 11.9, 19.4 Hz), 1.31 (d, 3H, J=6.2 Hz).

### Example 153

### 2-deuterio-5-[(2S,6R)-2-[[4-[6-[(1R,4R)-2,5-diazabicyclo[2.2.1]heptan-2-yl]-3-pyridyl]piperazin-1-yl]methyl]-6-methyl-morpholin-4-yl]quinoline-8-carbonitrile

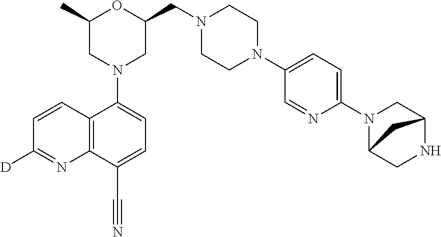 [SMILES: [2H]c1ccc2c(N3C[C@H](CN4CCN(c5ccc(N6C[C@H]7C[C@@H]6CN7)nc5)CC4)O[C@H](C)C3)ccc(C#N)c2n1]

The title compound was prepared in analogy to the preparation of Example 132 by using tert-butyl (1R,4R)-2,5-diazabicyclo[2.2.1]heptane-2-carboxylate (Pharmablock, PBN20120578, CAS: 134003-84-2) instead of tert-butyl (3-methylazetidin-3-yl)carbamate (compound 132c). Example 153 (3 mg) was obtained as a light yellow solid. MS: calc'd 526 (MH⁺), measured 526 (MH⁺). ¹H NMR (METHANOL-d₄, 400 MHz) δ 8.7-8.7 (m, 1H), 8.2-8.2 (m, 1H), 7.8-7.8 (m, 1H), 7.6-7.7 (m, 2H), 7.29 (d, 1H, J=8.1 Hz), 6.87 (d, 1H, J=9.4 Hz), 4.9-5.0 (m, 1H), 4.5-4.6 (m, 2H), 4.1-4.3 (m, 1H), 3.8-3.8 (m, 1H), 3.7-3.9 (m, 2H), 3.5-3.7 (m, 4H), 3.4-3.5 (m, 8H), 2.9-3.1 (m, 1H), 2.7-2.8 (m, 3H), 2.3-2.3 (m, 1H), 2.1-2.1 (m, 1H), 1.3-1.3 (m, 3H).

### Example 154

### 4-[(2S,6R)-2-[[4-[6-(3-amino-3-methyl-azetidin-1-yl)-3-pyridyl]piperazin-1-yl]methyl]-6-methyl-morpholin-4-yl]-3-fluoro-pyrazolo[1,5-a]pyridine-7-carbonitrile

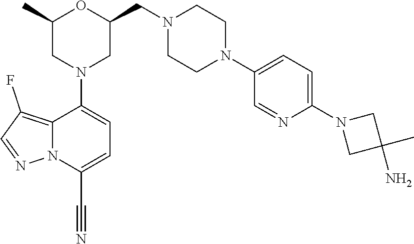 [SMILES: C[C@@H]1CN(c2ccc(C#N)n3ncc(F)c23)C[C@H](CN2CCN(c3ccc(N4CC(C)(N)C4)nc3)CC2)O1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) instead of [(2R,6R)-4-(8-cyano-2-deuterio-5-quinolyl)-6-methyl-morpholin-2-yl]methyl trifluoromethanesulfonate (intermediate L). Example 154 (19 mg) was obtained as a light yellow solid. MS: calc'd 520 (MH⁺), measured 520 (MH⁺). ¹H NMR (METHANOL-d₄, 400 MHz) δ 8.01 (d, 1H, J=3.7 Hz), 7.97 (dd, 1H, J=2.7, 9.5 Hz), 7.63 (d, 1H, J=2.6 Hz), 7.45 (d, 1H, J=7.9 Hz), 6.92 (d, 1H, J=9.5 Hz), 6.56 (d, 1H, J=7.9 Hz), 4.3-4.4 (m, 5H), 4.0-4.1 (m, 1H), 3.5-3.9 (m, 7H), 3.4-3.5 (m, 5H), 2.7-2.8 (m, 2H), 1.74 (s, 3H), 1.32 (d, 3H, J=6.2 Hz).

### Example 155

### 5-[(2S,6R)-2-[[4-[6-(3-amino-3-methyl-azetidin-1-yl)-2-pyridyl]piperazin-1-yl]methyl]-6-methyl-morpholin-4-yl]-2-deuterio-quinoline-8-carbonitrile

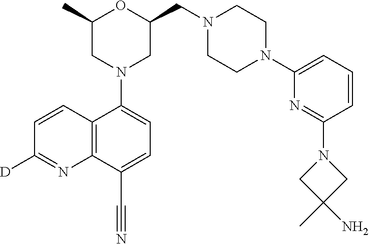 [SMILES: [2H]c1ccc2c(N3C[C@H](CN4CCN(c5cccc(N6CC(C)(N)C6)n5)CC4)O[C@H](C)C3)ccc(C#N)c2n1]

The title compound was prepared in analogy to the preparation of Example 132 by using 1-(6-bromo-2-pyridyl)piperazine instead of 1-(6-bromopyridin-3-yl)piperazine (compound 132a). Example 155 (5 mg) was obtained as a light yellow solid. MS: calc'd 514 (MH⁺), measured 514 (MH⁺). ¹H NMR (METHANOL-d₄, 400 MHz) δ 8.68 (d, 1H, J=8.7 Hz), 8.17 (d, 1H, J=8.1 Hz), 7.66 (d, 1H, J=8.6 Hz), 7.36 (t, 1H, J=8.0 Hz), 7.27 (d, 1H, J=8.1 Hz), 6.11 (d, 1H, J=8.1 Hz), 5.79 (d, 1H, J=7.8 Hz), 4.2-4.3 (m, 1H), 4.0-4.1 (m, 1H), 3.8-3.9 (m, 6H), 3.4-3.6 (m, 6H), 2.5-2.8 (m, 6H), 1.56 (s, 3H), 1.2-1.4 (m, 3H).

### Example 156

### 5-[(2S,6R)-2-[[3-[4-(3-amino-3-methyl-azetidin-1-yl)phenyl]-3-hydroxy-azetidin-1-yl]methyl]-6-methyl-morpholin-4-yl]-2-deuterio-quinoline-8-carbonitrile

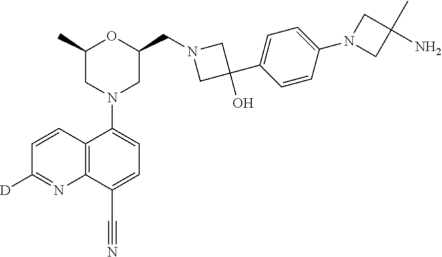 [SMILES: [2H]c1ccc2c(N3C[C@H](CN4CC(O)(c5ccc(N6CC(C)(N)C6)cc5)C4)O[C@H](C)C3)ccc(C#N)c2n1]

The title compound was prepared in analogy to the preparation of Example 132 by using 3-(4-bromophenyl)azetidin-3-ol instead of 1-(6-bromopyridin-3-yl)piperazine (compound 132a). Example 156 (7 mg) was obtained as a light yellow solid. MS: calc'd 500 (MH⁺), measured 500 (MH⁺). ¹H NMR (METHANOL-d₄, 400 MHz) δ 8.66 (d, 1H, J=8.7 Hz), 8.16 (d, 1H, J=8.1 Hz), 7.64 (d, 1H, J=8.7 Hz), 7.4-7.5 (m, 2H), 7.26 (d, 1H, J=8.1 Hz), 6.4-6.6 (m, 2H), 4.0-4.1 (m, 2H), 3.7-3.9 (m, 4H), 3.6-3.7 (m, 2H), 3.4-3.6 (m, 4H), 2.6-2.9 (m, 3H), 1.55 (s, 3H), 1.26 (d, 4H, J=6.2 Hz).

### Example 157

### 4-[(2S,6R)-2-[[3-[6-(3-amino-3-methyl-azetidin-1-yl)-3-pyridyl]azetidin-1-yl]methyl]-6-methyl-morpholin-4-yl]pyrazolo[1,5-a]pyridine-7-carbonitrile

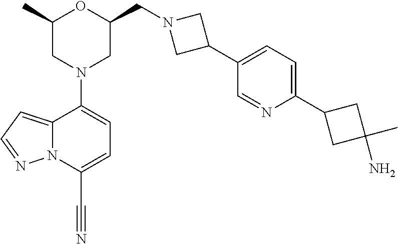 [SMILES: C[C@@H]1CN(c2ccc(C#N)n3nccc23)C[C@H](CN2CC(c3ccc(C4CC(C)(N)C4)nc3)C2)O1]

The title compound was prepared in analogy to the preparation of Example 138 by using (2R,6R)-4-(7-cyanopyrazolo[1,5-a]pyridin-4-yl)-6-methyl-morpholin-2-yl]methyl trifluoromethanesulfonate (intermediate D) instead of [(2R,6R)-4-(8-cyano-2-deuterio-5-quinolyl)-6-methyl-morpholin-2-yl]methyl trifluoromethanesulfonate (intermediate L). Example 157 (13 mg) was obtained as a light yellow solid. MS: calc'd 473 (MH⁺), measured 473 (MH⁺). ¹H NMR (METHANOL-d₄, 400 MHz) δ 8.02 (d, 1H, J=2.3 Hz), 7.91 (d, 1H, J=2.2 Hz), 7.63 (dd, 1H, J=2.4, 8.6 Hz), 7.46 (d, 1H, J=8.1 Hz), 6.86 (d, 1H, J=2.4 Hz), 6.58 (d, 1H, J=8.1 Hz), 6.44 (d, 1H, J=8.7 Hz), 3.7-3.9 (m, 10H), 3.6-3.7 (m, 1H), 3.25 (t, 2H, J=7.5 Hz), 2.6-2.7 (m, 4H), 1.49 (s, 3H), 1.24 (d, 3H, J=6.2 Hz).

### Example 158

### 4-[(2S,6R)-2-[[3-[6-(3-amino-3-methyl-azetidin-1-yl)-3-pyridyl]azetidin-1-yl]methyl]-6-methyl-morpholin-4-yl]-3-fluoro-pyrazolo[1,5-a]pyridine-7-carbonitrile

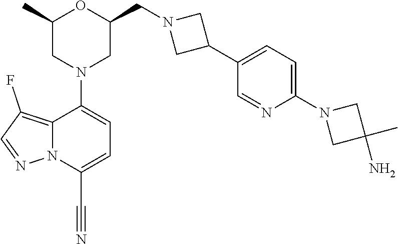 [SMILES: C[C@@H]1CN(c2ccc(C#N)n3ncc(F)c23)C[C@H](CN2CC(c3ccc(N4CC(C)(N)C4)nc3)C2)O1]

The title compound was prepared in analogy to the preparation of Example 138 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) instead of [(2R,6R)-4-(8-cyano-2-deuterio-5-quinolyl)-6-methyl-morpholin-2-yl]methyl trifluoromethanesulfonate (intermediate L). Example 158 (23 mg) was obtained as a light yellow solid. MS: calc'd 491 (MH⁺), measured 491 (MH⁺). ¹H NMR (METHANOL-d₄, 400 MHz) δ 7.99 (d, 1H, J=3.7 Hz), 7.91 (d, 1H, J=2.1 Hz), 7.63 (dd, 1H, J=2.4, 8.6 Hz), 7.41 (d, 1H, J=7.9 Hz), 6.50 (d, 1H, J=8.1 Hz), 6.44 (d, 1H, J=8.6 Hz), 3.7-3.9 (m, 8H), 3.5-3.7 (m, 3H), 3.23 (dt, 2H, J=3.6, 7.5 Hz), 2.69 (s, 2H), 2.6-2.7 (m, 2H), 1.49 (s, 3H), 1.23 (d, 3H, J=6.2 Hz).

### Example 159

### 5-[(2S,6R)-2-[[3-[6-(3-amino-3-methyl-azetidin-1-yl)-2-pyridyl]azetidin-1-yl]methyl]-6-methyl-morpholin-4-yl]-2-deuterio-quinoline-8-carbonitrile

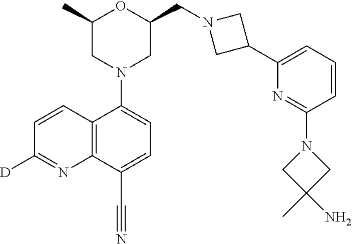 [SMILES: [2H]c1ccc2c(N3C[C@H](CN4CC(c5cccc(N6CC(C)(N)C6)n5)C4)O[C@H](C)C3)ccc(C#N)c2n1]

The title compound was prepared in analogy to the preparation of Example 132 by using 2-(azetidin-3-yl)-6-bromo-pyridine instead of 1-(6-bromopyridin-3-yl)piperazine (compound 132a). Example 159 (2 mg) was obtained as a light yellow solid. MS: calc'd 485 (MH⁺), measured 485 (MH⁺). ¹H NMR (METHANOL-d₄, 400 MHz) δ 8.64 (d, 1H, J=8.6 Hz), 8.15 (d, 1H, J=7.9 Hz), 7.64 (d, 1H, J=8.6 Hz), 7.44 (dd, 1H, J=7.4, 8.1 Hz), 7.24 (d, 1H, J=8.1 Hz), 6.58 (d, 1H, J=7.3 Hz), 6.26 (d, 1H, J=8.3 Hz), 4.5-4.7 (m, 1H), 4.0-4.2 (m, 2H), 3.7-4.0 (m, 6H), 3.4-3.6 (m, 6H), 2.57 (br dd, 4H, J=2.6, 3.5 Hz), 1.49 (s, 3H).

### Example 160

### 4-[(2S,6R)-2-[[3-[4-(3-amino-3-methyl-azetidin-1-yl)phenyl]azetidin-1-yl]methyl]-6-methyl-morpholin-4-yl]-3-fluoro-pyrazolo[1,5-a]pyridine-7-carbonitrile

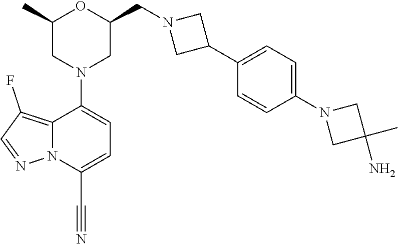 [SMILES: C[C@@H]1CN(c2ccc(C#N)n3ncc(F)c23)C[C@H](CN2CC(c3ccc(N4CC(C)(N)C4)cc3)C2)O1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and 3-(4-bromophenyl)azetidine instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a). Example 160 (4 mg) was obtained as a light yellow solid. MS: calc'd 490 (MH⁺), measured 490 (MH⁺). ¹H NMR (METHANOL-d₄, 400 MHz) δ 7.9-8.1 (m, 1H), 7.44 (d, 1H, J=7.9 Hz), 7.28 (br d, 2H, J=8.2 Hz), 6.4-6.7 (m, 3H), 4.3-4.7 (m, 2H), 4.0-4.3 (m, 4H), 3.8-4.0 (m, 4H), 3.4-3.7 (m, 5H), 2.6-2.8 (m, 2H), 1.66 (s, 3H), 1.29 (d, 3H, J=6.4 Hz).

### Example 161

### 2-deuterio-5-[(2R,6S)-2-methyl-6-[[4-[6-(6-methyl-2,6-diazaspiro[3.3]heptan-2-yl)-3-pyridyl]piperazin-1-yl]methyl]morpholin-4-yl]quinoline-8-carbonitrile

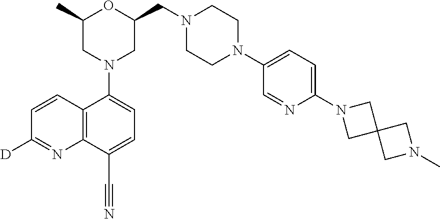 [SMILES: [2H]c1ccc2c(N3C[C@H](CN4CCN(c5ccc(N6CC7(CN(C)C7)C6)nc5)CC4)O[C@H](C)C3)ccc(C#N)c2n1]

The title compound was prepared in analogy to the preparation of Example 132 by using 2-methyl-2,6-diazaspiro[3.3]heptane instead of tert-butyl (3-methylazetidin-3-yl)carbamate (compound 132c). Example 161 (2 mg) was obtained as a light yellow solid. MS: calc'd 540 (MH⁺), measured 540 (MH⁺). ¹H NMR (METHANOL-d₄, 400 MHz) δ 8.66 (d, 1H, J=8.7 Hz), 8.15 (d, 1H, J=8.1 Hz), 7.72 (d, 1H, J=2.4 Hz), 7.63 (d, 1H, J=8.6 Hz), 7.38 (dd, 1H, J=2.9, 9.0 Hz), 7.25 (d, 1H, J=8.1 Hz), 6.42 (d, 1H, J=8.7 Hz), 4.1-4.2 (m, 1H), 4.0-4.1 (m, 1H), 4.0-4.0 (m, 3H), 3.5-3.5 (m, 1H), 3.4-3.4 (m, 4H), 3.4-3.4 (m, 1H), 3.4-3.4 (m, 1H), 3.06 (t, 3H, J=5.0 Hz), 2.8-2.9 (m, 2H), 2.7-2.7 (m, 3H), 2.6-2.7 (m, 2H), 2.5-2.6 (m, 1H), 2.3-2.3 (m, 4H), 1.25 (d, 3H, J=6.2 Hz).

### Example 162

### 2-deuterio-5-[(2S,6R)-2-[[4-[6-[[(3S,4R)-4-fluoropyrrolidin-3-yl]amino]-3-pyridyl]piperazin-1-yl]methyl]-6-methyl-morpholin-4-yl]quinoline-8-carbonitrile

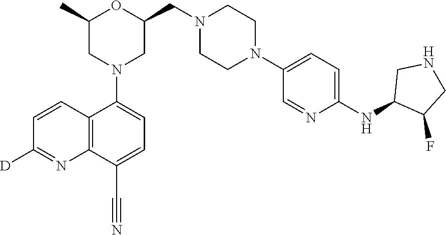 [SMILES: [2H]c1ccc2c(N3C[C@H](CN4CCN(c5ccc(N[C@H]6CNC[C@H]6F)nc5)CC4)O[C@H](C)C3)ccc(C#N)c2n1]

The title compound was prepared in analogy to the preparation of Example 132 by using tert-butyl (3S,4R)-3-amino-4-fluoro-pyrrolidine-1-carboxylate instead of tert-butyl (3-methylazetidin-3-yl)carbamate (compound 132c). Example 162 (6 mg) was obtained as a light yellow solid. MS: calc'd 532 (MH⁺), measured 532 (MH⁺). ¹H NMR (METHANOL-d₄, 400 MHz) δ 8.66 (d, 1H, J=8.6 Hz), 8.15 (d, 1H, J=8.1 Hz), 7.68 (d, 1H, J=2.4 Hz), 7.64 (d, 1H, J=8.7 Hz), 7.31 (dd, 1H, J=2.9, 9.1 Hz), 7.25 (d, 1H, J=8.1 Hz), 6.62 (d, 1H, J=9.0 Hz), 5.0-5.2 (m, 1H), 4.2-4.4 (m, 1H), 4.2-4.2 (m, 1H), 4.0-4.1 (m, 1H), 3.3-3.5 (m, 3H), 3.2-3.3 (m, 1H), 3.1-3.2 (m, 1H), 3.0-3.1 (m, 4H), 2.8-2.9 (m, 2H), 2.5-2.8 (m, 7H), 1.25 (d, 3H, J=6.2 Hz).

### Example 163

### 4-[(2S,6R)-2-[[3-[4-(3-amino-3-methyl-azetidin-1-yl)phenyl]azetidin-1-yl]methyl]-6-methyl-morpholin-4-yl]pyrazolo[1,5-a]pyridine-7-carbonitrile

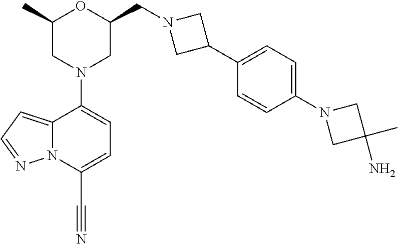 [SMILES: C[C@@H]1CN(c2ccc(C#N)n3nccc23)C[C@H](CN2CC(c3ccc(N4CC(C)(N)C4)cc3)C2)O1]

The title compound was prepared in analogy to the preparation of Example 132 by using [(2R,6R)-4-(7-cyanopyrazolo[1,5-a]pyridin-4-yl)-6-methyl-morpholin-2-yl]methyl trifluoromethanesulfonate (intermediate D) and 3-(4-bromophenyl)azetidine instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a). Example 163 (12 mg) was obtained as a light yellow solid. MS: calc'd 472 (MH⁺), measured 472 (MH⁺). ¹H NMR (METHANOL-d₄, 400 MHz) δ 8.04 (d, 1H, J=2.3 Hz), 7.49 (d, 1H, J=7.9 Hz), 7.29 (br d, 2H, J=8.3 Hz), 6.90 (d, 1H, J=2.4 Hz), 6.5-6.7 (m, 3H), 4.64 (s, 3H), 4.0-4.3 (m, 4H), 3.97 (d, 3H, J=8.3 Hz), 3.8-3.9 (m, 2H), 3.7-3.8 (m, 2H), 3.4-3.6 (m, 1H), 2.6-2.8 (m, 2H), 1.66 (s, 3H), 1.31 (br d, 3H, J=6.2 Hz)

### Example 164

### 4-[(2S,6R)-2-[[3-[6-[(3R,4R)-3-amino-4-methoxy-pyrrolidin-1-yl]-3-pyridyl]azetidin-1-yl]methyl]-6-methyl-morpholin-4-yl]-3-fluoro-pyrazolo[1,5-a]pyridine-7-carbonitrile

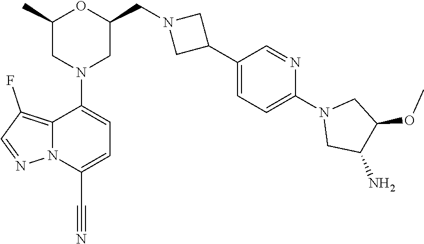 [SMILES: CO[C@@H]1CN(c2ccc(C3CN(C[C@H]4CN(c5ccc(C#N)n6ncc(F)c56)C[C@@H](C)O4)C3)cn2)C[C@H]1N]

The title compound was prepared in analogy to the preparation of Example 138 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and tert-butyl ((3R,4R)-4-methoxypyrrolidin-3-yl)carbamate instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 164 (5 mg) was obtained as a light yellow solid. MS: calc'd 521 (MH⁺), measured 521 (MH⁺). ¹H NMR (METHANOL-d₄, 400 MHz) δ 8.01 (d, 1H, J=3.7 Hz), 7.95 (d, 1H, J=2.2 Hz), 7.64 (dd, 1H, J=2.4, 8.7 Hz), 7.44 (d, 1H, J=7.9 Hz), 6.5-6.6 (m, 2H), 3.8-3.9 (m, 6H), 3.6-3.7 (m, 2H), 3.5-3.6 (m, 4H), 3.43 (s, 3H), 3.2-3.3 (m, 3H), 2.7-2.8 (m, 2H), 2.6-2.7 (m, 2H), 1.25 (d, 3H, J=6.2 Hz).

### Example 165

### 4-[(2S,6R)-2-[[3-[6-[(1S,4S)-2,5-diazabicyclo[2.2.1]heptan-2-yl]-3-pyridyl]azetidin-1-yl]methyl]-6-methyl-morpholin-4-yl]-3-fluoro-pyrazolo[1,5-a]pyridine-7-carbonitrile

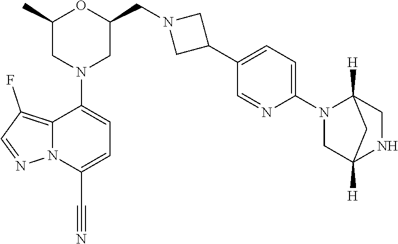 [SMILES: C[C@@H]1CN(c2ccc(C#N)n3ncc(F)c23)C[C@H](CN2CC(c3ccc(N4C[C@@H]5C[C@H]4CN5)nc3)C2)O1]

The title compound was prepared in analogy to the preparation of Example 138 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and (1S,4S)-2-Boc-2,5-diazabicyclo[2.2.1]heptane (Bidepharm, BD30269, CAS: 113451-59-5) instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 165 (10 mg) was obtained as a light yellow solid. MS: calc'd 503 (MH⁺), measured 503 (MH⁺). ¹H NMR (METHANOL-d₄, 400 MHz) δ 8.11 (d, 1H, J=2.0 Hz), 8.07 (dd, 1H, J=2.1, 9.1 Hz), 8.02 (d, 1H, J=3.5 Hz), 7.45 (d, 1H, J=7.9 Hz), 7.06 (br d, 1H, J=8.9 Hz), 6.56 (d, 1H, J=7.9 Hz), 5.11 (s, 1H), 4.66 (s, 1H), 4.57 (br t, 2H, J=9.2 Hz), 4.3-4.4 (m, 1H), 4.2-4.3 (m, 1H), 4.1-4.2 (m, 1H), 3.9-4.0 (m, 1H), 3.8-3.9 (m, 1H), 3.7-3.8 (m, 1H), 3.5-3.7 (m, 6H), 3.23 (d, 1H, J=7.3 Hz), 2.79 (br t, 1H, J=11.2 Hz), 2.7-2.7 (m, 1H), 2.36 (br d, 1H, J=11.5 Hz), 2.19 (br d, 1H, J=11.5 Hz), 1.30 (d, 3H, J=6.2 Hz).

### Example 166

### 4-[(2S,6R)-2-[[4-[6-[(3R,4R)-3-amino-4-methoxy-pyrrolidin-1-yl]-3-pyridyl]piperazin-1-yl]methyl]-6-methyl-morpholin-4-yl]-3-fluoro-pyrazolo[1,5-a]pyridine-7-carbonitrile

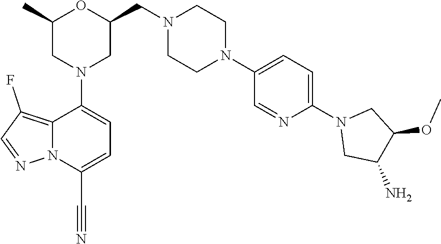 [SMILES: CO[C@@H]1CN(c2ccc(N3CCN(C[C@H]4CN(c5ccc(C#N)n6ncc(F)c56)C[C@@H](C)O4)CC3)cn2)C[C@H]1N]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and tert-butyl N-[(3R,4R)-4-methoxypyrrolidin-3-yl]carbamate instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 166 (10 mg) was obtained as a light yellow solid. MS: calc'd 550 (MH⁺), measured 550 (MH⁺). ¹H NMR (METHANOL-d₄, 400 MHz) δ 7.89 (d, 1H, J=3.7 Hz), 7.65 (d, 1H, J=2.7 Hz), 7.3-7.3 (m, 2H), 6.4-6.4 (m, 2H), 3.9-4.0 (m, 1H), 3.80 (ddd, 1H, J=2.3, 6.4, 10.1 Hz), 3.6-3.7 (m, 2H), 3.5-3.6 (m, 2H), 3.4-3.5 (m, 2H), 3.3-3.3 (m, 4H), 3.15 (dd, 1H, J=3.2, 10.1 Hz), 2.95 (t, 4H, J=4.9 Hz), 2.6-2.7 (m, 2H), 2.5-2.6 (m, 5H), 2.4-2.5 (m, 1H), 1.15 (d, 3H, J=6.4 Hz).

### Example 167

### 3-fluoro-4-[(2S,6R)-2-[[4-[6-[[(3S,4R)-4-fluoropyrrolidin-3-yl]amino]-3-pyridyl]piperazin-1-yl]methyl]-6-methyl-morpholin-4-yl]pyrazolo[1,5-a]pyridine-7-carbonitrile

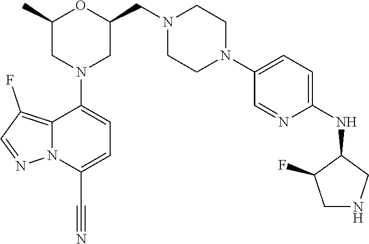 [SMILES: C[C@@H]1CN(c2ccc(C#N)n3ncc(F)c23)C[C@H](CN2CCN(c3ccc(N[C@H]4CNC[C@H]4F)nc3)CC2)O1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and tert-butyl (3S,4R)-3-amino-4-fluoro-pyrrolidine-1-carboxylate instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 167 (32 mg) was obtained as a light yellow solid. MS: calc'd 538 (MH⁺), measured 538 (MH⁺). ¹H NMR (METHANOL-d₄, 400 MHz) δ 8.10 (dd, 1H, J=2.8, 9.8 Hz), 8.04 (d, 1H, J=3.7 Hz), 7.56 (d, 1H, J=2.7 Hz), 7.47 (d, 1H, J=7.9 Hz), 7.29 (d, 1H, J=9.8 Hz), 6.60 (d, 1H, J=8.1 Hz), 5.4-5.6 (m, 1H), 4.4-4.4 (m, 1H), 4.0-4.1 (m, 1H), 3.7-4.0 (m, 7H), 3.6-3.7 (m, 3H), 3.4-3.5 (m, 5H), 3.37 (s, 1H), 3.2-3.3 (m, 1H), 2.7-2.9 (m, 2H), 1.34 (d, 3H, J=6.2 Hz).

### Example 168

### 4-[(2S,6R)-2-[[4-[6-[(4aR,7aR)-3,4,4a,5,7,7a-hexahydro-2H-pyrrolo[3,4-b][1,4]oxazin-6-yl]-3-pyridyl]piperazin-1-yl]methyl]-6-methyl-morpholin-4-yl]-3-fluoro-pyrazolo[1,5-a]pyridine-7-carbonitrile

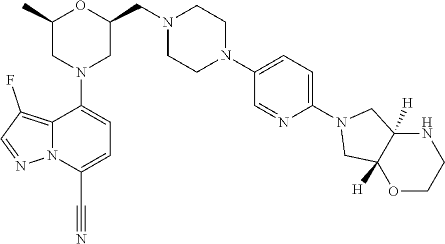 [SMILES: C[C@@H]1CN(c2ccc(C#N)n3ncc(F)c23)C[C@H](CN2CCN(c3ccc(N4C[C@H]5NCCO[C@@H]5C4)nc3)CC2)O1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and tert-butyl (4aR,7aR)-3,4a,5,6,7,7a-hexahydro-2H-pyrrolo[3,4-b][1,4]oxazine-4-carboxylate instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 168 (12 mg) was obtained as a light yellow solid. MS: calc'd 562 (MH⁺), measured 562 (MH⁺). ¹H NMR (METHANOL-d₄, 400 MHz) δ 7.89 (d, 1H, J=3.7 Hz), 7.65 (d, 1H, J=2.8 Hz), 7.3-7.3 (m, 2H), 6.41 (d, 2H, J=8.3 Hz), 3.9-4.0 (m, 1H), 3.9-3.9 (m, 1H), 3.8-3.8 (m, 1H), 3.5-3.7 (m, 5H), 3.4-3.5 (m, 1H), 2.9-3.1 (m, 10H), 2.7-2.7 (m, 2H), 2.6-2.6 (m, 2H), 2.5-2.6 (m, 3H), 1.15 (d, 3H, J=6.2 Hz).

### Example 169

### 3-fluoro-4-[(2R,6S)-2-methyl-6-[[4-[6-(5-oxa-2,8-diazaspiro[3.5]nonan-2-yl)-3-pyridyl]piperazin-1-yl]methyl]morpholin-4-yl]pyrazolo[1,5-a]pyridine-7-carbonitrile

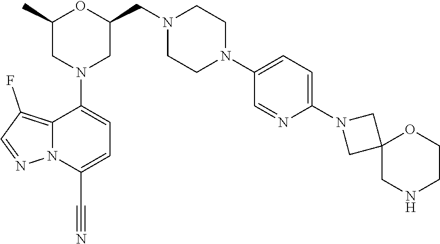 [SMILES: C[C@@H]1CN(c2ccc(C#N)n3ncc(F)c23)C[C@H](CN2CCN(c3ccc(N4CC5(CNCCO5)C4)nc3)CC2)O1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and tert-butyl 5-oxa-2,8-diazaspiro[3.5]nonane-8-carboxylate instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 169 (14 mg) was obtained as a light yellow solid. MS: calc'd 562 (MH⁺), measured 562 (MH⁺). ¹H NMR (METHANOL-d₄, 400 MHz) δ 7.88 (d, 1H, J=3.4 Hz), 7.64 (d, 1H, J=2.6 Hz), 7.2-7.4 (m, 2H), 6.38 (dd, 2H, J=8.6, 11.2 Hz), 3.9-4.0 (m, 1H), 3.8-3.9 (m, 3H), 3.6-3.7 (m, 2H), 3.5-3.6 (m, 3H), 3.41 (br d, 1H, J=11.9 Hz), 3.21 (br s, 2H), 2.97 (br t, 3H, J=4.5 Hz), 2.92 (s, 2H), 2.6-2.8 (m, 4H), 2.4-2.6 (m, 5H), 1.15 (d, 3H, J=6.2 Hz).

### Example 170

### 3-fluoro-4-[(2R,6S)-2-methyl-6-[[4-[6-(6-methyl-2,6-diazaspiro[3.3]heptan-2-yl)-3-pyridyl]piperazin-1-yl]methyl]morpholin-4-yl]pyrazolo[1,5-a]pyridine-7-carbonitrile

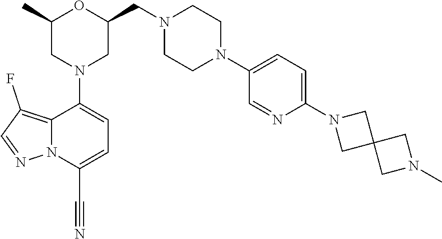 [SMILES: C[C@@H]1CN(c2ccc(C#N)n3ncc(F)c23)C[C@H](CN2CCN(c3ccc(N4CC5(CN(C)C5)C4)nc3)CC2)O1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and 2-methyl-2,6-diazaspiro[3.3]heptane instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 170 (28 mg) was obtained as a light yellow solid. MS: calc'd 546 (MH⁺), measured 546 (MH⁺). ¹H NMR (METHANOL-d₄, 400 MHz) δ 8.01 (d, 1H, J=3.7 Hz), 7.74 (d, 1H, J=2.6 Hz), 7.44 (d, 1H, J=7.9 Hz), 7.40 (dd, 1H, J=2.8, 9.0 Hz), 6.53 (d, 1H, J=8.1 Hz), 6.45 (d, 1H, J=8.9 Hz), 4.0-4.1 (m, 1H), 4.0-4.0 (m, 4H), 3.92 (ddd, 1H, J=2.3, 6.2, 10.1 Hz), 3.67 (br d, 1H, J=12.2 Hz), 3.54 (br d, 1H, J=12.0 Hz), 3.50 (s, 4H), 3.08 (t, 4H, J=4.9 Hz), 2.8-2.8 (m, 2H), 2.6-2.7 (m, 5H), 2.5-2.6 (m, 1H), 2.37 (s, 3H), 1.27 (d, 3H, J=6.2 Hz).

### Example 171

### 1-methyl-4-[(2R,6S)-2-methyl-6-[[3-(4-piperazin-1-ylphenyl)azetidin-1-yl]methyl]morpholin-4-yl]-1,8-naphthyridin-2-one

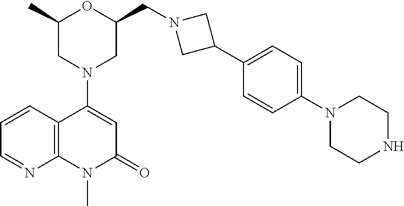 [SMILES: C[C@@H]1CN(c2cc(=O)n(C)c3ncccc23)C[C@H](CN2CC(c3ccc(N4CCNCC4)cc3)C2)O1]

The title compound was prepared in analogy to the preparation of Example 132 by using [(2R,6R)-6-methyl-4-(1-methyl-2-oxo-1,8-naphthyridin-4-yl)morpholin-2-yl]methyl trifluoromethanesulfonate (intermediate M) and 3-(4-bromophenyl)azetidine and tert-butyl piperazine-1-carboxylate instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a) and tert-butyl (3-methylazetidin-3-yl)carbamate (compound 132c). Example 171 (10 mg) was obtained as a light yellow solid. MS: calc'd 489 (MH⁺), measured 489 (MH⁺). ¹H NMR (METHANOL-d₄, 400 MHz) δ 8.65 (dd, 1H, J=1.7, 4.6 Hz), 8.26 (dd, 1H, J=1.8, 8.0 Hz), 7.3-7.4 (m, 3H), 7.07 (br d, 2H, J=8.6 Hz), 6.18 (s, 1H), 4.4-4.7 (m, 3H), 3.9-4.3 (m, 6H), 3.77 (s, 3H), 3.2-3.6 (m, 9H), 2.5-2.7 (m, 3H), 1.28 (d, 3H, J=6.1 Hz).

### Example 172

### 4-[(2S,6R)-2-[[3-[4-[(3R,4R)-3-amino-4-methoxy-pyrrolidin-1-yl]phenyl]azetidin-1-yl]methyl]-6-methyl-morpholin-4-yl]-1-methyl-1,8-naphthyridin-2-one

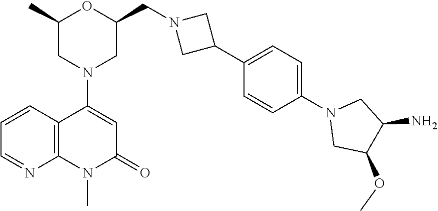 [SMILES: CO[C@H]1CN(c2ccc(C3CN(C[C@H]4CN(c5cc(=O)n(C)c6ncccc56)C[C@@H](C)O4)C3)cc2)C[C@H]1N]

The title compound was prepared in analogy to the preparation of Example 132 by using [(2R,6R)-6-methyl-4-(1-methyl-2-oxo-1,8-naphthyridin-4-yl)morpholin-2-yl]methyl trifluoromethanesulfonate (intermediate M) and 3-(4-bromophenyl)azetidine and tert-butyl N-[(3R,4R)-4-methoxypyrrolidin-3-yl]carbamate (CAS: 1932066-52-8) instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a) and tert-butyl (3-methylazetidin-3-yl)carbamate (compound 132c). Example 172 (10 mg) was obtained as a light yellow solid. MS: calc'd 519 (MH⁺), measured 519 (MH⁺). ¹H NMR (METHANOL-d₄, 400 MHz) δ 8.65 (dd, 1H, J=1.8, 4.6 Hz), 8.26 (dd, 1H, J=1.7, 8.1 Hz), 7.2-7.4 (m, 3H), 6.6-6.8 (m, 2H), 6.18 (s, 1H), 4.0-4.7 (m, 5H), 3.8-3.9 (m, 3H), 3.77 (s, 3H), 3.5-3.6 (m, 4H), 3.4-3.5 (m, 7H), 2.48 (br dd, 2H, J=8.5, 11.6 Hz), 1.28 (d, 4H, J=6.1 Hz).

### Example 173

### 1-methyl-4-[(2R,6S)-2-methyl-6-[[3-[4-(5-oxa-2,8-diazaspiro[3.5]nonan-2-yl)phenyl]azetidin-1-yl]methyl]morpholin-4-yl]-1,8-naphthyridin-2-one

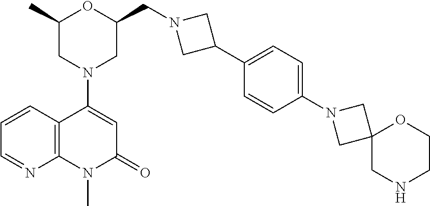 [SMILES: C[C@@H]1CN(c2cc(=O)n(C)c3ncccc23)C[C@H](CN2CC(c3ccc(N4CC5(CNCCO5)C4)cc3)C2)O1]

The title compound was prepared in analogy to the preparation of Example 132 by using [(2R,6R)-6-methyl-4-(1-methyl-2-oxo-1,8-naphthyridin-4-yl)morpholin-2-yl]methyl trifluoromethanesulfonate (intermediate M) and 3-(4-bromophenyl)azetidine and tert-butyl 5-oxa-2,8-diazaspiro[3.5]nonane-8-carboxylate instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a) and tert-butyl (3-methylazetidin-3-yl)carbamate (compound 132c). Example 173 (15 mg) was obtained as a light yellow solid. MS: calc'd 531 (MH⁺), measured 531 (MH⁺). ¹H NMR (METHANOL-d₄, 400 MHz) δ 8.65 (dd, 1H, J=1.7, 4.6 Hz), 8.26 (dd, 1H, J=1.8, 8.0 Hz), 7.2-7.4 (m, 3H), 6.5-6.7 (m, 2H), 6.18 (s, 1H), 4.68 (s, 3H), 4.1-4.3 (m, 3H), 3.9-4.1 (m, 4H), 3.6-3.8 (m, 7H), 3.3-3.5 (m, 7H), 2.5-2.8 (m, 2H), 1.28 (d, 3H, J=6.1 Hz).

### Example 174

### 4-[(2S,6R)-2-[[3-[4-[(3R,4R)-3-amino-4-fluoro-pyrrolidin-1-yl]phenyl]azetidin-1-yl]methyl]-6-methyl-morpholin-4-yl]-1-methyl-1,8-naphthyridin-2-one

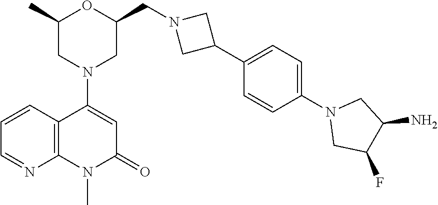 [SMILES: C[C@@H]1CN(c2cc(=O)n(C)c3ncccc23)C[C@H](CN2CC(c3ccc(N4C[C@@H](N)[C@@H](F)C4)cc3)C2)O1]

The title compound was prepared in analogy to the preparation of Example 132 by using [(2R,6R)-6-methyl-4-(1-methyl-2-oxo-1,8-naphthyridin-4-yl)morpholin-2-yl]methyl trifluoromethanesulfonate (intermediate M) and 3-(4-bromophenyl)azetidine and tert-butyl N-[(3R,4R)-4-fluoropyrrolidin-3-yl]carbamate (cas:2097061-04-4) instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a) and tert-butyl (3-methylazetidin-3-yl)carbamate (compound 132c). Example 174 (9 mg) was obtained as a light yellow solid. MS: calc'd 507 (MH⁺), measured 507 (MH⁺). ¹H NMR (METHANOL-d₄, 400 MHz) δ 8.65 (dd, 1H, J=1.7, 4.6 Hz), 8.26 (dd, 1H, J=1.7, 7.9 Hz), 7.32 (dd, 3H, J=4.5, 8.0 Hz), 6.73 (br d, 2H, J=8.4 Hz), 6.18 (s, 1H), 5.3-5.5 (m, 1H), 4.74 (br d, 3H, J=2.6 Hz), 4.0-4.3 (m, 4H), 3.8-4.0 (m, 1H), 3.7-3.8 (m, 4H), 3.4-3.7 (m, 7H), 2.4-2.8 (m, 2H), 1.28 (br d, 3H, J=6.2 Hz).

### Example 175

### 4-[(2S,6R)-2-[[4-[6-[(3R,4R)-4-amino-3-fluoro-1-piperidyl]-3-pyridyl]piperazin-1-yl]methyl]-6-methyl-morpholin-4-yl]-3-fluoro-pyrazolo[1,5-a]pyridine-7-carbonitrile

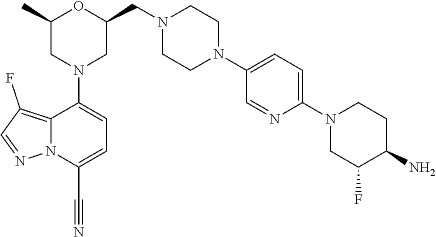 [SMILES: C[C@@H]1CN(c2ccc(C#N)n3ncc(F)c23)C[C@H](CN2CCN(c3ccc(N4CC[C@@H](N)[C@H](F)C4)nc3)CC2)O1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and tert-butyl ((3R,4R)-3-fluoropiperidin-4-yl)carbamate (CAS: 1523530-29-1) instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 175 (3 mg) was obtained as a light yellow solid. MS: calc'd 552 (MH⁺), measured 552 (MH⁺). ¹H NMR (METHANOL-d₄, 400 MHz) δ 7.98 (d, 1H, J=3.5 Hz), 7.83 (d, 1H, J=2.8 Hz), 7.41 (d, 1H, J=7.9 Hz), 7.36 (dd, 1H, J=3.1, 9.2 Hz), 6.85 (d, 1H, J=9.2 Hz), 6.51 (d, 1H, J=8.1 Hz), 4.2-4.4 (m, 2H), 3.9-4.1 (m, 2H), 3.9-3.9 (m, 1H), 3.6-3.7 (m, 1H), 3.5-3.6 (m, 1H), 3.0-3.1 (m, 4H), 2.9-3.0 (m, 1H), 2.7-2.9 (m, 4H), 2.5-2.7 (m, 6H), 1.9-2.0 (m, 1H), 1.50 (dq, 1H, J=4.0, 12.3 Hz), 1.24 (d, 3H, J=6.2 Hz).

### Example 176

### 4-[(2S,6R)-2-[[3-[4-(3-amino-3-methyl-azetidin-1-yl)phenyl]azetidin-1-yl]methyl]-6-methyl-morpholin-4-yl]-1-methyl-1,8-naphthyridin-2-one

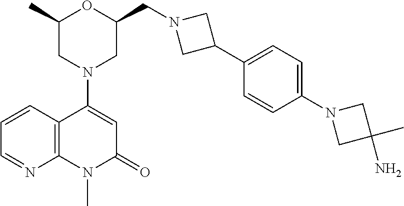 [SMILES: C[C@@H]1CN(c2cc(=O)n(C)c3ncccc23)C[C@H](CN2CC(c3ccc(N4CC(C)(N)C4)cc3)C2)O1]

The title compound was prepared in analogy to the preparation of Example 132 by using [(2R,6R)-6-methyl-4-(1-methyl-2-oxo-1,8-naphthyridin-4-yl)morpholin-2-yl]methyl trifluoromethanesulfonate (intermediate M) and 3-(4-bromophenyl)azetidine instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a). Example 176 (6 mg) was obtained as light yellow solid. MS: calc'd 489 (MH⁺), measured 489 (MH⁺). ¹H NMR (METHANOL-d₄, 400 MHz) δ 8.65 (dd, 1H, J=1.8, 4.6 Hz), 8.26 (dd, 1H, J=1.7, 8.1 Hz), 7.2-7.4 (m, 3H), 6.5-6.7 (m, 2H), 6.18 (s, 1H), 4.4-4.7 (m, 3H), 4.0-4.4 (m, 5H), 3.8-4.0 (m, 4H), 3.77 (s, 3H), 3.4-3.5 (m, 3H), 2.5-2.8 (m, 2H), 1.66 (s, 3H), 1.28 (d, 3H, J=6.1 Hz).

### Example 177

### 3-fluoro-4-[(2R,6S)-2-methyl-6-[[4-[4-[(2R)-morpholin-2-yl]phenyl]piperazin-1-yl]methyl]morpholin-4-yl]pyrazolo[1,5-a]pyridine-7-carbonitrile

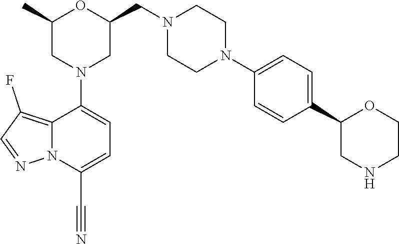 [SMILES: C[C@@H]1CN(c2ccc(C#N)n3ncc(F)c23)C[C@H](CN2CCN(c3ccc([C@@H]4CNCCO4)cc3)CC2)O1]

The title compound was prepared according to the following scheme:

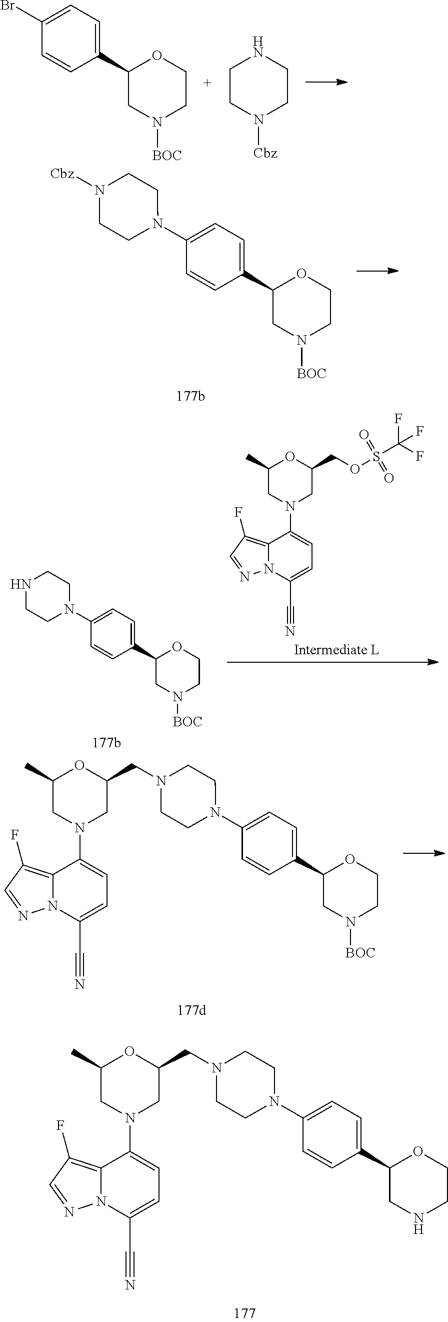

### Step 1: tert-butyl (R)-2-(4-(4-((benzyloxy)carbonyl)piperazin-1-yl)phenyl)-morpholine-4-carboxylate (Compound 177b)

To a solution of tert-butyl (R)-2-(4-bromophenyl)morpholine-4-carboxylate (compound 177a, CAS: 1312566-00-9, 100 mg, 292 μmol, synthesis refers to WO2012126922A1) in dioxane (2 mL) was added RuPhos-Pd-G2 (22.7 mg, 29.2 μmol), benzyl piperazine-1-carboxylate (84 mg, 380 μmol) and Cs₂CO₃ (286 mg, 877 μmol). After the reaction mixture was stirred for 16 hours at 120° C. under N₂, it was filtered and the filtrate was concentrated under reduced pressure. The crude product was purified by silica gel chromatography (EtOAc/PE from 0% to 40%) to afford tert-butyl (R)-2-(4-(4-((benzyloxy)carbonyl)piperazin-1-yl)phenyl)morpholine-4-carboxylate (compound 177b, 100 mg) as a white solid. MS: calc'd 482 (MH⁺), measured 482 (MH⁺).

### Step 2: tert-butyl (R)-2-(4-(piperazin-1-yl)phenyl)morpholine-4-carboxylate (Compound 177c)

The solution of tert-butyl (R)-2-(4-(4-((benzyloxy)carbonyl)piperazin-1-yl)phenyl)morpholine-4-carboxylate (compound 177b, 100 mg, 208 μmol) in MeOH (10 ml) was added PdOH/C (10 mg). Then the reaction mixture was charged with H₂ balloon and stirred at r.t. for 2 hours. The mixture was filtered and concentrated to afford crude product tert-butyl (R)-2-(4-(piperazin-1-yl)phenyl)morpholine-4-carboxylate (compound 177c, 70 mg), which was used for next step without further purification. MS: calc'd 348 (MH⁺), measured 348 (MH⁺).

### Step 3: tert-butyl (R)-2-(4-(4-(((2S,6R)-4-(7-cyano-3-fluoropyrazolo[1,5-a]pyridin-4-yl)-6-methylmorpholin-2-yl)methyl)piperazin-1-yl)phenyl)morpholine-4-carboxylate (Compound 177d)

A solution of tert-butyl (R)-2-(4-(piperazin-1-yl)phenyl)morpholine-4-carboxylate (compound 177c, 70 mg, 201 μmol), Et₃N (62 mg, 84.2 μL, 604 μmol) and ((2R,6R)-4-(7-cyano-3-fluoropyrazolo[1,5-a]pyridin-4-yl)-6-methylmorpholin-2-yl)methyl trifluoromethanesulfonate (intermediate E, 85 mg, 201 μmol) in acetonitrile (10 mL) was stirred at room temperature for 2 hrs. Then the mixture was concentrated and purified by silica gel Chromatography (MeOH/DCM from 2% to 10%) to afford tert-butyl (R)-2-(4-(4-(((2S,6R)-4-(7-cyano-3-fluoropyrazolo[1,5-a]pyridin-4-yl)-6-methylmorpholin-2-yl)methyl)piperazin-1-yl)phenyl)morpholine-4-carboxylate (compound 177d, 80 mg) as a yellow solid. MS: calc'd 620 (MH⁺), measured 620 (MH⁺).

### Step 4: tert-butyl (R)-2-(4-(4-(((2S,6R)-4-(7-cyano-3-fluoropyrazolo[1,5-a]pyridin-4-yl)-6-methylmorpholin-2-yl)methyl)piperazin-1-yl)phenyl)morpholine-4-carboxylate

### Example 177

To a solution of tert-butyl (R)-2-(4-(4-(((2S,6R)-4-(7-cyano-3-fluoropyrazolo[1,5-a]pyridin-4-yl)-6-methylmorpholin-2-yl)methyl)piperazin-1-yl)phenyl)morpholine-4-carboxylate (compound 177d, 80 mg) in DCM (2 mL) was added 2,2,2-trifluoroacetic acid (0.5 mL) at 0° C. The reaction mixture was stirred at rt for 2 hrs. Then the mixture was concentrated and purified by prep-HPLC to afford 3-fluoro-4-[(2R,6S)-2-methyl-6-[[4-[4-[(2R)-morpholin-2-yl]phenyl]piperazin-1-yl]methyl]morpholin-4-yl]pyrazolo[1,5-a]pyridine-7-carbonitrile (Example 177, 22 mg) as a light yellow solid. MS calc'd 520 (MH⁺), measured 520 (MH⁺). ¹H NMR (METHANOL-d₄, 400 MHz) δ 7.98 (d, 1H, J=3.5 Hz), 7.4-7.5 (m, 1H), 7.1-7.3 (m, 2H), 6.94 (br d, 2H, J=8.7 Hz), 6.50 (d, 1H, J=7.9 Hz), 4.3-4.4 (m, 1H), 3.9-4.1 (m, 3H), 3.7-3.8 (m, 1H), 3.6-3.7 (m, 1H), 3.5-3.5 (m, 1H), 3.1-3.2 (m, 4H), 2.5-2.9 (m, 12H), 1.2-1.3 (m, 3H).

### Example 178

### 3-fluoro-4-[(2R,6S)-2-methyl-6-[[4-[6-(5-oxa-2,8-diazaspiro[3.5]nonan-8-yl)-3-pyridyl]piperazin-1-yl]methyl]morpholin-4-yl]pyrazolo[1,5-a]pyridine-7-carbonitrile

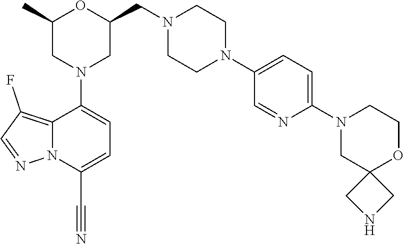 [SMILES: C[C@@H]1CN(c2ccc(C#N)n3ncc(F)c23)C[C@H](CN2CCN(c3ccc(N4CCOC5(CNC5)C4)nc3)CC2)O1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and tert-butyl 5-oxa-2,8-diazaspiro[3.5]nonane-2-carboxylate (Pharmablock, PBN20111063, CAS: 1251011-05-8) instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 178 (42 mg) was obtained as a light yellow solid. MS: calc'd 562 (MH⁺), measured 562 (MH⁺). ¹H NMR (METHANOL-d₄, 400 MHz) δ 7.99 (d, 1H, J=3.7 Hz), 7.87 (dd, 1H, J=3.0, 9.6 Hz), 7.78 (d, 1H, J=2.9 Hz), 7.43 (d, 1H, J=7.9 Hz), 7.26 (d, 1H, J=9.5 Hz), 6.54 (d, 1H, J=8.1 Hz), 4.3-4.4 (m, 1H), 4.13 (q, 4H, J=11.6 Hz), 4.0-4.1 (m, 1H), 3.9-3.9 (m, 2H), 3.81 (s, 2H), 3.5-3.8 (m, 6H), 3.3-3.5 (m, 7H), 3.1-3.3 (m, 1H), 2.7-2.8 (m, 2H), 1.30 (d, 3H, J=6.2 Hz).

### Example 179

### 4-[(2S,6R)-2-[[4-[6-(1,6-diazaspiro[3.3]heptan-6-yl)-3-pyridyl]piperazin-1-yl]methyl]-6-methyl-morpholin-4-yl]-3-fluoro-pyrazolo[1,5-a]pyridine-7-carbonitrile

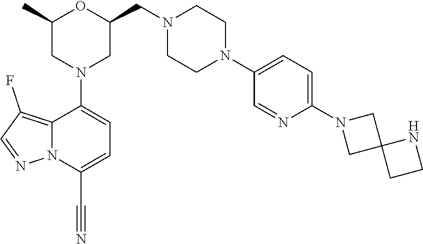 [SMILES: C[C@@H]1CN(c2ccc(C#N)n3ncc(F)c23)C[C@H](CN2CCN(c3ccc(N4CC5(CCN5)C4)nc3)CC2)O1]

The title compound was prepared in analogy to the preparation of Example 132 by using [(2R,6R)-4-(7-cyano-3-fluoro-pyrazolo[1,5-a]pyridin-4-yl)-6-methyl-morpholin-2-yl]methyl trifluoromethanesulfonate (intermediate E) and tert-butyl 1,6-diazaspiro[3.3]heptane-1-carboxylate (Pharmablock, PBN2011926-02, CAS:1394319-56-2) instead of [(2R,6R)-4-(8-cyano-2-deuterio-5-quinolyl)-6-methyl-morpholin-2-yl]methyl trifluoromethanesulfonate (intermediate L) and tert-butyl (3-methylazetidin-3-yl)carbamate (compound 132c). Example 179 (19 mg) was obtained as a light yellow solid. MS: calc'd 532 (MH⁺), measured 532 (MH⁺). ¹H NMR (METHANOL-d₄, 400 MHz) δ 7.99 (d, 1H, J=3.7 Hz), 7.92 (dd, 1H, J=2.8, 9.5 Hz), 7.62 (d, 1H, J=2.6 Hz), 7.43 (d, 1H, J=7.9 Hz), 6.8-6.9 (m, 1H), 6.54 (d, 1H, J=7.9 Hz), 4.73 (dd, 2H, J=1.4, 10.6 Hz), 4.54 (dd, 2H, J=1.3, 10.7 Hz), 4.3-4.4 (m, 1H), 4.0-4.1 (m, 3H), 3.5-3.8 (m, 8H), 3.4-3.5 (m, 4H), 2.9-2.9 (m, 2H), 2.7-2.8 (m, 2H), 1.30 (d, 3H, J=6.2 Hz).

### Example 180

### 1-methyl-4-[(2R,6S)-2-methyl-6-[[3-[4-(6-methyl-2,6-diazaspiro[3.3]heptan-2-yl)phenyl]azetidin-1-yl]methyl]morpholin-4-yl]-1,8-naphthyridin-2-one

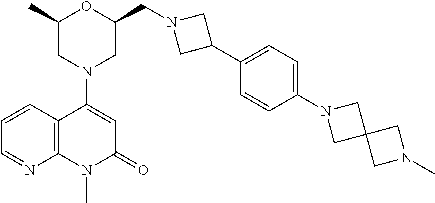 [SMILES: C[C@@H]1CN(c2cc(=O)n(C)c3ncccc23)C[C@H](CN2CC(c3ccc(N4CC5(CN(C)C5)C4)cc3)C2)O1]

The title compound was prepared in analogy to the preparation of Example 132 by using [(2R,6R)-6-methyl-4-(1-methyl-2-oxo-1,8-naphthyridin-4-yl)morpholin-2-yl]methyl trifluoromethanesulfonate (intermediate M) and 3-(4-bromophenyl)azetidine and 2-methyl-2,6-diazaspiro[3.3]heptane instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a) and tert-butyl (3-methylazetidin-3-yl)carbamate (compound 132c). Example 180 (18 mg) was obtained as a light yellow solid. MS: calc'd 515 (MH⁺), measured 515 (MH⁺). ¹H NMR (METHANOL-d₄, 400 MHz) δ 8.64 (dd, 1H, J=1.7, 4.6 Hz), 8.25 (dd, 1H, J=1.6, 7.9 Hz), 7.32 (dd, 1H, J=4.6, 7.9 Hz), 7.26 (br d, 2H, J=8.3 Hz), 6.53 (br d, 2H, J=8.3 Hz), 6.17 (s, 1H), 4.4-4.6 (m, 4H), 4.1-4.3 (m, 5H), 4.0-4.1 (m, 5H), 3.76 (s, 3H), 3.3-3.7 (m, 5H), 2.94 (s, 3H), 2.5-2.7 (m, 2H), 1.28 (br d, 3H, J=6.1 Hz).

### Example 182

### 4-[(2S,6R)-2-[[3-[4-[(4aR,7aR)-3,4,4a,5,7,7a-hexahydro-2H-pyrrolo[3,4-b][1,4]oxazin-6-yl]phenyl]azetidin-1-yl]methyl]-6-methyl-morpholin-4-yl]-1-methyl-1,8-naphthyridin-2-one

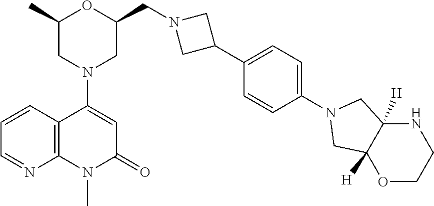 [SMILES: C[C@@H]1CN(c2cc(=O)n(C)c3ncccc23)C[C@H](CN2CC(c3ccc(N4C[C@H]5NCCO[C@@H]5C4)cc3)C2)O1]

The title compound was prepared in analogy to the preparation of Example 132 by using [(2R,6R)-6-methyl-4-(1-methyl-2-oxo-1,8-naphthyridin-4-yl)morpholin-2-yl]methyl trifluoromethanesulfonate (intermediate M) and 3-(4-bromophenyl)azetidine and tert-butyl rac-(4aR,7aR)-3,4a,5,6,7,7a-hexahydro-2H-pyrrolo[3,4-b][1,4]oxazine-4-carboxylate instead of [(2R,6R)-4-(8-cyano-2-deuterio-5-quinolyl)-6-methyl-morpholin-2-yl]methyl trifluoromethanesulfonate (intermediate L) and 1-(6-bromopyridin-3-yl)piperazine (compound 132a) and tert-butyl (3-methylazetidin-3-yl)carbamate (compound 132c). Example 182 (8 mg) was obtained as a white solid. MS: calc'd 531 (MH⁺), measured 531 (MH⁺). ¹H NMR (METHANOL-d₄, 400 MHz) δ 8.63 (dd, 1H, J=1.7, 4.6 Hz), 8.24 (dd, 1H, J=1.7, 7.9 Hz), 7.31 (dd, 1H, J=4.6, 8.1 Hz), 7.13 (d, 2H, J=8.6 Hz), 6.49 (d, 2H, J=8.6 Hz), 6.14 (s, 1H), 3.9-4.0 (m, 3H), 3.7-3.9 (m, 6H), 3.6-3.7 (m, 2H), 3.50 (dt, 2H, J=4.5, 7.5 Hz), 3.3-3.4 (m, 2H), 3.23 (dt, 2H, J=4.6, 7.8 Hz), 3.13 (t, 1H, J=8.8 Hz), 2.9-3.1 (m, 4H), 2.7-2.7 (m, 2H), 2.5-2.6 (m, 2H), 1.22 (d, 3H, J=6.2 Hz).

### Example 183

### 3-fluoro-4-[(2S,6R)-2-[[4-[2-fluoro-4-[(2S)-morpholin-2-yl]phenyl]piperazin-1-yl]methyl]-6-methyl-morpholin-4-yl]pyrazolo[1,5-a]pyridine-7-carbonitrile

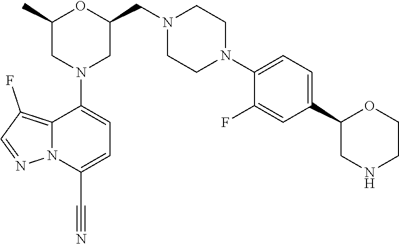 [SMILES: C[C@@H]1CN(c2ccc(C#N)n3ncc(F)c23)C[C@H](CN2CCN(c3ccc([C@@H]4CNCCO4)cc3F)CC2)O1]

The title compound was prepared in analogy to the preparation of Example 177 by using tert-butyl (2S)-2-(4-bromo-3-fluoro-phenyl)morpholine-4-carboxylate (CAS: 1447831-94-8, synthesis refers to WO2012126922A1) instead of tert-butyl (R)-2-(4-bromophenyl)morpholine-4-carboxylate (compound 177a). Example 183 (18 mg) was obtained as a white solid. MS: calc'd 538 (MH⁺), measured 538 (MH⁺). ¹H NMR (METHANOL-d₄, 400 MHz) δ 7.98 (d, 1H, J=3.7 Hz), 7.41 (d, 1H, J=8.1 Hz), 7.30 (t, 1H, J=8.6 Hz), 6.75 (dd, 1H, J=2.4, 8.7 Hz), 6.64 (dd, 1H, J=2.3, 13.9 Hz), 6.50 (d, 1H, J=7.9 Hz), 4.67 (dd, 1H, J=2.3, 10.5 Hz), 4.0-4.1 (m, 1H), 3.96 (dd, 1H, J=2.6, 11.6 Hz), 3.9-3.9 (m, 1H), 3.75 (dt, 1H, J=3.0, 11.5 Hz), 3.65 (br d, 1H, J=12.3 Hz), 3.52 (br d, 1H, J=12.1 Hz), 3.21 (t, 4H, J=5.1 Hz), 2.8-3.0 (m, 3H), 2.7-2.8 (m, 3H), 2.6-2.7 (m, 4H), 2.6-2.6 (m, 1H), 2.5-2.6 (m, 1H), 1.24 (d, 3H, J=6.2 Hz).

### Example 184

### 3-fluoro-4-[(2S,6R)-2-[[4-[3-fluoro-4-[(2R)-morpholin-2-yl]phenyl]piperazin-1-yl]methyl]-6-methyl-morpholin-4-yl]pyrazolo[1,5-a]pyridine-7-carbonitrile

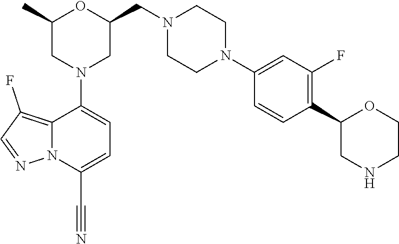 [SMILES: C[C@@H]1CN(c2ccc(C#N)n3ncc(F)c23)C[C@H](CN2CCN(c3ccc([C@@H]4CNCCO4)c(F)c3)CC2)O1]

The title compound was prepared in analogy to the preparation of Example 177 by using tert-butyl (2R)-2-(4-bromo-2-fluoro-phenyl)morpholine-4-carboxylate (CAS: 14400997-81-0, synthesis refers to WO2012126922A1) instead of tert-butyl (R)-2-(4-bromophenyl)morpholine-4-carboxylate (compound 177a). Example 184 (64 mg) was obtained as a white solid. MS: calc'd 538 (MH⁺), measured 538 (MH⁺). ¹H NMR (METHANOL-d₄, 400 MHz) δ 7.99 (d, 1H, J=3.7 Hz), 7.4-7.4 (m, 1H), 7.0-7.1 (m, 3H), 6.5-6.5 (m, 1H), 4.40 (dd, 1H, J=2.3, 10.5 Hz), 3.9-4.1 (m, 3H), 3.73 (dt, 1H, J=3.4, 11.3 Hz), 3.65 (br d, 1H, J=12.2 Hz), 3.52 (br d, 1H, J=12.0 Hz), 3.10 (t, 4H, J=4.6 Hz), 2.9-3.0 (m, 1H), 2.7-2.9 (m, 4H), 2.6-2.7 (m, 6H), 2.5-2.6 (m, 1H), 1.25 (d, 3H, J=6.4 Hz).

### Example 185

### 3-fluoro-4-[(2R,6S)-2-methyl-6-[[4-[4-(2-methylmorpholin-2-yl)phenyl]piperazin-1-yl]methyl]morpholin-4-yl]pyrazolo[1,5-a]pyridine-7-carbonitrile

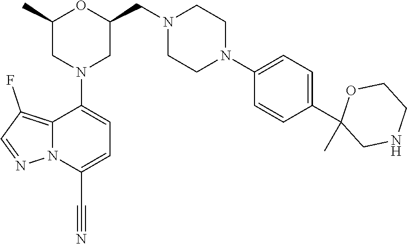 [SMILES: C[C@@H]1CN(c2ccc(C#N)n3ncc(F)c23)C[C@H](CN2CCN(c3ccc(C4(C)CNCCO4)cc3)CC2)O1]

The title compound was prepared in analogy to the preparation of Example 177 by using tert-butyl 2-(4-bromophenyl)-2-methyl-morpholine-4-carboxylate (CAS: 179821-59-1, synthesis refers to WO 9618615 A1) instead of tert-butyl (R)-2-(4-bromophenyl)morpholine-4-carboxylate (compound 177a). Example 185 (35 mg) was obtained as a white solid. MS: calc'd 534 (MH⁺), measured 534 (MH⁺). ¹H NMR (METHANOL-d₄, 400 MHz) δ 7.99 (d, 1H, J=3.5 Hz), 7.41 (d, 1H, J=7.9 Hz), 7.31 (d, 2H, J=8.8 Hz), 7.00 (d, 2H, J=8.9 Hz), 6.51 (d, 1H, J=7.9 Hz), 4.0-4.1 (m, 1H), 3.9-3.9 (m, 1H), 3.6-3.7 (m, 3H), 3.5-3.5 (m, 1H), 3.39 (d, 1H, J=13.6 Hz), 3.20 (t, 4H, J=5.0 Hz), 2.7-2.9 (m, 4H), 2.6-2.7 (m, 4H), 2.5-2.6 (m, 3H), 1.3 (s, 3H), 1.25 (d, 3H, J=6.4 Hz).

### Example 186

### 3-fluoro-4-[(2R,6S)-2-methyl-6-[[4-[4-[[(2R)-morpholin-2-yl]methyl]phenyl]piperazin-1-yl]methyl]morpholin-4-yl]pyrazolo[1,5-a]pyridine-7-carbonitrile

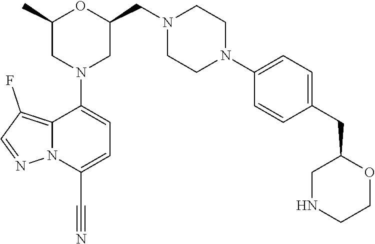 [SMILES: C[C@@H]1CN(c2ccc(C#N)n3ncc(F)c23)C[C@H](CN2CCN(c3ccc(C[C@@H]4CNCCO4)cc3)CC2)O1]

The title compound was prepared in analogy to the preparation of Example 177 by using tert-butyl (2R)-2-[(4-bromophenyl)methyl]morpholine-4-carboxylate (compound 186a) instead of tert-butyl (R)-2-(4-bromophenyl)morpholine-4-carboxylate (compound 177a). Example 186 (42 mg) was obtained as a white solid. MS: calc'd 534 (MH⁺), measured 534 (MH⁺). ¹H NMR (METHANOL-d₄, 400 MHz) δ 7.99 (d, 1H, J=3.7 Hz), 7.42 (d, 1H, J=7.9 Hz), 7.09 (d, 2H, J=8.6 Hz), 6.90 (d, 2H, J=8.7 Hz), 6.52 (s, 1H), 4.0-4.1 (m, 1H), 3.9-3.9 (m, 1H), 3.81 (br d, 1H, J=11.0 Hz), 3.5-3.7 (m, 4H), 3.16 (t, 4H, J=5.0 Hz), 2.7-2.8 (m, 5H), 2.5-2.7 (m, 9H), 1.25 (d, 3H, J=6.2 Hz).

### Preparation of tert-butyl (2R)-2-[(4-bromophenyl)methyl]morpholine-4-carboxylate (Compound 186a)

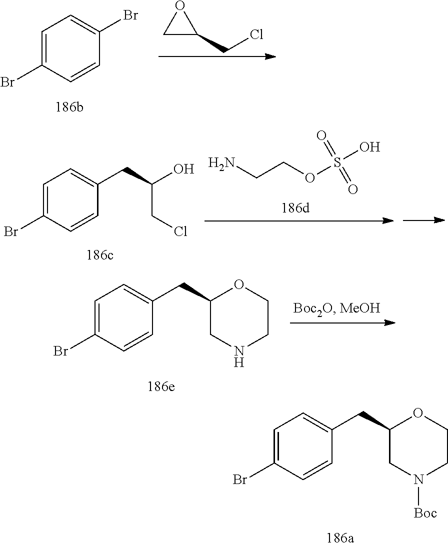

### Step 1: preparation of (2R)-1-(4-bromophenyl)-3-chloro-propan-2-ol (Compound 186c)

Magnesium (587 mg, 24.2 mmol) was placed in a three-necked flask, fitted with Ar-stream. 10 mL of THF was added. 1,4-dibromobenzene (compound 186b, 6 g, 25.4 mmol) dissolved in 20 mL of THF was slowly dropped into the flask. The reaction mixture was heated under reflux for 2 hours until all the magnesium disappeared. The solution was cooled down to 0° C. and (R)-2-(chloromethyl)oxirane (2.35 g, 1.99 mL, 25.4 mmol) dissolved in 10 mL THF was added dropwise in 10 minutes. After the reaction mixture was stirred for 1 hour, saturated NH₄Cl was added and the solution was extracted with EtOAC twice. The combined organic layer was dried over MgSO₄ and evaporated. Then the residue was purified by silica gel chromatography (EA/PE from 20% to 80%) to afford compound 186c (3.9 g). MS: calc'd 249 (MH⁺), measured 249 (MH⁺).

### Step 2: preparation of (2R)-2-[(4-bromophenyl)methyl]morpholine (Compound 186e)

(2R)-1-(4-bromophenyl)-3-chloro-propan-2-ol (compound 186c, 3.34 g, 13.4 mmol) was dissolved in methanol (6.7 mL). NaOH (3.21 g, 80.3 mmol) dissolved in water (3.5 mL) was added to form a suspension. After the reaction mixture was stirred at r.t. for another hour, 2-aminoethyl hydrogen sulfate (compound 186d, 7.56 g, 53.5 mmol) was added. The reaction mixture was heated at 45° C. for 2 hours and diluted with toluene (23 mL), then NaOH (3.37 g, 84.3 mmol) was added. After being stirred at 65° C. overnight, the reaction mixture was cooled down and diluted with water, extracted with EtOAc. The organic layer was washed with brine, dried over MgSO₄ and concentrated. The residue was purified by silica gel chromatography (EA/PE from 20% to 80%) to afford compound 186e (1.1 g). MS: calc'd 256 (MH⁺), measured 256 (MH⁺).

### Step 3: preparation of tert-butyl (2R)-2-[(4-bromophenyl)methyl]morpholine-4-carboxylate (Compound 186a)

(2R)-2-[(4-bromophenyl)methyl]morpholine (compound 186e, 1.042 g, 4.07 mmol) was dissolved in DCM (10 mL). Then di-tert-butyl dicarbonate (1.07 g, 4.88 mmol) was added and the reaction mixture was stirred at rt for 1 hour. After the reaction mixture was concentrated, the residue was purified by silica gel chromatography (EA/PE from 20% to 80%) to afford compound 186a (1.4 g). MS: calc'd 356 (MH⁺), measured 356 (MH⁺).

### Example 187

The following tests were carried out in order to determine the activity of the compounds of formula (I) and (Ia) in HEK293-Blue-hTLR-7/8/9 cells assay.

HEK293-Blue-hTLR-7 Cells Assay:

A stable HEK293-Blue-hTLR-7 cell line was purchased from InvivoGen (Cat. #: hkb-ht1r7, San Diego, Calif., USA). These cells were originally designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was declined by TLR7 antagonist under the stimulation of a ligand, such as R848 (Resiquimod), for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signaling pathway that TLR7 activation leads to downstream NF-κB activation has been widely accepted, and therefore similar reporter assay was modified for evaluating TLR7 antagonist.

HEK293-Blue-hTLR-8 Cells Assay:

A stable HEK293-Blue-hTLR-8 cell line was purchased from InvivoGen (Cat. #: hkb-ht1r8, San Diego, Calif., USA). These cells were originally designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR8 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 60 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signaling pathway that TLR8 activation leads to downstream NF-κB activation has been widely accepted, and therefore similar reporter assay was modified for evaluating TLR8 antagonist.

HEK293-Blue-hTLR-9 Cells Assay:

A stable HEK293-Blue-hTLR-9 cell line was purchased from InvivoGen (Cat. #: hkb-htlr9, San Diego, Calif., USA). These cells were originally designed for studying the stimulation of human TLR9 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR9 cells with TLR9 ligands. Therefore the reporter expression was declined by TLR9 antagonist under the stimulation of a ligand, such as ODN2006 (Cat. #: tlrl-2006-1, Invivogen, San Diego, Calif., USA), for incubation of 20 hrs. The cell culture supernatant SEAP reporter activity was determined using QUANTI-Blue™ kit (Cat. #: rep-qb1, Invivogen, San Diego, Calif., USA) at a wavelength of 640 nm, a detection medium that turns purple or blue in the presence of alkaline phosphatase.

HEK293-Blue-hTLR9 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM ODN2006 in above DMEM, perform incubation under 37° C. in a CO₂ incubator for 20 hrs. Then 20 μL of the supernatant from each well was incubated with 180 μL Quanti-blue substrate solution at 37° C. for 2 h and the absorbance was read at 620-655 nm using a spectrophotometer. The signaling pathway that TLR9 activation leads to downstream NF-κB activation has been widely accepted, and therefore similar reporter assay was modified for evaluating TLR9 antagonist.

The compounds of formula (I) or (Ia) have human TLR7 and/or TLR8 inhibitory activities (IC₅₀ value)<0.5 μM. Moreover, some compounds also have human TLR9 inhibitory activity <0.5 μM. Activity data of the compounds of the present invention were shown in Table 2.

TABLE 2
The activity of the compounds of present invention
in HEK293-Blue-hTLR-7/8/9 cells assays

| | HEK/hTLR7 | HEK/hTLR8 | HEK/hTLR9 |
|---|---|---|---|
| Example No | IC₅₀ (μM) | IC₅₀ (μM) | IC₅₀ (μM) |
| R1 | 0.095 | 0.142 | 6.623 |
| (reference compound) | | | |
| R2 | 0.210 | 0.342 | 7.034 |
| (reference compound) | | | |
| 1 | 0.084 | 0.040 | 0.136 |
| 2 | 0.093 | 0.026 | 0.074 |
| 3 | 0.011 | 0.009 | 0.052 |
| 4 | 0.016 | 0.017 | 0.068 |
| 5 | 0.054 | 0.033 | 0.198 |
| 6 | 0.051 | 0.007 | 0.120 |
| 7 | 0.034 | 0.009 | 0.051 |
| 8 | 0.050 | 0.010 | 0.093 |
| 9 | 0.019 | 0.003 | 0.032 |
| 10 | 0.033 | 0.010 | 0.105 |
| 11 | 0.023 | 0.013 | 0.092 |
| 12 | 0.012 | 0.003 | 0.032 |
| 13 | 0.018 | 0.005 | 0.142 |
| 14 | 0.006 | 0.003 | 0.032 |
| 15 | 0.007 | 0.003 | 0.036 |
| 17 | 0.031 | 0.006 | 0.186 |
| 18 | 0.011 | 0.006 | 0.052 |
| 19 | 0.025 | 0.012 | 0.091 |
| 20 | 0.020 | 0.010 | 0.096 |
| 21 | 0.016 | 0.010 | 0.219 |
| 22 | 0.010 | 0.003 | 0.045 |
| 23 | 0.050 | 0.018 | 0.232 |
| 24 | 0.038 | 0.011 | 0.032 |
| 25 | 0.026 | 0.007 | 0.033 |
| 26 | 0.008 | 0.003 | 0.032 |
| 27 | 0.015 | 0.003 | 0.043 |
| 28 | 0.011 | 0.004 | 0.065 |
| 29 | 0.016 | 0.010 | 0.051 |
| 30 | 0.016 | 0.006 | 0.250 |
| 31 | 0.018 | 0.005 | 0.046 |
| 32 | 0.006 | 0.003 | 0.073 |
| 33 | 0.024 | 0.007 | 0.116 |
| 34 | 0.027 | 0.004 | 0.071 |
| 35 | 0.065 | 0.033 | 0.147 |
| 36 | 0.028 | 0.011 | 0.118 |
| 37 | 0.070 | 0.030 | 0.234 |
| 38 | 0.016 | 0.011 | 0.097 |
| 39 | 0.051 | 0.023 | 0.104 |
| 40 | 0.035 | 0.017 | 0.134 |
| 41 | 0.081 | 0.032 | 0.150 |
| 42 | 0.023 | 0.013 | 0.147 |
| 43 | 0.022 | 0.011 | 0.119 |
| 44 | 0.028 | 0.010 | 0.164 |
| 45 | 0.015 | 0.004 | 0.188 |
| 46 | 0.018 | 0.009 | 0.157 |
| 47 | 0.013 | 0.003 | 0.067 |
| 48 | 0.009 | 0.004 | 0.121 |
| 49 | 0.029 | 0.010 | 0.146 |
| 50 | 0.016 | 0.003 | 0.126 |
| 51 | 0.028 | 0.003 | 0.121 |
| 52 | 0.015 | 0.003 | 0.032 |
| 53 | 0.008 | 0.003 | 0.032 |
| 54 | 0.009 | 0.003 | 0.034 |
| 55 | 0.014 | 0.003 | 0.032 |
| 56 | 0.056 | 0.006 | 0.034 |
| 57 | 0.027 | 0.004 | 0.038 |
| 58 | 0.030 | 0.004 | 0.045 |
| 59 | 0.011 | 0.003 | 0.032 |
| 60 | 0.011 | 0.003 | 0.032 |
| 61 | 0.008 | 0.003 | 0.032 |
| 62 | 0.018 | 0.014 | 0.092 |
| 63 | 0.054 | 0.013 | 0.246 |
| 64 | 0.005 | 0.002 | 0.035 |
| 65 | 0.015 | 0.009 | 0.129 |
| 66 | 0.017 | 0.008 | 0.032 |
| 67 | 0.024 | 0.023 | 0.081 |
| 68 | 0.009 | 0.002 | 0.095 |
| 69A | 0.011 | 0.002 | 0.054 |
| 69B | 0.007 | 0.002 | 0.070 |
| 70 | 0.070 | 0.012 | 0.080 |
| 71 | 0.045 | 0.015 | 0.066 |
| 72 | 0.006 | 0.003 | 0.046 |
| 73 | 0.049 | 0.017 | 0.069 |
| 74 | 0.024 | 0.003 | 0.072 |
| 75 | 0.025 | 0.003 | 0.057 |
| 76 | 0.020 | 0.003 | 0.050 |
| 77 | 0.026 | 0.002 | 0.056 |
| 78 | 0.020 | 0.001 | 0.065 |
| 79 | 0.021 | 0.002 | 0.063 |
| 80 | 0.107 | 0.006 | 0.120 |
| 81 | 0.022 | 0.002 | 0.046 |
| 82 | 0.029 | 0.001 | 0.056 |
| 83 | 0.032 | 0.003 | 0.096 |
| 84 | 0.010 | 0.007 | 0.104 |
| 86 | 0.022 | 0.006 | 0.189 |
| 87 | 0.008 | 0.002 | 0.075 |
| 88 | 0.021 | 0.002 | 0.071 |
| 89 | 0.006 | 0.029 | 0.099 |
| 90 | 0.021 | 0.002 | 0.054 |
| 91 | 0.013 | 0.008 | 0.102 |
| 92 | 0.005 | 0.002 | 0.042 |
| 93 | 0.012 | 0.003 | 0.031 |
| 94 | 0.013 | 0.006 | 0.051 |
| 95 | 0.007 | 0.003 | 0.031 |
| 96 | 0.018 | 0.004 | 0.109 |
| 97 | 0.027 | 0.001 | 0.034 |
| 98 | 0.006 | 0.001 | 0.049 |
| 99 | 0.011 | 0.001 | 0.063 |
| 101 | 0.011 | 0.004 | 0.031 |
| 102 | 0.018 | 0.002 | 0.080 |
| 103 | 0.010 | 0.001 | 0.100 |
| 104 | 0.022 | 0.001 | 0.070 |
| 105 | 0.013 | 0.000 | 0.069 |
| 106 | 0.009 | 0.001 | 0.046 |
| 107 | 0.008 | 0.001 | 0.080 |
| 108 | 0.005 | 0.001 | 0.068 |
| 109 | 0.016 | 0.002 | 0.046 |
| 110 | 0.014 | 0.002 | 0.068 |
| 111 | 0.003 | 0.001 | 0.037 |
| 112 | 0.004 | 0.001 | 0.065 |
| 113 | 0.008 | 0.001 | 0.098 |
| 114 | 0.003 | 0.001 | 0.062 |
| 115 | 0.004 | 0.001 | 0.038 |
| 116 | 0.021 | 0.005 | 0.089 |
| 117 | 0.013 | 0.007 | 0.109 |
| 118 | 0.004 | 0.001 | 0.051 |
| 119 | 0.016 | 0.001 | 0.094 |
| 120 | 0.005 | 0.001 | 0.031 |
| 121 | 0.008 | 0.001 | 0.044 |
| 122 | 0.005 | 0.001 | 0.031 |
| 123 | 0.003 | 0.001 | 0.031 |
| 124 | 0.005 | 0.001 | 0.031 |
| 125 | 0.047 | 0.002 | 0.083 |
| 126 | 0.021 | 0.006 | 0.146 |
| 127 | 0.029 | 0.009 | 0.149 |
| 128 | 0.030 | 0.012 | 0.182 |
| 129 | 0.020 | 0.007 | 0.171 |
| 130 | 0.016 | 0.008 | 0.155 |
| 131 | 0.032 | 0.004 | 0.113 |
| 132 | 0.023 | 0.016 | 0.140 |
| 133 | 0.041 | 0.017 | 0.068 |
| 134 | 0.025 | 0.001 | 0.048 |
| 135 | 0.016 | 0.001 | 0.056 |
| 136 | 0.007 | 0.001 | 0.033 |
| 137 | 0.019 | 0.011 | 0.066 |
| 138 | 0.005 | 0.019 | 0.040 |
| 139 | 0.020 | 0.013 | 0.078 |
| 140 | 0.022 | 0.025 | 0.162 |
| 141 | 0.004 | 0.009 | 0.044 |
| 142 | 0.020 | 0.017 | 0.064 |
| 143 | 0.021 | 0.008 | 0.047 |
| 144 | 0.011 | 0.001 | 0.031 |
| 145 | 0.014 | 0.014 | 0.031 |
| 146 | 0.013 | 0.014 | 0.031 |
| 147 | 0.053 | 0.011 | 0.181 |
| 148 | 0.056 | 0.049 | 0.075 |
| 149 | 0.016 | 0.012 | 0.068 |
| 150 | 0.035 | 0.007 | 0.175 |
| 151 | 0.035 | 0.023 | 0.096 |
| 152 | 0.015 | 0.017 | 0.099 |
| 153 | 0.032 | 0.019 | 0.113 |
| 154 | 0.032 | 0.050 | 0.112 |
| 155 | 0.018 | 0.008 | 0.071 |
| 156 | 0.021 | 0.024 | 0.046 |
| 157 | 0.008 | 0.118 | 0.031 |
| 158 | 0.003 | 0.037 | 0.041 |
| 159 | 0.030 | 0.052 | 0.100 |
| 160 | 0.011 | 0.068 | 0.163 |
| 161 | 0.008 | 0.008 | 0.059 |
| 162 | 0.011 | 0.011 | 0.036 |
| 163 | 0.022 | 0.068 | 0.101 |
| 164 | 0.010 | 0.030 | 0.086 |
| 165 | 0.007 | 0.092 | 0.108 |
| 166 | 0.021 | 0.031 | 0.046 |
| 167 | 0.014 | 0.034 | 0.046 |
| 168 | 0.020 | 0.057 | 0.116 |
| 169 | 0.009 | 0.030 | 0.078 |
| 170 | 0.006 | 0.013 | 0.070 |
| 171 | 0.038 | 0.129 | 0.035 |
| 172 | 0.041 | 0.093 | 0.057 |
| 173 | 0.029 | 0.074 | 0.06 |
| 174 | 0.047 | 0.159 | 0.086 |
| 175 | 0.010 | 0.012 | 0.066 |
| 176 | 0.036 | 0.323 | 0.101 |
| 177 | 0.016 | 0.027 | 0.098 |
| 178 | 0.038 | 0.091 | 0.116 |
| 179 | 0.030 | 0.054 | 0.157 |
| 180 | 0.053 | 0.077 | 0.041 |
| 182 | 0.026 | 0.051 | 0.039 |
| 183 | 0.028 | 0.013 | 0.12 |
| 184 | 0.023 | 0.014 | 0.124 |
| 185 | 0.039 | 0.033 | 0.186 |
| 186 | 0.018 | 0.023 | 0.124 |

### Example 188

hERG Channel Inhibition Assay:

The hERG channel inhibition assay is a highly sensitive measurement that identifies compounds exhibiting hERG inhibition related to cardiotoxicity in vivo. The hERG K⁺ channels were cloned in humans and stably expressed in a CHO (Chinese hamster ovary) cell line. CHO_{hERG} cells were used for patch-clamp (voltage-clamp, whole-cell) experiments. Cells were stimulated by a voltage pattern to activate hERG channels and conduct I_{KhERG} currents (rapid delayed outward rectifier potassium current of the hERG channel). After the cells were stabilized for a few minutes, the amplitude and kinetics of I_{KhERG} were recorded at a stimulation frequency of 0.1 Hz, (6 bpm). Thereafter, the test compound was added to the preparation at increasing concentrations. For each concentration, an attempt was made to reach a steady-state effect, usually, this was achieved within 3-10 min at which time the next highest concentration was applied. The amplitude and kinetics of I_{KhERG} are recorded in each concentration of the drug which were compared to the control values (taken as 100%). (references: Redfern W S, Carlsson L, Davis A S, Lynch W G, MacKenzie I, Palethorpe S, Siegl P K, Strang I, Sullivan A T, Wallis R, Camm A J, Hammond T G. 2003; Relationships between preclinical cardiac electrophysiology, clinical QT interval prolongation and torsade de pointes for a broad range of drugs: evidence for a provisional safety margin in drug development. Cardiovasc. Res. 58:32-45, Sanguinetti M C, Tristani-Firouzi M. 2006; hERG potassium channels and cardiac arrhythmia. Nature 440:463-469, Webster R, Leishman D, Walker D. 2002; Towards a drug concentration effect relationship for QT prolongation and torsades de pointes. Curr. Opin. Drug Discov. Devel. 5:116-26).

Results of hERG are given in Table 3. A safety ratio (hERG IC₂₀/EC₅₀)>30 suggests a sufficient window to differentiate the pharmacology by inhibiting TLR7/8/9 pathways from the potential hERG related cardiotoxicity. According to the calculation of hERG IC₂₀/TLR7/8/9 IC₅₀ below which serves as early selectivity index to assess hERG liability, obviously reference compounds ER-887258, ER-888285, ER-888286, R1 and R2 have much narrower safety window compared to the compounds of this invention.

TABLE 3
hERG and safety ratio results

| | | | hERG | hERG | hERG |
|---|---|---|---|---|---|
| | hERG | hERG | IC₂₀/ | IC₂₀/ | IC₂₀/ |
| | IC₂₀ | IC₅₀ | TLR7 | TLR8 | TLR9 |
| Example No | (μM) | (μM) | IC₅₀ | IC₅₀ | IC₅₀ |
| ER-887258 | 0.687 | 2.784 | 8.1 | N.A. | 0.3 |
| ER-888285 | 1.006 | 3.105 | 8.4 | N.A. | 0.5 |
| ER-888286 | 0.348 | 1.297 | 0.3 | N.A. | 0.2 |
| R1 | 0.879 | 2.745 | 9.3 | 6.2 | 0.1 |
| R2 | 0.280 | 0.770 | 1.3 | 0.8 | 0.04 |
| 7 | >10 | >20 | >294 | >1111 | >196 |
| 15 | >10 | >20 | >1429 | >3333 | >278 |
| 38 | 5.5 | >20 | 344 | 500 | 57 |
| 98 | >10 | >20 | >1666 | >10000 | >204 |
| 99 | 9 | >20 | 818 | 9000 | 142 |
| 104 | >10 | >20 | >454 | >10000 | >142 |
| 114 | >10 | >20 | >3333 | >10000 | >161 |
| 118 | >10 | >20 | >2500 | >10000 | >196 |
| 124 | >10 | >20 | >2000 | >10000 | >322 |
| 132 | >10 | >20 | >434 | >625 | >71 |
| 143 | >10 | >20 | >476 | >1250 | >212 |
| 144 | >10 | >20 | >909 | >10000 | >322 |
| 154 | >10 | >20 | >312 | >200 | >89 |
| 166 | 8 | >20 | 380 | 258 | 173 |
| 167 | >10 | >20 | >714 | >294 | >217 |
| 168 | 8.6 | >20 | 430 | 150 | 74 |

### Example 189

The compounds would be desirable to have minimal DDI liabilities. Therefore, the effects of compounds of formula (I) of (Ia) on major CYP isoforms, e.g. CYP2C9, CYP2D6 and CYP3A4, are determined.

### CYP Inhibition Assay

This is a high throughput screening assay used for assessment of reversible inhibition of CYP2C9, CYP2D6, and CYP3A4 activity of test compounds in human liver microsome (HLM) in early discovery stage.

TABLE 4
Chemicals and materials used in the CYP inhibition assay

| | | | | Final | |
|---|---|---|---|---|---|
| | Descrip- | | Cat. | Concentration | |
| Substances | tion | Source | No. | in incubation | |
| Human Liver | | BD- | 452117 | 0.2 | mg/mL |
| Microsomes | | Gentest | | | |
| Diclofenac | CYP2C9 | Sigma | D-6899 | 5 | μM |
| | substrate | | | | |
| 4′-Hydroxy- | CYP2C9 | | | | |
| diclofenac | product | | | | |
| 4′-OH- | CYP2C9 | Becton | 451006 | | |
| Diclofenac- | internal | Dickinson | | | |
| 13C6 | standard | | | | |
| Dextromethorphan | CYP2D6 | Sigma | D-2531 | 5 | μM |
| | substrate | | | | |
| Dextrorphan | CYP2D6 | | | | |
| | product | | | | |
| Dextrorphan-D3 | CYP2D6 | Promochem | CERD- | | |
| | internal | | 041 | | |
| | standard | | | | |
| Midazolam | CYP3A4 | Roche | | 5 | μM |
| | substrate | | | | |
| 1′- | CYP3A4 | | | | |
| Hydroxymidazolam | product | | | | |
| 1′-OH | CYP3A4 | Roche | | | |
| Midazolam-D4 | internal | | | | |
| | standard | | | | |
| Sulfaphenazole | CYP2C9 | | | 2 | μM |
| | inhibitor | | | | |
| Quinidine | CYP2D6 | | | 0.5 | μM |
| | inhibitor | | | | |
| Ketoconazole | CYP3A4 | | | 0.5 | μM |
| | inhibitor | | | | |

### Procedure

10 mM DMSO stock solutions of test compounds were diluted in DMSO to generate 2 mM intermediate stock solution. 250 nL, of intermediate stock solution were transferred in duplicate into 3 separate 384 well microtitre plates (assay-ready plates). A mixture of HLM and each substrate was made up. 45 μL of HLM substrate mix was then transferred to each well of an assay ready plate and mixed. The negative (solvent) and positive controls (standard inhibitor for each CYP) were included in each assay ready plate. The assay ready plate was warmed to 37° C. in an incubator over 10 minutes. 5 μL pre-warmed NADPH regenerating system was added to each incubation well to start the reaction. Final incubation volume was 50 μL. The assay plate then was placed back in the 37° C. incubator. After incubation (10 minutes for CYP2D6) for 5 minutes, incubates were quenched by addition of 50 μL 100% acetonitrile containing internal standards (400 ng/mL 13C6-4′-OH-Diclofenac, 20 ng/mL D3-Dextrorphan and 20 ng/mL D4-1′OH-Midazolam). The supernatants were collected for RapidFire/MS/MS analysis.

RapidFire online solid phase extraction/sample injection system (Agilent) coupled with API4000 triple quadrupole mass spectrometer (AB Sciex) were used for sample analysis. The mobile phase composed of acetonitrile and water supplemented with 0.1% formic acid. A C4 solid phase extraction cartridge is used for sample separation. MS detection is achieved in positive ion MRM mode.

### Data Analysis

Peak areas for substrate, metabolite and internal standard are determined using the RapidFire integrator software (version 3.6.12009.12296). Peak area ratios (PAR) of metabolite and internal standard (stable-labelled metabolite) are then calculated. The measurement window for each experiment is then defined:

PAR (0% activity)=average PAR for all incubations containing concentrated inhibitor;
Par (100% activity)=average PAR for all incubations containing no inhibitor (DMSO controls);
% Activity (test inhibitor)=[PAR (test inhibitor)−PAR (0% activity)]/[PAR (100% activity)−PAR (0% activity)];
% Inhibition (test inhibitor)=100-% Activity (test inhibitor).

The compounds of present invention were found to have low CYP inhibition for CYP2D6 determined in the assays described above.

TABLE 5
CYP inhibition of the compounds of this invention for CYP2D6

| Example No | CYP (%) 2C9/2D6/3A4 |
|---|---|
| ER-888286 | 29.5/52.5/5.5 |
| 1 | −7/5/14 |
| 2 | 2/20/18 |
| 3 | −18/13.5/4.5 |
| 4 | 5.5/11/0.5 |
| 7 | 2/7.5/15.5 |
| 8 | 1/6/−3.5 |
| 9 | −7.5/2/7.5 |
| 11 | 0.5/0/11 |
| 14 | 0/16.5/−2 |
| 15 | 9/−2.5/11.5 |
| 19 | 11.5/0.5/12.5 |
| 21 | −3/2.5/−1.5 |
| 22 | 20/2/−16 |
| 24 | 3.5/6/4 |
| 25 | −5.5/5.5/−4 |
| 29 | −2/8.5/−5.5 |
| 38 | 20/20.5/20 |
| 39 | 23/−4/23.5 |
| 43 | 2.5/−11/16 |
| 45 | −3.5/5/−5 |
| 52 | 3.5/10.5/2 |
| 53 | −4.5/21.5/7.5 |
| 104 | −1/15/−12 |
| 106 | 17/3/20.5 |
| 107 | 19.5/16/34.5 |
| 110 | 22/13.5/44 |
| 112 | 0.5/−2/−5 |
| 114 | 7.5/32/16 |
| 116 | −8.5/10/8 |
| 117 | 8.5/4.5/10 |
| 119 | −12.5/−8.5/23.5 |
| 124 | −2/9.5/16 |
| 132 | −1/−2.5/7.5 |
| 134 | 44.5/−4/47.5 |
| 143 | −9.5/−30/18 |
| 152 | −1/12.5/35 |
| 154 | 2.5/−10.5/10.5 |
| 158 | −5.5/−7.5/−0.5 |
| 160 | −1.5/17/4.5 |
| 162 | −2.5/1/42.5 |
| 163 | −4.5/6.5/0.5 |
| 164 | −1/26/30.5 |
| 165 | −8.5/17.5/9 |
| 166 | −3.5/20/35.5 |
| 167 | −1/11.5/44 |
| 168 | −8/1.5/11 |
| 170 | −1/0/−7 |

ND: not detected;
percentage inhibition < 0: not or weak inhibitor

1. A compound of formula (I), 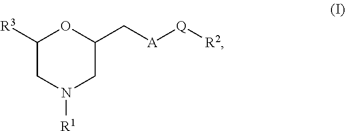
wherein
R¹ is 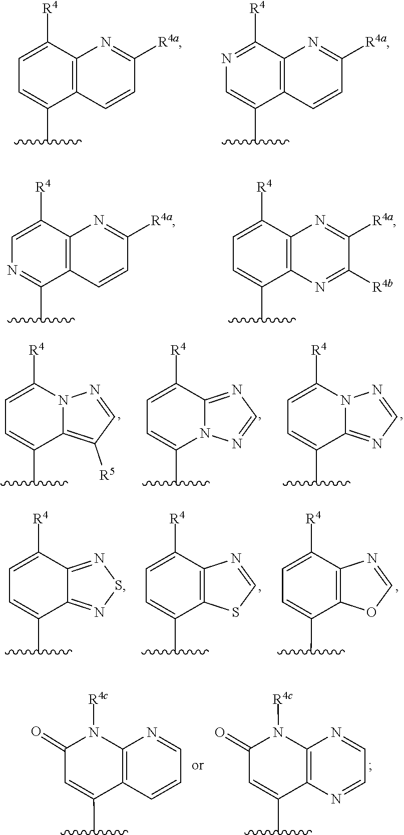
wherein R⁴ is C₁₋₆alkyl, C₁₋₆alkoxy, haloC₁₋₆alkyl, halogen, nitro or cyano; R^{4a} is H or deuterium; R^{4b} is H, deuterium or C₁₋₆alkyl; R^{4c} is C₁₋₆alkyl or C₃₋₇cycloalkyl; R⁵ is H or halogen;
R² is ((C₁₋₆alkyl)₂amino)C₁₋₆ alkoxy;
(C₁₋₆alkoxypyrrolidinyl)amino;
(cyanopyrrolidinyl)amino;
1,4-diazepanyl substituted by hydroxy;
1,6-diazaspiro[3.3]heptanyl;
2,5-diazabicyclo[2.2.1]heptanyl;
2,6-diazaspiro[3.3]heptanyl substituted by C₁₋₆alkyl;
3,4,4a,5,7,7a-hexahydro-2H-pyrrolo[3,4-b][1,4]oxazinyl;
3-oxa-7,9-diazabicyclo[3.3.1]nonanyl;
5-oxa-2,8-diazaspiro[3.5]nonanyl;
aminoC₁₋₆alkyl;
aminooxazepanyl;
azetidinyl substituted once or twice by substituents independently selected from amino and C₁₋₆alkyl;
azetidinylamino;
halopyrrolidinylamino;
morpholinyl unsubstituted or substituted by C₁₋₆alkyl;
morpholinylC₁₋₆alkyl;
piperazinyl unsubstituted or substituted by C₁₋₆alkyl, hydroxyC₁₋₆alkyl, pyrrolidinylcarbonyl, ((C₁₋₆alkyl)₂amino)C₁₋₆alkylcarbonyl or azetidinylcarbonyl;
piperidinyl unsubstituted or substituted once or twice by substituents independently selected from amino, halogen and C₁₋₆ alkoxy; or
pyrrolidinyl substituted once, twice or three times by substituents independently selected from amino, halogen, hydroxy, C₁₋₆alkyl and C₁₋₆ alkoxy;
R³ is C₁₋₆alkyl;
A is azetidinyl, hydroxyazetidinyl, piperazinyl, pyrrolidinyl, piperidinyl or cyanopiperidinyl;
Q is phenyl unsubstituted or substituted by halogen;
pyrazinyl;
pyridazinyl;
pyridinyl unsubstituted or substituted by C₁₋₆alkyl;
pyrimidinyl unsubstituted or substituted once or twice by substituents independently selected from halogen, C₁₋₆alkyl, C₁₋₆ alkoxy and cyano; or
quinazolinyl;
or a pharmaceutically acceptable salt thereof.

2. A compound of formula (Ia), 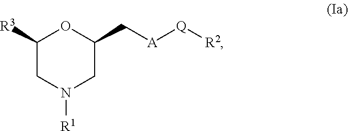
wherein
R¹ is 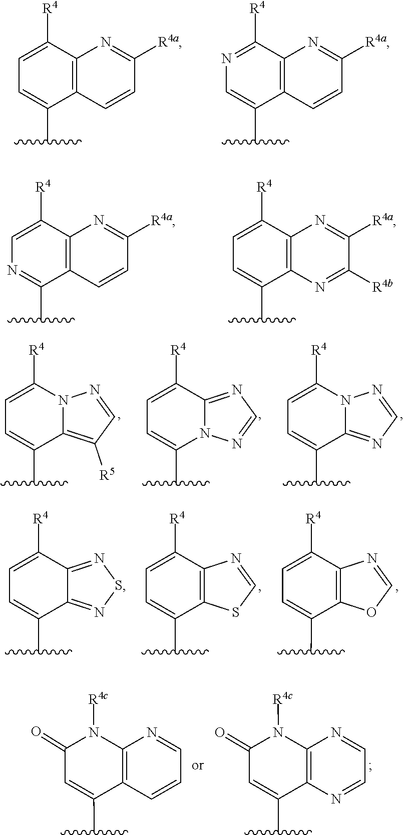
wherein R⁴ is C₁₋₆alkyl, C₁₋₆alkoxy, haloC₁₋₆alkyl, halogen, nitro or cyano; R^{4a} is H or deuterium; R^{4b} is H, deuterium or C₁₋₆alkyl; R^{4c} is C₁₋₆alkyl or C₃₋₇cycloalkyl; R⁵ is H or halogen;
R² is ((C₁₋₆alkyl)₂amino)C₁₋₆ alkoxy;
(C₁₋₆alkoxypyrrolidinyl)amino;
(cyanopyrrolidinyl)amino;
1,4-diazepanyl substituted by hydroxy;
1,6-diazaspiro[3.3]heptanyl;
2,5-diazabicyclo[2.2.1]heptanyl;
2,6-diazaspiro[3.3]heptanyl substituted by C₁₋₆alkyl;
3,4,4a,5,7,7a-hexahydro-2H-pyrrolo[3,4-b][1,4]oxazinyl;
3-oxa-7,9-diazabicyclo[3.3.1]nonanyl;
5-oxa-2,8-diazaspiro[3.5]nonanyl;
aminoC₁₋₆alkyl;
aminooxazepanyl;
azetidinyl substituted once or twice by substituents independently selected from amino and C₁₋₆alkyl;
azetidinylamino;
halopyrrolidinylamino;
morpholinyl unsubstituted or substituted by C₁₋₆alkyl;
morpholinylC₁₋₆alkyl;
piperazinyl unsubstituted or substituted by C₁₋₆alkyl, hydroxyC₁₋₆alkyl, pyrrolidinylcarbonyl, ((C₁₋₆alkyl)₂amino)C₁₋₆alkylcarbonyl or azetidinylcarbonyl;
piperidinyl unsubstituted or substituted once or twice by substituents independently selected from amino, halogen and C₁₋₆ alkoxy; or
pyrrolidinyl substituted once, twice or three times by substituents independently selected from amino, halogen, hydroxy, C₁₋₆alkyl and C₁₋₆ alkoxy;
R³ is C₁₋₆alkyl;
A is azetidinyl, hydroxyazetidinyl, piperazinyl, pyrrolidinyl, piperidinyl or cyanopiperidinyl;
Q is phenyl unsubstituted or substituted by halogen;
pyrazinyl;
pyridazinyl;
pyridinyl unsubstituted or substituted by C₁₋₆alkyl;
pyrimidinyl unsubstituted or substituted once or twice by substituents independently selected from halogen, C₁₋₆alkyl, C₁₋₆ alkoxy and cyano; or
quinazolinyl;
or a pharmaceutically acceptable salt thereof.

3. A compound according to claim 1 or 2, or a pharmaceutically acceptable salt thereof, wherein
R¹ is 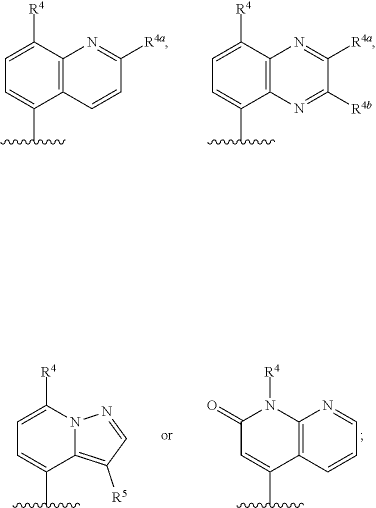
wherein R⁴ is C₁₋₆alkyl, haloC₁₋₆alkyl or cyano; R^{4a} is H or deuterium; R^{4b} is H or deuterium; R^{4c} is C₁₋₆alkyl; R⁵ is H or halogen.

4. A compound according to claim 3, or a pharmaceutically acceptable salt thereof, wherein R⁴ is methyl, trifluoromethyl or cyano; R^{4a} is H or deuterium; R^{4b} is H or deuterium; R^{4c} is methyl; R⁵ is H or fluoro.

5. A compound according to claim 3, or a pharmaceutically acceptable salt thereof, wherein R² is (((C₁₋₆alkyl)₂amino)C₁₋₆alkylcarbonyl)piperazinyl; ((C₁₋₆alkyl)₂amino)C₁₋₆ alkoxy; (azetidinylcarbonyl)piperazinyl; (C₁₋₆ alkoxypyrrolidinyl)amino; (C₁₋₆alkyl)morpholinyl; (cyanopyrrolidinyl)amino; (hydroxyC₁₋₆alkyl)piperazinyl; (pyrrolidinylcarbonyl)piperazinyl; 1,6-diazaspiro[3.3]heptanyl; 2,5-diazabicyclo[2.2.1]heptanyl; 3,4,4a,5,7,7a-hexahydro-2H-pyrrolo[3,4-b][1,4]oxazinyl; 3-oxa-7,9-diazabicyclo[3.3.1]nonanyl; 5-oxa-2,8-diazaspiro[3.5]nonanyl; amino(C₁₋₆ alkoxy)piperidinyl; amino(C₁₋₆ alkoxy)pyrrolidinyl; amino(C₁₋₆alkyl)azetidinyl; amino(hydroxy)(C₁₋₆alkyl)pyrrolidinyl; aminoazetidinyl; aminoC₁₋₆alkyl; aminohalopiperidinyl; aminohalopyrrolidinyl; aminooxazepanyl; aminopiperidinyl; azetidinylamino; C₁₋₆alkyl-2,6-diazaspiro[3.3]heptanyl; C₁₋₆alkylpiperazinyl; halopyrrolidinylamino; hydroxy-1,4-diazepanyl; morpholinyl; morpholinylC₁₋₆alkyl; piperazinyl or piperidinyl.

6. A compound according to claim 5, or a pharmaceutically acceptable salt thereof, wherein R² is (1-hydroxy-1-methyl-ethyl)piperazin-1-yl; (2-pyrrolidinylcarbonyl)piperazin-1-yl; (4-cyanopyrrolidin-3-yl)amino; (4-fluoropyrrolidin-3-yl)amino; (4-methoxypyrrolidin-3-yl)amino; (dimethylamino)ethoxy; 1,6-diazaspiro[3.3]heptan-6-yl; 2,5-diazabicyclo[2.2.1]heptan-2-yl; 2-methylmorpholin-2-yl; 2-morpholin-2-yl; 3-(hydroxymethyl)piperazin-1-yl; 3,4,4a,5,7,7a-hexahydro-2H-pyrrolo[3,4-b][1,4]oxazin-6-yl; 3-amino-3-methyl-azetidin-1-yl; 3-amino-4-fluoro-1-piperidinyl; 3-amino-4-fluoro-pyrrolidin-1-yl; 3-amino-4-methoxy-1-piperidinyl; 3-amino-4-methoxy-pyrrolidin-1-yl; 3-amino-5-fluoro-1-piperidinyl; 3-aminoazetidin-1-yl; 3-methylpiperazin-1-yl; 3-oxa-7,9-diazabicyclo[3.3.1]nonan-7-yl; 4-(azetidinyl-2-carbonyl)piperazin-1-yl; 4-[2-(dimethylamino)acetyl]piperazin-1-yl; 4-amino-1-piperidinyl; 4-amino-3,3-difluoro-1-piperidinyl; 4-amino-3,3-difluoro-pyrrolidin-1-yl; 4-amino-3-fluoro-1-piperidinyl; 4-amino-3-hydroxy-3-methyl-pyrrolidin-1-yl; 4-amino-3-methoxy-1-piperidinyl; 4-methylpiperazin-1-yl; 4-piperidinyl; 5-amino-3,3-difluoro-1-piperidinyl; 5-oxa-2,8-diazaspiro[3.5]nonan-2-yl; 5-oxa-2,8-diazaspiro[3.5]nonan-8-yl; 6-amino-1,4-oxazepan-4-yl; 6-hydroxy-1,4-diazepan-1-yl; 6-methyl-2,6-diazaspiro[3.3]heptan-2-yl; aminomethyl; azetidin-3-ylamino; morpholin-2-yl; morpholin-2-ylmethyl or piperazin-1-yl.

7. A compound according to claim 6, or a pharmaceutically acceptable salt thereof, wherein A is

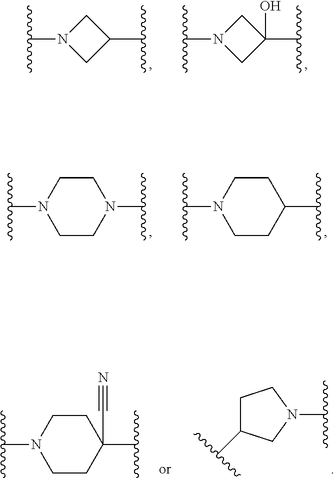

8. A compound according to claim 7, or a pharmaceutically acceptable salt thereof, wherein Q is phenyl, fluorophenyl, pyrazinyl, pyridazinyl, pyridinyl, methylpyridinyl, pyrimidinyl, cyanopyrimidinyl, fluoropyrimidinyl, methoxypyrimidinyl, methylpyrimidinyl, dimethylpyrimidinyl or quinazolinyl.

9. A compound according to claim 8, or a pharmaceutically acceptable salt thereof, wherein R³ is methyl.

10. A compound according to claim 1 or 2, or a pharmaceutically acceptable salt thereof, wherein
R¹ is 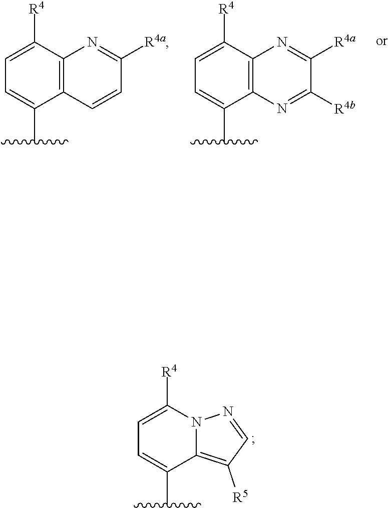
wherein R⁴ is C₁₋₆alkyl, haloC₁₋₆alkyl or cyano; R^{4a} and R^{4b} are simultaneously H or deuterium; R⁵ is H or halogen.

11. A compound according to claim 10, or a pharmaceutically acceptable salt thereof, wherein R⁴ is methyl, trifluoromethyl or cyano; R^{4a} and R^{4b} are simultaneously H or deuterium; R⁵ is H or fluoro.

12. A compound according to claim 10, or a pharmaceutically acceptable salt thereof, wherein R² is 3,4,4a,5,7,7a-hexahydro-2H-pyrrolo[3,4-b][1,4]oxazinyl; 5-oxa-2,8-diazaspiro[3.5]nonanyl; amino(C₁₋₆ alkoxy)pyrrolidinyl; amino(C₁₋₆alkyl)azetidinyl; aminoazetidinyl; aminopiperidinyl; C₁₋₆alkyl-2,6-diazaspiro[3.3]heptanyl; halopyrrolidinylamino; morpholinyl; morpholinylC₁₋₆alkyl or piperazinyl.

13. A compound according to claim 12, or a pharmaceutically acceptable salt thereof, wherein R² is (4-fluoropyrrolidin-3-yl)amino; 3,4,4a,5,7,7a-hexahydro-2H-pyrrolo[3,4-b][1,4]oxazin-6-yl; 3-amino-3-methyl-azetidin-1-yl; 3-amino-4-methoxy-pyrrolidin-1-yl; 3-aminoazetidin-1-yl; 4-amino-1-piperidinyl; 5-oxa-2,8-diazaspiro[3.5]nonan-2-yl; 6-methyl-2,6-diazaspiro[3.3]heptan-2-yl; morpholin-2-yl; morpholin-2-ylmethyl or piperazin-1-yl.

14. A compound according to claim 12, or a pharmaceutically acceptable salt thereof, wherein A is

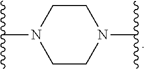 [SMILES: *N1CCN(*)CC1]

15. A compound according to claim 14, or a pharmaceutically acceptable salt thereof, wherein Q is phenyl, pyridinyl, pyrimidinyl or C₁₋₆alkylpyrimidinyl.

16. A compound according to claim 15, or a pharmaceutically acceptable salt thereof, wherein Q is phenyl, pyridinyl, pyrimidinyl or methylpyrimidinyl.

17. A compound according to claim 3, wherein
R¹ is 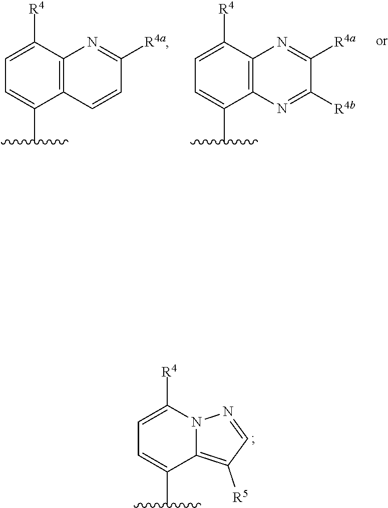
wherein R⁴ is C₁₋₆alkyl, haloC₁₋₆alkyl or cyano; R^{4a} and R^{4b} are simultaneously H or deuterium; R⁵ is H or halogen;
R² is 3,4,4a,5,7,7a-hexahydro-2H-pyrrolo[3,4-b][1,4]oxazinyl; 5-oxa-2,8-diazaspiro[3.5]nonanyl; amino(C₁₋₆ alkoxy)pyrrolidinyl; amino(C₁₋₆alkyl)azetidinyl; aminoazetidinyl; aminopiperidinyl; C₁₋₆ alkyl-2,6-diazaspiro[3.3]heptanyl; halopyrrolidinylamino; morpholinyl; morpholinylC₁₋₆alkyl or piperazinyl;
R³ is C₁₋₆alkyl;
A is 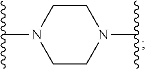 [SMILES: *N1CCN(*)CC1]
Q is phenyl, pyridinyl, pyrimidinyl or C₁₋₆alkylpyrimidinyl;
or a pharmaceutically acceptable salt thereof.

18. A compound according to claim 17, wherein
R¹ is 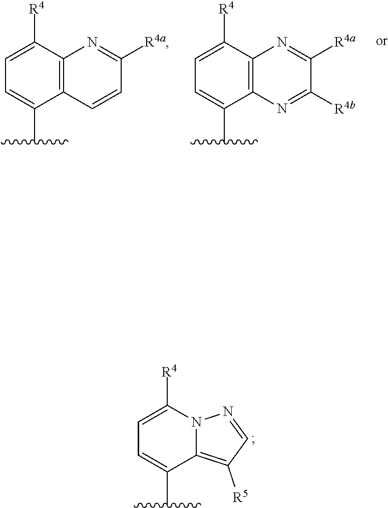
wherein R⁴ is methyl, trifluoromethyl or cyano; R^{4a} and R^{4b} are simultaneously H or deuterium; R⁵ is H or fluoro;
R² is (4-fluoropyrrolidin-3-yl)amino; 3,4,4a,5,7,7a-hexahydro-2H-pyrrolo[3,4-b][1,4]oxazin-6-yl; 3-amino-3-methyl-azetidin-1-yl; 3-amino-4-methoxy-pyrrolidin-1-yl; 3-aminoazetidin-1-yl; 4-amino-1-piperidinyl; 5-oxa-2,8-diazaspiro[3.5]nonan-2-yl; 6-methyl-2,6-diazaspiro[3.3]heptan-2-yl; morpholin-2-yl; morpholin-2-ylmethyl or piperazin-1-yl;
R³ is methyl;
A is 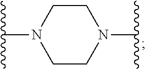 [SMILES: *N1CCN(*)CC1]
Q is phenyl, pyridinyl, pyrimidinyl or methylpyrimidinyl;
or a pharmaceutically acceptable salt thereof.

19. A compound selected from:
5-[(2R,6S)-2-methyl-6-[[3-(3-piperazin-1-ylphenyl)pyrrolidin-1-yl]methyl]morpholin-4-yl]quinoline-8-carbonitrile;
5-[(2R,6S)-2-methyl-6-[[4-[3-(4-piperidyl)phenyl]-1-piperidyl]methyl]morpholin-4-yl]quinoline-8-carbonitrile;
5-[(2R,6S)-2-methyl-6-[[3-(4-piperazin-1-ylphenyl)azetidin-1-yl]methyl]morpholin-4-yl]quinoline-8-carbonitrile;
5-[(2R,6S)-2-methyl-6-[[3-(3-piperazin-1-ylphenyl)azetidin-1-yl]methyl]morpholin-4-yl]quinoline-8-carbonitrile;
5-[(2S,6R)-2-[[4-[4-(aminomethyl)phenyl]piperazin-1-yl]methyl]-6-methyl-morpholin-4-yl]quinoline-8-carbonitrile;
5-[(2R,6S)-2-methyl-6-[[4-(5-piperazin-1-ylpyrazin-2-yl)piperazin-1-yl]methyl]morpholin-4-yl]quinoline-8-carbonitrile;
5-[(2R,6S)-2-methyl-6-[[4-(5-piperazin-1-yl-3-pyridyl)piperazin-1-yl]methyl]morpholin-4-yl]quinoline-8-carbonitrile;
5-[(2R,6S)-2-methyl-6-[[4-(5-piperazin-1-yl-2-pyridyl)piperazin-1-yl]methyl]morpholin-4-yl]quinoline-8-carbonitrile;
5-[(2R,6S)-2-methyl-6-[[4-(4-piperazin-1-yl-2-pyridyl)piperazin-1-yl]methyl]morpholin-4-yl]quinoline-8-carbonitrile;
5-[(2R,6S)-2-methyl-6-[[4-(6-piperazin-1-yl-2-pyridyl)piperazin-1-yl]methyl]morpholin-4-yl]quinoline-8-carbonitrile;
5-[(2S,6R)-2-[[4-cyano-4-(4-piperazin-1-ylphenyl)-1-piperidyl]methyl]-6-methyl-morpholin-4-yl]quinoline-8-carbonitrile;
5-[(2R,6S)-2-methyl-6-[[4-(2-piperazin-1-ylpyrimidin-5-yl)piperazin-1-yl]methyl]morpholin-4-yl]quinoline-8-carbonitrile;
5-[(2R,6S)-2-methyl-6-[[4-(6-piperazin-1-yl-3-pyridyl)piperazin-1-yl]methyl]morpholin-4-yl]quinoline-8-carbonitrile;
5-[(2R,6S)-2-methyl-6-[[4-(4-piperazin-1-ylpyrimidin-2-yl)piperazin-1-yl]methyl]morpholin-4-yl]quinoline-8-carbonitrile;
5-[(2R,6S)-2-methyl-6-[[4-(2-piperazin-1-ylpyrimidin-4-yl)piperazin-1-yl]methyl]morpholin-4-yl]quinoline-8-carbonitrile;
5-[(2R,6S)-2-methyl-6-[[4-(6-piperazin-1-ylpyridazin-3-yl)piperazin-1-yl]methyl]morpholin-4-yl]quinoline-8-carbonitrile;
5-[(2R,6S)-2-methyl-6-[[4-(5-piperazin-1-ylpyrimidin-2-yl)piperazin-1-yl]methyl]morpholin-4-yl]quinoline-8-carbonitrile;
5-[(2R,6S)-2-methyl-6-[[4-(6-piperazin-1-ylpyrimidin-4-yl)piperazin-1-yl]methyl]morpholin-4-yl]quinoline-8-carbonitrile;
5-[(2R,6S)-2-methyl-6-[[4-[4-[(2R)-morpholin-2-yl]phenyl]piperazin-1-yl]methyl]morpholin-4-yl]quinoline-8-carbonitrile;
5-[(2R,6S)-2-methyl-6-[[4-[4-[(2S)-morpholin-2-yl]phenyl]piperazin-1-yl]methyl]morpholin-4-yl]quinoline-8-carbonitrile;
5-[(2R,6S)-2-methyl-6-[[4-[6-(4-methylpiperazin-1-yl)pyrimidin-4-yl]piperazin-1-yl]methyl]morpholin-4-yl]quinoline-8-carbonitrile;
5-[(2S,6R)-2-[[4-[2-(3-aminoazetidin-1-yl)pyrimidin-4-yl]piperazin-1-yl]methyl]-6-methyl-morpholin-4-yl]quinoline-8-carbonitrile;
5-[(2S,6R)-2-[[4-[2-(3-aminoazetidin-1-yl)pyrimidin-4-yl]piperazin-1-yl]methyl]-6-methyl-morpholin-4-yl]quinoline-8-carbonitrile;
5-[(2S,6R)-2-[[4-[2-[[(3S,4R)-4-fluoropyrrolidin-3-yl]amino]pyrimidin-4-yl]piperazin-1-yl]methyl]-6-methyl-morpholin-4-yl]quinoline-8-carbonitrile;
5-[(2S,6R)-2-[[4-[2-(6-hydroxy-1,4-diazepan-1-yl)pyrimidin-4-yl]piperazin-1-yl]methyl]-6-methyl-morpholin-4-yl]quinoline-8-carbonitrile;
5-[(2S,6R)-2-[[4-[2-trans-(3-amino-4-methoxy-pyrrolidin-1-yl)pyrimidin-4-yl]piperazin-1-yl]methyl]-6-methyl-morpholin-4-yl]quinoline-8-carbonitrile;
5-[(2S,6R)-2-[[4-[2-(4-amino-1-piperidyl)pyrimidin-4-yl]piperazin-1-yl]methyl]-6-methyl-morpholin-4-yl]quinoline-8-carbonitrile;
5-[(2S,6R)-2-[[4-[2-(azetidin-3-ylamino)pyrimidin-4-yl]piperazin-1-yl]methyl]-6-methyl-morpholin-4-yl]quinoline-8-carbonitrile;
5-[(2S,6R)-2-[[4-[2-[(3R)-3-(hydroxymethyl)piperazin-1-yl]pyrimidin-4-yl]piperazin-1-yl]methyl]-6-methyl-morpholin-4-yl]quinoline-8-carbonitrile;
5-[(2S,6R)-2-[[4-[2-[2-(dimethylamino)ethoxy]pyrimidin-4-yl]piperazin-1-yl]methyl]-6-methyl-morpholin-4-yl]quinoline-8-carbonitrile;
5-[(2R,6S)-2-methyl-6-[[4-[2-[4-[(2S)-pyrrolidine-2-carbonyl]piperazin-1-yl]pyrimidin-4-yl]piperazin-1-yl]methyl]morpholin-4-yl]quinoline-8-carbonitrile;
5-[(2S,6R)-2-[[4-[2-[4-[2-(dimethylamino)acetyl]piperazin-1-yl]pyrimidin-4-yl]piperazin-1-yl]methyl]-6-methyl-morpholin-4-yl]quinoline-8-carbonitrile;
5-[(2R,6S)-2-methyl-6-[[4-[6-(4-methylpiperazin-1-yl)-2-pyridyl]piperazin-1-yl]methyl]morpholin-4-yl]quinoline-8-carbonitrile;
5-[(2S,6R)-2-[[4-[2-[4-(azetidine-2-carbonyl)piperazin-1-yl]pyrimidin-4-yl]piperazin-1-yl]methyl]-6-methyl-morpholin-4-yl]quinoline-8-carbonitrile;
5-[(2S,6R)-2-[[4-[6-[[(3S,4S)-4-fluoropyrrolidin-3-yl]amino]-2-pyridyl]piperazin-1-yl]methyl]-6-methyl-morpholin-4-yl]quinoline-8-carbonitrile;
5-[(2S,6R)-2-[[4-[6-(4-amino-1-piperidyl)-2-pyridyl]piperazin-1-yl]methyl]-6-methyl-morpholin-4-yl]quinoline-8-carbonitrile;
5-[(2S,6R)-2-[[4-[6-[[(3S,4R)-4-fluoropyrrolidin-3-yl]amino]-2-pyridyl]piperazin-1-yl]methyl]-6-methyl-morpholin-4-yl]quinoline-8-carbonitrile;
5-[(2S,6R)-2-[[4-[6-(4-amino-1-piperidyl)-2-pyridyl]piperazin-1-yl]methyl]-6-methyl-morpholin-4-yl]quinoline-8-carbonitrile;
5-[(2S,6R)-2-[[4-[6-[[(3R,4R)-4-methoxypyrrolidin-3-yl]amino]-2-pyridyl]piperazin-1-yl]methyl]-6-methyl-morpholin-4-yl]quinoline-8-carbonitrile;
5-[(2S,6R)-2-[[4-[6-(3-amino-4-fluoro-pyrrolidin-1-yl)-2-pyridyl]piperazin-1-yl]methyl]-6-methyl-morpholin-4-yl]quinoline-8-carbonitrile;
5-[(2S,6R)-2-[[4-[6-(6-hydroxy-1,4-diazepan-1-yl)-2-pyridyl]piperazin-1-yl]methyl]-6-methyl-morpholin-4-yl]quinoline-8-carbonitrile;
5-[(2S,6R)-2-[[4-[6-[(3R,4S)-3-amino-4-methoxy-pyrrolidin-1-yl]-2-pyridyl]piperazin-1-yl]methyl]-6-methyl-morpholin-4-yl]quinoline-8-carbonitrile;
5-[(2S,6R)-2-[[4-[6-(3-amino-4-methoxy-pyrrolidin-1-yl)-3-pyridyl]piperazin-1-yl]methyl]-6-methyl-morpholin-4-yl]quinoline-8-carbonitrile;
5-[(2S,6R)-2-[[4-[6-(3-amino-4-fluoro-pyrrolidin-1-yl)-3-pyridyl]piperazin-1-yl]methyl]-6-methyl-morpholin-4-yl]quinoline-8-carbonitrile;
5-[(2S,6R)-2-[[4-(5-fluoro-2-piperazin-1-yl-pyrimidin-4-yl)piperazin-1-yl]methyl]-6-methyl-morpholin-4-yl]quinoline-8-carbonitrile;
5-[(2S,6R)-2-[[4-(5,6-dimethyl-2-piperazin-1-yl-pyrimidin-4-yl)piperazin-1-yl]methyl]-6-methyl-morpholin-4-yl]quinoline-8-carbonitrile;
5-[(2R,6S)-2-methyl-6-[[4-(6-methyl-2-piperazin-1-yl-pyrimidin-4-yl)piperazin-1-yl]methyl]morpholin-4-yl]quinoline-8-carbonitrile;
5-[(2S,6R)-2-[[4-(5-methoxy-2-piperazin-1-yl-pyrimidin-4-yl)piperazin-1-yl]methyl]-6-methyl-morpholin-4-yl]quinoline-8-carbonitrile;
5-[(2R,6S)-2-methyl-6-[[4-(2-piperazin-1-ylquinazolin-4-yl)piperazin-1-yl]methyl]morpholin-4-yl]quinoline-8-carbonitrile;
5-[(2S,6R)-2-[[4-(5-cyano-2-piperazin-1-yl-pyrimidin-4-yl)piperazin-1-yl]methyl]-6-methyl-morpholin-4-yl]quinoline-8-carbonitrile;
5-[(2S,6R)-2-[[4-(5-cyano-4-piperazin-1-yl-pyrimidin-2-yl)piperazin-1-yl]methyl]-6-methyl-morpholin-4-yl]quinoline-8-carbonitrile;
5-[(2S,6R)-2-[[4-[2-[(3R,4R)-3-amino-4-methoxy-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[2-[(3S,4S)-3-amino-4-methoxy-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[2-[(3S,4R)-3-amino-4-fluoro-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4S)-3-amino-4-fluoro-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-(6-amino-1,4-oxazepan-4-yl)-6-methyl-pyrimidin-4-yl]piperazin-1-yl]methyl]-6-methyl-morpholin-4-yl]quinoline-8-carbonitrile;
5-[(2S,6R)-2-[[4-[2-[(4-cyanopyrrolidin-3-yl)amino]-6-methyl-pyrimidin-4-yl]piperazin-1-yl]methyl]-6-methyl-morpholin-4-yl]quinoline-8-carbonitrile;
5-[(2R,6S)-2-methyl-6-[[4-[6-methyl-2-[(1R,5R)-3-oxa-7,9-diazabicyclo[3.3.1]nonan-7-yl]pyrimidin-4-yl]piperazin-1-yl]methyl]morpholin-4-yl]quinoline-8-carbonitrile;
5-[(2R,6S)-2-methyl-6-[[4-[6-methyl-2-[trans-3-amino-4-methoxy-pyrrolidin-1-yl]pyrimidin-4-yl]piperazin-1-yl]methyl]morpholin-4-yl]quinoline-8-carbonitrile;
5-[(2R,6S)-2-methyl-6-[[4-[6-methyl-2-[cis-3-amino-4-methoxy-pyrrolidin-1-yl]pyrimidin-4-yl]piperazin-1-yl]methyl]morpholin-4-yl]quinoline-8-carbonitrile;
5-[(2R,6S)-2-methyl-6-[[4-[6-methyl-2-(5-oxa-2,8-diazaspiro[3.5]nonan-2-yl)pyrimidin-4-yl]piperazin-1-yl]methyl]morpholin-4-yl]quinoline-8-carbonitrile;
5-[(2R,6S)-2-methyl-6-[[4-(3-morpholin-2-ylphenyl)piperazin-1-yl]methyl]-morpholin-4-yl]quinoline-8-carbonitrile;
5-[(2S,6R)-2-[[4-[2-(4-amino-3,3-difluoro-pyrrolidin-1-yl)-6-methyl-pyrimidin-4-yl]piperazin-1-yl]methyl]-6-methyl-morpholin-4-yl]quinoline-8-carbonitrile;
8-[(2R,6S)-2-methyl-6-[[4-(6-methyl-2-piperazin-1-yl-pyrimidin-4-yl)piperazin-1-yl]methyl]morpholin-4-yl]quinoxaline-5-carbonitrile;
(2R,6S)-2-methyl-6-[[4-(6-methyl-2-piperazin-1-yl-pyrimidin-4-yl)piperazin-1-yl]methyl]-4-[8-(trifluoromethyl)quinoxalin-5-yl]morpholine;
5-[(2S,6R)-2-[[4-[2-(6-hydroxy-1,4-diazepan-1-yl)-6-methyl-pyrimidin-4-yl]piperazin-1-yl]methyl]-6-methyl-morpholin-4-yl]quinoline-8-carbonitrile;
3-Fluoro-4-[(2R,6S)-2-methyl-6-[[4-(6-methyl-2-piperazin-1-yl-pyrimidin-4-yl)piperazin-1-yl]methyl]morpholin-4-yl]pyrazolo[1,5-a]pyridine-7-carbonitrile;
5-[(2R,6S)-2-methyl-6-[[4-(4-methyl-6-piperazin-1-yl-pyrimidin-2-yl)piperazin-1-yl]methyl]morpholin-4-yl]quinoline-8-carbonitrile;
5-[(2S,6R)-2-[[4-[2-trans-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3S,4S)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4R)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[[(3R,4R)-4-methoxypyrrolidin-3-yl]amino]-6-methyl-pyrimidin-4-yl]piperazin-1-yl]methyl]-6-methyl-morpholin-4-yl]quinoline-8-carbonitrile;
5-[(2S,6R)-2-[[4-[2-[[(3R,4S)-4-fluoropyrrolidin-3-yl]amino]-6-methyl-pyrimidin-4-yl]piperazin-1-yl]methyl]-6-methyl-morpholin-4-yl]quinoline-8-carbonitrile;
5-[(2S,6R)-2-[[4-[2-[(3S,4R)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[[(3S,4R)-4-fluoropyrrolidin-3-yl]amino]-6-methyl-pyrimidin-4-yl]piperazin-1-yl]methyl]-6-methyl-morpholin-4-yl]quinoline-8-carbonitrile;
5-[(2R,6S)-2-methyl-6-[[4-[6-methyl-2-[cis-3-amino-5-fluoro-1-piperidyl]pyrimidin-4-yl]piperazin-1-yl]methyl]morpholin-4-yl]quinoline-8-carbonitrile;
5-[(2R,6S)-2-methyl-6-[[4-[6-methyl-2-[trans-3-amino-5-fluoro-1-piperidyl]pyrimidin-4-yl]piperazin-1-yl]methyl]morpholin-4-yl]quinoline-8-carbonitrile;
5-[(2S,6R)-2-[[4-[2-[[(3R,4R)-4-fluoropyrrolidin-3-yl]amino]-6-methyl-pyrimidin-4-yl]piperazin-1-yl]methyl]-6-methyl-morpholin-4-yl]quinoline-8-carbonitrile;
5-[(2S,6R)-2-[[4-[2-[(3S,5R)-3-amino-5-fluoro-1-piperidyl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3S,4R)-4-amino-3-fluoro-1-piperidyl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4R)-3-amino-4-fluoro-pyrrolidin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-(5-amino-3,3-difluoro-1-piperidyl)-6-methyl-pyrimidin-4-yl]piperazin-1-yl]methyl]-6-methyl-morpholin-4-yl]quinoline-8-carbonitrile;
5-[(2S,6R)-2-[[4-[2-[3-(1-hydroxy-1-methyl-ethyl)piperazin-1-yl]-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4R)-4-amino-3-fluoro-1-piperidyl]-6-methyl-pyrimidin-4-yl]piperazin-1-yl]methyl]-6-methyl-morpholin-4-yl]quinoline-8-carbonitrile;
5-[(2R,6S)-2-methyl-6-[[4-[6-methyl-2-[(3S,4R)-3-amino-4-fluoro-1-piperidyl]-pyrimidin-4-yl]piperazin-1-yl]methyl]morpholin-4-yl]quinoline-8-carbonitrile;
5-[(2R,6S)-2-methyl-6-[[4-[6-methyl-2-[(3R,4R)-3-amino-4-fluoro-1-piperidyl]pyrimidin-4-yl]piperazin-1-yl]methyl]morpholin-4-yl]quinoline-8-carbonitrile;
5-[(2S,6R)-2-[[4-[2-(azetidin-3-ylamino)-6-methyl-pyrimidin-4-yl]piperazin-1-yl]methyl]-6-methyl-morpholin-4-yl]quinoline-8-carbonitrile;
5-[(2S,6R)-2-[[4-[2-(4-amino-3,3-difluoro-1-piperidyl)-6-methyl-pyrimidin-4-yl]piperazin-1-yl]methyl]-6-methyl-morpholin-4-yl]quinoline-8-carbonitrile;
5-[(2S,6R)-2-[[4-[2-(3-aminoazetidin-1-yl)-6-methyl-pyrimidin-4-yl]piperazin-1-yl]methyl]-6-methyl-morpholin-4-yl]quinoline-8-carbonitrile;
5-[(2S,6R)-2-[[4-[2-[(3R,4S)-3-amino-4-methoxy-pyrrolidin-1-yl]-6-methyl-pyrimidin-4-yl]piperazin-1-yl]methyl]-6-methyl-morpholin-4-yl]quinoline-8-carbonitrile;
5-[(2R,6S)-2-methyl-6-[[4-(2-morpholin-2-ylphenyl)piperazin-1-yl]methyl]morpholin-4-yl]quinoline-8-carbonitrile;
5-[(2S,6R)-2-[[4-[2-[(3S,4S)-4-amino-3-methoxy-1-piperidyl]-6-methyl-pyrimidin-4-yl]piperazin-1-yl]methyl]-6-methyl-morpholin-4-yl]quinoline-8-carbonitrile;
5-[(2S,6R)-2-[[4-(6-methoxy-2-piperazin-1-yl-pyrimidin-4-yl)piperazin-1-yl]methyl]-6-methyl-morpholin-4-yl]quinoline-8-carbonitrile;
5-[(2S,6R)-2-[[4-[2-[(3S,4R)-3-amino-4-methoxy-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[2-[(3R,4S)-3-amino-4-methoxy-pyrrolidin-1-yl]pyrimidin-4-yl]piperazin-1-yl]methyl]-6-methyl-morpholin-4-yl]quinoline-8-carbonitrile;
5-[(2S,6R)-2-[[4-(5-fluoro-4-piperazin-1-yl-pyrimidin-2-yl)piperazin-1-yl]methyl]-6-methyl-morpholin-4-yl]quinoline-8-carbonitrile;
5-[(2S,6R)-2-[[4-[2-[(3S,4R)-3-amino-4-fluoro-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[2-[(3R,4R)-3-amino-4-fluoro-pyrrolidin-1-yl]pyrimidin-4-yl]piperazin-1-yl]methyl]-6-methyl-morpholin-4-yl]quinoline-8-carbonitrile;
5-[(2S,6R)-2-[[4-[4-[(3S,4S)-3-amino-4-methoxy-pyrrolidin-1-yl]pyrimidin-2-yl]piperazin-1-yl]methyl]-6-methyl-morpholin-4-yl]quinoline-8-carbonitrile;
8-[(2S,6R)-2-[[4-[2-[(3R,4R)-3-amino-4-methoxy-pyrrolidin-1-yl]pyrimidin-4-yl]piperazin-1-yl]methyl]-6-methyl-morpholin-4-yl]quinoxaline-5-carbonitrile;
8-[(2S,6R)-2-[[4-[2-[(3S,4S)-3-amino-4-methoxy-pyrrolidin-1-yl]pyrimidin-4-yl]piperazin-1-yl]methyl]-6-methyl-morpholin-4-yl]quinoxaline-5-carbonitrile;
5-[(2S,6R)-2-[[4-[2-[(3R,4S)-3-amino-4-fluoro-pyrrolidin-1-yl]pyrimidin-4-yl]piperazin-1-yl]methyl]-6-methyl-morpholin-4-yl]quinoline-8-carbonitrile;
5-[(2R,6S)-2-methyl-6-[[4-(5-methyl-4-piperazin-1-yl-pyrimidin-2-yl)piperazin-1-yl]methyl]morpholin-4-yl]quinoline-8-carbonitrile;
5-[(2S,6R)-2-[[4-[2-[(4aR,7aR)-3,4,4a,5,7,7a-hexahydro-2H-pyrrolo[3,4-b][1,4]oxazin-6-yl]pyrimidin-4-yl]piperazin-1-yl]methyl]-6-methyl-morpholin-4-yl]quinoline-8-carbonitrile;
8-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile;
8-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]-2,3-dideuterio-quinoxaline-5-carbonitrile;
8-[(2S,6R)-2-[[4-[2-[(3S,4S)-3-amino-4-methoxy-pyrrolidin-1-yl]-6-methyl-pyrimidin-4-yl]piperazin-1-yl]methyl]-6-methyl-morpholin-4-yl]quinoxaline-5-carbonitrile;
8-[(2S,6R)-2-[[4-[2-[(3S,4S)-3-amino-4-fluoro-pyrrolidin-1-yl]pyrimidin-4-yl]piperazin-1-yl]methyl]-6-methyl-morpholin-4-yl]quinoxaline-5-carbonitrile;
8-[(2S,6R)-2-[[4-[2-[(3R,4S)-3-amino-4-fluoro-pyrrolidin-1-yl]pyrimidin-4-yl]piperazin-1-yl]methyl]-6-methyl-morpholin-4-yl]quinoxaline-5-carbonitrile;
5-[(2S,6R)-2-[[4-[2-(3-amino-4-methoxy-1-piperidyl)pyrimidin-4-yl]piperazin-1-yl]methyl]-6-methyl-morpholin-4-yl]quinoline-8-carbonitrile;
5-[(2S,6R)-2-[[4-[2-[(3S,4S)-3-amino-4-fluoro-pyrrolidin-1-yl]pyrimidin-4-yl]piperazin-1-yl]methyl]-6-methyl-morpholin-4-yl]quinoline-8-carbonitrile;
8-[(2S,6R)-2-[[4-[2-[(3R,4S)-4-amino-3-fluoro-1-piperidyl]pyrimidin-4-yl]piperazin-1-yl]methyl]-6-methyl-morpholin-4-yl]quinoxaline-5-carbonitrile;
8-[(2S,6R)-2-[[4-[2-(3-aminoazetidin-1-yl)-6-methyl-pyrimidin-4-yl]piperazin-1-yl]methyl]-6-methyl-morpholin-4-yl]quinoxaline-5-carbonitrile;
8-[(2S,6R)-2-[[4-[2-[(3R,4R)-4-amino-3-fluoro-1-piperidyl]pyrimidin-4-yl]piperazin-1-yl]methyl]-6-methyl-morpholin-4-yl]quinoxaline-5-carbonitrile;
8-[(2R,6S)-2-methyl-6-[[4-(4-methyl-6-piperazin-1-yl-pyrimidin-2-yl)piperazin-1-yl]methyl]morpholin-4-yl]quinoxaline-5-carbonitrile;
8-[(2S,6R)-2-[[4-[4-[(3R,4S)-3-amino-4-fluoro-pyrrolidin-1-yl]-6-methyl-pyrimidin-2-yl]piperazin-1-yl]methyl]-6-methyl-morpholin-4-yl]quinoxaline-5-carbonitrile;
(2R,6S)-2-methyl-4-(8-methylquinoxalin-5-yl)-6-[[4-(4-piperazin-1-ylpyrimidin-2-yl)piperazin-1-yl]methyl]morpholine;
(2R,6S)-2-methyl-4-(8-methylquinoxalin-5-yl)-6-[[4-(2-piperazin-1-ylpyrimidin-4-yl)piperazin-1-yl]methyl]morpholine;
8-[(2S,6R)-2-[[4-[4-(3-aminoazetidin-1-yl)pyrimidin-2-yl]piperazin-1-yl]methyl]-6-methyl-morpholin-4-yl]quinoxaline-5-carbonitrile;
8-[(2S,6R)-2-[[4-[4-[(3R,4R)-3-amino-4-fluoro-pyrrolidin-1-yl]pyrimidin-2-yl]piperazin-1-yl]methyl]-6-methyl-morpholin-4-yl]quinoxaline-5-carbonitrile;
8-[(2S,6R)-2-[[4-[4-[(3S,4R)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile;
8-[(2S,6R)-2-[[4-[4-[(3R,4R)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile;
8-[(2S,6R)-2-[[4-[4-[(3R,4S)-3-amino-4-methoxy-pyrrolidin-1-yl]-6-methyl-pyrimidin-2-yl]piperazin-1-yl]methyl]-6-methyl-morpholin-4-yl]quinoxaline-5-carbonitrile;
8-[(2S,6R)-2-[[4-[4-(3-aminoazetidin-1-yl)-6-methyl-pyrimidin-2-yl]piperazin-1-yl]methyl]-6-methyl-morpholin-4-yl]quinoxaline-5-carbonitrile;
8-[(2S,6R)-2-[[4-[4-(3-aminoazetidin-1-yl)-6-methyl-pyrimidin-2-yl]piperazin-1-yl]methyl]-6-methyl-morpholin-4-yl]-2,3-dideuterio-quinoxaline-5-carbonitrile;
8-[(2S,6R)-2-[[4-[4-[(3R,4R)-4-amino-3-hydroxy-3-methyl-pyrrolidin-1-yl]-6-methyl-pyrimidin-2-yl]piperazin-1-yl]methyl]-6-methyl-morpholin-4-yl]quinoxaline-5-carbonitrile;
2-[4-[4-[[(2S,6R)-6-methyl-4-(8-methylquinoxalin-5-yl)morpholin-2-yl]methyl]piperazin-1-yl]pyrimidin-2-yl]-5-oxa-2,8-diazaspiro[3.5]nonan;
(2R,6S)-2-methyl-6-[[4-[2-[(3S)-3-methylpiperazin-1-yl]pyrimidin-4-yl]piperazin-1-yl]methyl]-4-(8-methylquinoxalin-5-yl)morpholine;
(2R,6S)-2-methyl-6-[[4-[2-[(3R)-3-methylpiperazin-1-yl]pyrimidin-4-yl]piperazin-1-yl]methyl]-4-(8-methylquinoxalin-5-yl)morpholine;
(4aR,7aR)-6-[4-[4-[[(2S,6R)-6-methyl-4-(8-methylquinoxalin-5-yl)morpholin-2-yl]methyl]piperazin-1-yl]pyrimidin-2-yl]-3,4,4a,5,7,7a-hexahydro-2H-pyrrolo[3,4-b][1,4]oxazine;
1-[4-[4-[[(2S,6R)-6-methyl-4-(8-methylquinoxalin-5-yl)morpholin-2-yl]methyl]piperazin-1-yl]pyrimidin-2-yl]azetidin-3-amine;
5-[(2S,6R)-2-[[4-[4-(3-amino-3-methyl-azetidin-1-yl)-6-methoxy-pyrimidin-2-yl]piperazin-1-yl]methyl]-6-methyl-morpholin-4-yl]-2-deuterio-quinoline-8-carbonitrile;
5-[(2S,6R)-2-[[4-[6-(3-amino-3-methyl-azetidin-1-yl)-3-pyridyl]piperazin-1-yl]methyl]-6-methyl-morpholin-4-yl]-2-deuterio-quinoline-8-carbonitrile;
5-[(2S,6R)-2-[[4-[6-(3-amino-3-methyl-azetidin-1-yl)-4-methyl-3-pyridyl]piperazin-1-yl]methyl]-6-methyl-morpholin-4-yl]-2-deuterio-quinoline-8-carbonitrile;
5-[(2S,6R)-2-[[4-[4-[(3S,4S)-3-amino-4-methoxy-pyrrolidin-1-yl]-6-methyl-pyrimidin-2-yl]piperazin-1-yl]methyl]-6-methyl-morpholin-4-yl]-2-deuterio-quinoline-8-carbonitrile;
5-[(2S,6R)-2-[[4-[4-(3-amino-3-methyl-azetidin-1-yl)-6-methyl-pyrimidin-2-yl]piperazin-1-yl]methyl]-6-methyl-morpholin-4-yl]-2-deuterio-quinoline-8-carbonitrile;
5-[(2S,6R)-2-[[4-[4-(3-aminoazetidin-1-yl)-6-methyl-pyrimidin-2-yl]piperazin-1-yl]methyl]-6-methyl-morpholin-4-yl]-2-deuterio-quinoline-8-carbonitrile;
5-[(2S,6R)-2-[[4-[6-(3-amino-3-methyl-azetidin-1-yl)-2-methyl-3-pyridyl]piperazin-1-yl]methyl]-6-methyl-morpholin-4-yl]-2-deuterio-quinoline-8-carbonitrile;
5-[(2S,6R)-2-[[3-[6-(3-amino-3-methyl-azetidin-1-yl)-3-pyridyl]azetidin-1-yl]methyl]-6-methyl-morpholin-4-yl]-2-deuterio-quinoline-8-carbonitrile;
5-[(2S,6R)-2-[[4-[5-(3-amino-3-methyl-azetidin-1-yl)-3-methyl-2-pyridyl]piperazin-1-yl]methyl]-6-methyl-morpholin-4-yl]-2-deuterio-quinoline-8-carbonitrile;
5-[(2S,6R)-2-[[3-[4-[(3S,4S)-3-amino-4-methoxy-pyrrolidin-1-yl]phenyl]azetidin-1-yl]methyl]-6-methyl-morpholin-4-yl]-2-deuterio-quinoline-8-carbonitrile;
5-[(2S,6R)-2-[[3-[4-(3-amino-3-methyl-azetidin-1-yl)phenyl]azetidin-1-yl]methyl]-6-methyl-morpholin-4-yl]-2-deuterio-quinoline-8-carbonitrile;
5-[(2S,6R)-2-[[4-[5-[(3R,4R)-3-amino-4-methoxy-pyrrolidin-1-yl]-3-methyl-2-pyridyl]piperazin-1-yl]methyl]-6-methyl-morpholin-4-yl]-2-deuterio-quinoline-8-carbonitrile;
5-[(2S,6R)-2-[[4-[6-[(3R,4R)-3-amino-4-methoxy-pyrrolidin-1-yl]-3-pyridyl]piperazin-1-yl]methyl]-6-methyl-morpholin-4-yl]-2-deuterio-quinoline-8-carbonitrile;
5-[(2S,6R)-2-[[4-[4-[(3R,4R)-3-amino-4-methoxy-pyrrolidin-1-yl]-6-methyl-pyrimidin-2-yl]piperazin-1-yl]methyl]-6-methyl-morpholin-4-yl]-2-deuterio-quinoline-8-carbonitrile;
5-[(2S,6R)-2-[[3-[6-(3-amino-3-methyl-azetidin-1-yl)-4-methyl-3-pyridyl]azetidin-1-yl]methyl]-6-methyl-morpholin-4-yl]-2-deuterio-quinoline-8-carbonitrile;
5-[(2S,6R)-2-[[3-[6-[(3R,4R)-3-amino-4-methoxy-pyrrolidin-1-yl]-4-methyl-3-pyridyl]azetidin-1-yl]methyl]-6-methyl-morpholin-4-yl]-2-deuterio-quinoline-8-carbonitrile;
5-[(2S,6R)-2-[[4-[5-(3-amino-3-methyl-azetidin-1-yl)-6-methyl-2-pyridyl]piperazin-1-yl]methyl]-6-methyl-morpholin-4-yl]-2-deuterio-quinoline-8-carbonitrile;
4-[(2S,6R)-2-[[4-[5-(3-amino-3-methyl-azetidin-1-yl)-3-methyl-2-pyridyl]piperazin-1-yl]methyl]-6-methyl-morpholin-4-yl]-3-fluoro-pyrazolo[1,5-a]pyridine-7-carbonitrile;
5-[(2S,6R)-2-[[3-[6-[(3R,4R)-3-amino-4-methoxy-pyrrolidin-1-yl]-3-pyridyl]azetidin-1-yl]methyl]-6-methyl-morpholin-4-yl]-2-deuterio-quinoline-8-carbonitrile;
5-[(2S,6R)-2-[[4-[6-[(3S,4S)-3-amino-4-fluoro-pyrrolidin-1-yl]-3-pyridyl]piperazin-1-yl]methyl]-6-methyl-morpholin-4-yl]-2-deuterio-quinoline-8-carbonitrile;
2-deuterio-5-[(2S,6R)-2-[[4-[6-[(1S,4S)-2,5-diazabicyclo[2.2.1]heptan-2-yl]-3-pyridyl]piperazin-1-yl]methyl]-6-methyl-morpholin-4-yl]quinoline-8-carbonitrile;
2-deuterio-5-[(2R,6S)-2-methyl-6-[[4-[6-(5-oxa-2,8-diazaspiro[3.5]nonan-2-yl)-3-pyridyl]piperazin-1-yl]methyl]morpholin-4-yl]quinoline-8-carbonitrile;
2-deuterio-5-[(2S,6R)-2-[[4-[6-[(1R,4R)-2,5-diazabicyclo[2.2.1]heptan-2-yl]-3-pyridyl]piperazin-1-yl]methyl]-6-methyl-morpholin-4-yl]quinoline-8-carbonitrile;
4-[(2S,6R)-2-[[4-[6-(3-amino-3-methyl-azetidin-1-yl)-3-pyridyl]piperazin-1-yl]methyl]-6-methyl-morpholin-4-yl]-3-fluoro-pyrazolo[1,5-a]pyridine-7-carbonitrile;
5-[(2S,6R)-2-[[4-[6-(3-amino-3-methyl-azetidin-1-yl)-2-pyridyl]piperazin-1-yl]methyl]-6-methyl-morpholin-4-yl]-2-deuterio-quinoline-8-carbonitrile;
5-[(2S,6R)-2-[[3-[4-(3-amino-3-methyl-azetidin-1-yl)phenyl]-3-hydroxy-azetidin-1-yl]methyl]-6-methyl-morpholin-4-yl]-2-deuterio-quinoline-8-carbonitrile;
4-[(2S,6R)-2-[[3-[6-(3-amino-3-methyl-azetidin-1-yl)-3-pyridyl]azetidin-1-yl]methyl]-6-methyl-morpholin-4-yl]pyrazolo[1,5-a]pyridine-7-carbonitrile;
4-[(2S,6R)-2-[[3-[6-(3-amino-3-methyl-azetidin-1-yl)-3-pyridyl]azetidin-1-yl]methyl]-6-methyl-morpholin-4-yl]-3-fluoro-pyrazolo[1,5-a]pyridine-7-carbonitrile;
5-[(2S,6R)-2-[[3-[6-(3-amino-3-methyl-azetidin-1-yl)-2-pyridyl]azetidin-1-yl]methyl]-6-methyl-morpholin-4-yl]-2-deuterio-quinoline-8-carbonitrile;
4-[(2S,6R)-2-[[3-[4-(3-amino-3-methyl-azetidin-1-yl)phenyl]azetidin-1-yl]methyl]-6-methyl-morpholin-4-yl]-3-fluoro-pyrazolo[1,5-a]pyridine-7-carbonitrile;
2-deuterio-5-[(2R,6S)-2-methyl-6-[[4-[6-(6-methyl-2,6-diazaspiro[3.3]heptan-2-yl)-3-pyridyl]piperazin-1-yl]methyl]morpholin-4-yl]quinoline-8-carbonitrile;
2-deuterio-5-[(2S,6R)-2-[[4-[6-[[(3S,4R)-4-fluoropyrrolidin-3-yl]amino]-3-pyridyl]piperazin-1-yl]methyl]-6-methyl-morpholin-4-yl]quinoline-8-carbonitrile;
4-[(2S,6R)-2-[[3-[4-(3-amino-3-methyl-azetidin-1-yl)phenyl]azetidin-1-yl]methyl]-6-methyl-morpholin-4-yl]pyrazolo[1,5-a]pyridine-7-carbonitrile;
4-[(2S,6R)-2-[[3-[6-[(3R,4R)-3-amino-4-methoxy-pyrrolidin-1-yl]-3-pyridyl]azetidin-1-yl]methyl]-6-methyl-morpholin-4-yl]-3-fluoro-pyrazolo[1,5-a]pyridine-7-carbonitrile;
4-[(2S,6R)-2-[[3-[6-[(1S,4S)-2,5-diazabicyclo[2.2.1]heptan-2-yl]-3-pyridyl]azetidin-1-yl]methyl]-6-methyl-morpholin-4-yl]-3-fluoro-pyrazolo[1,5-a]pyridine-7-carbonitrile;
4-[(2S,6R)-2-[[4-[6-[(3R,4R)-3-amino-4-methoxy-pyrrolidin-1-yl]-3-pyridyl]piperazin-1-yl]methyl]-6-methyl-morpholin-4-yl]-3-fluoro-pyrazolo[1,5-a]pyridine-7-carbonitrile;
3-fluoro-4-[(2S,6R)-2-[[4-[6-[[(3S,4R)-4-fluoropyrrolidin-3-yl]amino]-3-pyridyl]piperazin-1-yl]methyl]-6-methyl-morpholin-4-yl]pyrazolo[1,5-a]pyridine-7-carbonitrile;
4-[(2S,6R)-2-[[4-[6-[(4aR,7aR)-3,4,4a,5,7,7a-hexahydro-2H-pyrrolo[3,4-b][1,4]oxazin-6-yl]-3-pyridyl]piperazin-1-yl]methyl]-6-methyl-morpholin-4-yl]-3-fluoro-pyrazolo[1,5-a]pyridine-7-carbonitrile;
3-fluoro-4-[(2R,6S)-2-methyl-6-[[4-[6-(5-oxa-2,8-diazaspiro[3.5]nonan-2-yl)-3-pyridyl]piperazin-1-yl]methyl]morpholin-4-yl]pyrazolo[1,5-a]pyridine-7-carbonitrile;
3-fluoro-4-[(2R,6S)-2-methyl-6-[[4-[6-(6-methyl-2,6-diazaspiro[3.3]heptan-2-yl)-3-pyridyl]piperazin-1-yl]methyl]morpholin-4-yl]pyrazolo[1,5-a]pyridine-7-carbonitrile;
1-methyl-4-[(2R,6S)-2-methyl-6-[[3-(4-piperazin-1-ylphenyl)azetidin-1-yl]methyl]morpholin-4-yl]-1,8-naphthyridin-2-one;
4-[(2S,6R)-2-[[3-[4-[(3R,4R)-3-amino-4-methoxy-pyrrolidin-1-yl]phenyl]azetidin-1-yl]methyl]-6-methyl-morpholin-4-yl]-1-methyl-1,8-naphthyridin-2-one;
1-methyl-4-[(2R,6S)-2-methyl-6-[[3-[4-(5-oxa-2,8-diazaspiro[3.5]nonan-2-yl)phenyl]azetidin-1-yl]methyl]morpholin-4-yl]-1,8-naphthyridin-2-one;
4-[(2S,6R)-2-[[3-[4-[(3R,4R)-3-amino-4-fluoro-pyrrolidin-1-yl]phenyl]azetidin-1-yl]methyl]-6-methyl-morpholin-4-yl]-1-methyl-1,8-naphthyridin-2-one;
4-[(2S,6R)-2-[[4-[6-[(3R,4R)-4-amino-3-fluoro-1-piperidyl]-3-pyridyl]piperazin-1-yl]methyl]-6-methyl-morpholin-4-yl]-3-fluoro-pyrazolo[1,5-a]pyridine-7-carbonitrile;
4-[(2S,6R)-2-[[3-[4-(3-amino-3-methyl-azetidin-1-yl)phenyl]azetidin-1-yl]methyl]-6-methyl-morpholin-4-yl]-1-methyl-1,8-naphthyridin-2-one;
3-fluoro-4-[(2R,6S)-2-methyl-6-[[4-[4-[(2R)-morpholin-2-yl]phenyl]piperazin-1-yl]methyl]morpholin-4-yl]pyrazolo[1,5-a]pyridine-7-carbonitrile;
3-fluoro-4-[(2R,6S)-2-methyl-6-[[4-[6-(5-oxa-2,8-diazaspiro[3.5]nonan-8-yl)-3-pyridyl]piperazin-1-yl]methyl]morpholin-4-yl]pyrazolo[1,5-a]pyridine-7-carbonitrile;
4-[(2S,6R)-2-[[4-[6-(1,6-diazaspiro[3.3]heptan-6-yl)-3-pyridyl]piperazin-1-yl]methyl]-6-methyl-morpholin-4-yl]-3-fluoro-pyrazolo[1,5-a]pyridine-7-carbonitrile;
1-methyl-4-[(2R,6S)-2-methyl-6-[[3-[4-(6-methyl-2,6-diazaspiro[3.3]heptan-2-yl)phenyl]azetidin-1-yl]methyl]morpholin-4-yl]-1,8-naphthyridin-2-one;
4-[(2S,6R)-2-[[3-[4-[(4aR,7aR)-3,4,4a,5,7,7a-hexahydro-2H-pyrrolo[3,4-b][1,4]oxazin-6-yl]phenyl]azetidin-1-yl]methyl]-6-methyl-morpholin-4-yl]-1-methyl-1,8-naphthyridin-2-one;
3-fluoro-4-[(2S,6R)-2-[[4-[2-fluoro-4-[(2S)-morpholin-2-yl]phenyl]piperazin-1-yl]methyl]-6-methyl-morpholin-4-yl]pyrazolo[1,5-a]pyridine-7-carbonitrile;
3-fluoro-4-[(2S,6R)-2-[[4-[3-fluoro-4-[(2R)-morpholin-2-yl]phenyl]piperazin-1-yl]methyl]-6-methyl-morpholin-4-yl]pyrazolo[1,5-a]pyridine-7-carbonitrile;
3-fluoro-4-[(2R,6S)-2-methyl-6-[[4-[4-(2-methylmorpholin-2-yl)phenyl]piperazin-1-yl]methyl]morpholin-4-yl]pyrazolo[1,5-a]pyridine-7-carbonitrile; and
3-fluoro-4-[(2R,6S)-2-methyl-6-[[4-[4-[[(2R)-morpholin-2-yl]methyl]phenyl]piperazin-1-yl]methyl]morpholin-4-yl]pyrazolo[1,5-a]pyridine-7-carbonitrile;
or a pharmaceutically acceptable salt thereof.

20. A process for the preparation of a compound according to any one of claims 1 to 19 comprising any of the following steps:
a) the substitution of compound of formula (IV), 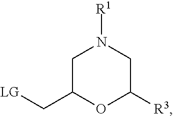
with compound of formula (VIII), 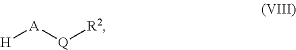
in the presence of a base;
b) the coupling of compound of formula (XIII), 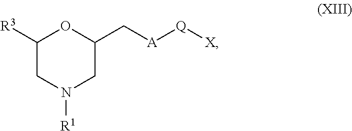
with compound of formula (VI) in the presence of a base or under Buchwald-Hartwig amination condition;
wherein the base in step a) and b) is selected from K₂CO₃, DIPEA and Cs₂CO₃; the Buchwald-Hartwig amination condition in step b) includes a catalyst and a base, wherein the catalyst is selected from tBuXPhos Pd G3, RuPhos Pd G2, BrettPhos Pd G3, XPhos Pd G3, Pd₂(dba)₃/BINAP and Pd₂(dba)₃/XantPhos; the base is Cs₂CO₃ or t-BuONa; LG is a leaving group; R¹, R², R³, A, Q and X are defined as in any one of claims 1 to 18.

21. A compound or pharmaceutically acceptable salt according to any one of claims 1 to 19 for use as therapeutically active substance.

22. A pharmaceutical composition comprising a compound in accordance with any one of claims 1 to 19 and a therapeutically inert carrier.

23. The use of a compound according to any one of claims 1 to 19 for the treatment or prophylaxis of systemic lupus erythematosus or lupus nephritis.

24. The use of a compound according to any one of claims 1 to 19 for the preparation of a medicament for the treatment or prophylaxis of systemic lupus erythematosus or lupus nephritis.

25. The use of a compound according to any one of claims 1 to 19 as the TLR7 or TLR8 or TLR9 antagonist.

26. The use of a compound according to any one of claims 1 to 19 as the TLR7 and TLR8 antagonist.

27. The use of a compound according to any one of claims 1 to 19 for the preparation of a medicament for TLR7 and TLR8 and TLR9 antagonist.

28. A compound or pharmaceutically acceptable salt, enantiomer or diastereomer according to any one of claims 1 to 19 for the treatment or prophylaxis of systemic lupus erythematosus or lupus nephritis.

29. A compound or pharmaceutically acceptable salt, enantiomer or diastereomer according to any one of claims 1 to 19, when manufactured according to a process of claim 20.

30. A method for the treatment or prophylaxis of systemic lupus erythematosus or lupus nephritis, which method comprises administering a therapeutically effective amount of a compound as defined in any one of claims 1 to 19.
